(19) United States
(12) Patent Application Publication
(10) Pub. No.: US 2024/0116926 A1
(43) Pub. Date: Apr. 11, 2024

(54) HETEROAROMATIC COMPOUNDS

(71) Applicant: AstraZeneca AB, Södertälje (SE)

(72) Inventors: Scott Nathan MLYNARSKI, Gaithersburg, MD (US); Jeffrey Wallace JOHANNES, Gaithersburg, MD (US); Alyssa Leigh VERANO, Gaithersburg, MD (US); Xiaolan ZHENG, Gaithersburg, MD (US); Robert Evans ZIEGLER, Gaithersburg, MD (US); Alisha Danielle CALIMAN, Gaithersburg, MD (US); Yufan LIANG, Gaithersburg, MD (US); Jonathan Anthony READ, Cambridge (GB)

(21) Appl. No.: 18/308,009

(22) Filed: Apr. 27, 2023

Related U.S. Application Data
(60) Provisional application No. 63/363,751, filed on Apr. 28, 2022.

(51) Int. Cl. C07D 471/04; A61P 35/00; C07D 401/14; C07D 403/12

(52) U.S. Cl. CPC C07D 471/04; A61P 35/00; C07D 401/14; C07D 403/12

(57) ABSTRACT

The specification relates to compounds of Formula (1):

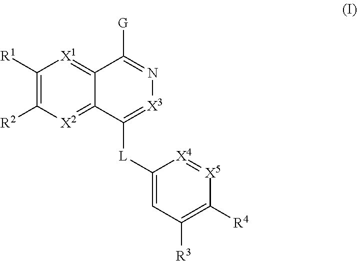

and to pharmaceutically acceptable salts thereof, to processes and intermediates used for their preparation, to pharmaceutical compositions containing them and to their use in the treatment of cancer.

This specification relates to certain heteroaromatic compounds and pharmaceutically acceptable salts thereof that inhibit TEAD, and their use in treating cancer. This specification also relates to processes and intermediate compounds involved in the preparation of the heteroaromatic compounds and to pharmaceutical compositions containing them.

### INTRODUCTION

The Hippo pathway is a highly conserved signaling pathway that controls organ size and tissue maintenance through the regulation of gene expression programs involved in cell proliferation, survival, and differentiation (Dong et al., Cell 2007, 1120-33; Ma et al., Ann Rev Biochem 2018, 577-604 and references therein). Hippo ultimately regulates the transcription coactivators Yes-associated protein (YAP) and transcriptional coactivator with PDZ-binding motif (TAZ) which bind to DNA-bound Transcriptional Enhanced Associate Domain proteins (TEAD1-4) to form bipartite transcription complexes that activate TEAD-dependent gene expression. The core of the Hippo pathway consists of a tightly regulated kinase signaling cascade. When Hippo signaling is active, the kinases LATS1/2 phosphorylate YAP/TAZ which causes these proteins to be sequestered in the cytoplasm or degraded by the proteasome. When Hippo signaling is inactive, LATS1/2 are inactivated resulting in YAP/TAZ to be dephosphorylated and subsequently translocated into the nucleus to interact with and activate TEAD-dependent transcription (Meng et al., Genes&Dev 2016, 1-17).

Hippo signaling is a well-established tumor suppressor pathway and data from The Cancer Genome Atlas show that the Hippo pathway is one of eight signaling pathways that are frequently altered in human cancer (Sanchez-Vega et al., Cell 2018, 321-337). Both genetic and epigenetic alterations of Hippo components can result in aberrant activation of YAP/TAZ and TEAD-dependent transcription and have been implicated in several human malignancies (Wang et al., 2018, 1304-1317). NF2 (aka Merlin) is encoded by the neurofibromatosis type 2 gene and is a key upstream regulator of the Hippo core kinase cascade consisting of STE20-like protein kinase 1 (STK3, aka MST2, and STK4, aka MST1), the large tumor suppressors (LATS1 and LATS2), and adaptor proteins Salvador homolog 1 (SAV1) and MOB kinase activators (MOB1A/MOB1B) (Tapon et al., Cell 2002, 467-478). Loss of function mutations or deletions in pathway components have been reported in several cancer types including mesothelioma, breast, liver, lung, prostate, gastric, and colorectal tumors (Poma et al., Scientific Reports 2018, volume 8, Article number: 10623; Zancanato et al., Cancer Cell 2016, 783-803 and references therein).

Because several Hippo pathway components are tumor suppressors where dysfunction results in aberrant TEAD-dependent transcription, targeting TEAD offers a potential opportunity for therapy.

The compounds of the specification provide an anti-cancer effect by, as a minimum, acting as TEAD inhibitors.

The compounds of the specification may also exhibit advantageous physical properties (for example, lower lipophilicity, higher aqueous solubility, higher permeability, lower plasma protein binding, and/or greater chemical stability), and/or favourable toxicity profiles (for example a decreased activity at hERG), and/or favourable metabolic or pharmacokinetic profiles, in comparison with other known TEAD inhibitors. Such compounds may therefore be especially suitable as therapeutic agents, particularly for the treatment of cancer.

### General Description

According to one aspect of the specification there is provided a compound of Formula (I):

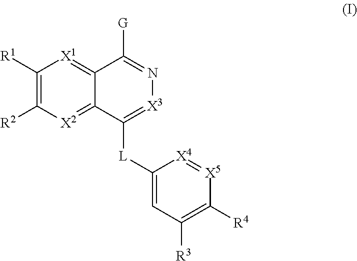

wherein:
X¹ is CH, C(C₁₋₄ alkyl), CF or N;
X² and X³ are independently selected from CH and N;
either X⁴ is CH and X⁵ is selected from CR⁵ and N, or X⁴ is N and X⁵ is CR⁵;
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and
Rⁱ, wherein Rⁱ is C₁₋₄ alkyl optionally substituted with —CN or C₁₋₄ alkoxy;
R³, R⁴ and R⁵ are independently selected from H, C₁₋₄ fluoroalkyl, C₁₋₄alkoxy, —S(C₁₋₄ alkyl), —O(C₁₋₄ fluoroalkyl), —S(C₁₋₄ fluoroalkyl), F, Cl, C₃₋₄ fluorocycloalkyl, R^{j} and R^{k}, wherein R^{j} is C₃₋₄ cycloalkyl optionally substituted with —CN, C₁₋₄ alkoxy or C₁₋₄ fluoroalkyl and R^{k} is C₁₋₄ alkyl optionally substituted with —CN or C₁₋₄ alkoxy;
G is selected from

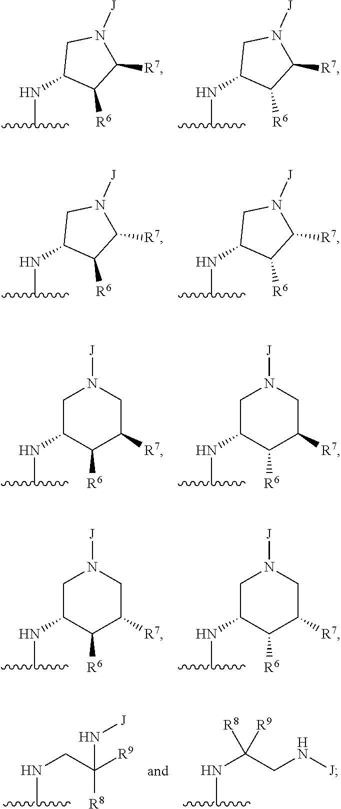

R⁶ is H, OH, F, —CN, C(═O)NH₂, N(C₁₋₄ alkyl)₂, C₁₋₄ alkoxy, C₁₋₄ fluoroalkyl, R¹⁰ or R¹¹;
R⁷ is H, C₁₋₄ alkoxy, R¹⁰ or R¹¹;
either R⁸ and R⁹ are independently selected from H, R¹⁰ and R¹¹;
or R⁸ and R⁹, together with the carbon atom to which they are attached, form a cyclopropane or cyclobutane ring;
each R¹⁰ is independently C₁₋₄alkyl optionally substituted with C₁₋₄alkoxy, N(C₁₋₄alkyl)₂ or OH;
each R¹¹ is independently C₃₋₄ cycloalkyl optionally substituted with C₁₋₄ alkoxy or OH;
J is selected from

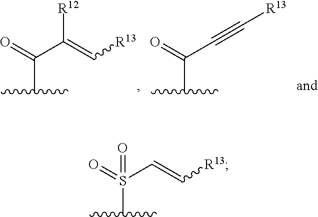

R¹² is H or F;
R¹³ is H, CH₂F or CH₃;
wherein a C₁₋₄ fluoroalkyl is a saturated linear or branched hydrocarbon radical having 1 to 4 carbon atoms, with at least one hydrogen atom substituted for a fluorine atom; and
wherein a C₃₋₄ fluorocycloalkyl is a saturated cyclic hydrocarbon radical having 3 or 4 carbon atoms, with at least one hydrogen atom substituted for a fluorine atom,
or a pharmaceutically acceptable salt thereof.

In a further aspect there is provided a pharmaceutical composition comprising a compound of Formula (I) or a pharmaceutically acceptable salt thereof, and a pharmaceutically acceptable excipient.

In a further aspect there is provided a compound of Formula (I) or a pharmaceutically acceptable salt thereof, for use in therapy.

In a further aspect there is provided a compound of Formula (I) or a pharmaceutically acceptable salt thereof, for use in the treatment of cancer.

In a further aspect there is provided the use of a compound of Formula (I) or a pharmaceutically acceptable salt thereof, in the manufacture of a medicament.

In a further aspect there is provided the use of a compound of Formula (I) or a pharmaceutically acceptable salt thereof, in the manufacture of a medicament for the treatment of cancer.

In a further aspect there is provided a method of treating cancer in a patient comprising administering to the patient an effective amount of a compound of Formula (I) or a pharmaceutically acceptable salt thereof.

### Definitions

So that the present specification may be more readily understood, certain terms are explicitly defined below. In addition, definitions are set forth as appropriate throughout the detailed description.

As used herein the term “alkyl” refers to both straight and branched chain saturated hydrocarbon radicals having the specified number of carbon atoms.

In this specification the prefix C_{x-y}, as used in terms such as “C_{x-y} alkyl” and the like where x and y are integers, indicates the numerical range of carbon atoms that are present in the group. Examples of suitable C₁₋₃ alkyl groups include methyl, ethyl, n-propyl, and i-propyl. Examples of suitable C₁₋₄alkyl groups include methyl, ethyl, n-propyl, and i-propyl, n-butyl, i-butyl, s-butyl and t-butyl.

As used herein the term “cycloalkyl” refers to a saturated, cyclic hydrocarbon radical having the specified number of carbon atoms. Examples of C₃₋₄cycloalkyl groups are cyclopropyl and cyclobutyl.

As used herein the term “fluoroalkyl” refers to saturated linear or branched hydrocarbon radicals having the specified number of carbon atoms, wherein at least one hydrogen atom is substituted for a fluorine atom. Examples of suitable C₁₋₄ fluoroalkyl groups include fluoromethyl (CFH₂), difluoromethyl (CF₂H), trifluoromethyl (CF₃), 1,1-difluoroethyl (CF₂CH₃), 2,2,2-trifluoroethyl (CH₂CF₃) and 3-fluoropropyl (CH₂CH₂CH₂F).

As used herein the term “fluorocycloalkyl” refers to saturated cyclic hydrocarbon radicals having the specified number of carbon atoms, wherein at least one hydrogen atom is substituted for a fluorine atom. Examples of suitable C₃₋₄ fluorocycloalkyl groups include 2-fluorocyclopropyl, 2,2-difluorocyclopropyl, 2,2-difluorocyclopropyl, 2,3-difluorocyclopropyl, 2,2,3-trifluorocyclopropyl, 2,2,3,3-tetrafluorocyclopropyl, 2-fluorocyclobutyl, 3-fluorocyclobutyl, 2,3-difluorocyclobutyl, 2,4-difluorocyclobutyl and 2,3,4-trifluorocyclobutyl.

As used herein the term “alkoxy” refers to a saturated group comprising the specified number of carbon atoms and one oxygen atom. For the avoidance of doubt, the alkoxy group may be a straight chain or a branched chain. Examples of suitable C₁₋₄alkoxy groups include methoxy (OMe), ethoxy (OEt), n-propoxy (OⁿPr) and i-propoxy (OⁱPr), n-butoxy (OⁿBu), i-butoxy (OⁱBu), s-butoxy (O^{s}Bu) and t-butoxy (O^{t}Bu).

Unless specifically stated, the bonding of an atom or group may be any suitable atom of that group; for example, propyl includes prop-1-yl and prop-2-yl.

For the avoidance of doubt, where multiple substituents are independently selected from a given group, the selected substituents may comprise the same substituents or different substituents from within the given group.

For the avoidance of doubt, the use of

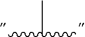

in formulas of this specification denotes the point of attachment between different groups. By way of illustration,

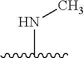 [SMILES: *NC]

denotes a methylamide group which is attached to a different group through the nitrogen atom.

Where any embodiment within this specification includes a group which is said to be “optionally substituted”, then a further embodiment will include that embodiment wherein the said group is unsubstituted.

For the avoidance of doubt, the use of a wavy bond attached to an alkene indicates that the stereochemistry of the alkene may be either E- or Z-. By way of illustration,

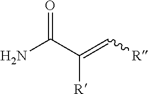

can be either

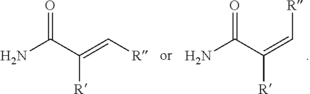

Units, prefixes, and symbols are denoted in their International System of Units (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

### DETAILED DESCRIPTION

As noted above, this specification provides a compound of Formula (I):

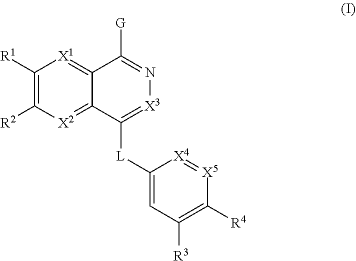

wherein:
X¹ is CH, C(C₁₋₄ alkyl), CF or N;
X² and X³ are independently selected from CH and N;
either X⁴ is CH and X⁵ is selected from CR⁵ and N, or X⁴ is N and X⁵ is CR⁵;
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and
Rⁱ, wherein Rⁱ is C₁₋₄ alkyl optionally substituted with —CN or C₁₋₄ alkoxy;
R³, R⁴ and R⁵ are independently selected from H, C₁₋₄ fluoroalkyl, C₁₋₄alkoxy, —S(C₁₋₄ alkyl), —O(C₁₋₄ fluoroalkyl), —S(C₁₋₄ fluoroalkyl), F, Cl, C₃₋₄ fluorocycloalkyl, R^{j} and R^{k}, wherein R^{j} is C₃₋₄ cycloalkyl optionally substituted with —CN, C₁₋₄ alkoxy or C₁₋₄ fluoroalkyl and R^{k} is C₁₋₄ alkyl optionally substituted with —CN or C₁₋₄ alkoxy;
G is selected from

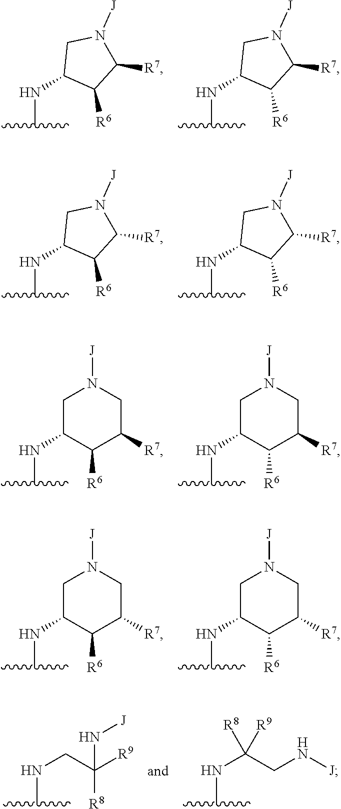

R⁶ is H, OH, F, —CN, C(═O)NH₂, N(C₁₋₄ alkyl)₂, C₁₋₄ alkoxy, C₁₋₄ fluoroalkyl, R¹⁰ or R¹¹;
R⁷ is H, C₁₋₄ alkoxy, R¹⁰ or R¹¹;
either R⁸ and R⁹ are independently selected from H, R¹⁰ and R¹¹;
or R⁸ and R⁹, together with the carbon atom to which they are attached, form a cyclopropane or cyclobutane ring;
each R¹⁰ is independently C₁₋₄alkyl optionally substituted with C₁₋₄alkoxy, N(C₁₋₄alkyl)₂ or OH;
each R¹¹ is independently C₃₋₄ cycloalkyl optionally substituted with C₁₋₄ alkoxy or OH;
J is selected from

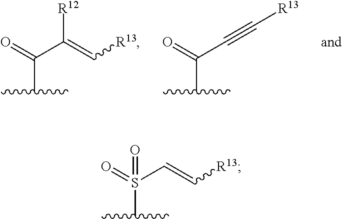

R¹² is H or F;
R¹³ is H, CH₂F or CH₃;
wherein a C₁₋₄ fluoroalkyl is a saturated linear or branched hydrocarbon radical having 1 to 4 carbon atoms, with at least one hydrogen atom substituted for a fluorine atom; and
wherein a C₃₋₄ fluorocycloalkyl is a saturated cyclic hydrocarbon radical having 3 or 4 carbon atoms, with at least one hydrogen atom substituted for a fluorine atom, or a pharmaceutically acceptable salt thereof.

In embodiments, there is provided a compound of Formula (I) or a pharmaceutically acceptable salt thereof, wherein X¹ is CH, C(C₁₋₄alkyl) or CF, such as CH.

In embodiments, there is provided a compound of Formula (I) or a pharmaceutically acceptable salt thereof, wherein X¹ is N.

In embodiments, there is provided a compound of Formula (I) or a pharmaceutically acceptable salt thereof, wherein X² is CH.

In embodiments, there is provided a compound of Formula (I) or a pharmaceutically acceptable salt thereof, wherein X² is N.

In embodiments, there is provided a compound of Formula (I) or a pharmaceutically acceptable salt thereof, wherein X³ is CH.

In embodiments, there is provided a compound of Formula (I) or a pharmaceutically acceptable salt thereof, wherein X³ is N.

In embodiments, there is provided a compound of Formula (I) or a pharmaceutically acceptable salt thereof, wherein X⁴ and X⁵ are both CH.

In embodiments, there is provided a compound of Formula (I) or a pharmaceutically acceptable salt thereof, wherein X⁴ is N and X⁵ is CH.

In embodiments, there is provided a compound of Formula (I) or a pharmaceutically acceptable salt thereof, wherein X⁴ is CH and X⁵ is N.

In embodiments, the compound of Formula (I) is a compound of Formula (II):

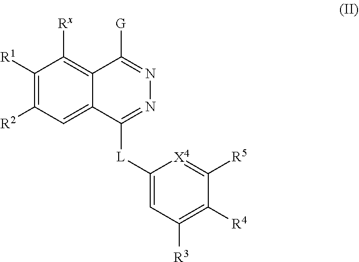

wherein R^{x} is H, F or C₁₋₄ alkyl, and R¹, R², R³, R⁴, R⁵, X⁴, L and G are as defined above, or a pharmaceutically acceptable salt thereof.

In embodiments, the compound of Formula (I) is a compound of Formula (III):

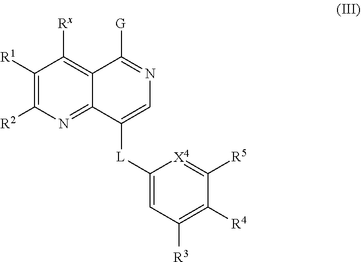

wherein R^{x} is H, F or C₁₋₄ alkyl, and R¹, R², R³, R⁴, R⁵, X⁴, L and G are as defined above, or a pharmaceutically acceptable salt thereof.

In embodiments, the compound of Formula (I) is a compound of Formula (IV):

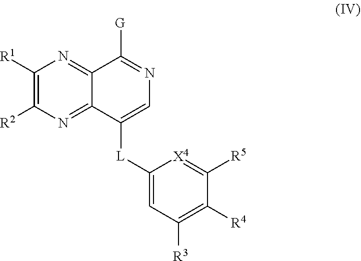

wherein R¹, R², R³, R⁴, R⁵, X⁴, L and G are as defined above, or a pharmaceutically acceptable salt thereof.

In embodiments, the compound of Formula (I) is a compound of Formula (V):

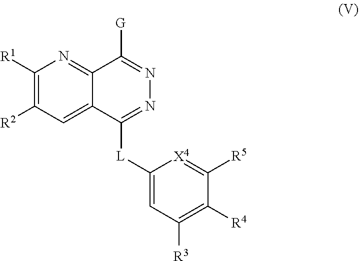

wherein R¹, R², R³, R⁴, R⁵, X⁴, L and G are as defined above, or a pharmaceutically acceptable salt thereof.

In embodiments, the compound of Formula (I) is a compound of Formula (VI):

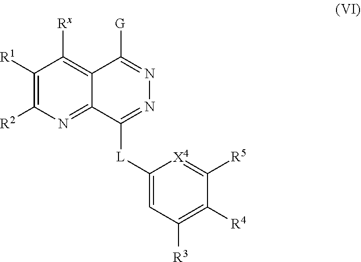

wherein R^{x} is H, F or C₁₋₄ alkyl, and R¹, R², R³, R⁴, R⁵, X⁴, L and G are as defined above, or a pharmaceutically acceptable salt thereof.

In embodiments, there is provided a compound of Formula (II), (III), (IV), (V) or (VI), or a pharmaceutically acceptable salt thereof, wherein X⁴ is CH.

In embodiments, there is provided a compound of Formula (II), (III), (IV), (V) or (VI), or a pharmaceutically acceptable salt thereof, wherein X⁴ is N.

In embodiments, there is provided a compound of Formula (I), (II), (III), (IV), (V) or (VI), or a pharmaceutically acceptable salt thereof, wherein R¹ is H or C₁₋₄ alkyl optionally substituted with —CN or C₁₋₄ alkoxy (i.e. C₁₋₄ alkyl, C₁₋₄ alkyl substituted with —CN, or C₁₋₄ alkyl substituted with C₁₋₄ alkoxy).

In embodiments, there is provided a compound of Formula (I), (II), (III), (IV), (V) or (VI), or a pharmaceutically acceptable salt thereof, wherein R¹ is H or C₁₋₄ alkyl, such as CH₃.

In embodiments, there is provided a compound of Formula (I), (II), (III), (IV), (V) or (VI), or a pharmaceutically acceptable salt thereof, wherein R² is H or C₁₋₄ alkyl optionally substituted with —CN or C₁₋₄ alkoxy.

In embodiments, there is provided a compound of Formula (I), (II), (III), (IV), (V) or (VI), or a pharmaceutically acceptable salt thereof, wherein R² is H or CH₃, such as H.

In embodiments, there is provided a compound of Formula (I), (II), (III), (IV), (V) or (VI), or a pharmaceutically acceptable salt thereof, wherein R¹ and R² independently selected from H and C₁₋₄ alkyl, such as CH₃.

In embodiments, there is provided a compound of Formula (I), (II), (III), (IV), (V) or (VI), or a pharmaceutically acceptable salt thereof, wherein R¹ and R² are both H.

In embodiments, there is provided a compound of Formula (I), (II), (III), (IV), (V) or (VI), or a pharmaceutically acceptable salt thereof, wherein L is a covalent bond.

In embodiments, there is provided a compound of Formula (I), (II), (III), (IV), (V) or (VI), or a pharmaceutically acceptable salt thereof, wherein L is O.

In embodiments, there is provided a compound of Formula (I), (II), (III), (IV), (V) or (VI), or a pharmaceutically acceptable salt thereof, wherein L is NH.

In embodiments, there is provided a compound of Formula (I), (II), (III), (IV), (V) or (VI), or a pharmaceutically acceptable salt thereof, wherein L is CH₂.

In embodiments, there is provided a compound of Formula (I), (II), (III), (IV), (V) or (VI), or a pharmaceutically acceptable salt thereof, wherein L is CH₂CH₂.

In embodiments, the compound of Formula (I) is a compound of Formula (IA):

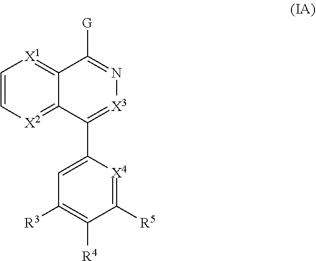

wherein:
X¹ is CH, C(C₁₋₄alkyl), CF or N;
X² is CH or N;
X³ is CH or N;
X⁴ is CH or N;
R³, R⁴, R⁵ and G are as defined above;
or a pharmaceutically acceptable salt thereof.

In embodiments, the compound of Formula (I) is a compound of Formula (IIA):

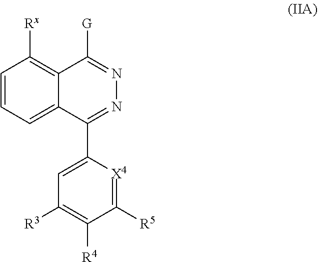

wherein R^{x}, R³, R⁴, R⁵, X⁴ and G are as defined above, or a pharmaceutically acceptable salt thereof.

In embodiments, the compound of Formula (I) is a compound of Formula (IIIA):

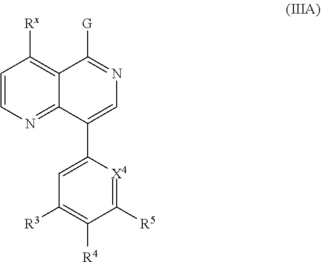

wherein R^{x}, R³, R⁴, R⁵, X⁴ and G are as defined above, or a pharmaceutically acceptable salt thereof.

In embodiments, the compound of Formula (I) is a compound of Formula (IVA):

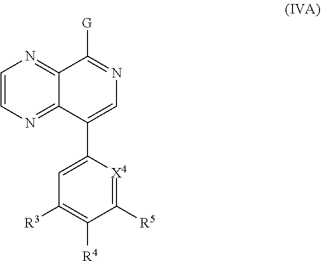

wherein R³, R⁴, R⁵, X⁴ and G are as defined above, or a pharmaceutically acceptable salt thereof.

In embodiments, the compound of Formula (I) is a compound of Formula (VA):

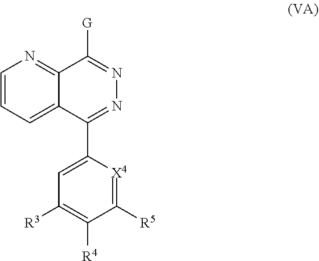

wherein R³, R⁴, R⁵, X⁴ and G are as defined above, or a pharmaceutically acceptable salt thereof.

In embodiments, the compound of Formula (I) is a compound of Formula (VIA):

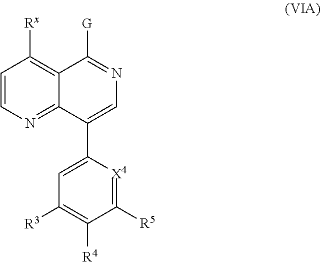

wherein R^{x}, R³, R⁴, R⁵, X⁴ and G are as defined above, or a pharmaceutically acceptable salt thereof.

In embodiments, there is provided a compound of Formula (II), (IIA), (III), (IIIA), (VI) or (VIA), or a pharmaceutically acceptable salt thereof, wherein R^{x} is H.

In embodiments, there is provided a compound of Formula (II), (IIA), (III), (IIIA), (VI) or (VIA), or a pharmaceutically acceptable salt thereof, wherein R^{x} is F.

In embodiments, there is provided a compound of Formula (II), (IIA), (III), (IIIA), (VI) or (VIA), or a pharmaceutically acceptable salt thereof, wherein R^{x} is C₁₋₄ alkyl, such as CH₃.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R³ is selected from H, F, Cl and C₁₋₄alkyl (such as CH₃).

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R³ is H.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁴ is selected from H, C₁₋₄ fluoroalkyl, C₁₋₄alkoxy, —S(C₁₋₄ alkyl), —O(C₁₋₄ fluoroalkyl), —S(C₁₋₄ fluoroalkyl), F, Cl, C₃₋₄ fluorocycloalkyl, R^{j} and R^{k}, wherein R^{j} is C₃₋₄ cycloalkyl optionally substituted with —CN, C₁₋₄ alkoxy or C₁₋₄ fluoroalkyl (i.e. C₃₋₄ cycloalkyl, C₃₋₄ cycloalkyl substituted with —CN, C₃₋₄ cycloalkyl substituted with C₁₋₄ alkoxy, or C₃₋₄ cycloalkyl substituted with C₁₋₄ fluoroalkyl), and wherein R^{k} is C₁₋₄alkyl optionally substituted with —CN or C₁₋₄ alkoxy (i.e. C₁₋₄ alkyl, C₁₋₄alkyl substituted with —CN, or C₁₋₄alkyl substituted with C₁₋₄ alkoxy).

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁴ is C₁₋₄ fluoroalkyl, —O(C₁₋₄ fluoroalkyl) or —S(C₁₋₄ fluoroalkyl).

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁴ is CF₂H, CF₂CH₃, CF₃, OCF₃, OCF₂H or SCF₃, such as CF₃.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁵ is selected from H, F, Cl and C₁₋₄alkyl (such as CH₃).

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁵ is H.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R³ and R⁵ are independently selected from H, Cl, F and C₁₋₄alkyl (such as CH₃), and R⁴ is C₁₋₄ fluoroalkyl (such as CF₃, CF₂CH₃ or CF₂H), —O(C₁₋₄ fluoroalkyl) (such as OCF₃ or OCF₂H) and —S(C₁₋₄ fluoroalkyl) (such as SCF₃).

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R³ and R⁵ are both H, and R⁴ is C₁₋₄ fluoroalkyl (such as CF₃, CF₂CH₃ or CF₂H), —O(C₁₋₄ fluoroalkyl) (such as OCF₃ or OCF₂H) and —S(C₁₋₄ fluoroalkyl) (such as SCF₃).

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R³ is H, R⁴ is CF₃ and R⁵ is H.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

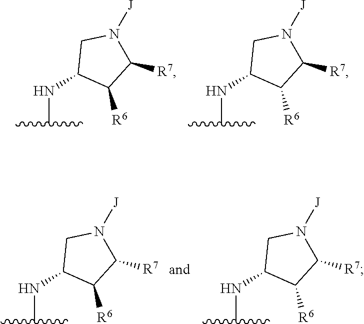

wherein R⁶, R⁷ and J are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

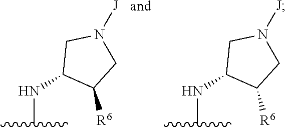

wherein R⁶ and J are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

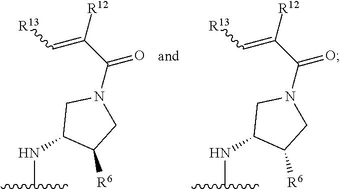

wherein R⁶, R¹² and R¹³ are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

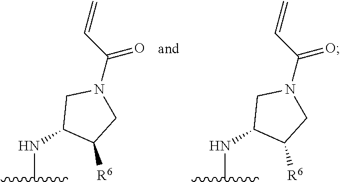

wherein R⁶ is as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

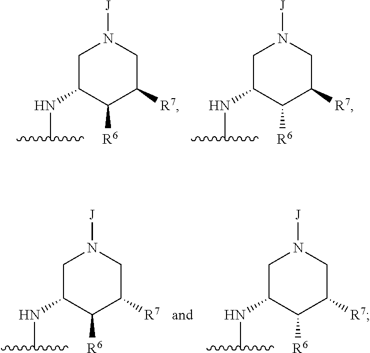

wherein R⁶, R⁷ and J are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

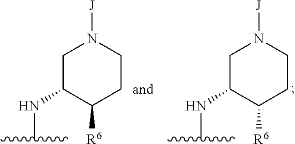

wherein R⁶ and J are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

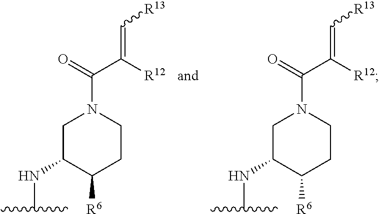

wherein R⁶, R¹² and R¹³ are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

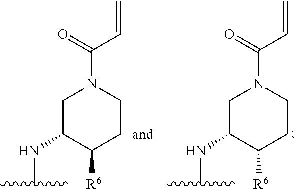

wherein R⁶ are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁶ is H, OH, F or R¹⁰.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁶ is H, OH, F, CH₂OH or CH₂OCH₃.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁷ is C₁₋₄ alkyl optionally substituted with OH, such as CH₂OH.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁷ is H.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

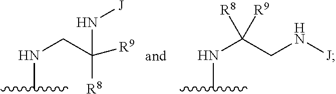

wherein R⁸, R⁹ and J are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

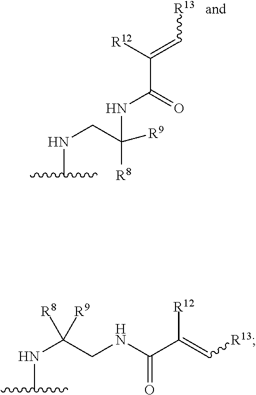

wherein R⁸, R⁹ R¹² and R¹³ are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

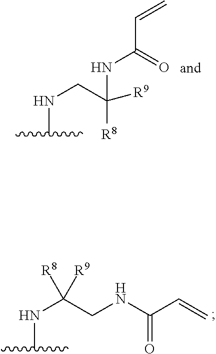

wherein R⁸ and R⁹ are as defined herein.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁸ and R⁹ are independently selected from H, R¹⁰ and R¹¹, wherein R¹⁰ is C₁₋₄ alkyl optionally substituted with C₁₋₄ alkoxy or OH (i.e. C₁₋₄ alkyl, C₁₋₄ alkyl substituted with C₁₋₄alkoxy, or C₁₋₄ alkyl substituted with OH), and wherein R¹¹ is C₃₋₄ cycloalkyl optionally substituted with C₁₋₄ alkoxy or OH (i.e. C₃₋₄ cycloalkyl, C₃₋₄ cycloalkyl substituted with C₁₋₄ alkoxy, or C₃₋₄ cycloalkyl substituted with OH).

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁸ is H and R⁹ is C₁₋₄ alkyl, such as CH₃.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein R⁸ and R⁹, together with the carbon atom to which they are attached, form a cyclopropane or cyclobutane ring.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein J is

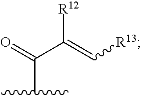

wherein R¹² is H or F, and wherein R¹³ is H, CH₂F or CH₃.

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein J is selected from

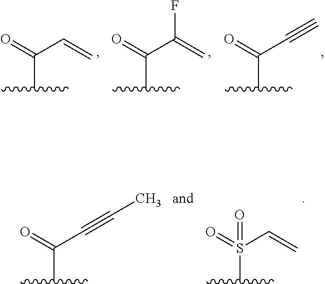

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein J is

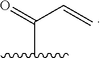 [SMILES: *C(=O)C=C ; "."]

In embodiments, there is provided a compound of Formula (I), (IA), (II), (IIA), (III), (IIIA), (IV), (IVA), (V), (VA), (VI) or (VIA), or a pharmaceutically acceptable salt thereof, wherein G is selected from

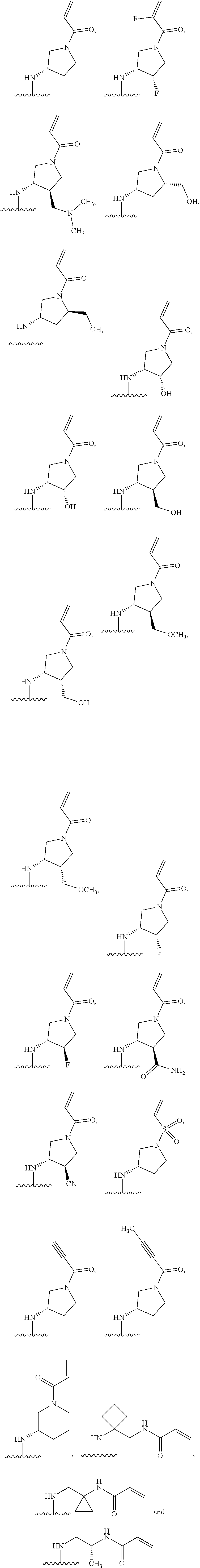

In embodiments, the compound of Formula (I) is a compound of Formula (IB):

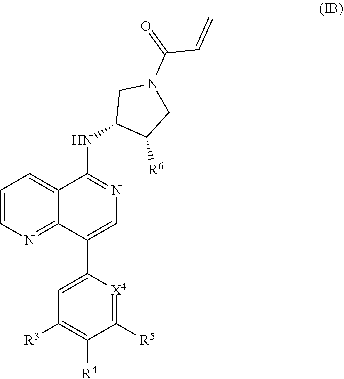

wherein:
X⁴ is CH or N;
R³ and R⁵ are independently selected from H, F, Cl and C₁₋₄alkyl;
R⁴ is C₁₋₄ fluoroalkyl, —O(C₁₋₄ fluoroalkyl) or —S(C₁₋₄ fluoroalkyl);
R⁶ is H, OH, F, —CN, C(═O)NH₂, N(C₁₋₄alkyl)₂, C₁₋₄ alkoxy, C₁₋₄ fluoroalkyl, R¹⁰ or R¹¹;
each R¹⁰ is independently C₁₋₄alkyl optionally substituted with C₁₋₄alkoxy, N(C₁₋₄alkyl)₂ or OH; and
R¹¹ is C₃₋₄ cycloalkyl optionally substituted with C₁₋₄ alkoxy or OH,
or a pharmaceutically acceptable salt thereof.

In embodiments, there is provided a compound of Formula (IB) or a pharmaceutically acceptable salt thereof, wherein X⁴ is CH.

In embodiments, there is provided a compound of Formula (IB) or a pharmaceutically acceptable salt thereof, wherein X⁴ is N.

In embodiments, there is provided a compound of Formula (IB) or a pharmaceutically acceptable salt thereof, wherein R³ and R⁵ are H, and optionally R⁴ is CF₂H, CF₃, OCF₃ or SCF₃.

In embodiments, there is provided a compound of Formula (IB) or a pharmaceutically acceptable salt thereof, wherein R³ and R⁵ are H, and R⁴ is CF₃.

In embodiments, there is provided a compound of Formula (IB) or a pharmaceutically acceptable salt thereof, wherein R⁶ is H, OH, F, CH₂OH or CH₂OCH₃, such as H.

In embodiments, there is provided a compound of Formula (I), or a pharmaceutically acceptable salt thereof, wherein the compound is selected from:
1-((3S,4R)-3-fluoro-4-((8-(4-(trifluoromethyl) phenyl)pyrido[2,3-d]pyridazin-5-yl)amino)pyrrolidin-1-yl)prop-2-en-1-one;
1-((3S,4R)-3-fluoro-4-((5-(4-(trifluoromethyl)phenyl)pyrido[2,3-d]pyridazin-8-yl)amino)pyrrolidin-1-yl)prop-2-en-1-one;
1-((3R,4R)-3-fluoro-4-((8-(4-(trifluoromethyl)phenyl)pyrido[2,3-d]pyridazin-5-yl)amino)pyrrolidin-1-yl)prop-2-en-1-one;
1-((3R,4R)-3-fluoro-4-((5-(4-(trifluoromethyl)phenyl)pyrido[2,3-d]pyridazin-8-yl)amino)pyrrolidin-1-yl)prop-2-en-1-one;
2-fluoro-1-((3S,4R)-3-fluoro-4-((8-(4-(trifluoromethyl)phenyl)pyrido[2,3-d]pyridazin-5-yl)amino)pyrrolidin-1-yl)prop-2-en-1-one;
2-fluoro-1-((3S,4R)-3-fluoro-4-((5-(4-(trifluoromethyl)phenyl)pyrido[2,3-d]pyridazin-8-yl)amino)pyrrolidin-1-yl)prop-2-en-1-one;
(S)-1-(3-((8-(4-(trifluoromethyl)phenyl)pyrido[2,3-d]pyridazin-5-yl)amino)pyrrolidin-1-yl)prop-2-en-1 one;
(S)-1-(3-((5-(4-(trifluoromethyl)phenyl)pyrido[2,3-d]pyridazin-8-yl)amino)pyrrolidin-1-yl)prop-2-en-1-one;
(S)-1-(3-((4-(4-(trifluoromethyl)phenyl)phthalazin-1-yl)amino)pyrrolidin-1-yl)prop-2-en-1-one;
1-((3S,4R)-3-fluoro-4-((4-(4-(trifluoromethyl)phenyl)phthalazin-1-yl)amino)pyrrolidin-1-yl)prop-2-en-1-one;
1-((3R,4R)-3-fluoro-4-((4-(4-(trifluoromethyl)phenyl)phthalazin-1-yl)amino)pyrrolidin-1-yl)prop-2-en
1-one;
1-((3S,4R)-3-hydroxy-4-((4-(4-(trifluoromethyl)phenyl)phthalazin-1-yl)amino)pyrrolidin-1-yl)prop-2-en-1-one;
(S)-1-(3-((8-fluoro-4-(4-(trifluoromethyl)phenyl) phthalazin-1-yl)amino)pyrrolidin-1-yl)prop-2-en-1-one;
1-((3R,4S)-3-((8-fluoro-4-(4-(trifluoromethyl)phenyl)phthalazin-1-yl)amino)-4-hydroxypyrrolidin-1-yl)prop-2-en-1-one;
1-((3S,4R)-3-((8-fluoro-4-(4-(trifluoromethyl)phenyl)phthalazin-1-yl)amino)-4-(hydroxymethyl)pyrrolidin-1-yl)prop-2-en-1-one;
(S)-1-(3-((5-(5-(trifluoromethyl)pyridin-2-yl)pyrido[2,3-d]pyridazin-8-yl)amino)pyrrolidin-1-yl)prop-2-en-1-one;
(S)-1-(3-((8-(5-(trifluoromethyl)pyridin-2-yl)pyrido[3,4-b]pyrazin-5-yl)amino)pyrrolidin-1-yl)prop-2-en-1-one;
(S)-1-(3-((4-(5-(trifluoromethyl)pyridin-2-yl)phthalazin-1-yl)amino)pyrrolidin-1-yl)prop-2-en-1-one;
(S)-1-(3-((4-(4-(trifluoromethyl)phenyl) phthalazin-1-yl)amino)pyrrolidin-1-yl)prop-2-yn-1-one;
(S)-1-(3-((4-(4-(trifluoromethyl)phenyl) phthalazin-1-yl)amino)pyrrolidin-1-yl)but-2-yn-1-one;
(S)-4-(4-(trifluoromethyl)phenyl)-N-(1-(vinylsulfonyl)pyrrolidin-3-yl)phthalazin-1-amine;
1-((3S,4R)-3-(hydroxymethyl)-4-((4-(4-(trifluoromethyl)phenyl)phthalazin-1-yl)amino)pyrrolidin-1-yl)prop-2-en-1-one;
1-((3R,4S)-3-(hydroxymethyl)-4-((4-(4-(trifluoromethyl)phenyl)phthalazin-1-yl)amino)pyrrolidin-1-yl)prop-2-en-1-one;
1-((3R,4S)-3-(hydroxymethyl)-4-((8-(4-(trifluoromethyl)phenyl)-1,6-naphthyridin-5-yl)amino)pyrrolidin-1-yl)prop-2-en-1-one;
1-((3R,4S)-3-(hydroxymethyl)-4-((8-(4-(trifluoromethyl)phenyl)pyrido[2,3-d]pyridazin-5-yl)amino)pyrrolidin-1-yl)prop-2-en-1-one;
1-((3R,4S)-3-(hydroxymethyl)-4-((5-(4-(trifluoromethyl)phenyl)pyrido[2,3-d]pyridazin-8-yl)amino)pyrrolidin-1-yl)prop-2-en-1-one;
1-((3R,4S)-3-(hydroxymethyl)-4-((8-(4-(trifluoromethyl)phenyl)pyrido[3,4-b]pyrazin-5-yl)amino)pyrrolidin-1-yl)prop-2-en-1-one;
1-((3R,4S)-3-(methoxymethyl)-4-((4-(4-(trifluoromethyl)phenyl)phthalazin-1-yl)amino)pyrrolidin-1-yl)prop-2-en-1-one;
1-acryloyl-4-((4-(4-(trifluoromethyl)phenyl)phthalazin-1-yl)amino)pyrrolidine-3-carbonitrile;
1-acryloyl-4-((4-(4-(trifluoromethyl)phenyl)phthalazin-1-yl)amino)pyrrolidine-3-carboxamide;
1-((3S,4R)-3-hydroxy-4-((8-(4-(trifluoromethyl)phenyl)pyrido[2,3-d]pyridazin-5-yl)amino)pyrrolidin-1-yl)prop-2-en-1-one;
1-((3S,4R)-3-hydroxy-4-((5-(4-(trifluoromethyl)phenyl)pyrido[2,3-d]pyridazin-8-yl)amino)pyrrolidin-1-yl)prop-2-en-1-one;
1-((3S,4R)-3-hydroxy-4-((8-(4-(trifluoromethyl)phenyl)-1,6-naphthyridin-5-yl)amino)pyrrolidin-1-yl)prop-2-en-1-one;
1-((3S,4R)-3-hydroxy-4-((8-(5-(trifluoromethyl)pyridin-2-yl)-1,6-naphthyridin-5-yl)amino)pyrrolidin-1-yl)prop-2-en-1-one;
1-((3S,4R)-3-fluoro-4-((8-(5-(trifluoromethyl) pyridin-2-yl)-1,6-naphthyridin-5-yl)amino)pyrrolidin-1-yl)prop-2-en-1-one;
1-((3R,4S)-3-(hydroxymethyl)-4-((8-(5-(trifluoromethyl)pyridin-2-yl)-1,6-naphthyridin-5-yl)amino)pyrrolidin-1-yl)prop-2-en-1-one;
(S)-1-(3-((8-(5-(trifluoromethyl)pyridin-2-yl)-1,6-naphthyridin-5-yl)amino)pyrrolidin-1-yl)prop-2-en-1-one;
(S)-1-(3-((8-(4-(trifluoromethyl)phenyl)-1,6-naphthyridin-5-yl)amino)pyrrolidin-1-yl)prop-2-en-1-one
N-(1-(((4-(4-(trifluoromethyl)phenyl)phthalazin-1-yl)amino)methyl)cyclopropyl)acrylamide;
N-(1-(((4-(4-(trifluoromethyl)phenyl)phthalazin-1-yl)amino)methyl)cyclobutyl)acrylamide;
N-((1-((4-(4-(trifluoromethyl)phenyl)phthalazin-1-yl)amino)cyclobutyl)methyl)acrylamide;
1-((3S,4R)-3-fluoro-4-((8-(4-(trifluoromethyl)phenyl)pyrido[3,4-b]pyrazin-5-yl)amino)pyrrolidin-1-yl)prop-2-en-1-one;
1-((3S,4R)-3-fluoro-4-((8-(4-(trifluoromethyl)phenyl)-1,6-naphthyridin-5-yl)amino)pyrrolidin-1-yl)prop-2-en-1-one;
(S)-1-(3-((8-(4-(trifluoromethyl)phenyl)pyrido[3,4-b]pyrazin-5-yl)amino)pyrrolidin-1-yl)prop-2-en-1-one;
1-((3R,4S)-3-((dimethylamino)methyl)-4-((4-(4-(trifluoromethyl)phenyl)phthalazin-1-yl)amino)pyrrolidin-1-yl)prop-2-en-1-one;
1-((2R,4S)-2-(hydroxymethyl)-4-((4-(4-(trifluoromethyl)phenyl)phthalazin-1-yl)amino)pyrrolidin-1-yl)prop-2-en-1-one;
1-((2S,4S)-2-(hydroxymethyl)-4-((4-(4-(trifluoromethyl)phenyl)phthalazin-1-yl)amino)pyrrolidin-1-yl)prop-2-en-1-one;
1-((3S,4S)-3-(hydroxymethyl)-4-((4-(4-(trifluoromethyl)phenyl)phthalazin-1-yl)amino)pyrrolidin-1-yl)prop-2-en-1-one; and
(S)-1-(3-((4-(4-(trifluoromethyl)phenyl)phthalazin-1-yl)amino)piperidin-1-yl)prop-2-en-1-one.

A further feature is any of the embodiments described in the specification with the proviso that any of the specific Examples are individually disclaimed. A further feature is any of the embodiments described in the specification with the proviso that any one or more of the compounds selected from the above list of Examples of compounds of the specification are individually disclaimed.

The compounds disclosed herein may contain one or more chiral centers. Accordingly, if desired, such compounds can be prepared or isolated as pure stereoisomers, i.e. as individual enantiomers, diastereoisomers, or as a stereoisomerically enriched mixture. AII such stereoisomer (and enriched) mixtures are included within the scope of the embodiments, unless otherwise stated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Unless stereochemistry is explicitly indicated in a chemical structure or chemical name, the chemical structure or chemical name is intended to embrace all possible stereoisomers, diastereoisomers, conformers, rotamers and tautomers of the compound depicted. For example, a compound containing a chiral carbon atom is intended to embrace both the (R) enantiomer and the (S) enantiomer, as well as mixtures of the enantiomers, including racemic mixtures; and a compound containing two chiral carbons is intended to embrace all enantiomers and diastereoisomers including (R,R), (S,S), (R,S) and (S,R).

In embodiments, there is provided a pharmaceutical composition which comprises a compound of the Formula (I) or a pharmaceutically acceptable salt thereof, in association with a pharmaceutically acceptable excipient, optionally further comprising one or more of the other stereoisomeric forms of the compound of Formula (I) or pharmaceutically acceptable salt thereof, wherein the compound of Formula (I) or pharmaceutically acceptable salt thereof is present within the composition with an enantiomeric excess (% ee) of 90% and a diastereomeric excess (% de) of ≥90%.

The compound of Formula (I), and pharmaceutically acceptable salts thereof, may be prepared, used or supplied in amorphous form, crystalline form, or semicrystalline form and any given compound of Formula (I), or pharmaceutically acceptable salt thereof, may be capable of being formed into more than one crystalline/polymorphic form, including hydrated (e.g. hemi hydrate, a mono hydrate, a di hydrate, a tri hydrate or other stoichiometry of hydrate) and/or solvated forms. It is to be understood that the present specification encompasses any and all such solid forms of the compound of Formula (I) and pharmaceutically acceptable salts thereof.

In further embodiments there is provided a compound of Formula (I), which is obtainable by the methods described in the ‘Examples” section hereinafter.

The present specification is intended to include all isotopes of atoms occurring in the present compounds. Isotopes will be understood to include those atoms having the same atomic number but different mass numbers. For example, isotopes of hydrogen include tritium and deuterium. Isotopes of carbon include ¹³C and ¹⁴C. Isotopes of nitrogen include ¹⁵N.

A suitable pharmaceutically acceptable salt of a compound of Formula (I) is, for example, an acid addition salt. An acid addition salt of a compound of Formula (I) may be formed by bringing the compound into contact with a suitable inorganic or organic acid under conditions known to the skilled person. An acid addition salt may for example be formed using an inorganic acid selected from hydrochloric acid, hydrobromic acid, sulphuric acid and phosphoric acid. An acid addition salt may also be formed using an organic acid selected from trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

A further suitable pharmaceutically acceptable salt of a compound of Formula (I) is, for example, a salt formed within a patient's body after administration of a compound of Formula (I) to the patient.

The compound of Formula (I), or pharmaceutically acceptable salt thereof, may be prepared as a co-crystal solid form. It is to be understood that a pharmaceutically acceptable co-crystal of an compound of Formula (I), or pharmaceutically acceptable salts thereof, form an aspect of the present specification.

In a further aspect there is provided a pharmaceutical composition comprising a compound of Formula (I), (IA), (II), (IIA), (III), (IIIA), (IV), (IVA), (V), (VA), (VI), (VIA) or (IB), or a pharmaceutically acceptable salt thereof and a pharmaceutically acceptable excipient.

The term “pharmaceutical composition” refers to a preparation which is in such form as to permit the biological activity of the active ingredient, and which contains no additional components which are unacceptably toxic to a patient to which the composition would be administered. Such compositions can be sterile. A pharmaceutical composition according to the present specification will comprise a compound of Formula (I) or a pharmaceutically acceptable salt thereof, and a pharmaceutically acceptable excipient. For example, the composition may be in a form suitable for oral use (for example as tablets, lozenges, hard or soft capsules, aqueous or oily suspensions, emulsions, dispersible powders or granules, syrups or elixirs) or for parenteral administration (for example as a sterile aqueous or oily solution for intravenous, subcutaneous, intramuscular or intramuscular dosing or as a suppository for rectal dosing). Such compositions may be obtained by conventional procedures using conventional pharmaceutical excipients, well known in the art. Thus, compositions intended for oral use may contain, for example, one or more colouring, sweetening, flavouring and/or preservative agents. An effective amount of the compound of Formula (I), or a pharmaceutically acceptable salt thereof, will normally be present in the composition.

The compound of Formula (I), or a pharmaceutically acceptable salt thereof, will normally be administered via the oral route though parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable salt or solvate thereof, or a solvate of such a salt, in a pharmaceutically acceptable dosage form may be possible. Depending upon the disorder and patient to be treated and the route of administration, the compositions may be administered at varying doses.

The pharmaceutical formulations of the compound of Formula (I) described above may be prepared e.g. for parenteral, subcutaneous, intramuscular or intravenous administration.

The pharmaceutical formulations of the compound of Formula (I) described above may conveniently be administered in unit dosage form and may be prepared by any of the methods well-known in the pharmaceutical art, for example as described in Remington's Pharmaceutical Sciences, 17th ed., Mack Publishing Company, Easton, PA., (1985).

Pharmaceutical formulations suitable for oral administration may comprise one or more physiologically compatible carriers and/or excipients and may be in solid or liquid form. Tablets and capsules may be prepared with binding agents; fillers; lubricants; and surfactants. Liquid compositions may contain conventional additives such as suspending agents; emulsifying agents; and preservatives Liquid compositions may be encapsulated in, for example, gelatin to provide a unit dosage form. Solid oral dosage forms include tablets, two-piece hard shell capsules and soft elastic gelatin (SEG) capsules. An exemplary oral composition would comprise a compound of Formula (I) and at least one pharmaceutically acceptable excipient filled into a two-piece hard shell capsule or a soft elastic gelatin (SEG) capsule.

As a result of their TEAD inhibitory activity, the compounds of Formula (I), and pharmaceutically acceptable salts thereof are expected to be useful in therapy, for example in the treatment of diseases or medical conditions mediated at least in part by TEAD, including cancer.

In one aspect of the present specification there is provided a compound of Formula (I), (IA), (II), (IIA), (III), (IIIA), (IV), (IVA), (V), (VA), (VI), (VIA) or (IB), or a pharmaceutically acceptable salt thereof, for use in therapy.

In one aspect of the present specification there is provided a compound of Formula (I), (IA), (II), (IIA), (III), (IIIA), (IV), (IVA), (V), (VA), (VI), (VIA), or (IB), or a pharmaceutically acceptable salt thereof, for use in the treatment of cancer.

Where “cancer” is mentioned, this includes both non-metastatic cancer and also metastatic cancer, such that treating cancer involves treatment of both primary tumours and also tumour metastases.

The term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

The term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in providing an inhibitory effect on TEAD.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of a disease mediated by TEAD, such as cancer.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of cancer, wherein the cancer is selected from ovarian cancer, cervical cancer, colorectal cancer, breast cancer, pancreatic cancer, glioma, glioblastoma, melanoma, prostate cancer, gastric cancer, lung cancer, hepatocellular cancer (HCC), gastrointestinal stromal tumour (GIST), thyroid cancer, bile duct cancer, endometrial cancer, renal cancer, melanoma and mesothelioma (such as malignant pleural mesothelioma).

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of hippo mutation-positive cancer, such as NF2 mutation-positive or LATS1/2 mutation-positive cancer.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of hippo mutation-positive cancer, such as YAP1 and/or WWTR1 amplified cancer.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of hippo mutation-positive cancer, such as FAT1 mutant cancer.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of a cancer driven by YAP or TAZ fusions.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of a cancer that exhibits an elevated TEAD transcriptional signature. In further embodiments, the cancer that exhibits an elevated TEAD transcriptional signature is hepatocellular cancer (HCC), gastric cancer or prostate cancer.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of hippo mutation-positive mesothelioma, such as NF2 mutation-positive or LATS1/2 mutation-positive mesothelioma.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of hippo mutation-positive malignant pleural mesothelioma, such as NF2 mutation-positive or LATS1/2 mutation-positive malignant pleural mesothelioma.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of lung cancer.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of non-small cell lung cancer.

In embodiment there is provided a compound of Formula (I), (IA), (II), (IIA), (III), (IIIA), (IV), (IVA), (V), (VA), (VI), (VIA) or (IB), or a pharmaceutically acceptable salt thereof for use in the treatment of EGFR mutation-positive cancer (such as non-small cell lung cancer). In further embodiments, the EGFR mutation-positive cancer comprises at least one activating mutation in EGFR selected from exon 19 deletions and L858R substitution mutations. In still further embodiments, the EGFR mutation-positive cancer comprises an EGFR T790M resistance mutation.

In one aspect of the present specification there is provided the use of a compound of Formula (I), (IA), (II), (IIA), (III), (IIIA), (IV), (IVA), (V), (VA), (VI), (VIA) or (IB), or a pharmaceutically acceptable salt thereof, as described herein, in the manufacture of a medicament, such as a medicament for the treatment of cancer.

In one aspect of the present specification there is provided a method of treating cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof.

Terms such as “treating” or “treatment” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a patient is successfully “treated” for cancer according to the methods of the present disclosure if the patient shows, e.g., total, partial, or transient remission of a certain type of cancer.

The term “effective amount” means an amount of an active ingredient which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

The term “patient” refers to any animal (e.g., a mammal), including, but not limited to humans, non-human primates, rodents, and the like, which is to be the recipient of a particular treatment. Typically, the term “patient” refers to a human subject.

In embodiments, there is provided a method of treating cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof, wherein the cancer is selected from ovarian cancer, cervical cancer, colorectal cancer, breast cancer, pancreatic cancer, glioma, glioblastoma, melanoma, prostate cancer, gastric cancer, lung cancer, hepatocellular cancer, gastrointestinal stromal tumour (GIST), thyroid cancer, bile duct cancer, endometrial cancer, renal cancer, melanoma and mesothelioma (such as malignant pleural mesothelioma).

In embodiments, there is provided a method of treating hippo mutation-positive cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof. In further embodiments, the hippo mutation-positive cancer is hippo mutation-positive mesothelioma.

In embodiments, there is provided a method of treating lung cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof.

In embodiments, there is provided a method of treating non-small cell lung cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof.

In embodiments, a compound of Formula (I), (IA), (II), (IIA), (III), (IIIA), (IV), (IVA), (V), (VA), (VI), (VIA) or (IB), or a pharmaceutically acceptable salt thereof is for use in combination with conventional surgery, radiotherapy, chemotherapy and/or immunotherapy. Such chemotherapy could be administered concurrently, simultaneously, sequentially or separately to treatment with the TEAD inhibitor of the present disclosure.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, and an additional anti-tumour substance for the conjoint treatment of cancer.

In embodiments, there is provided a combination for use in the treatment of cancer comprising a compound of the Formula (I), (IA), (II), (IIA), (III), (IIIA), (IV), (IVA), (V), (VA), (VI), (VIA) or (IB), or a pharmaceutically acceptable salt thereof and an additional anti-tumour agent.

In embodiments, there is provided a compound of the Formula (I), (IA), (II), (IIA), (III), (IIIA), (IV), (IVA), (V), (VA), (VI), (VIA) or (IB), or a pharmaceutically acceptable salt thereof, in combination with an additional anti-tumour agent.

In embodiments, the additional anti-tumour agent is a selected from an EGFR inhibitor, KRAS inhibitor, BRAF inhibitor, CDK4/6 inhibitor, MEK inhibitor, MET inhibitor, P13K inhibitor, AKT inhibitor or ALK inhibitor.

The additional anti-tumour agent may be a third generation EGFR TKI.

Third-generation EGFR TKIs are inhibitors of EGFR bearing activating mutations that also significantly inhibit EGFR bearing the T790M mutation and do not significantly inhibit wild-type EGFR. Examples of third-generation TKIs include compounds of Formula (I), osimertinib, AZD3759, lazertinib, nazartinib, C01686 (rociletinib), HM61713, ASP8273, EGF816, PF-06747775 (mavelertinib), avitinib (abivertinib), alflutinib (AST2818) and CXCK-101 (RX-518), HS-10296 and BPI-7711. Further examples include oritinib (SH-1028), Befotertinib (D-0316), ASK-120067, ZN-e4, YZJ-0318, TL007 XZP (kenaitinib), YK-029A, SLC005-I, TY-9591, XZP-5809-TT1, ZSP0391, and TQB3456.

In any embodiment where “third generation EGFR TKI” is mentioned in a general sense, the third-generation EGFR TKI is selected from osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, CXCK-101 or a pharmaceutically acceptable salt thereof, HS-10296 or a pharmaceutically acceptable salt thereof and BPI-7711 or a pharmaceutically acceptable salt thereof. In embodiments, the third generation EGFR TKI is osimertinib or a pharmaceutically acceptable salt thereof.

Osimertinib: The free base of osimertinib is known by the chemical name: N-(2-{2-dimethylamino ethyl-methylamino}-4-methoxy-5-{[4-(1-methylindol-3-yl)pyrimidin-2-yl]amino}phenyl) prop-2-enamide. Osimertinib is described in WO 2013/014448, the contents of which is incorporated by reference. Osimertinib is also known as AZD9291. Osimertinib may be found in the form of the mesylate salt: N-(2-{2-dimethylamino ethyl-methylamino}-4-methoxy-5-{[4-(1-methylindol-3-yl)pyrimidin-2-yl]amino}phenyl) prop-2-enamide mesylate salt. Osimertinib mesylate is also known as TAGRISSO™.

Osimertinib mesylate is currently approved as an oral once daily tablet formulation, at a dose of 80 mg (expressed as free base, equivalent to 95.4 mg osimertinib mesylate), for the treatment of metastatic EGFR T790M mutation positive NSCLC patients. A 40 mg oral once daily tablet formulation (expressed as free base, equivalent to 47.7 mg osimertinib mesylate) is available should dose modification be required. The tablet core comprises pharmaceutical diluents (such as mannitol and microcrystalline cellulose), disintegrants (such as low-substituted hydroxypropyl cellulose) and lubricants (such as sodium stearyl fumarate). The tablet formulation is described in WO 2015/101791, the contents of which is incorporated by reference.

In an aspect, the composition is in the form of a tablet, wherein the tablet core comprises: (a) about 19 parts of osimertinib mesylate; (b) about 59 parts of mannitol; (c) about 15 parts of microcrystalline cellulose; (d) about 5 parts of low-substituted hydroxypropyl cellulose; and (e) about 2 parts of sodium stearyl fumarate; and wherein all parts are by weight and the sum of the parts (a)+(b)+(c)+(d)+(e)=100.

AZD3759: The free base of AZD3759 is known by the chemical name: 4-[(3-chloro-2-fluorophenyl)amino]-7-methoxy-6-quinazolinyl (2R)-2,4-dimethyl-1-piperazinecarboxylate. AZD3759 is described in WO 2014/135876, the contents of which is incorporated by reference.

Lazertinib: The free base of lazertinib is known by the chemical name N-{5-[(4-{4-[(dimethylamino)methyl]-3-phenyl-1H-pyrazol-1-yl}-2-pyrimidinyl)amino]-4-methoxy-2-(4-morpholinyl)phenyl}acrylamide. Lazertinib is described in WO 2016/060443, the contents of which is incorporated by reference. Lazertinib is also known by the names YH25448 and GNS-1480.

Nazartinib: The free base of Nazartinib is known by the chemical name: N-(7-chloro-1-(1-(4-(dimethylamino)but-2-enoyl)azepan-3-yl)-1H-benzordlimidazol-2-yl)-2-methylisonicotinamide. Nazartinib is disclosed in WO 2013/184757, the contents of which is incorporated by reference.

Avitinib (abivertinib): The free base of avitinib is known by the chemical name: N-(3-((2-((3-fluoro-4-(4-methylpiperazin-1-yl)phenyl)amino)-7H-pyrrolo(2,3-d)pyrimidin-4-yl)oxy)phenyl)prop-2-enamide. Avitinib is disclosed in US2014038940, the contents of which is incorporated by reference. Avitinib is also known as abivertinib.

Alflutinib (furmonertinib): The free base of alflutinib is known by the chemical name: N-{2-{[2-(dimethylamino)ethyl](methyl)amino}-6-(2,2,2-trifluoroethoxyl)-5-{[4-(1-methyl-1H-indol-3-yl)pyrimidin-2-yl]amino}pyridin-3-yl}acrylamide. Alflutinib is disclosed in WO 2016/15453, the contents of which is incorporated by reference. Alflutinib is also known as AST2818.

Afatinib: The free base of afatinib is known by the chemical name: N-[4-(3-chloro-4-fluoroanilino)-7-[(3S)-oxolan-3-yl] oxyquinazolin-6-yl]-4-(dimethylamino)but-2-enamide. Afatinib is disclosed in WO 02/50043, the contents of which is incorporated by reference. Afatinib is also known as Gilotrif.

CK-101: The free base of CK-101 is known by the chemical name: N-(3-(2-((2,3-difluoro-4-(4-(2-hydroxyethyl)piperazin-1-yl)phenyl)amino)quinazolin-8-yl)phenyl)acrylamide. CK-101 is disclosed in WO 2015/027222, the contents of which is incorporated by reference. CK-101 is also known as RX-518.

HS-10296 (aumolertinib): The free base of HS-10296 is known by the chemical name: N-[5-[[4-(1-cyclopropylindol-3-yl)pyrimidin-2-yl]amino]-2-[2-(dimethylamino)ethyl-methyl-amino]-4-methoxy-phenyl]prop-2-enamide. HS-10296 is disclosed in WO 2016/054987, the contents of which is incorporated by reference.

BPI-7711: The free base of BPI-7711 is known by the chemical name: N-[2-[2-(dimethylamino)ethoxy]-4-methoxy-5-[[4-(1-methylindol-3-yl)pyrimidin-2-yl]amino]phenyl]prop-2-enamide. BPI-7711 is disclosed in WO 2016/94821, the contents of which is incorporated by reference.

Dacomitinib: The free form of dacomitinib is known by the chemical name: (2E)-N-{4-[(3-chloro-4-fluorophenyl)amino]-7-methoxyquinazolin-6-yl}-4-(piperidin-1-yl)but-2-enamide. Dacomitinib is described in WO 2005/107758, the contents of which is incorporated by reference. Dacomitinib is also known by the name PF-00299804.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, and a third generation EGFR TKI for the conjoint treatment of cancer, such as non-small cell lung cancer.

In embodiments, there is provided a combination for use in the treatment of cancer, such as non-small cell lung cancer, comprising a compound of the Formula (I), (IA), (II), (IIA), (III), (IIIA), (IV), (IVA), (V), (VA), (VI), (VIA) or (IB), or a pharmaceutically acceptable salt thereof and a third generation EGFR TKI.

In embodiments, there is provided a compound of the Formula (I), (IA), (II), (IIA), (III), (IIIA), (IV), (IVA), (V), (VA), (VI), (VIA) or (IB), or a pharmaceutically acceptable salt thereof, in combination with a third generation EGFR TKI.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, and osimertinib, or a pharmaceutically acceptable salt thereof, for the conjoint treatment of cancer, such as non-small cell lung cancer.

In embodiments, there is provided a combination for use in the treatment of cancer, such as non-small cell lung cancer, comprising a compound of the Formula (I), (IA), (II), (IIA), (III), (IIIA), (IV), (IVA), (V), (VA), (VI), (VIA) or (IB), or a pharmaceutically acceptable salt thereof and osimertinib, or a pharmaceutically acceptable salt thereof.

In embodiments, there is provided a compound of the Formula (I), (IA), (II), (IIA), (III), (IIIA), (IV), (IVA), (V), (VA), (VI), (VIA) or (IB), or a pharmaceutically acceptable salt thereof, in combination with osimertinib, or a pharmaceutically acceptable salt thereof.

Herein, where the term “conjoint treatment” is used in reference to a combination treatment, it is to be understood that this may refer to simultaneous, separate or sequential administration. In one aspect, “conjoint treatment” refers to simultaneous administration. In another aspect, “conjoint treatment” refers to separate administration. In a further aspect, “conjoint treatment” refers to sequential administration.

In embodiments, there is provided a method of treating cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof, and simultaneously, separately or sequentially administering at least one additional anti-tumour substance to said patient, where the amounts of the compound of Formula (I), (IA), (II), (IIA), (III), (IIIA), (IV), (IVA), (V), (VA), (VI), (VIA) or (IB), or pharmaceutically acceptable salt thereof, and the additional anti-tumour substance are jointly effective in producing an anti-cancer effect.

In embodiments, there is provided a method of treating cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof, and simultaneously, separately or sequentially administering a third generation EGFR TKI to said patient, where the amounts of the compound of Formula (I), (IA), (II), (IIA), (III), (IIIA), (IV), (IVA), (V), (VA), (VI), (VIA) or (IB), or pharmaceutically acceptable salt thereof, and the third generation EGFR TKI are jointly effective in producing an anti-cancer effect.

In embodiments, there is provided a method of treating cancer, such as non-small cell lung cancer, in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof, and simultaneously, separately or sequentially administering osimertinib, or a pharmaceutically acceptable salt thereof, to said patient, where the amounts of the compound of Formula (I), (IA), (II), (IIA), (III), (IIIA), (IV), (IVA), (V), (VA), (VI), (VIA) or (IB), or pharmaceutically acceptable salt thereof, and the osimertinib, or a pharmaceutically acceptable salt thereof substance are jointly effective in producing an anti-cancer effect.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of cancer, such as non-small cell lung cancer, wherein the cancer is resistant to treatment with an EGFR TKI.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of cancer, such as non-small cell lung cancer, wherein the cancer is resistant to treatment with a third generation EGFR TKI. In further embodiments, the third-generation EGFR TKI is selected from osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, CXCK-101 or a pharmaceutically acceptable salt thereof, HS-10296 or a pharmaceutically acceptable salt thereof and BPI-7711 or a pharmaceutically acceptable salt thereof.

In embodiments, there is provided a compound of Formula (I), (IA), (II), (IIA), (III), (IIIA), (IV), (IVA), (V), (VA), (VI), (VIA) or (IB), or a pharmaceutically acceptable salt thereof, for use in the treatment of non-small cell lung cancer, wherein the non-small cell lung cancer is resistant to treatment with osimertinib or a pharmaceutically acceptable salt thereof.

In embodiments, there is provided a method of treating cancer in a patient comprising administering to the patient an effective amount of a compound of Formula (I), (IA), (II), (IIA), (III), (IIIA), (IV), (IVA), (V), (VA), (VI), (VIA) or (IB), or a pharmaceutically acceptable salt thereof, wherein the cancer is resistant to treatment with an EGFR TKI.

Although the compounds of the Formula (I) are primarily of value as therapeutic agents for use in patients, they are also useful whenever it is required to inhibit TEAD. Thus, they are useful as pharmacological standards for use in the development of new biological tests and in the search for new pharmacological agents.

Certain compounds of Formula (I) may be prepared via the reaction of a suitable aromatic electrophile (for example a compound of Formula (AI), (AII), (AIII), (AIV) or (AV) as defined below) and a suitable nucleophile, optionally in the presence of a catalyst. A non-limiting example of such a reaction is the reaction of Intermediate 5 and Intermediate 11 in the synthesis of Example 24.

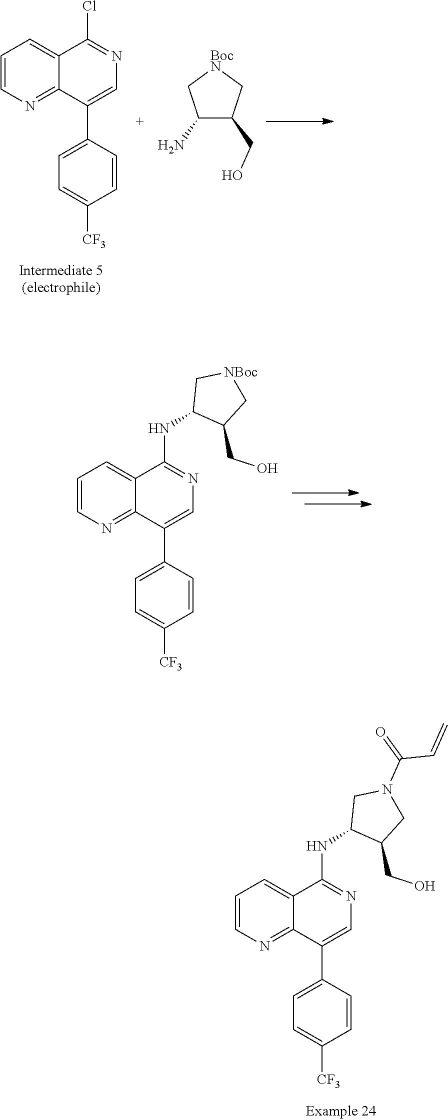 [SMILES: FC(F)(F)c1ccc(-c2cnc(Cl)c3cccnc23)cc1 ; CC(C)(C)OC(=O)N1C[C@@H](CO)[C@H](N)C1 ; CC(C)(C)OC(=O)N1C[C@@H](CO)[C@H](Nc2ncc(-c3ccc(C(F)(F)F)cc3)c3ncccc23)C1 ; C=CC(=O)N1C[C@@H](CO)[C@H](Nc2ncc(-c3ccc(C(F)(F)F)cc3)c3ncccc23)C1]

In one aspect, there is provided a compound of Formula (AI),

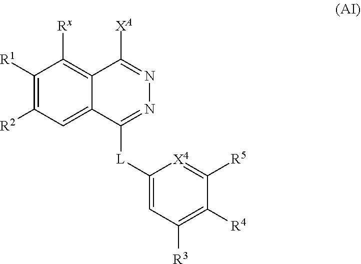

wherein
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and
Rⁱ, wherein Rⁱ is C₁₋₄ alkyl optionally substituted with —CN or C₁₋₄ alkoxy;
R³ and R⁵ are independently selected from H, F, Cl and C₁₋₄alkyl;
R⁴ is C₁₋₄ fluoroalkyl, —O(C₁₋₄ fluoroalkyl) or —S(C₁₋₄ fluoroalkyl);
R^{x} is H, F or C₁₋₄ alkyl;
X⁴ is CH or N; and
X^{A} is selected from F, Cl, Br, I, OSO₂CF₃, OSO₂Ph and O(4-toluenesulfonyl), or a salt thereof.

In one aspect, there is provided a compound of Formula (AII),

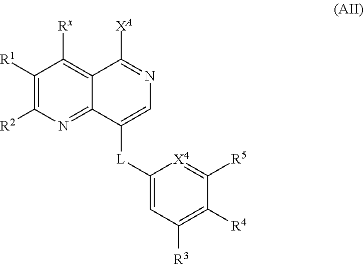

wherein
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and
Rⁱ, wherein Rⁱ is C₁₋₄ alkyl optionally substituted with —CN or C₁₋₄ alkoxy;
R³ and R⁵ are independently selected from H, F, Cl and C₁₋₄alkyl;
R⁴ is C₁₋₄ fluoroalkyl, —O(C₁₋₄ fluoroalkyl) or —S(C₁₋₄ fluoroalkyl);
R^{x} is H, F or C₁₋₄ alkyl,
X⁴ is CH or N; and
X^{A} is selected from F, Cl, Br, I, OSO₂CF₃, OSO₂Ph and O(4-toluenesulfonyl),
or a salt thereof.

In one aspect, there is provided a compound of Formula (AIII),

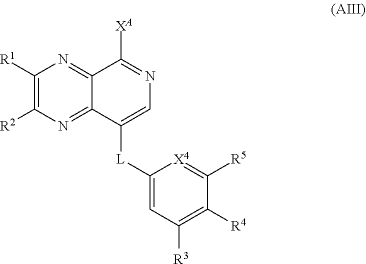

wherein
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and
Rⁱ, wherein Rⁱ is C₁₋₄ alkyl optionally substituted with —CN or C₁₋₄ alkoxy;
R³ and R⁵ are independently selected from H, F, Cl and C₁₋₄alkyl;
R⁴ is C₁₋₄ fluoroalkyl, —O(C₁₋₄ fluoroalkyl) or —S(C₁₋₄ fluoroalkyl);
X⁴ is CH or N; and
X^{A} is selected from F, Cl, Br, I, OSO₂CF₃, OSO₂Ph and O(4-toluenesulfonyl), or a salt thereof.

In one aspect, there is provided a compound of Formula (AIV),

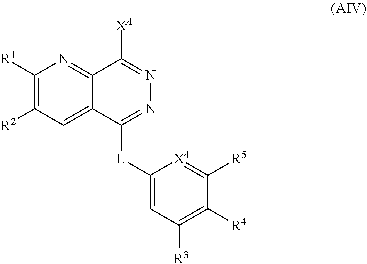

wherein
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and
Rⁱ, wherein Rⁱ is C₁₋₄ alkyl optionally substituted with —CN or C₁₋₄ alkoxy;
R³ and R⁵ are independently selected from H, F, Cl and C₁₋₄alkyl;
R⁴ is C₁₋₄ fluoroalkyl, —O(C₁₋₄ fluoroalkyl) or —S(C₁₋₄ fluoroalkyl);
X⁴ is CH or N; and
X^{A} is selected from F, Cl, Br, I, OSO₂CF₃, OSO₂Ph and O(4-toluenesulfonyl), or a salt thereof.

In one aspect, there is provided a compound of Formula (AV),

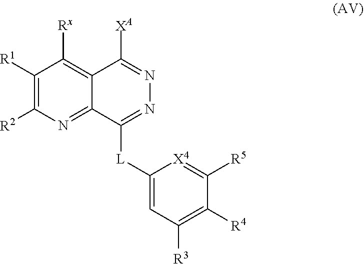

wherein
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and
Rⁱ, wherein Rⁱ is C₁₋₄ alkyl optionally substituted with —CN or C₁₋₄ alkoxy;
R³ and R⁵ are independently selected from H, F, Cl and C₁₋₄alkyl;
R⁴ is C₁₋₄ fluoroalkyl, —O(C₁₋₄ fluoroalkyl) or —S(C₁₋₄ fluoroalkyl);
R^{x} is H, F or C₁₋₄ alkyl;
X⁴ is CH or N; and
X^{A} is selected from F, Cl, Br, I, OSO₂CF₃, OSO₂Ph and O(4-toluenesulfonyl),
or a salt thereof.

Compound of Formula (AI), (AII), (AIII), (AIV) or (AV) may be made from the reaction of a corresponding compound of Formula (BI), (BII), (BIII), (BIV) or (BV) (as defined below) and a suitable activating agent. A non-limiting example of such a reaction is the reaction of 8-(4-(trifluoromethyl)phenyl)-1,6-naphthyridin-5(6H)-one and POCl₃ to give Intermediate 5.

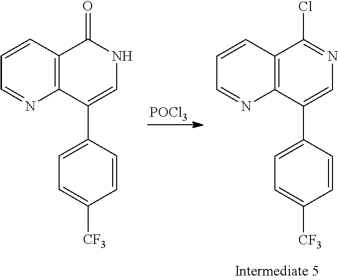

In one aspect, there is provided a compound of Formula (BI),

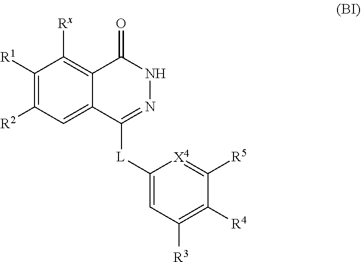

wherein L, R¹, R², R³, R⁴, R⁵, R^{x} and X⁴ are as defined for a compound of Formula (AI), or a salt thereof.

In one aspect, there is provided a compound of Formula (BII),

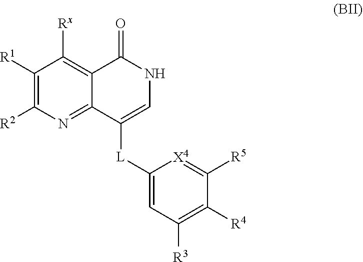

wherein L, R¹, R², R³, R⁴, R⁵, R^{x} and X⁴ are as defined for a compound of Formula (AII), or a salt thereof.

In one aspect, there is provided a compound of Formula (BIII),

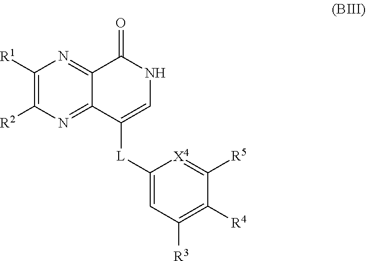

wherein L, R¹, R², R³, R⁴, R⁵ and X⁴ are as defined for a compound of Formula (AIII), or a salt thereof.

In one aspect, there is provided a compound of Formula (BIV),

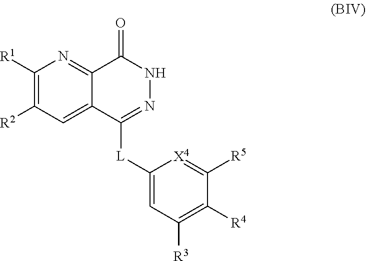

wherein L, R¹, R², R³, R⁴, R⁵ and X⁴ are as defined for a compound of Formula (AIV), or a salt thereof.

In one aspect, there is provided a compound of Formula (BV),

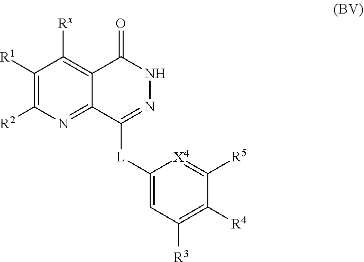

wherein L, R¹, R², R³, R⁴, R⁵, R^{x} and X⁴ are as defined for a compound of Formula (AV), or a salt thereof.

In embodiments, there is provided a compound of Formula (AI), (BI), (AII), (BII), (AIII), (BIII), (AIV) (BIV), (AV) or (BV), or a salt thereof, wherein L is a covalent bond.

In embodiments, there is provided a compound of Formula (AI), (BI), (AII), (BII), (AIII), (BIII), (AIV) (BIV), (AV) or (BV), or a salt thereof, wherein L is O.

In embodiments, there is provided a compound of Formula (AI), (BI), (AII), (BII), (AIII), (BIII), (AIV) (BIV), (AV) or (BV), or a salt thereof, wherein L is NH.

In embodiments, there is provided a compound of Formula (AI), (BI), (AII), (BII), (AIII), (BIII), (AIV) (BIV), (AV) or (BV), or a salt thereof, wherein L is CH₂.

In embodiments, there is provided a compound of Formula (AI), (BI), (AII), (BII), (AIII), (BIII), (AIV) (BIV), (AV) or (BV), or a salt thereof, wherein L is CH₂CH₂.

In embodiments, there is provided a compound of Formula (AI), (BI), (AII), (BII), (AIII), (BIII), (AIV) (BIV), (AV) or (BV), or a salt thereof, wherein R¹ is H or C₁₋₄ alkyl, such as CH₃.

In embodiments, there is provided a compound of Formula (AI), (BI), (AII), (BII), (AIII), (BIII), (AIV) (BIV), (AV) or (BV), or a salt thereof, wherein R² is H or CH₃, such as H.

In embodiments, there is provided a compound of Formula (AI), (BI), (AII), (BII), (AIII), (BIII), (AIV) (BIV), (AV) or (BV), or a salt thereof, wherein R¹ and R² independently selected from H and C₁₋₄ alkyl, such as CH₃. In further embodiments, R¹ and R² are both H.

In embodiments, there is provided a compound of Formula (AI), (BI), (AII), (BII), (AIII), (BIII), (AIV) (BIV), (AV) or (BV), or a salt thereof, wherein R³ and R⁵ are H, and optionally R⁴ is CF₂H, CF₂CH₃, CF₃, OCF₃, OCF₂H or SCF₃.

In embodiments, there is provided a compound of Formula (AI), (BI), (AII), (BII), (AIII), (BIII), (AIV) (BIV), (AV) or (BV), or a salt thereof, wherein R³ and R⁵ are H and R⁴ is CF₃.

In embodiments, there is provided a compound of Formula (AI), (BI), (AII), (BII), (AIII), (BIII), (AIV) (BIV), (AV) or (BV), or a salt thereof, wherein X⁴ is CH.

In embodiments, there is provided a compound of Formula (AI), (BI), (AII), (BII), (AIII), (BIII), (AIV) (BIV), (AV) or (BV), or a salt thereof, wherein X⁴ is N.

In embodiments, there is provided a compound of Formula (AI), (BI), (AII), (BII), (AIII), (BIII), (AIV) (BIV), (AV) or (BV), or a salt thereof, wherein L is a covalent bond, R¹ and R² are both H, X⁴ is CH, R³ and R⁵ are H, and R⁴ is CF₂H, CF₂CH₃, CF₃, OCF₃, OCF₂H or SCF₃ (such as CF₃).

In embodiments, there is provided a compound of Formula (AI), (BI), (AII), (BII), (AIII), (BIII), (AIV) (BIV), (AV) or (BV), or a salt thereof, wherein L is a covalent bond, R¹ and R² are both H, X⁴ is N, R³ and R⁵ are H, and R⁴ is CF₂H, CF₂CH₃, CF₃, OCF₃, OCF₂H or SCF₃ (such as CF₃).

In embodiments, there is provided a compound of Formula (AI), (BI), (AII), (BII), (AV) or (BV), or a salt thereof, wherein R^{x} is H.

In embodiments, there is provided a compound of Formula (AI), (BI), (AII), (BII), (AIII), (BIII), (AIV) (BIV), (AV) or (BV), or a salt thereof, wherein X^{A} is F or Cl.

In one aspect, there is provided a method of manufacturing a compound of Formula (CIII) from a compound of Formula (CI) and a compound of Formula (CII)

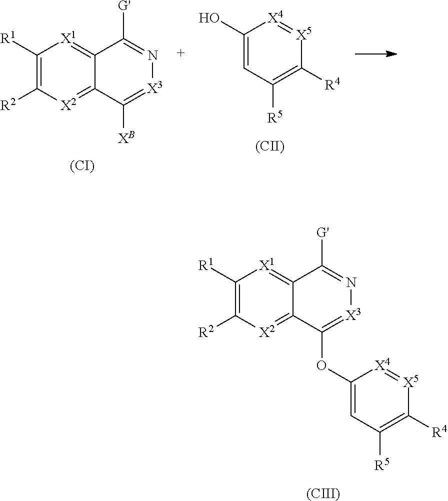

wherein, R¹, R², R³, R⁴, R⁵, X¹, X², X³, X⁴, X⁵ are as defined for a compound of Formula (I), G′ is G or a precursor thereof and X^{B} is selected from F, Cl, Br, I, OSO₂CF₃, OSO₂Ph or O(4-toluenesulfonyl). In embodiments, the reaction is performed in the presence of a Cu(I) catalyst, such as Cu(I)I, such as 50 mol % Cu(I)I. In further embodiments, the reaction is performed in the presence of dimethyl glycine, such as 150 mol % dimethyl glycine. In further embodiments, the reaction is performed in the presence of a base such as cesium carbonate, such as 150 mol % cesium carbonate. In further embodiments, the reaction is performed in 1,4-dioxane, optionally at 90° C. In further embodiments, X^{B} is Br.

### EXAMPLES

The specification will now be illustrated by the following non-limiting Examples in which, generally:
(i) operations were carried out at ambient temperature, i.e. in the range 17 to 25° C. and under an atmosphere of an inert gas such as nitrogen unless otherwise stated;
(ii) evaporations were carried out by rotary evaporation or utilising GENEVAC equipment or BIOTAGE v10 evaporator or ROTAVAPOR BUCHI and FREEZEMOBILE 35EL from SP SCIENTIFIC in vacuo and workup procedures were carried out after removal of residual solids by filtration and quenching with appropriate solvent;
(iii) flash chromatography purifications were performed on an automated BIOTAGE ISOLERA ONE or BIOTAGE SELEKT or TELEDYNE ISCO COMBIFLASH Rf using prepacked BIOTAGE SFAR SILICA HC (20 μm) and BUCHI SILICA ECOFLEX (50 am);
(iv) preparative chromatography was performed on a AGILENT MDAP 1290 Prep system, fractions were collected when both detectors (UV and MS) detect a peak, or via supercritical fluid chromatography performed on a WATERS Prep 100 SFC-MS instrument with MS- and UV-triggered collection or a SEPIATEC PREP SFC 100 instrument with UV collection;
(v) yields, where present, are not necessarily the maximum attainable;
(vi) in general, the structures of compounds of Formula (I) were confirmed by nuclear magnetic resonance (NMR) spectroscopy; NMR chemical shift values were measured on the delta scale (proton magnetic resonance spectra were acquired using a BRUKER NEO 500 (500 MHz) or BRUKER nano AVIIIHD 400 (400 MHz) instrument); measurements were taken at 27° C. (300 K) unless otherwise specified; the following abbreviations have been used: s, singlet; d, doublet; t, triplet; q, quartet; m, multiplet; dd, doublet of doublets; ddd, doublet of doublet of doublet; dt, doublet of triplets; br, broad signal;
(vii) in general, compounds of Formula (I) were also characterised by mass spectroscopy following liquid chromatography (UPLC); UPLC was carried out using a UPLC-MS was carried out using a WATERS ACQUITY UPLC and WATERS SQD mass spectrometer (column temp 30° C., UV detection=210-400 nm, mass spec=ESI with positive/negative switching) at a flow rate of 1 mL/min using a solvent gradient of 2 to 98% B over 1.5 mins (total runtime with equilibration back to starting conditions 2 min), where A=0.1% formic acid in water and B=0.1% formic acid in acetonitrile (for acid work) or A=0.1% ammonium hydroxide in water and B=acetonitrile (for base work). For acid analysis the column used was WATERS ACQUITY HSS T3, 1.8 mm, 2.1×30 mm; for base analysis the column used was WATERS ACQUITY BEH C18, 1.7 mm, 2.1×30 mm;
(viii) intermediate purity was assessed by thin layer chromatographic, mass spectral, HPLC (high performance liquid chromatography) and/or NMR analysis;
(ix) Where reactions were conducted in a microwave reactor, this was done using a BIOTAGE INITIATOR and BIOTAGE Microwave Vials;
(x) The skilled person will be aware that the chemical name of a given compound may vary depending on the software package used to name it. For this specification, PERKIN ELMER E-NOTEBOOK was used to name compounds.
(xi) the following abbreviations have been used:
   Aq aqueous
   Boc tert-butyloxycarbonyl
   CDCl₃ deuterochloroform
   DCM dichloromethane
   DIPEA N,N-diisopropylethylamine
   DMF N,N-dimethylformamide
   DMSO dimethyl sulfoxide
   dppf 1,1′-bis(diphenylphosphino)ferrocene
   EtOAc ethyl acetate
   HPLC high performance liquid chromatography
   MeCN acetonitrile
   MeOH methanol
   Ms methanesulfonyl
   rt room temperature
   SEM trimethylsilylethoxymethyl
   SFC Supercritical fluid chromatography
   TFA trifluoroacetic acid
   THF tetrahydrofuran.

### Intermediate 1: 1,4-dichlorophthalazine

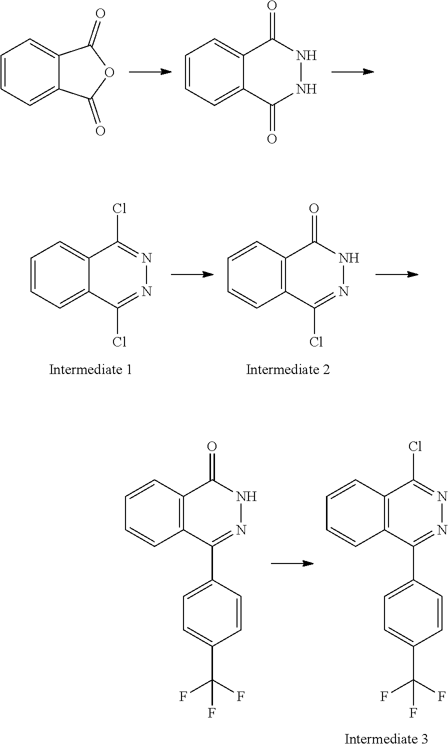 [SMILES: O=C1OC(=O)c2ccccc21 ; O=c1[nH][nH]c(=O)c2ccccc12 ; Clc1nnc(Cl)c2ccccc12 ; O=c1[nH]nc(Cl)c2ccccc12 ; O=c1[nH]nc(-c2ccc(C(F)(F)F)cc2)c2ccccc12 ; FC(F)(F)c1ccc(-c2nnc(Cl)c3ccccc23)cc1]

### 2,3-dihydrophthalazine-1,4-dione

Hydrazine monohydrate (45.35 mL, 607.6 mmol) was added slowly to a solution of isobenzofuran-1,3-dione (60.0 g, 405 mmol) in acetic acid (120 mL) at 0° C. The reaction mixture was heated to reflux and stirred for 30 min forming a white suspension. The reaction mixture was cooled to rt and the precipitate was diluted with water. The solids were filtered and washed with water. The filter cake was dried under vacuum to afford 2,3-dihydrophthalazine-1,4-dione (60.7 g, 92% yield) as a white solid. ¹H NMR (500 MHz, DMSO-d6) δ 7.69-7.94 (2H, m), 8.07 (2H, br s), 11.51 (2H, br s); m/z: (ES⁺) [M+H]⁺=163.

### Intermediate 1: 1,4-dichlorophthalazine

POCl₃ (163.0 mL, 175.1 mmol) was added to a mixture of 2,3-dihydrophthalazine-1,4-dione (56.80 g, 350.3 mmol) in acetonitrile (500 mL) and pyridine (28.3 mL, 350.3 mmol) at 0° C. The reaction mixture was heated to 80° C. and stirred for 15 hrs. The reaction mixture was cooled to rt and the volatiles were removed under reduced pressure. The resulting residue was cooled to 0° C. and suspended in saturated aq. NaHCO₃. The formed precipitate was collected by filtration and the solid was washed with water, hexanes and ether. The solid was dried under vacuum to afford 1,4-dichlorophthalazine (Intermediate 1, 60 g, 86% yield) as a light-yellow solid. ¹H NMR (500 MHz, DMSO-d6) δ 8.17-8.32 (2H, m), 8.33-8.46 (2H, m); m/z: (ES⁺) [M+H]⁺=199.

### Intermediate 2: 4-chlorophthalazin-1(2H)-one

A solution of 1,4-dihlorophthalazine (Intermediate 1, 67.0 g, 337 mmol) in acetic acid (300 mL) was heated to 80° C. and stirred for 2 hrs, forming a white suspension. The reaction mixture was cooled to rt and the majority of the acetic acid was removed under reduced pressure. The resulting suspension was diluted with water (˜600 mL) and the solid was collected by filtration. The solid was washed with water and then dried under vacuum to afford 4-chlorophthalazin-1(2H)-one (Intermediate 2, 60.5 g, 100% yield) as a pink solid. ¹H NMR (500 MHz, DMSO-d6) δ 7.86-8.12 (3H, m), 8.28 (1H, dd), 12.86 (1H, br s); m/z: (ES⁺) [M+H]⁺=181.

### Intermediate 3: 1-chloro-4-(4-(trifluoromethyl)phenyl)phthalazine

### 4-(4-(trifluoromethyl)phenyl)phthalazin-1(2H)-one

4-Chlorophthalazin-1(2H)-one (Intermediate 2, 30.10 g, 166.7 mmol), (4-(trifluoromethyl)phenyl)boronic acid (34.80 g, 183.3 mmol), PdCl₂(dppf) (6.10 g, 8.33 mmol) and Cs₂CO₃ (109.0 g, 333.4 mmol) were diluted in 1,4-dioxane (440 mL) and H₂O (110 mL) under an atmosphere of N₂. The reaction mixture was heated to 80° C. and stirred for 20 hrs. The reaction mixture was cooled to rt and diluted with H₂O (200 mL). The phases were separated and the organic layer was diluted with EtOAc (500 mL) and washed with H₂O (500 mL). The organic layer was a suspension and the solid was collected by filtration. The filter cake was washed with DCM and acetonitrile and then dried under vacuum to afford 4-(4-(trifluoromethyl)phenyl)phthalazin-1(2H)-one (41.7 g, 86% yield) as a light grey solid. ¹H NMR (500 MHz, DMSO-d6) δ 7.55-7.72 (1H, m), 7.84 (2H, d), 7.87-7.98 (4H, m), 8.26-8.52 (1H, m), 12.96 (1H, s); m/z: (ES⁺) [M+H]⁺=291.

### Intermediate 3: 1-chloro-4-(4-(trifluoromethyl)phenyl)phthalazine

POCl₃ (78.0 mL, 834 mmol) was added to a suspension of 4-(4-(trifluoromethyl)phenyl)phthalazin-1(2H)-one (48.40 g, 166.8 mmol) in acetonitrile (200 mL). Pyridine (13.49 mL, 166.8 mmol) was slowly added and the reaction mixture was heated to 80° C. and stirred for 20 hrs. The reaction mixture was cooled to rt and the volatiles were removed under reduced pressure. The solid residue was suspended into H₂O at 0° C. and the solid was collected by filtration and washed with water. The solid was dried under vacuum to afford 1-chloro-4-(4-(trifluoromethyl)phenyl)phthalazine (Intermediate 3, 38.0 g, 74% yield) as a light-yellow solid. ¹H NMR (500 MHz, DMSO-d6) δ 7.90-8.08 (5H, m), 8.15 (1H, td), 8.24 (1H, td), 8.44 (1H, d); m/z: (ES⁺) [M+H]⁺=309.

### Intermediate 4: 8-bromo-6-((2-(trimethylsilyl)ethoxy)methyl)-1,6-naphthyridin-5(6H)-one

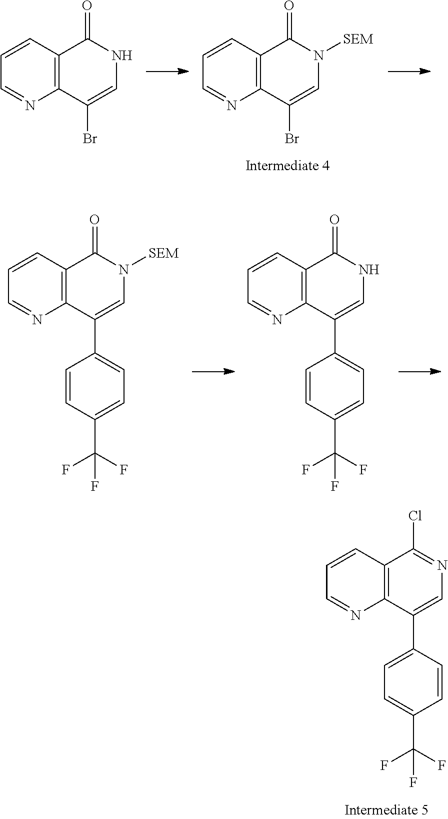 [SMILES: O=c1[nH]cc(Br)c2ncccc12 ; C[Si](C)(C)CCOCn1cc(Br)c2ncccc2c1=O ; C[Si](C)(C)CCOCn1cc(-c2ccc(C(F)(F)F)cc2)c2ncccc2c1=O ; O=c1[nH]cc(-c2ccc(C(F)(F)F)cc2)c2ncccc12 ; FC(F)(F)c1ccc(-c2cnc(Cl)c3cccnc23)cc1]

NaH (60% dispersion in mineral oil, 1.71 g, 42.7 mmol) was added to a mixture of 8-bromo-1,6-naphthyridin-5(6H)-one (8.00 g, 35.6 mmol) in THF (100 mL) at 0° C. and the reaction mixture was stirred for 30 min. (2-(Chloromethoxy)ethyl)trimethylsilane (7.55 mL, 42.7 mmol) was slowly added to the reaction flask and the reaction was warmed to rt and stirred for 16 hrs. The mixture was carefully quenched with saturated aq. NaHCO₃ (25 mL). The phases were separated and the aqueous layer was extracted with EtOAc (3×80 mL). The combined organics were washed with brine (40 mL), dried over Na₂SO₄, filtered and concentrated to dryness. The crude material was purified by flash silica chromatography (0 to 30% EtOAc in hexanes) to afford 8-bromo-6-((2-(trimethylsilyl)ethoxy)methyl)-1,6-naphthyridin-5(6H)-one (Intermediate 4, 1.09 g, 8% yield) as a white solid. ¹H NMR (500 MHz, CDCl₃) δ 0.03 (9H, s), 0.95-1.05 (2H, m), 3.63-3.73 (2H, m), 5.44 (2H, s), 7.52 (1H, dd), 7.81 (1H, s), 8.73 (1H, d), 9.05-9.11 (1H, m); m/z: (ES⁺) [M+H]⁺=355.

### Intermediate 5: 5-chloro-8-(4-(trifluoromethyl)phenyl)-1,6-naphthyridine

### 8-(4-(trifluoromethyl)phenyl)-6-((2-(trimethylsilyl)ethoxy)methyl)-1,6-naphthyridin-5(6H)-one

8-Bromo-6-((2-(trimethylsilyl)ethoxy)methyl)-1,6-naphthyridin-5(6H)-one (Intermediate 4, 7.00 g, 19.7 mmol), (4-(trifluoromethyl)phenyl)boronic acid (4.90 g, 25.8 mmol), PdCl₂(dppf) (720 mg, 0.99 mmol) and Cs₂CO₃ (12.84 g, 39.40 mmol) were diluted with dioxane (65 mL) and H₂O (13 mL) under an atmosphere of N₂. The reaction mixture was heated to 65° C. and stirred for 2 hrs. The reaction mixture was cooled to rt and filtered through diatomaceous earth and the filtrate was concentrated to dryness. The resulting residue was suspended in H₂O (10 mL) and extracted with EtOAc (3×50 mL). The combined organics were washed with H₂O (20 mL) and brine (20 mL). The organic layer was dried over Na₂SO₄, filtered and concentrated to dryness. The crude material was purified by flash silica chromatography (0 to 15% EtOAc in hexanes) to afford 8-(4-(trifluoromethyl)phenyl)-6-((2-(trimethylsilyl)ethoxy)methyl)-1,6-naphthyridin-5(6H)-one (7.5 g, 91% yield) as an amber oil. ¹H NMR (400 MHz, DMSO-d6) 5-0.05 (9H, s), 0.89 (2H, t), 3.66 (2H, t), 5.45 (2H, s), 7.62 (1H, dd), 7.79-7.86 (4H, m), 7.97 (1H, s), 8.67 (1H, dd), 8.96 (1H, dd); m/z: (ES⁺) [M+H]⁺=421.

### 8-(4-(trifluoromethyl)phenyl)-1,6-naphthyridin-5(6H)-one

TFA (26.00 mL, 337.7 mmol) was added to a solution of 8-(4-(trifluoromethyl)phenyl)-6-((2-(trimethylsilyl)ethoxy)methyl)-1,6-naphthyridin-5(6H)-one (7.10 g, 16.9 mmol) in DCM (24 mL) at 0° C. and the reaction mixture was stirred for 1 h. The reaction mixture was concentrated and then diluted in MeOH (24 mL). The mixture was cooled to 0° C. and ammonia in MeOH (7 M, 24.12 mL, 168.8 mmol) was added dropwise. The reaction mixture was warmed to rt and stirred for 2 hrs. The mixture was diluted with water (10 mL) and extracted with 3:1 DCM/iPrOH (3×30 mL). The combined organics were dried over Na₂SO₄, filtered and concentrated to dryness to afford 8-(4-(trifluoromethyl)phenyl)-1,6-naphthyridin-5(6H)-one (2.0 g, 41% yield) as a tan solid. ¹H NMR (400 MHz, CDCl₃) δ 7.43-7.49 (1H, m), 7.51-7.55 (1H, m), 7.75 (4H, s), 8.79 (1H, br d), 9.00-9.05 (1H, m), 10.57 (1H, br s); m/z: (ES⁺) [M+H]⁺=291.

### Intermediate 5: 5-chloro-8-(4-(trifluoromethyl)phenyl)-1,6-naphthyridine

POCl₃ (3.40 mL, 36.5 mmol) and pyridine (0.59 mL, 7.3 mmol) were added to a solution of 8-(4-(trifluoromethyl)phenyl)-1,6-naphthyridin-5(6H)-one (2.12 g, 7.30 mmol) in acetonitrile (48 mL). The reaction mixture was heated to 80° C. and stirred for 16 hrs. The reaction mixture was cooled to rt and the volatiles were removed under reduced pressure. Crushed ice was added to the residue followed by water (10 mL), and the formed solid was collected by filtration. The crude solid was purified by flash silica chromatography (0 to 20% EtOAc in hexanes) to afford 5-chloro-8-(4-(trifluoromethyl)phenyl)-1,6-naphthyridine (Intermediate 5, 1.10 g, 49% yield) as a white solid. ¹H NMR (500 MHz, DMSO-d6) δ 7.87-7.99 (5H, m), 8.67 (1H, s), 8.79 (1H, br d), 9.20-9.29 (1H, m); m/z: (ES⁺) [M+H]⁺=309.

### Intermediate 6: 8-bromo-5-chloropyrido[3,4-b]pyrazine

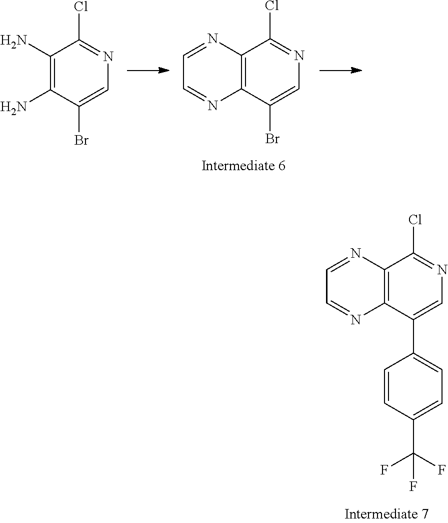 [SMILES: Nc1c(Br)cnc(Cl)c1N ; Clc1ncc(Br)c2nccnc12 ; FC(F)(F)c1ccc(-c2cnc(Cl)c3nccnc23)cc1]

5-Bromo-2-chloropyridine-3,4-diamine (25.00 g, 112.4 mmol) was dissolved in butan-1-ol (130 mL). Oxalaldehyde (40% in H₂O, 19.76 mL, 172.3 mmol) was added and the reaction mixture was heated to 85° C. and stirred for 2 hrs. The reaction mixture was cooled to rt and the solid that precipitated out was collected by filtration. The solid was washed with H₂O (200 mL) and heptane (500 mL) and then dried under vacuum to afford 8-bromo-5-chloropyrido[3,4-b]pyrazine (Intermediate 6, 24 g, 87% yield) as a cream solid which was used without further purification. ¹H NMR (500 MHz, DMSO-d6) δ 8.95 (1H, s), 9.25 (1H, d), 9.35 (1H, d); m/z: (ES⁺) [M+H]⁺=246.

### Intermediate 7: 5-chloro-8-(4-(trifluoromethyl)phenyl)pyrido[3,4-b]pyrazine

8-Bromo-5-chloropyrido[3,4-b]pyrazine (Intermediate 6, 36.20 g, 148.1 mmol), (4-(trifluoromethyl)phenyl)boronic acid (28.10 g, 148.1 mmol), Cs₂CO₃ (145.0 g, 444.2 mmol) and bis(di-tert-butyl(4-dimethylaminophenyl)phosphine)dichloropalladium(II) (5.24 g, 7.40 mmol) were diluted in 1,4-dioxane (1.35 L) and H₂O (135 mL) under an atmosphere of N₂. The reaction mixture was heated to 40° C. and stirred for 2 hrs. The reaction mixture was cooled to rt and the mixture was concentrated under reduced pressure to remove ^{˜}80% of 1,4-dioxane. The residue was partitioned between water (100 mL) and EtOAc (200 mL). The organic phase was washed with brine, dried over MgSO₄, filtered and concentrated to dryness to afford the crude product (60 g) as a brown solid. The crude solid was suspended in EtOH (400 mL) and heated to 80° C. Water (200 mL) was added and heating maintained until a solution reformed. The solution was filtered through a short pad of diatomaceous earth to remove a black residue, and the solution was allowed to cool to rt over 30 minutes, during which time a precipitate formed. The precipitate was collected by filtration, washed with cold EtOH/H₂O (50:50 v/v) and dried under vacuum to afford the product (33.0 g, 72% yield) as a beige solid. Mother liquors were evaporated and the residue was purified by flash silica chromatography (0 to 25% EtOAc in heptane) to afford a second batch as a beige solid. Combined yield 5-chloro-8-(4-(trifluoromethyl)phenyl)pyrido[3,4-b]pyrazine (Intermediate 7, 39.0 g, 85% yield) as a beige solid. ¹H NMR (500 MHz, DMSO-d6) δ 7.91 (2H, d), 7.95 (2H, d), 8.78 (1H, s), 9.24 (1H, d), 9.27 (1H, d); m/z: ES⁺[M+H]⁺=310.

### Intermediate 8: 5,8-dichloropyrido[2,3-d]pyridazine

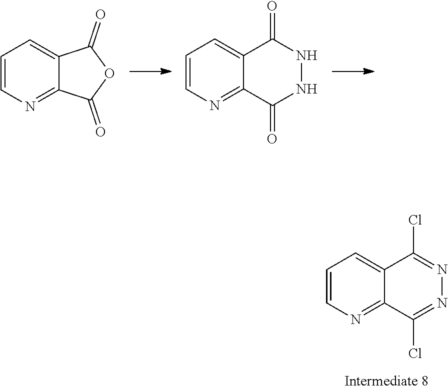 [SMILES: O=C1OC(=O)c2ncccc21 ; O=c1[nH][nH]c(=O)c2ncccc12 ; Clc1nnc(Cl)c2ncccc12]

### 6,7-dihydropyrido[2,3-d]pyridazine-5,8-dione

Hydrazine (16.44 mL, 335.3 mmol) was added to a stirred suspension of furo[3,4-b]pyridine-5,7-dione (25.00 g, 167.7 mmol) in acetic acid (120 mL). The reaction mixture was heated to reflux and stirred for 2 hrs. The reaction mixture was cooled to rt generating a white precipitate. The solids were collected by filtration and washed with water (3×50 mL). The solids were dried under vacuum to afford 6,7-dihydropyrido[2,3-d]pyridazine-5,8-dione (22.9 g, 84% yield) as a white solid which was used without further purification. ¹H NMR (500 MHz, DMSO-d6) δ 7.72-8.01 (1H, m), 8.51 (1H, br s), 9.11 (1H, br s), 11.10-11.57 (1H, m), 11.69-12.25 (1H, m); m/z: (ES⁺) [M+H]⁺=163.

### Intermediate 8: 5,8-dichloropyrido[2,3-d]pyridazine

POCl₃ (62.60 mL, 671.2 mmol) was added to a solution of 6,7-dihydropyrido[2,3-d]pyridazine-5,8-dione (21.90 g, 134.2 mmol) and pyridine (10.86 mL, 134.2 mmol) in acetonitrile (60 mL). The reaction mixture was heated to 80° C. and stirred for 4 hrs. The reaction mixture was cooled to rt and the solvent was removed under reduced pressure. The resulting residue was diluted with crushed ice and cooled to 0° C. Saturated aq. NaHCO₃ and solid NaHCO₃ were added until the pH measured ^{˜}7. The solids were collected by filtration, washed with H₂O, and then dried under vacuum to afford 5,8-dichloropyrido[2,3-d]pyridazine (Intermediate 8, 18.73 g, 70% yield) as a light brown solid which was used without further purification. ¹H NMR (500 MHz, DMSO-d6) δ 8.24 (1H, dd), 8.76 (1H, dd), 9.48 (1H, dd); m/z: (ES⁺) [M+H]⁺=201.

### Intermediate 9: 5-chloro-8-(5-(trifluoromethyl)pyridin-2-yl)-1,6-naphthyridine

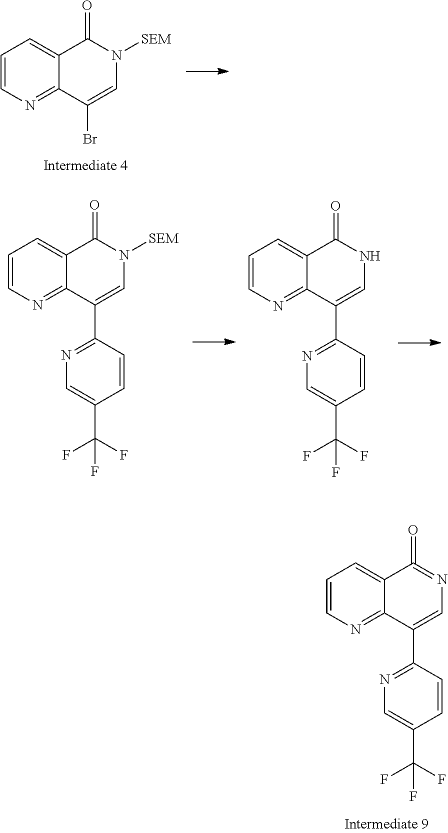

### 8-(5-(trifluoromethyl)pyridin-2-yl)-6-((2-(trimethylsilyl)ethoxy)methyl)-1,6-naphthyridin-5(6H)-one

8-Bromo-6-((2-(trimethylsilyl)ethoxy)methyl)-1,6-naphthyridin-5(6H)-one (Intermediate 4, 403.7 mg, 1.136 mmol) and bis(pinacolato)diboron (346 mg, 1.36 mmol) were dissolved in 1,4-dioxane (8 mL) under an atmosphere of N₂. PdCl₂(dppf) (83 mg, 0.11 mmol) and potassium acetate (223 mg, 2.27 mmol) were added and the resulting mixture was refluxed at 110° C. for 5 hrs. The reaction mixture was cooled to 85° C. and a solution of 2-chloro-5-(trifluoromethyl)pyridine (309 mg, 1.70 mmol) and PdCl₂(dppf) (83 mg, 0.11 mmol) in 1,4-dioxane (3 mL) was added. Cs₂CO₃ (740 mg, 2.3 mmol) and water (2 mL) were added and the resulting mixture was stirred at 85° C. for 1 hr. The reaction mixture was cooled to rt and diluted with water (15 mL). The layers were separated and the aqueous layer was extracted with EtOAc (2×20 mL). The combined organics were dried over Na₂SO₄, filtered and concentrated to dryness. The crude material was purified by flash silica chromatography (0-50% EtOAc in hexanes) to afford 8-(5-(trifluoromethyl)pyridin-2-yl)-6-((2-(trimethylsilyl)ethoxy)methyl)-1,6-naphthyridin-5(6H)-one (0.238 g, 50% yield) as an amber oil. ¹H NMR (500 MHz, CDCl₃) δ 0.00-0.04 (9H, m), 0.99-1.04 (2H, m), 3.70-3.76 (2H, m), 5.56 (2H, s), 7.53 (1H, dd), 8.00-8.06 (1H, m), 8.39 (1H, s), 8.62 (1H, br d), 8.80-8.85 (1H, m), 8.94 (1H, br s), 9.02 (1H, br s); m/z: (ES⁺) [M+H]⁺=422.

### 8-(5-(trifluoromethyl)pyridin-2-yl)-1,6-naphthyridin-5(6H)-one

TFA (1.0 mL, 13 mmol) was added to a solution of 8-(5-(trifluoromethyl)pyridin-2-yl)-6-((2-(trimethylsilyl)ethoxy)methyl)-1,6-naphthyridin-5(6H)-one (238 mg, 0.564 mmol) in DCM (5 mL) at 0° C. The reaction mixture was gradually warmed to rt and stirred for 15 hrs. The reaction mixture was concentrated to dryness and the residue was redissolved in methanol (5 mL) and cooled to 0° C. Ammonia (2.5 M in MeOH, 5.0 mL, 230 mmol) was added dropwise and the resulting mixture was stirred at 0° C. for 10 min, then rt for 30 min. The reaction mixture was diluted with water (10 mL) and extracted with 3:1 CHCl₃/iPrOH (3×15 mL). The combined organics were dried over Na₂SO₄ and concentrated to dryness to afford 8-(5-(trifluoromethyl)pyridin-2-yl)-1,6-naphthyridin-5(6H)-one (0.162 g, 98% yield) as a beige solid, which was carried forward without purification. m/z: (ES⁺) [M+H]⁺=292.

### Intermediate 9: 5-chloro-8-(5-(trifluoromethyl)pyridin-2-yl)-1,6-naphthyridine

8-(5-(Trifluoromethyl)pyridin-2-yl)-1,6-naphthyridin-5(6H)-one (493 mg, 1.69 mmol) was dissolved in acetonitrile (36 mL). POCl₃ (1.58 mL, 16.9 mmol) and pyridine (205 μL, 2.54 mmol) were added and the resulting mixture was heated to 80° C. and stirred for 5 hrs. The reaction mixture was cooled to rt and concentrated in vacuo. Ice was added to the residue, followed by careful neutralization with saturated aq. NaHCO₃ to give a suspension. The solid was collected by filtration, washed with H₂O, and dried under vacuum to give 5-chloro-8-(5-(trifluoromethyl)pyridin-2-yl)-1,6-naphthyridine (Intermediate 9, 0.370 g, 71% yield) as a gray-brown solid, which was carried forward without purification. m/z: (ES⁺) [M+H]⁺=310.

### Intermediate 10: 4-chloro-5-fluoro-1-(4-(trifluoromethyl)phenyl)phthalazine

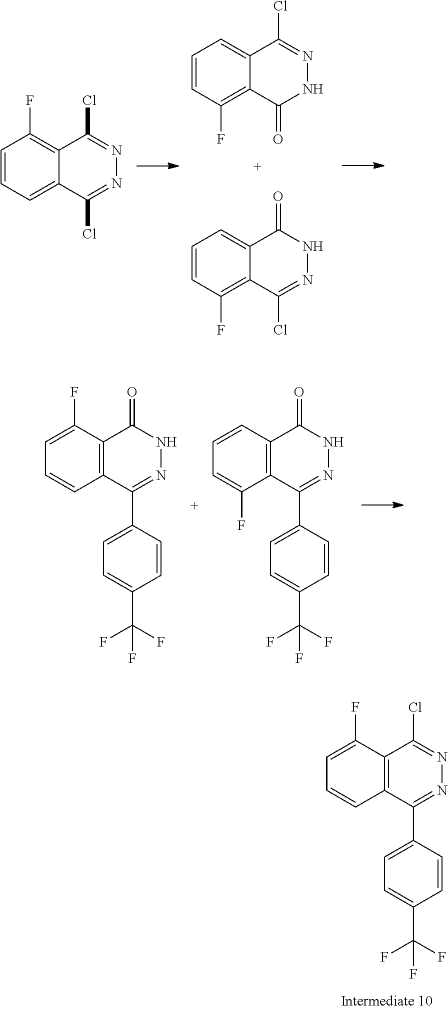

### 4-chloro-8-fluorophthalazin-1(2H)-one and 4-chloro-5-fluorophthalazin-1(2H)-one

Purity of the 1,4-dichloro-5-fluorophthalazine starting material was analyzed by ¹H NMR and LCMS and the spectra showed that the material was hydrolyzed during storage to a mixture of 4-chloro-8-fluorophthalazin-1(2H)-one and 4-chloro-5-fluorophthalazin-1(2H)-one. m/z: (ES⁺) [M+H]⁺=199.

### 5-fluoro-4-(4-(trifluoromethyl)phenyl)phthalazin-1(2H)-one

(4-(Trifluoromethyl)phenyl)boronic acid (2.63 g, 13.6 mmol), a mixture of 4-chloro-8-fluorophthalazin-1(2H)-one and the regioisomer 4-chloro-5-fluorophthalazin-1(2H)-one (2.50 g, 12.6 mmol), PdCl₂(dppf) (460 mg, 0.63 mmol) and Cs₂CO₃ (8.20 g, 25.2 mmol) were diluted in 1,4-dioxane (70 mL) and H₂O (17.5 mL) under an atmosphere of N₂. The reaction mixture was heated to 80° C. and stirred for 16 hrs. The reaction mixture was cooled to rt and diluted with H₂O (100 mL) and EtOAc (100 mL). The solid was collected by filtration and the layers of the filtrate were separated. The aqueous layer was extracted with EtOAc (3×50 mL) and the combined organics were dried over Na₂SO₄, filtered and concentrated to a suspension. The solid was collected by filtration. Both batches of solid were combined and suspended in DCM and the solid was collected by filtration. The solid was washed with DCM and then dried under vacuum to afford the product as a mixture of 8-fluoro-4-(4-(trifluoromethyl)phenyl)phthalazin-1(2H)-one (minor) and 5-fluoro-4-(4-(trifluoromethyl)phenyl)phthalazin-1(2H)-one (major) (2.82 g) as a white solid. The filtrate from the DCM suspension was concentrated to dryness and purified by flash silica gel chromatography (20 to 60% EtOAc in hexanes) to yield the second batch of product as a single regioisomer of 5-fluoro-4-(4-(trifluoromethyl)phenyl)phthalazin-1(2H)-one (1.03 g, 3.34 mmol, 27% yield) as a white solid. m/z: (ES⁺) [M+H]⁺=309.

### Intermediate 10: 4-chloro-5-fluoro-1-(4-(trifluoromethyl)phenyl)phthalazine

POCl₃ (1.56 mL, 16.7 mmol) and pyridine (0.27 mL, 3.3 mmol) were added to a suspension of 8-fluoro-4-(4-(trifluoromethyl)phenyl)phthalazin-1(2H)-one (1.03 g, 3.34 mmol) in acetonitrile (10 mL). The reaction mixture was heated to 80° C. and stirred for 20 hrs. The reaction mixture was cooled to rt and the volatiles were removed under reduced pressure. The solid residue was suspended into ice cold H₂O. The solid was collected by filtration, washed with water, and then dried under vacuum to afford 4-chloro-5-fluoro-1-(4-(trifluoromethyl)phenyl)phthalazine (Intermediate 17, 1.09 g, 99% yield) as a red solid. ¹H NMR (500 MHz, DMSO-d6) δ 7.69 (2H, d), 7.82 (1H, br dd), 7.93-7.97 (1H, m), 7.98 (2H, d), 8.02 (1H, d); m/z: (ES⁺) [M+H]⁺=327.

### Example 1: 1-((3S,4R)-3-fluoro-4-((8-(4-(trifluoromethyl)phenyl)pyrido[2,3-d]pyridazin-5-yl)amino)pyrrolidin-1-yl)prop-2-en-1-one

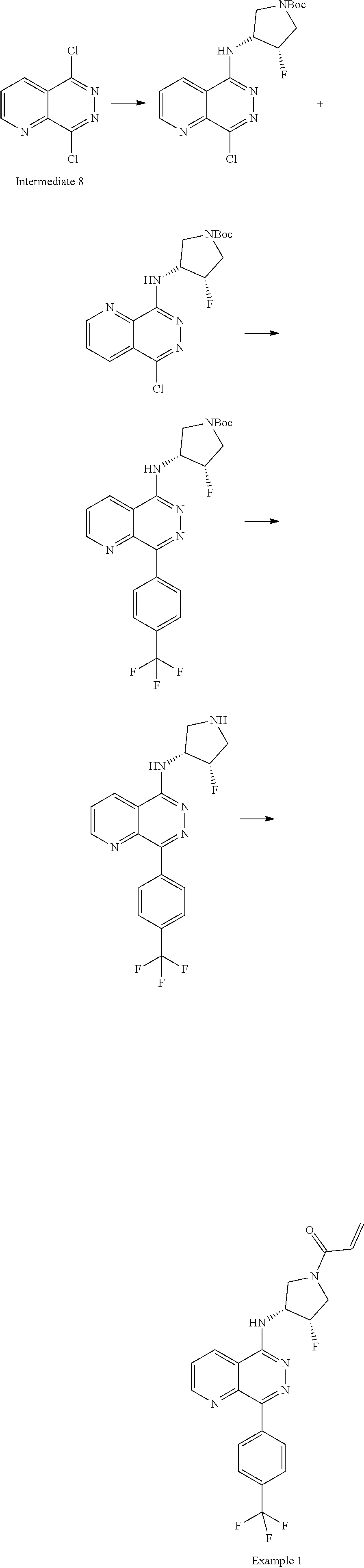

### tert-butyl (3R,4S)-3-((8-chloropyrido[2,3-d]pyridazin-5-yl)amino)-4-fluoropyrrolidine-1-carboxylate and tert-butyl (3R,4S)-3-((5-chloropyrido[2,3-d]pyridazin-8-yl)amino)-4-fluoropyrrolidine-1-carboxylate

DIPEA (437 μL, 2.50 mmol) was added to a solution of tert-butyl (3R,4S)-3-amino-4-fluoropyrrolidine-1-carboxylate (306 mg, 1.50 mmol) and 5,8-dichloropyrido[2,3-d]pyridazine (Intermediate 8, 250 mg, 1.25 mmol) in DMSO (3 mL). The reaction mixture was heated to 85° C. and stirred for 20 hrs. The reaction mixture was cooled to rt and directly purified on reverse phase C18 column (0 to 100% MeCN in H₂O w/0.1% HCO₂H) to yield two regioisomers: Peak A=tert-butyl (3R,4S)-3-((8-chloropyrido[2,3-d]pyridazin-5-yl)amino)-4-fluoropyrrolidine-1-carboxylate (165 mg, 36% yield) as a white solid. m/z: (ES⁺) [M+H]⁺=351; Peak B=tert-butyl (3R,4S)-3-((5-chloropyrido[2,3-d]pyridazin-8-yl)amino)-4-fluoropyrrolidine-1-carboxylate (90 mg, 20% yield) as a white solid. m/z: (ES⁺) [M+H]⁺=351.

### N-((3R,4S)-4-fluoropyrrolidin-3-yl)-8-(4-(trifluoromethyl)phenyl)pyrido[2,3-d]pyridazin-5-amine, HCl

(4-(Trifluoromethyl)phenyl)boronic acid (102 mg, 0.540 mmol), tert-butyl (3R,4S)-3-((8-chloropyrido[2,3-d]pyridazin-5-yl)amino)-4-fluoropyrrolidine-1-carboxylate (165 mg, 0.450 mmol), PdCl₂(dppf) (33 mg, 0.040 mmol) and Cs₂CO₃ (292 mg, 0.900 mmol) were diluted in 1,4-dioxane (6 mL) and water (1.5 mL) under an atmosphere of N₂. The reaction mixture was heated to 90° C. and stirred for 4 hrs. The reaction mixture was cooled to rt, diluted with DCM (^{˜}100 mL), and washed with water (20 mL). The organic layer was dried over Na₂SO₄, filtered and concentrated to dryness. The crude material was purified by flash silica chromatography (0 to 100% EtOAc in hexanes) to afford tert-butyl (3S,4R)-3-fluoro-4-((8-(4-(trifluoromethyl)phenyl)pyrido[2,3-d]pyridazin-5-yl)amino)pyrrolidine-1-carboxylate (163 mg, 76% yield) as a yellow solid. ¹H NMR (500 MHz, CDCl₃) δ 1.33-1.55 (9H, m), 3.51-3.77 (3H, m), 3.80-3.93 (1H, m), 4.90-5.14 (1H, m), 5.35-5.63 (1H, m), 7.77-7.91 (3H, m), 7.97 (1H, dd), 8.19 (2H, d), 8.84-9.04 (1H, m), 9.20 (1H, dd); m/z: (ES⁺) [M+H]⁺=478.

### N-((3R,4S)-4-fluoropyrrolidin-3-yl)-8-(4-(trifluoromethyl)phenyl)pyrido[2,3-d]pyridazin-5-amine, HCl

HCl (4 M in dioxane, 5.0 mL, 20 mmol) was added to a solution of tert-butyl (3S,4R)-3-fluoro-4-((8-(4-(trifluoromethyl)phenyl)pyrido[2,3-d]pyridazin-5-yl)amino)pyrrolidine-1-carboxylate (163 mg, 0.340 mmol) in MeOH (1 mL) and the reaction stirred at rt for 1 h. The volatiles were removed under reduced pressure to yield N-((3R,4S)-4-fluoropyrrolidin-3-yl)-8-(4-(trifluoromethyl)phenyl)pyrido[2,3-d]pyridazin-5-amine, HCl (140 mg, 99% yield). ¹H NMR (500 MHz, DMSO-d6) δ 3.43-3.52 (1H, m), 3.66-3.77 (3H, m), 4.97-5.20 (1H, m), 5.48-5.74 (1H, m), 7.89 (2H, d), 8.05 (1H, dd), 8.17 (2H, d), 8.51-8.83 (1H, m), 9.16 (1H, br d), 9.26 (1H, dd), 9.45-9.63 (1H, m), 9.77-9.98 (1H, m); m/z: (ES⁺) [M+H]⁺=377.

### 1-((3S,4R)-3-fluoro-4-((8-(4-(trifluoromethyl)phenyl)pyrido[2,3-d]pyrrolidin-5-yl)amino) pyrrolidine-1-yl)prop-2-en-1-one

A solution of acryloyl chloride (32 mg, 0.36 mmol) in DCM (1 mL) was added dropwise to a solution of N-((3R,4S)-4-fluoropyrrolidin-3-yl)-8-(4-(trifluoromethyl)phenyl)pyrido[2,3-d]pyridazin-5-amine, HCl (140 mg, 0.34 mmol) and Et₃N (189 μL, 1.35 mmol) in DCM (10 mL) at 0° C. The reaction mixture was stirred at 0° C. for 1 h. The reaction mixture was quenched with water (˜20 mL) and extracted with DCM (3×20 mL). The organics were dried over Na₂SO₄, filtered and concentrated to dryness. The crude material was purified by flash silica chromatography (0 to 100% EtOAc in hexanes) to afford 1-((3S,4R)-3-fluoro-4-((8-(4-(trifluoromethyl)phenyl)pyrido[2,3-d]pyridazin-5-yl)amino)pyrrolidin-1-yl)prop-2-en-1-one (Example 1, 75 mg, 51% yield) as a white amorphous solid. ¹H NMR (500 MHz, DMSO-d6) δ 3.63-3.92 (2H, m), 3.95-4.32 (2H, m), 4.92-5.26 (1H, m), 5.42-5.67 (1H, m), 5.74 (1H, ddd), 6.12-6.29 (1H, m), 6.54-6.74 (1H, m), 7.85 (2H, d), 7.92-8.03 (2H, m), 8.19 (2H, d), 8.98 (1H, ddd), 9.20 (1H, dt); m/z: (ES⁺) [M+H]⁺=432.

### Example 2: 1-((3S,4R)-3-fluoro-4-((5-(4-(trifluoromethyl)phenyl)pyrido[2,3-d]pyridazin-8-yl)amino)pyrrolidin-1-yl)prop-2-en-1-one

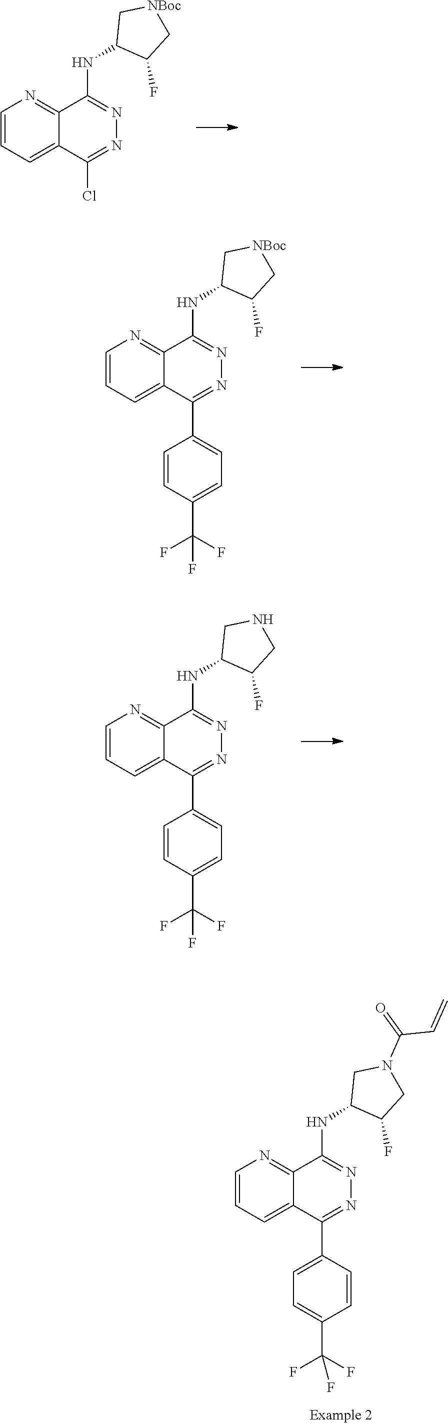

### tert-butyl (3S,4R)-3-fluoro-4-((5-(4-(trifluoromethyl)phenyl)pyrido[2,3-d]pyridazin-8-yl)amino)pyrrolidine-1-carboxylate

(4-(Trifluoromethyl)phenyl)boronic acid (56 mg, 0.29 mmol), tert-butyl (3R,4S)-3-((5-chloropyrido[2,3-d]pyridazin-8-yl)amino)-4-fluoropyrrolidine-1-carboxylate (90 mg, 0.24 mmol), PdCl₂(dppf) (18 mg, 0.020 mmol) and Cs₂CO₃ (159 mg, 0.490 mmol) were diluted in 1,4-dioxane (6 mL) and water (1.5 mL) under an atmosphere of N₂. The reaction mixture was heated to 90° C. and stirred for 4 hrs. The reaction mixture was cooled to rt, diluted with DCM (30 mL), and washed with water. The organic layer was dried over Na₂SO₄, filtered and concentrated to dryness. The crude material was purified by flash silica chromatography (0 to 100% EtOAc in hexanes) to afford tert-butyl (3S,4R)-3-fluoro-4-((5-(4-(trifluoromethyl)phenyl)pyrido[2,3-d]pyridazin-8-yl)amino)pyrrolidine-1-carboxylate (96 mg, 82% yield) as a yellow solid. ¹H NMR (500 MHz, CDCl₃) δ 1.43 (9H, s), 3.43-3.56 (1H, m), 3.58-3.81 (2H, m), 3.84-4.00 (1H, m), 4.87-5.13 (1H, m), 5.35-5.58 (1H, m), 7.53-7.74 (1H, m), 7.84-8.02 (5H, m), 8.29 (1H, dd), 9.20 (1H, dd); m/z: (ES⁺) [M+H]⁺=479.

### N-((3R,4S)-4-fluoropyrrolidin-3-yl)-5-(4-(trifluoromethyl)phenyl)pyrido[2,3-d]pyridazin-8-amine

HCl (4 M in dioxane, 6.0 mL, 24 mmol) was added to a solution of tert-butyl (3S,4R)-3-fluoro-4-((5-(4-(trifluoromethyl)phenyl)pyrido[2,3-d]pyridazin-8-yl)amino)pyrrolidine-1-carboxylate (96 mg, 0.20 mmol) in MeOH (1 mL) and the reaction mixture stirred at rt for 1 h. The volatiles were removed under reduced pressure to yield N-((3R,4S)-4-fluoropyrrolidin-3-yl)-5-(4-(trifluoromethyl)phenyl)pyrido[2,3-d]pyridazin-8-amine, HCl (81 mg, 98% yield). ¹H NMR (500 MHz, DMSO-d6) δ 3.39-3.52 (1H, m), 3.63-3.70 (3H, m), 5.02-5.20 (1H, m), 5.49-5.67 (1H, m), 7.84-7.94 (2H, m), 7.96 (2H, s), 8.02 (1H, dd), 8.34 (1H, dd), 8.44-8.76 (1H, m), 9.26 (1H, dd), 9.51-9.62 (1H, m), 9.65-9.79 (1H, m); m/z: (ES⁺) [M+H]⁺=377.

### 1-((3S,4R)-3-fluoro-4-((5-(4-(trifluoromethyl)phenyl)pyrido[2,3-d]pyridazin-8-yl)amino)pyrrolidin-1-yl)prop-2-en-1-one

A solution of acryloyl chloride (18 mg, 0.20 mmol) in DCM (1 mL) was added dropwise to a solution of N-((3R,4S)-4-fluoropyrrolidin-3-yl)-5-(4-(trifluoromethyl)phenyl)pyrido[2,3-d]pyridazin-8-amine, HCl (80 mg, 0.19 mmol) and Et₃N (0.11 mL, 0.77 mmol) in DCM (10 mL) at 0° C. The reaction mixture was stirred at 0° C. for 1 h. The reaction mixture was quenched with water (10 mL) and extracted with DCM (3×10 mL). The combined organics were dried over Na₂SO₄, filtered and concentrated to dryness. The crude material was purified by flash silica chromatography (0 to 100% EtOAc in hexanes) to afford 1-((3S,4R)-3-fluoro-4-((5-(4-(trifluoromethyl)phenyl)pyrido[2,3-d]pyridazin-8-yl)amino)pyrrolidin-1-yl)prop-2-en-1-one (Example 2, 44 mg, 52% yield) as a white amorphous solid. ¹H NMR (500 MHz, DMSO-d6) δ 3.57-3.92 (2H, m), 3.95-4.08 (1H, m), 4.07-4.31 (1H, m), 4.93-5.25 (1H, m), 5.41-5.63 (1H, m), 5.67-5.80 (1H, m), 6.19 (1H, dt), 6.60 (1H, ddd), 7.58-7.80 (1H, m), 7.86-8.00 (5H, m), 8.29 (1H, ddd), 9.20 (1H, dt); m/z: (ES⁺) [M+H]⁺=432.

### Example 3: 1-((3R,4R)-3-fluoro-4-((8-(4-(trifluoromethyl)phenyl)pyrido[2,3-d]pyridazin-5-yl)amino)pyrrolidin-1-yl)prop-2-en-1-one

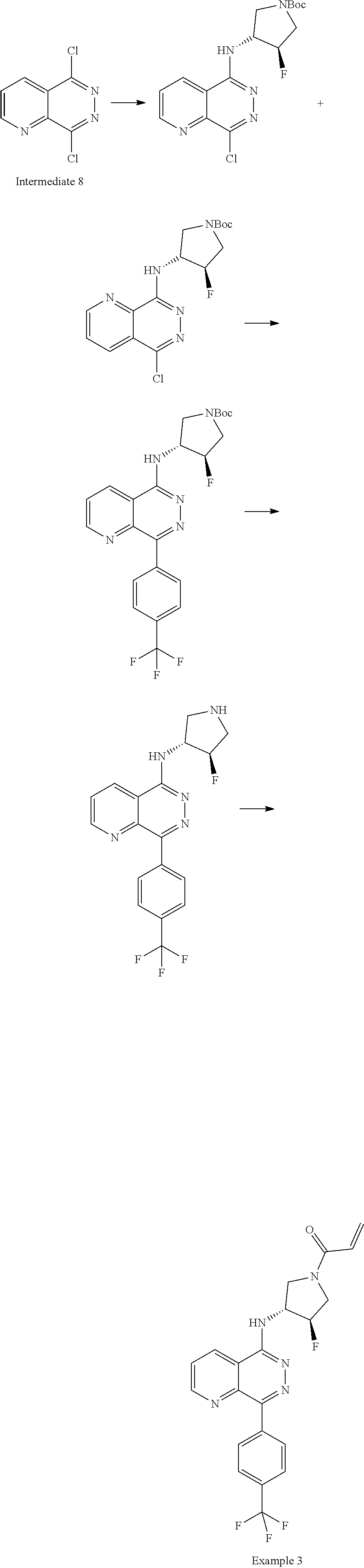

### tert-butyl (3R,4R)-3-((8-chloropyrido[2,3-d]pyridazin-5-yl)amino)-4-fluoropyrrolidine-1-carboxylate and tert-butyl (3R,4R)-3-((5-chloropyrido[2,3-d]pyridazin-8-yl)amino)-4-fluoropyrrolidine-1-carboxylate

DIPEA (0.30 mL, 1.7 mmol) was added to a solution of tert-butyl (3R,4R)-3-amino-4-fluoropyrrolidine-1-carboxylate (174 mg, 0.850 mmol) and 5,8-dichloropyrido[2,3-d]pyridazine (Intermediate 8, 170 mg, 0.850 mmol) in DMSO (3 mL). The reaction mixture was heated to 85° C. and stirred for 20 hrs. The reaction mixture was cooled to rt and directly purified on reverse phase C18 column (0 to 100% MeCN in H₂O w/0.1% HCO₂H) to yield two regioisomers: Peak A=tert-butyl (3R,4R)-3-((8-chloropyrido[2,3-d]pyrrolidin-5-yl)amino)-4-fluoropyrrolidine-1-carboxylate (82 mg, 26% yield) as a white solid, m/z: (ES⁺) [M+H]⁺=368; Peak B=tert-butyl (3R,4R)-3-((5-chloropyrido[2,3-d]pyrrolidin-8-yl)amino)-4-fluoropyrrolidine-1-carboxylate (110 mg, 35% yield) as a white solid, m/z: (ES⁺) [M+H]⁺=368.

### tert-butyl (3R,4R)-3-fluoro-4-((8-(4-(trifluoromethyl)phenyl)pyrido[2,3-d]pyridazin-5-yl)amino)pyrrolidine-1-carboxylate

(4-(Trifluoromethyl)phenyl)boronic acid (51 mg, 0.27 mmol), tert-butyl (3R,4R)-3-((8-chloropyrido[2,3-d]pyrrolidin-5-yl)amino)-4-fluoropyrrolidine-1-carboxylate (82 mg, 0.22 mmol), PdCl₂(dppf) (16 mg, 0.020 mmol) and Cs₂CO₃ (145 mg, 0.450 mmol) were diluted in 1,4-dioxane (6 mL) and water (1.5 mL) under an atmosphere of N₂. The reaction mixture was heated to 90° C. and stirred for 4 hrs. The reaction mixture was cooled to rt, diluted with DCM (40 mL), and washed with water (10 mL). The organic layer was dried over Na₂SO₄, filtered and concentrated to dryness. The crude material was purified by flash silica chromatography (0 to 100% EtOAc in hexanes) to afford tert-butyl (3R,4R)-3-fluoro-4-((8-(4-(trifluoromethyl)phenyl)pyrido[2,3-d]pyridazin-5-yl)amino)pyrrolidine-1-carboxylate (43 mg, 40% yield) as a yellow solid. ¹H NMR (500 MHz, CDCl₃) δ 1.53 (9H, s), 3.56-3.94 (3H, m), 3.94-4.07 (1H, m), 4.97-5.25 (1H, m), 5.27-5.80 (1H, m), 6.04-6.60 (1H, m), 7.79 (3H, br d), 8.25 (2H, br d), 8.37-8.82 (1H, m), 9.22 (1H, d); m/z: (ES⁺) [M+H]⁺=478.

### N-((3R,4R)-4-fluoropyrrolidin-3-yl)-8-(4-(trifluoromethyl)phenyl)pyrido[2,3-d]pyridazin-5-amine

HCl (4 M in dioxane, 3.0 mL, 12 mmol) was added to a solution of tert-butyl (3R,4R)-3-fluoro-4-((8-(4-(trifluoromethyl)phenyl)pyrido[2,3-d]pyridazin-5-yl)amino)pyrrolidine-1-carboxylate (43 mg, 0.090 mmol) in MeOH (1 mL) and the reaction mixture stirred at rt for 1 hr. The volatiles were removed under reduced pressure to afford N-((3R,4R)-4-fluoropyrrolidin-3-yl)-8-(4-(trifluoromethyl)phenyl)pyrido[2,3-d]pyridazin-5-amine, HCl (36 mg, 100% yield). ¹H NMR (500 MHz, DMSO-d6) δ 3.43-3.50 (1H, m), 3.66-3.73 (2H, m), 3.75-3.84 (1H, m), 4.98-5.14 (1H, m), 5.45-5.69 (1H, m), 7.88 (2H, d), 8.02 (1H, dd), 8.20 (2H, d), 8.25-8.39 (1H, m), 9.01 (1H, br d), 9.17-9.32 (1H, m), 9.48-9.73 (2H, m); m/z: (ES⁺) [M+H]⁺=378.

### 1-((3R,4R)-3-fluoro-4-((8-(4-(trifluoromethyl)phenyl)pyrido[2,3-d]pyridazin-5-yl)amino)pyrrolidin-1-yl)prop-2-en-1-one

A solution of acryloyl chloride (8 mg, 0.09 mmol) in DCM (1 mL) was added dropwise to a solution of N-((3R,4R)-4-fluoropyrrolidin-3-yl)-8-(4-(trifluoromethyl)phenyl)pyrido[2,3-d]pyridazin-5-amine, HCl (35 mg, 0.080 mmol) and Et₃N (47 μl, 0.34 mmol) in CH₂Cl₂ (6 mL) at 0° C. The mixture was stirred at 0° C. for 1 hr. The reaction mixture was quenched with water (5 mL) and extracted with DCM (3×10 mL). The combined organics were dried over Na₂SO₄, filtered and concentrated to dryness. The crude material was purified by flash silica chromatography (0 to 100% EtOAc in hexanes) to afford 1-((3R,4R)-3-fluoro-4-((8-(4-(trifluoromethyl)phenyl)pyrido[2,3-d]pyridazin-5-yl)amino)pyrrolidin-1-yl)prop-2-en-1-one (Example 3, 18 mg, 49% yield) as a white amorphous solid. ¹H NMR (500 MHz, DMSO-d6) δ 3.65-3.89 (2H, m), 3.91-4.17 (2H, m), 4.85-5.10 (1H, m), 5.28-5.51 (1H, m), 5.73 (1H, ddd), 6.21 (1H, dt), 6.65 (1H, dt), 7.86 (3H, br d), 7.95 (1H, ddd), 8.19 (2H, d), 8.89 (1H, ddd), 9.11-9.29 (1H, m); m/z: (ES⁺) [M+H]⁺=432.

### Example 4: 1-((3R,4R)-3-fluoro-4-((5-(4-(trifluoromethyl)phenyl)pyrido[2,3-d]pyridazin-8-yl)amino)pyrrolidin-1-yl)prop-2-en-1-one

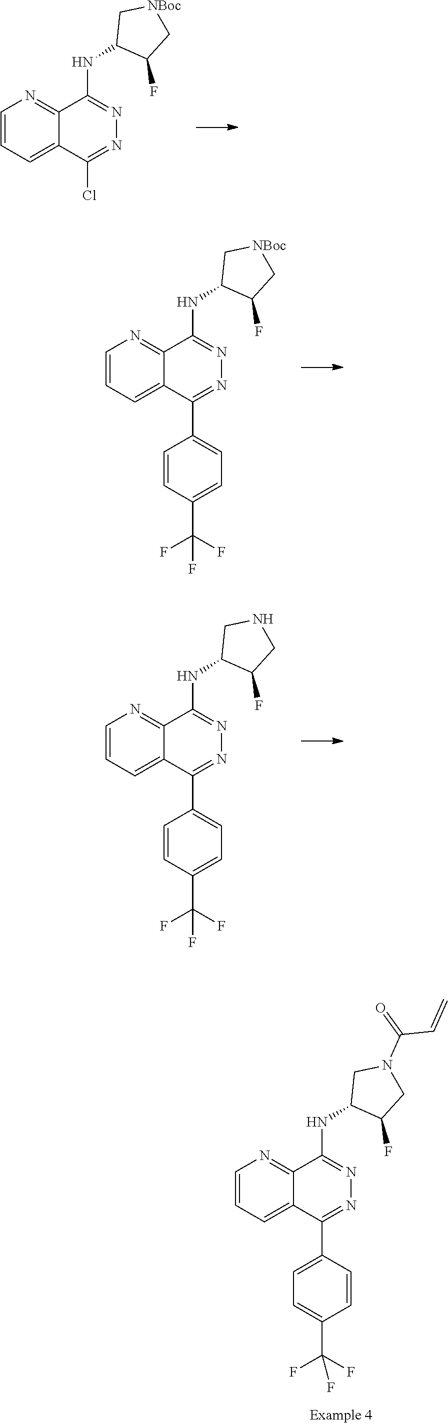

### tert-butyl (3R,4R)-3-fluoro-4-((5-(4-(trifluoromethyl)phenyl)pyrido[2,3-d]pyridazin-8-yl)amino)pyrrolidine-1-carboxylate

(4-(Trifluoromethyl)phenyl)boronic acid (68 mg, 0.36 mmol), tert-butyl (3R,4R)-3-fluoro-4-((5-(4-(trifluoromethyl)phenyl)pyrido[2,3-d]pyridazin-8-yl)amino)pyrrolidine-1-carboxylate (110 mg, 0.300 mmol), PdCl₂(dppf) (22 mg, 0.030 mmol) and Cs₂CO₃ (195 mg, 0.600 mmol) were diluted in 1,4-dioxane (6 mL) and water (1.5 mL) under an atmosphere of N₂. The reaction mixture was heated to 90° C. and stirred for 4 hrs. The reaction mixture was cooled to rt, diluted with DCM (50 mL), and washed with water (10 mL). The organics were dried over Na₂SO₄, filtered and concentrated to dryness. The crude material was purified by flash silica chromatography (0 to 100% EtOAc in hexanes) to afford tert-butyl (3R,4R)-3-fluoro-4-((5-(4-(trifluoromethyl)phenyl)pyrido[2,3-d]pyridazin-8-yl)amino)pyrrolidine-1-carboxylate (65 mg, 46% yield) as a yellow solid. ¹H NMR (500 MHz, CDCl₃) δ 1.53 (9H, s), 3.64-3.88 (3H, m), 3.94-4.04 (1H, m), 4.91-5.14 (1H, m), 5.31-5.56 (1H, m), 6.66 (1H, br d), 7.54-8.05 (6H, m), 8.22-8.32 (1H, m), 9.08 (1H, br d); m/z: (ES⁺) [M+H]⁺=477.

### N-((3R,4R)-4-fluoropyrrolidin-3-yl)-5-(4-(trifluoromethyl)phenyl)pyrido[2,3-d]pyridazin-8-amine

HCl (4 M in dioxane, 3.0 mL, 12 mmol) was added to a solution of tert-butyl (3R,4R)-3-fluoro-4-((5-(4-(trifluoromethyl)phenyl)pyrido[2,3-d]pyridazin-8-yl)amino)pyrrolidine-1-carboxylate (65 mg, 0.14 mmol) in MeOH (1 mL) and the reaction mixture was stirred at rt for 1 h. The volatiles were removed under reduced pressure to afford N-((3R,4R)-4-fluoropyrrolidin-3-yl)-5-(4-(trifluoromethyl)phenyl)pyrido[2,3-d]pyridazin-8-amine, HCl (56 mg, 100% yield). ¹H NMR (500 MHz, DMSO-d6) δ 3.41-3.52 (1H, m), 3.66-3.83 (3H, m), 5.05-5.20 (1H, m), 5.44-5.68 (1H, m), 7.83-7.95 (2H, m), 7.95-7.99 (2H, m), 8.01 (1H, dd), 8.35 (1H, dd), 8.50-8.69 (1H, m), 9.25 (1H, dd), 9.38-9.49 (1H, m), 9.69-9.88 (1H, m); m/z: (ES⁺) [M+H]⁺=377.

### 1-((3R,4R)-3-fluoro-4-((5-(4-(trifluoromethyl)phenyl)pyrido[2,3-d]pyridazin-8-yl)amino)pyrrolidin-1-yl)prop-2-en-1-one

A solution of acryloyl chloride (13 mg, 0.14 mmol) in DCM (1 mL) was added slowly to a solution of N-((3R,4R)-4-fluoropyrrolidin-3-yl)-5-(4-(trifluoromethyl)phenyl)pyrido[2,3-d]pyridazin-8-amine, HCl (55 mg, 0.13 mmol) and Et₃N (74 μl, 0.53 mmol) in DCM (8 mL) at 0° C. The reaction mixture was stirred at 0° C. for 1 h. The reaction mixture was quenched with water (10 mL) and extracted with DCM (2×20 mL). The combined organics were dried over Na₂SO₄, filtered and concentrated to dryness. The crude material was purified by flash silica chromatography (0 to 100% EtOAc in hexanes) to afford 1-((3R,4R)-3-fluoro-4-((5-(4-(trifluoromethyl)phenyl)pyrido[2,3-d]pyridazin-8-yl)amino)pyrrolidin-1-yl)prop-2-en-1-one (Example 4, 26 mg, 45% yield) as a white amorphous solid. ¹H NMR (500 MHz, DMSO-d6) δ 3.66-3.93 (2H, m), 3.93-4.18 (2H, m), 4.88-5.08 (1H, m), 5.37-5.60 (1H, m), 5.73 (1H, dt), 6.20 (1H, dt), 6.64 (1H, ddd), 7.84-7.99 (5H, m), 8.08-8.26 (1H, m), 8.28-8.36 (1H, m), 9.15-9.28 (1H, m); m/z: (ES⁺) [M+H]⁺=432.

### Example 5: 2-fluoro-1-((3S,4R)-3-fluoro-4-((8-(4-(trifluoromethyl)phenyl)pyrido[2,3-d]pyridazin-5-yl)amino)pyrrolidin-1-yl)prop-2-en-1-one

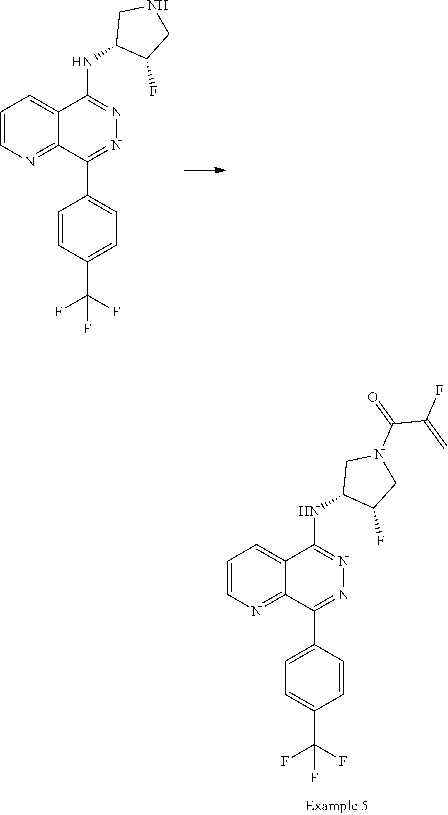 [SMILES: F[C@H]1CNC[C@H]1Nc1nnc(-c2ccc(C(F)(F)F)cc2)c2ncccc12 ; C=C(F)C(=O)N1C[C@H](F)[C@H](Nc2nnc(-c3ccc(C(F)(F)F)cc3)c3ncccc23)C1]

DIPEA (0.13 mL, 0.75 mmol) was added to a solution of N-((3R,4S)-4-fluoropyrrolidin-3-yl)-8-(4-(trifluoromethyl)phenyl)pyrido[2,3-d]pyridazin-5-amine, HCl (78 mg, 0.19 mmol), 2-fluoroacrylic acid (17 mg, 0.19 mmol) and 2-(3H-[1,2,3]triazolo[4,5-b]pyridin-3-yl)-1,1,3,3-tetramethylisouronium hexafluorophosphate(V) (107 mg, 0.280 mmol) in DMF (5 mL) at rt and the resulting mixture was stirred for 15 hrs. The reaction mixture was diluted with DCM (50 mL) and saturated aq. NaHCO₃ (15 mL). The phases were separated and the aqueous layer was extracted with DCM (2×15 mL). The combined organics were dried over Na₂SO₄, filtered and concentrated to dryness. The crude material was purified on a reverse phase C18 column (0 to 100% MeCN in H₂O w/0.1% HCO₂H) to afford 2-fluoro-1-((3S,4R)-3-fluoro-4-((8-(4-(trifluoromethyl)phenyl)pyrido[2,3-d]pyridazin-5-yl)amino)pyrrolidin-1-yl)prop-2-en-1-one (Example 5, 35 mg, 41% yield) as a white amorphous solid. ¹H NMR (500 MHz, DMSO-d6) δ 3.69-4.32 (4H, m), 4.96-5.22 (1H, m), 5.33-5.68 (3H, m), 7.86 (2H, d), 7.92-8.05 (2H, m), 8.19 (2H, d), 8.98 (1H, t), 9.21 (1H, d); m/z: (ES⁺) [M+H]⁺=450.

### Example 6: 2-fluoro-1-((3S,4R)-3-fluoro-4-((5-(4-(trifluoromethyl)phenyl)pyrido[2,3-d]pyridazin-8-yl)amino)pyrrolidin-1-yl)prop-2-en-1-one

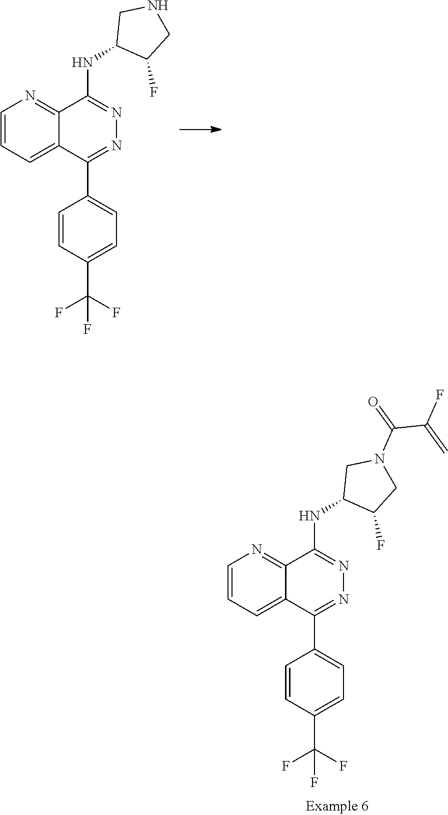 [SMILES: F[C@H]1CNC[C@H]1Nc1nnc(-c2ccc(C(F)(F)F)cc2)c2cccnc12 ; C=C(F)C(=O)N1C[C@H](F)[C@H](Nc2nnc(-c3ccc(C(F)(F)F)cc3)c3cccnc23)C1]

DIPEA (0.194 mL, 1.11 mmol) was added to a solution of N-((3R,4S)-4-fluoropyrrolidin-3-yl)-5-(4-(trifluoromethyl)phenyl)pyrido[2,3-d]pyridazin-8-amine (105 mg, 0.280 mmol), 2-fluoroacrylic acid (25 mg, 0.28 mmol) and 2-(3H-[1,2,3]triazolo[4,5-b]pyridin-3-yl)-1,1,3,3-tetramethylisouronium hexafluorophosphate(V) (159 mg, 0.420 mmol) in DMF (5 mL) at rt and the resulting mixture was stirred for 15 hrs. The reaction mixture was diluted with DCM (50 mL) and saturated aq. NaHCO₃ (10 mL). The phases were separated and the aqueous layer was extracted with DCM (2×15 mL). The combined organics were dried over Na₂SO₄, filtered and concentrated to dryness. The crude material was purified on a reverse phase C18 column (0 to 100% MeCN in H₂O w/0.1% HCO₂H) to afford 2-fluoro-1-((3S,4R)-3-fluoro-4-((5-(4-(trifluoromethyl)phenyl)pyrido[2,3-d]pyridazin-8-yl)amino)pyrrolidin-1-yl)prop-2-en-1-one (Example 6, 41 mg, 33% yield) as a white amorphous solid. ¹H NMR (500 MHz, DMSO-d6) δ 3.69-4.38 (4H, m), 4.97-5.19 (1H, m), 5.32-5.64 (3H, m), 7.67-7.86 (1H, m), 7.87-8.03 (5H, m), 8.29 (1H, dd), 9.21 (1H, d); m/z: (ES⁺) [M+H]⁺=450.

### Example 7: (S)-1-(3-((8-(4-(trifluoromethyl)phenyl)pyrido[2,3-d]pyridazin-5-yl)amino)pyrrolidin-1-yl)prop-2-en-1-one

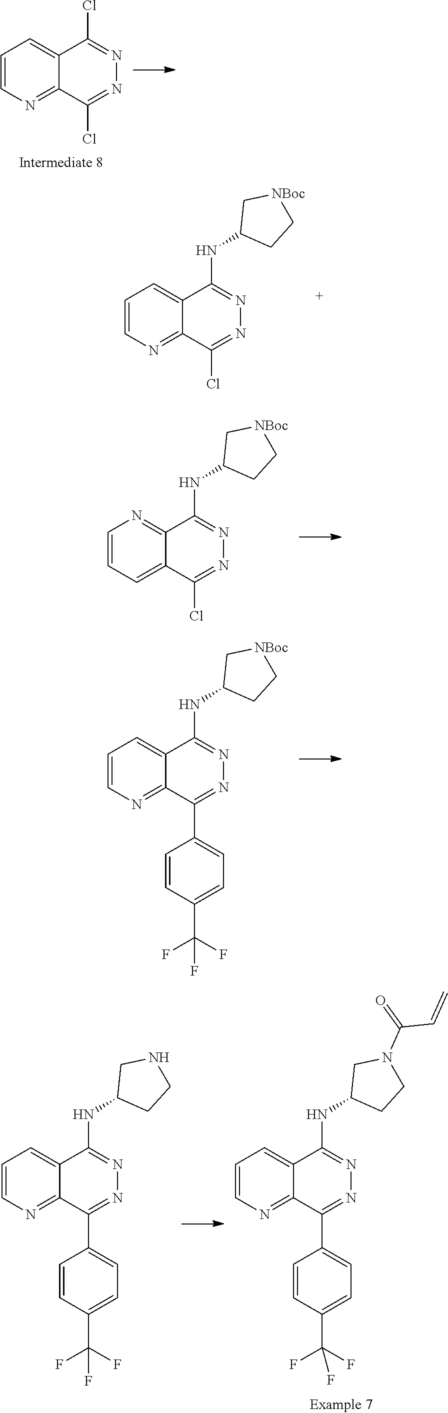

DIPEA (1.05 mL, 6.00 mmol) was added to a solution of tert-butyl (S)-3-aminopyrrolidine-1-carboxylate (559 mg, 3.00 mmol) and 5,8-dichloropyrido[2,3-d]pyridazine (Intermediate 8, 600 mg, 3.00 mmol) in DMSO (6 mL). The reaction mixture was heated to 85° C. and stirred for 20 hrs. The reaction mixture was cooled to rt and directly purified on a reverse phase C18 column (0 to 100% MeCN in H₂O w/0.1% HCO₂H) to yield two regioisomers: tert-butyl (S)-3-((8-chloropyrido[2,3-d]pyridazin-5-yl)amino)pyrrolidine-1-carboxylate (Peak A, 212 mg, 20% yield) as a yellow solid and tert-butyl (S)-3-((5-chloropyrido[2,3-d]pyridazin-8-yl)amino)pyrrolidine-1-carboxylate (Peak B, 149 mg, 14% yield) as a white solid.

### tert-butyl (S)-3-((8-chloropyrido[2,3-d]pyridazin-5-yl)amino)pyrrolidine-1-carboxylate

¹H NMR (500 MHz, DMSO-d6) δ 1.42 (9H, br d), 1.99-2.12 (1H, m), 2.18-2.33 (1H, m), 3.34-3.43 (2H, m), 3.45-3.57 (1H, m), 3.63-3.81 (1H, m), 4.68 (1H, br dd), 7.81 (1H, d), 8.01 (1H, dd), 8.92 (1H, br d), 9.24 (1H, dd); m/z: (ES⁺) [M+H]⁺=350.

### tert-butyl (S)-3-((5-chloropyrido[2,3-d]pyridazin-8-yl)amino)pyrrolidine-1-carboxylate

¹H NMR (500 MHz, DMSO-d6) δ 1.41 (9H, br d), 2.06-2.32 (2H, m), 3.33-3.41 (2H, m), 3.48 (1H, br s), 3.62-3.78 (1H, m), 4.60-4.79 (1H, m), 7.87 (1H, br d), 8.06 (1H, dd), 8.49 (1H, dd), 9.21 (1H, dd); m/z: (ES⁺) [M+H]⁺=350.

### tert-butyl (S)-3-((8-(4-(trifluoromethyl)phenyl)pyrido[2,3-d]pyridazin-5-yl)amino)pyrrolidine-1-carboxylate

(4-(Trifluoromethyl)phenyl)boronic acid (87 mg, 0.46 mmol), tert-butyl (S)-3-((8-chloropyrido[2,3-d]pyridazin-5-yl)amino)pyrrolidine-1-carboxylate (134 mg, 0.380 mmol), PdCl₂(dppf) (27 mg, 0.040 mmol) and Cs₂CO₃ (374 mg, 1.15 mmol) were diluted in 1,4-dioxane (6 mL) and water (1.5 mL) under an atmosphere of N₂. The reaction mixture was heated to 80° C. and stirred for 5 hrs. The reaction mixture was cooled to rt, diluted with water (20 mL), and extracted with DCM (3×30 mL). The combined organics were dried over Na₂SO₄, filtered and concentrated to dryness. The crude material was purified by flash silica chromatography (0 to 100% EtOAc in hexanes) to afford tert-butyl (S)-3-((8-(4-(trifluoromethyl)phenyl)pyrido[2,3-d]pyridazin-5-yl)amino)pyrrolidine-1-carboxylate (142 mg, 81% yield) as a light yellow solid. m/z: (ES⁺) [M+H]⁺=459.

### (S)-1-(3-((8-(4-(trifluoromethyl)phenyl)pyrido[2,3-d]pyridazin-5-yl)amino)pyrrolidin-1-yl)prop-2-en-1-one

HCl (4 M in dioxane, 4.0 mL, 16 mmol) was added to a solution of tert-butyl (S)-3-((8-(4-(trifluoromethyl)phenyl)pyrido[2,3-d]pyridazin-5-yl)amino)pyrrolidine-1-carboxylate (142 mg, 0.310 mmol) in MeOH (2 mL) at rt and the resulting mixture was stirred for 1 hr. The volatiles were removed under reduced pressure to afford (S)—N-(pyrrolidin-3-yl)-8-(4-(trifluoromethyl)phenyl)pyrido[2,3-d]pyridazin-5-amine, HCl which was used in next step without further purification.

(S)—N-(pyrrolidin-3-yl)-8-(4-(trifluoromethyl)phenyl)pyrido[2,3-d]pyridazin-5-amine, HCl from above was suspended in THF (6 mL) and saturated aq. NaHCO₃ (2 mL). Acryloyl chloride (38 μL, 0.46 mmol) was added and the reaction mixture was stirred at rt for 15 hrs. The reaction mixture was diluted with water (20 mL) and extracted with DCM (3×30 mL). The combined organics were dried over Na₂SO₄, filtered and concentrated to dryness. The crude material was purified on a reverse phase C18 column (0 to 100% MeCN in H₂O w/0.2% NH₄OH) to afford (S)-1-(3-((8-(4-(trifluoromethyl)phenyl)pyrido[2,3-d]pyridazin-5-yl)amino)pyrrolidin-1-yl)prop-2-en-1-one (Example 7, 85 mg, 67% yield) as a white amorphous solid. ¹H NMR (500 MHz, DMSO-d6) δ 2.12-2.44 (2H, m), 3.50-3.92 (3.5H, m), 4.09 (0.5H, dd), 4.78-5.01 (1H, m), 5.69 (1H, ddd), 6.17 (1H, ddd), 6.49-6.79 (1H, m), 7.74-7.89 (3H, m), 7.95 (1H, dt), 8.21 (2H, d), 8.83-9.00 (1H, m), 9.12-9.28 (1H, m); m/z: (ES⁺) [M+H]⁺=414.

### Example 8: (S)-1-(3-((5-(4-(trifluoromethyl)phenyl)pyrido[2,3-d]pyridazin-8-yl)amino)pyrrolidin-1-yl)prop-2-en-1-one

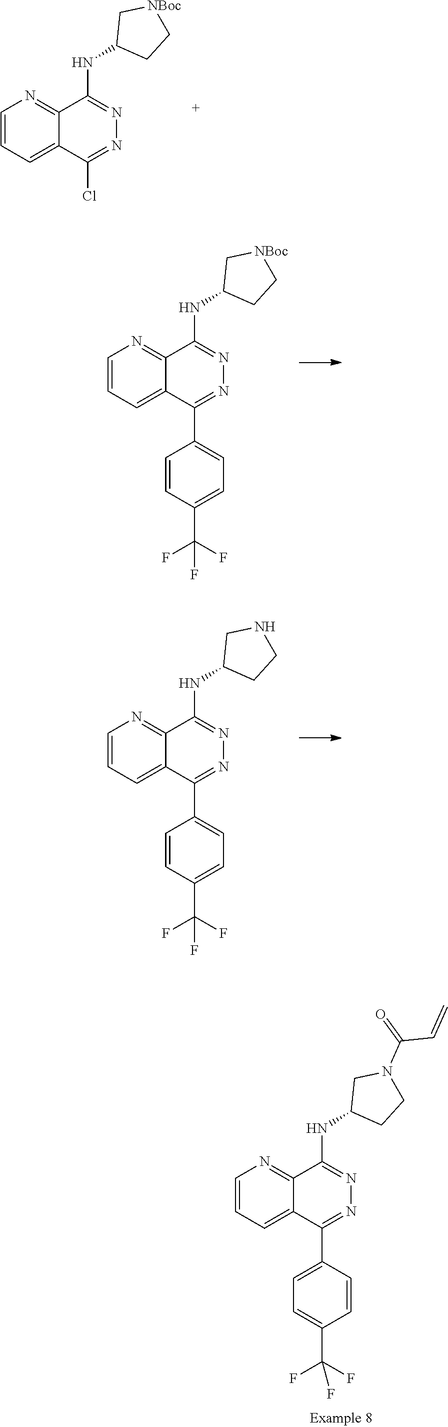

### tert-butyl (S)-3-((5-(4-(trifluoromethyl)phenyl)pyrido[2,3-d]pyridazin-8-yl)amino)pyrrolidine-1-carboxylate

(4-(Trifluoromethyl)phenyl)boronic acid (87 mg, 0.46 mmol), tert-butyl (S)-3-((5-chloropyrido[2,3-d]pyridazin-8-yl)amino)pyrrolidine-1-carboxylate (134 mg, 0.380 mmol), PdCl₂(dppf) (27 mg, 0.040 mmol) and Cs₂CO₃ (374 mg, 1.15 mmol) were diluted in 1,4-dioxane (6 mL) and water (1.5 mL) under an atmosphere of N₂. The reaction mixture was heated to 80° C. and stirred for 16 hrs. The reaction mixture was cooled to rt, diluted with water (20 mL), and extracted with DCM (3×30 mL). The combined organics were dried over Na₂SO₄, filtered and concentrated to dryness. The crude material was purified by flash silica chromatography (0 to 100% EtOAc in hexanes) to afford tert-butyl (S)-3-((5-(4-(trifluoromethyl)phenyl)pyrido[2,3-d]pyridazin-8-yl)amino)pyrrolidine-1-carboxylate (174 mg, 99% yield) as a white solid. m/z: (ES⁺) [M+H]⁺=460.

### (S)-1-(3-((5-(4-(trifluoromethyl)phenyl)pyrido[2,3-d]pyridazin-8-yl)amino)pyrrolidin-1-yl)prop-2-en-1-one

HCl (4 M in dioxane, 4.0 mL, 16 mmol) was added to a solution of tert-butyl (S)-3-((5-(4-(trifluoromethyl)phenyl)pyrido[2,3-d]pyridazin-8-yl)amino)pyrrolidine-1-carboxylate (174 mg, 0.380 mmol) in MeOH (2 mL) and the reaction was stirred at rt for 1 h. The volatiles were removed under reduced pressure to afford (S)—N-(pyrrolidin-3-yl)-5-(4-(trifluoromethyl)phenyl)pyrido[2,3-d]pyridazin-8-amine, HCl which was used directly without purification. (S)—N-(pyrrolidin-3-yl)-5-(4-(trifluoromethyl)phenyl)pyrido[2,3-d]pyridazin-8-amine, HCl from above was suspended in THF (6 mL) and saturated aq. NaHCO₃ (2 mL). Acryloyl chloride (46 μL, 0.57 mmol) was added and the reaction mixture stirred at rt for 15 hrs. The reaction mixture was diluted with water and extracted with DCM (3×20 mL). The combined organics were dried over Na₂SO₄, filtered and concentrated to dryness. The crude material was purified on a reverse phase C18 column (0 to 100% MeCN in H₂O w/0.2% NH₄OH) to afford (S)-1-(3-((5-(4-(trifluoromethyl)phenyl)pyrido[2,3-d]pyridazin-8-yl)amino)pyrrolidin-1-yl)prop-2-en-1-one (Example 8, 97 mg, 62% yield) as a white amorphous solid. ¹H NMR (500 MHz, DMSO-d6) δ 2.12-2.42 (2H, m), 3.41-4.14 (4H, m), 4.65-5.02 (1H, m), 5.66 (1H, ddd), 6.14 (1H, ddd), 6.48-6.74 (1H, m), 7.79-8.00 (6H, m), 8.26 (1H, dt), 9.05-9.35 (1H, m); m/z: (ES⁺) [M+H]⁺=414.

### Example 9: (S)-1-(3-((4-(4-(trifluoromethyl)phenyl)phthalazin-1-yl)amino)pyrrolidin-1-yl)prop-2-en-1-one

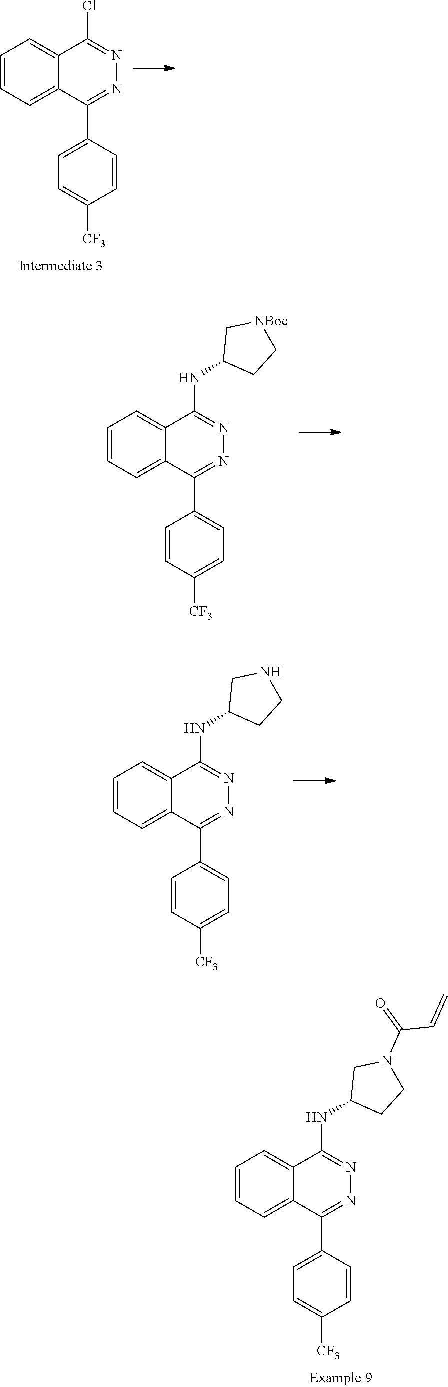 [SMILES: FC(F)(F)c1ccc(-c2nnc(Cl)c3ccccc23)cc1 ; CC(C)(C)OC(=O)N1CC[C@H](Nc2nnc(-c3ccc(C(F)(F)F)cc3)c3ccccc23)C1 ; FC(F)(F)c1ccc(-c2nnc(N[C@H]3CCNC3)c3ccccc23)cc1 ; C=CC(=O)N1CC[C@H](Nc2nnc(-c3ccc(C(F)(F)F)cc3)c3ccccc23)C1]

### tert-butyl (S)-3-((4-(4-(trifluoromethyl)phenyl)phthalazin-1-yl)amino)pyrrolidine-1-carboxylate

DIPEA (0.453 mL, 2.59 mmol) was added to a solution of tert-butyl (S)-3-aminopyrrolidine-1-carboxylate (338 mg, 1.81 mmol) and 1-chloro-4-(4-(trifluoromethyl)phenyl) phthalazine (Intermediate 3, 400 mg, 1.30 mmol) in DMSO (4 mL). The reaction mixture was heated to 85° C. and stirred for 72 hrs. The reaction mixture was cooled to rt, diluted with DCM (50 mL), and washed with water (15 mL). The organic layer was dried over Na₂SO₄, filtered and concentrated to dryness. The crude material was purified by flash silica chromatography (0 to 100% EtOAc in hexanes) to afford tert-butyl (S)-3-((4-(4-(trifluoromethyl)phenyl)phthalazin-1-yl)amino)pyrrolidine-1-carboxylate (593 mg, 100% yield) as a light-yellow solid. ¹H NMR (500 MHz, DMSO-d6) δ 1.42 (9H, br d), 2.04-2.20 (1H, m), 2.29 (1H, dt), 3.33-3.45 (2H, m), 3.52 (1H, dt), 3.66-3.84 (1H, m), 4.71-4.88 (1H, m), 7.57 (1H, br s), 7.80 (1H, d), 7.83-7.99 (6H, m), 8.50 (1H, d); m/z: (ES⁺) [M+H]⁺=459.

### (S)—N-(pyrrolidin-3-yl)-4-(4-(trifluoromethyl)phenyl)phthalazin-1-amine

HCl (4 M in dioxane, 10 mL, 40 mmol) was added to a solution of tert-butyl (S)-3-((4-(4-(trifluoromethyl)phenyl)phthalazin-1-yl)amino)pyrrolidine-1-carboxylate (593 mg, 1.30 mmol) in MeOH (3 mL) and the reaction mixture was stirred at rt for 1 h. The volatiles were removed under reduced pressure to afford (S)—N-(pyrrolidin-3-yl)-4-(4-(trifluoromethyl)phenyl)phthalazin-1-amine, HCl (463 mg, 90% yield) as an off white solid which was used without purification. ¹H NMR (500 MHz, DMSO-d6) δ 2.27-2.37 (1H, m), 2.37-2.45 (1H, m), 3.59-3.73 (4H, m), 4.84-5.00 (1H, m), 7.92 (3H, d), 8.02 (2H, d), 8.06-8.14 (1H, m), 8.14-8.25 (1H, m), 9.15 (1H, br d), 9.38-9.63 (2H, m); m/z: (ES⁺) [M+H]⁺=359.

### (S)-1-(3-((4-(4-(trifluoromethyl)phenyl)phthalazin-1-yl)amino)pyrrolidin-1-yl)prop-2-en-1-one

A solution of acryloyl chloride (77 μL, 0.96 mmol) in DCM (1 mL) was added dropwise to a solution of (S)—N-(pyrrolidin-3-yl)-4-(4-(trifluoromethyl)phenyl)phthalazin-1-amine, HCl (360 mg, 0.910 mmol) and Et₃N (381 μL, 2.74 mmol) in DCM (12 mL) and the reaction mixture was stirred at rt for 2 hrs.

The reaction mixture was diluted with water (10 mL) and extracted with DCM (3×20 mL). The combined organics were dried over Na₂SO₄, filtered and concentrated to dryness. The crude material was purified by flash silica chromatography (0 to 100% EtOAc in hexanes, then 0 to 15% MeOH in DCM) to afford impure material which was further purified on a reverse C18 column (0 to 100% MeCN in H₂O w/0.1% HCO₂H) to afford (S)-1-(3-((4-(4-(trifluoromethyl)phenyl)phthalazin-1-yl)amino)pyrrolidin-1-yl)prop-2-en-1-one (Example 9, 199 mg, 53% yield) as a white amorphous solid. ¹H NMR (500 MHz, DMSO-d6) δ 2.07-2.43 (2H, m), 3.52-3.76 (2.5H, m), 3.77-3.92 (1H, m), 4.07 (0.5H, dd), 4.72-5.01 (1H, m), 5.67 (1H, ddd), 6.16 (1H, ddd), 6.47-6.79 (1H, m), 7.60 (1H, br dd), 7.79 (1H, br d), 7.83-8.04 (5H, m), 8.49 (1H, dd); (m/z) (ES⁺) [M+H]⁺=413.

### Example 10: 1-((3S,4R)-3-fluoro-4-((4-(4-(trifluoromethyl)phenyl)phthalazin-1-yl)amino)pyrrolidin-1-yl)prop-2-en-1-one

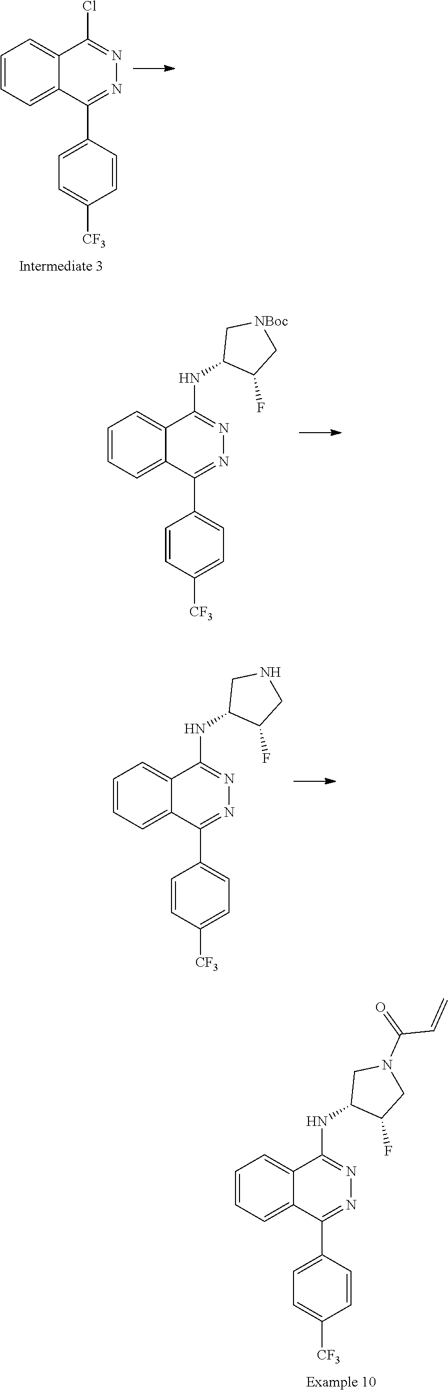 [SMILES: FC(F)(F)c1ccc(-c2nnc(Cl)c3ccccc23)cc1 ; CC(C)(C)OC(=O)N1C[C@H](F)[C@H](Nc2nnc(-c3ccc(C(F)(F)F)cc3)c3ccccc23)C1 ; F[C@H]1CNC[C@H]1Nc1nnc(-c2ccc(C(F)(F)F)cc2)c2ccccc12 ; C=CC(=O)N1C[C@H](F)[C@H](Nc2nnc(-c3ccc(C(F)(F)F)cc3)c3ccccc23)C1]

### tert-butyl (3S,4R)-3-fluoro-4-((4-(4-(trifluoromethyl)phenyl)phthalazin-1-yl)amino)pyrrolidine-1-carboxylate

1-Chloro-4-(4-(trifluoromethyl)phenyl)phthalazine (Intermediate 3, 150 mg, 0.490 mmol), tert-butyl (3R,4S)-3-amino-4-fluoropyrrolidine-1-carboxylate (119 mg, 0.580 mmol), Cs₂CO₃ (317 mg, 0.970 mmol) and Xphos Pd G4 (42 mg, 0.050 mmol) were diluted in 1,4-dioxane (10 mL) under an atmosphere of N₂. The reaction mixture was heated to 100° C. and stirred for 20 hrs. The reaction mixture was cooled to rt, diluted with water (20 mL), and extracted with DCM (3×40 mL). The combined organics were dried over Na₂SO₄, filtered and concentrated to dryness. The crude material was purified by flash silica chromatography (0 to 100% EtOAc in hexanes) to afford tert-butyl (3S,4R)-3-fluoro-4-((4-(4-(trifluoromethyl)phenyl)phthalazin-1-yl)amino)pyrrolidine-1-carboxylate as a yellow solid. m/z: (ES⁺) [M+H]⁺=477.

### N-((3R,4S)-4-fluoropyrrolidin-3-yl)-4-(4-(trifluoromethyl)phenyl)phthalazin-1-amine

HCl (4 M in dioxanes, 5 mL, 20 mmol) was added to solution of tert-butyl (3S,4R)-3-fluoro-4-((4-(4-(trifluoromethyl)phenyl)phthalazin-1-yl)amino)pyrrolidine-1-carboxylate (material from above) in MeOH (1 mL) at rt and the reaction mixture was stirred for 1 h. The volatiles were removed under reduced pressure and the resulting residue was purified on a reverse phase C18 column (0 to 100% MeCN in H₂O w/0.1% HCO₂H) to afford N-((3R,4S)-4-fluoropyrrolidin-3-yl)-4-(4-(trifluoromethyl)phenyl)phthalazin-1-amine, formic acid (68 mg, 34% yield over two steps) as a white solid. m/z: (ES⁺) [M+H]⁺=377.

### 1-((3S,4R)-3-fluoro-4-((4-(4-(trifluoromethyl)phenyl)phthalazin-1-yl)amino)pyrrolidin-1-yl)prop-2-en-1-one

A solution of acryloyl chloride (13 μL, 0.17 mmol) in DCM (1 mL) was added dropwise to a solution of N-((3R,4S)-4-fluoropyrrolidin-3-yl)-4-(4-(trifluoromethyl)phenyl)phthalazin-1-amine, formic acid salt (68 mg, 0.16 mmol) and Et₃N (67 μL, 0.48 mmol) in DCM (5 mL) at 0° C. The reaction mixture was stirred at 0° C. for 1 h. The reaction mixture was quenched with water (20 mL) and extracted with DCM (3×30 mL). The combined organics were dried over Na₂SO₄, filtered and concentrated to dryness. The crude material was purified by HPLC (Waters Xselect CSH C18 OBD column: 30 mm×100 mm, 5 μm; Mobile phase=30-60% MeCN:H₂O(w/0.2% NH₄OH); UV detection @ 254 nm; Flow rate=50 mL/min) to afford 1-((3S,4R)-3-fluoro-4-((4-(4-(trifluoromethyl)phenyl)phthalazin-1-yl)amino)pyrrolidin-1-yl)prop-2-en-1-one (Example 10, 16 mg, 23% yield) as a white amorphous solid. ¹H NMR (500 MHz, DMSO-d6) δ 3.64-3.92 (2H, m), 3.96-4.28 (2H, m), 4.90-5.25 (1H, m), 5.39-5.67 (1H, m), 5.73 (1H, td), 6.20 (1H, ddd), 6.53-6.76 (1H, m), 7.69 (1H, dd), 7.81 (1H, d), 7.84-7.95 (5H, m), 7.95-8.09 (1H, m), 8.56 (1H, t); m/z: (ES⁺) [M+H]⁺=431.

### Example 11: 1-((3R,4R)-3-fluoro-4-((4-(4-(trifluoromethyl)phenyl)phthalazin-1-yl)amino)pyrrolidin-1-yl)prop-2-en-1-one

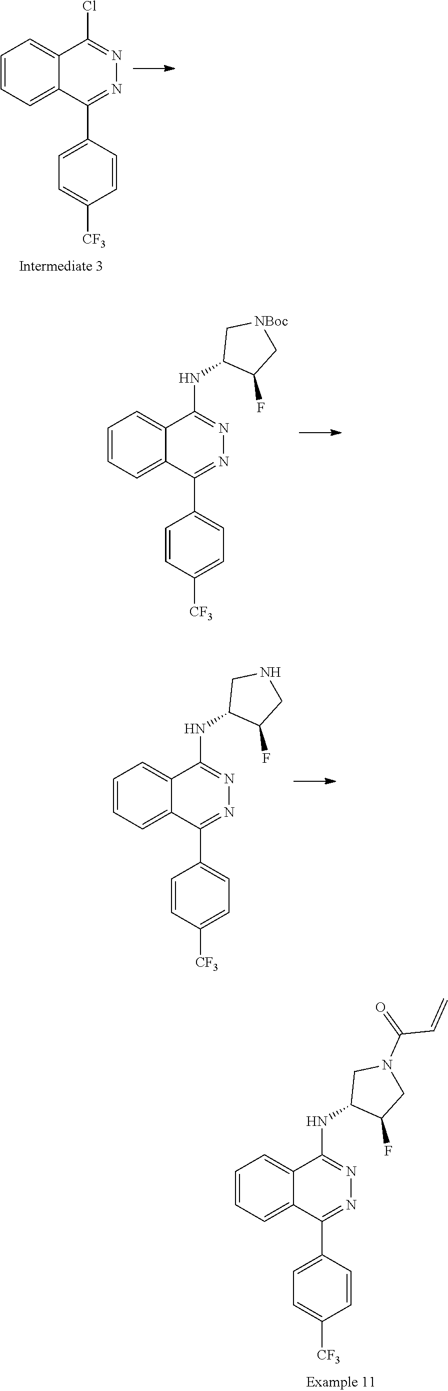 [SMILES: FC(F)(F)c1ccc(-c2nnc(Cl)c3ccccc23)cc1 ; CC(C)(C)OC(=O)N1C[C@@H](F)[C@H](Nc2nnc(-c3ccc(C(F)(F)F)cc3)c3ccccc23)C1 ; F[C@@H]1CNC[C@H]1Nc1nnc(-c2ccc(C(F)(F)F)cc2)c2ccccc12 ; C=CC(=O)N1C[C@@H](F)[C@H](Nc2nnc(-c3ccc(C(F)(F)F)cc3)c3ccccc23)C1]

### tert-butyl (3R,4R)-3-fluoro-4-((4-(4-(trifluoromethyl)phenyl)phthalazin-1-yl)amino)pyrrolidine-1-carboxylate

DIPEA (113 μL, 0.650 mmol) was added to a solution of tert-butyl (3R,4R)-3-amino-4-fluoropyrrolidine-1-carboxylate (66 mg, 0.32 mmol), 1-chloro-4-(4-(trifluoromethyl)phenyl)phthalazine (Intermediate 3, 100 mg, 0.32 mmol) in DMSO (3 mL). The reaction mixture was heated to 85° C. and stirred for 140 hrs. The reaction mixture was cooled to rt and diluted with water (10 mL) and DCM (60 mL). The phases were separated and the aqueous layer was extracted with DCM (2×20 mL). The combined organics were dried over Na₂SO₄, filtered and concentrated to dryness. The crude material was purified by flash silica chromatography (0 to 100% EtOAc in hexanes) to afford tert-butyl (3R,4R)-3-fluoro-4-((4-(4-(trifluoromethyl)phenyl)phthalazin-1-yl)amino)pyrrolidine-1-carboxylate. m/z: (ES⁺) [M+H]⁺=477.

### N-((3R,4R)-4-fluoropyrrolidin-3-yl)-4-(4-(trifluoromethyl)phenyl)phthalazin-1-amine

HCl (4 M in dioxane, 4 mL, 16 mmol) was added to a solution of tert-butyl (3R,4R)-3-fluoro-4-((4-(4-(trifluoromethyl)phenyl)phthalazin-1-yl)amino)pyrrolidine-1-carboxylate (material from above) in MeOH (1 mL) at rt and the reaction mixture stirred for 1 h. The volatiles were removed under reduced pressure and the resulting residue was purified on a reverse phase column (30 to 60% MeCN in H₂O w/0.2% NH₄OH) to afford N-((3R,4R)-4-fluoropyrrolidin-3-yl)-4-(4-(trifluoromethyl)phenyl)phthalazin-1-amine (7 mg, 6% yield over two steps) as a white solid. m/z: (ES⁺) [M+H]⁺=377.

### 1-((3R,4R)-3-fluoro-4-((4-(4-(trifluoromethyl)phenyl)phthalazin-1-yl)amino)pyrrolidin-1-yl)prop-2-en-1-one

A solution of acryloyl chloride (2 μL, 0.02 mmol) in DCM (0.1 mL) was added dropwise to a solution of N-((3R,4R)-4-fluoropyrrolidin-3-yl)-4-(4-(trifluoromethyl)phenyl)phthalazin-1-amine (7 mg, 0.02 mmol) and Et₃N (5 μL, 0.04 mmol) in DCM (1 mL) at 0° C. The reaction mixture was warmed to rt and stirred for 1 h. The reaction mixture was directly purified by flash silica chromatography (0 to 100% EtOAc in hexanes) to afford 1-((3R,4R)-3-fluoro-4-((4-(4-(trifluoromethyl)phenyl)phthalazin-1-yl)amino)pyrrolidin-1-yl)prop-2-en-1-one (Example 11, 4 mg, 48% yield) as a white amorphous solid. ¹H NMR (500 MHz, DMSO-d6) δ 3.68-4.21 (4H, m), 4.84-5.08 (1H, m), 5.26-5.54 (1H, m), 5.74 (1H, ddd), 6.22 (1H, dt), 6.66 (1H, dt), 7.66 (1H, br t), 7.81 (1H, br d), 7.83-8.07 (6H, m), 8.48 (1H, dd); m/z: (ES⁺) [M+H]⁺=431.

### Example 12: 1-((3S,4R)-3-hydroxy-4-((4-(4-(trifluoromethyl)phenyl)phthalazin-1-yl)amino)pyrrolidin-1-yl)prop-2-en-1-one

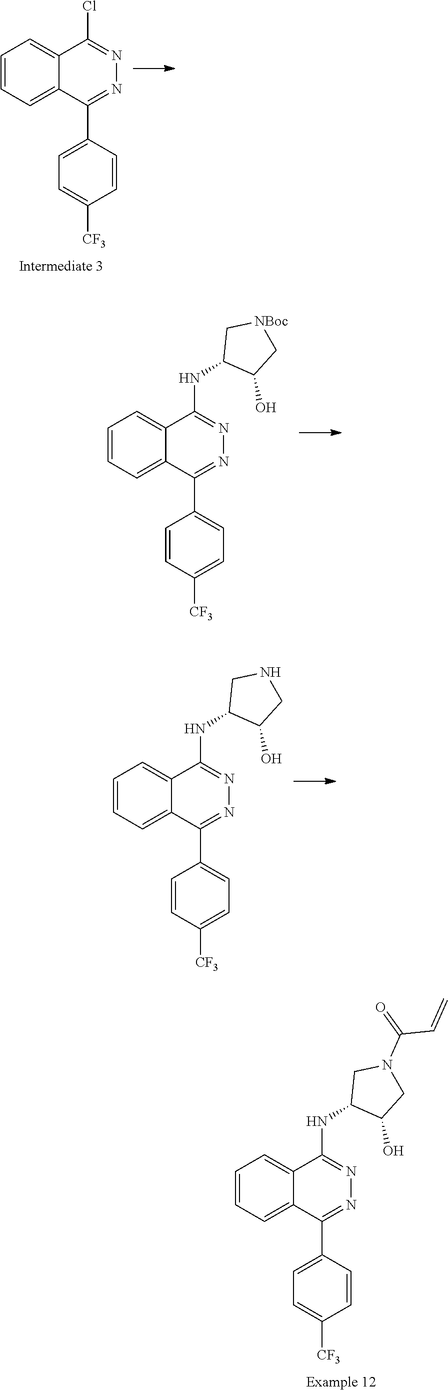 [SMILES: FC(F)(F)c1ccc(-c2nnc(Cl)c3ccccc23)cc1 ; CC(C)(C)OC(=O)N1C[C@H](O)[C@H](Nc2nnc(-c3ccc(C(F)(F)F)cc3)c3ccccc23)C1 ; O[C@H]1CNC[C@H]1Nc1nnc(-c2ccc(C(F)(F)F)cc2)c2ccccc12 ; C=CC(=O)N1C[C@H](O)[C@H](Nc2nnc(-c3ccc(C(F)(F)F)cc3)c3ccccc23)C1]

### tert-butyl (3S,4R)-3-hydroxy-4-((4-(4-(trifluoromethyl)phenyl)phthalazin-1-yl)amino)pyrrolidine-1-carboxylate

DIPEA (0.17 mL, 0.97 mmol) was added to a solution of tert-butyl (3R,4S)-3-amino-4-hydroxypyrrolidine-1-carboxylate (118 mg, 0.580 mmol) and 1-chloro-4-(4-(trifluoromethyl)phenyl)phthalazine (Intermediate 3, 150 mg, 0.490 mmol) in DMSO (2 mL). The reaction mixture was heated to 85° C. and stirred for 72 hrs. An additional portion of tert-butyl (3R,4S)-3-amino-4-hydroxypyrrolidine-1-carboxylate (118 mg, 0.580 mmol) and DIPEA (0.17 mL, 0.97 mmol) were added and the reaction mixture was stirred at 85° C. for another 16 hrs. The reaction mixture was cooled to rt, diluted with DCM (100 mL), and washed with water (20 mL). The organic layer was dried over Na₂SO₄, filtered and concentrated to dryness. The crude material was purified by flash silica chromatography (0 to 100% EtOAc in hexanes) to afford tert-butyl (3S,4R)-3-hydroxy-4-((4-(4-(trifluoromethyl)phenyl)phthalazin-1-yl)amino)pyrrolidine-1-carboxylate (135 mg, 59% yield) as a light-yellow solid. m/z: (ES⁺) [M+H]⁺=475.

### (3S,4R)-4-((4-(4-(trifluoromethyl)phenyl)phthalazin-1-yl)amino)pyrrolidin-3-ol

HCl (4 M in dioxane, 5.0 mL, 20 mmol) was added to a solution of tert-butyl (3S,4R)-3-hydroxy-4-((4-(4-(trifluoromethyl)phenyl)phthalazin-1-yl)amino)pyrrolidine-1-carboxylate (135 mg, 0.290 mmol) in MeOH (3 mL) at rt and the reaction mixture stirred for 1 h. The volatiles were removed under reduced pressure to afford (3S,4R)-4-((4-(4-(trifluoromethyl)phenyl)phthalazin-1-yl)amino)pyrrolidin-3-ol, HCl (115 mg, 58% yield) as an off-white solid which was used directly in the next step without purification. m/z: (ES⁺) [M+H]⁺=375.

### 1-((3S,4R)-3-hydroxy-4-((4-(4-(trifluoromethyl)phenyl)phthalazin-1-yl)amino)pyrrolidin-1-yl)prop-2-en-1-one

A solution of acryloyl chloride (25 mg, 0.28 mmol) in DCM (1 mL) was added dropwise to a solution of (3S,4R)-4-((4-(4-(trifluoromethyl)phenyl)phthalazin-1-yl)amino)pyrrolidin-3-ol, HCl (115 mg, 0.28 mmol) and DIPEA (98 μL, 0.56 mmol) in DCM (5 mL) at 0° C. The reaction mixture was stirred at 0° C. for 1 h. The reaction mixture was diluted with DCM (60 mL) and washed with water (15 mL) and brine (10 mL). The organic layer was dried over Na₂SO₄, filtered and concentrated to dryness. The crude material was purified on a reverse phase C18 column (0 to 100% MeCN in H₂O w/0.1% HCO₂H) to afford 6868 1-((3S,4R)-3-hydroxy-4-((4-(4-(trifluoromethyl)phenyl)phthalazin-1-yl)amino)pyrrolidin-1-yl)prop-2-en-1-one (Example 12, 47 mg, 39% yield) as a white amorphous solid. ¹H NMR (500 MHz, DMSO-d6) δ 3.55-3.69 (2H, m), 3.75 (0.5H, t), 3.81-3.96 (1H, m), 4.10 (0.5H, t), 4.45-4.67 (1H, m), 4.70-4.99 (1H, m), 5.21-5.56 (1H, m), 5.70 (1H, dt), 6.18 (1H, dd), 6.50-6.74 (1H, m), 7.35 (1H, br d), 7.68-8.04 (7H, m), 8.54 (1H, dd); m/z: (ES⁺) [M+H]⁺=429.

### Example 13: (S)-1-(3-((8-fluoro-4-(4-(trifluoromethyl)phenyl)phthalazin-1-yl)amino)pyrrolidin-1-yl)prop-2-en-1-one

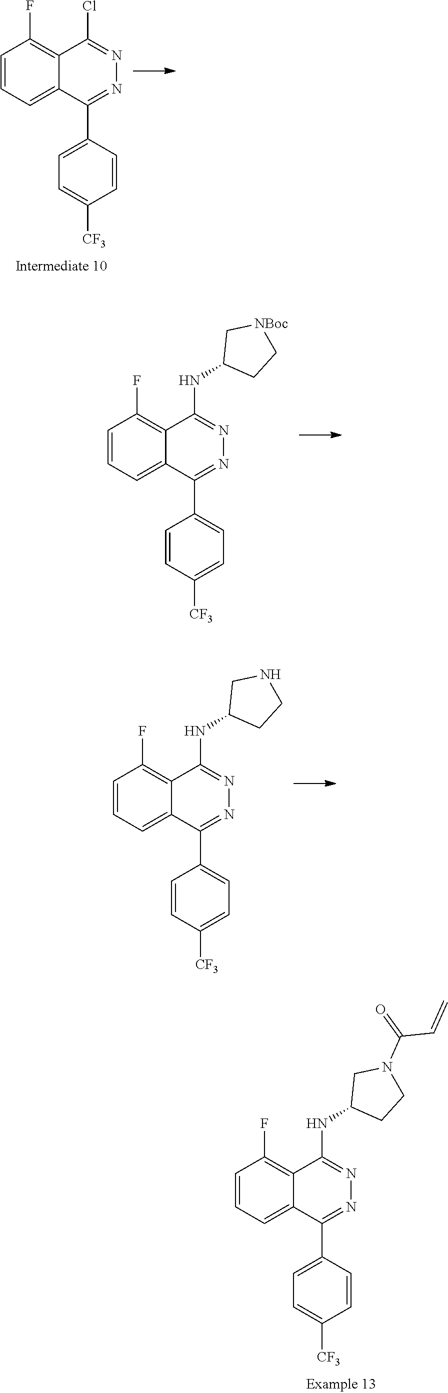 [SMILES: Fc1cccc2c(-c3ccc(C(F)(F)F)cc3)nnc(Cl)c12 ; CC(C)(C)OC(=O)N1CC[C@H](Nc2nnc(-c3ccc(C(F)(F)F)cc3)c3cccc(F)c23)C1 ; Fc1cccc2c(-c3ccc(C(F)(F)F)cc3)nnc(N[C@H]3CCNC3)c12 ; C=CC(=O)N1CC[C@H](Nc2nnc(-c3ccc(C(F)(F)F)cc3)c3cccc(F)c23)C1]

### tert-butyl (S)-3-((8-fluoro-4-(4-(trifluoromethyl)phenyl)69hthalazine-1-yl)amino)pyrrolidine-1-carboxylate

DIPEA (0.428 mL, 2.45 mmol) was added to a solution of tert-butyl (S)-3-aminopyrrolidine-1-carboxylate (148 mg, 0.800 mmol) and 4-chloro-5-fluoro-1-(4-(trifluoromethyl)phenyl)phthalazine (Intermediate 10, 200 mg, 0.61 mmol) in DMSO (2.5 mL). The reaction mixture was heated to 90° C. and stirred for 16 hrs. The reaction mixture was cooled to rt, diluted with water (20 mL), and extracted with DCM (3×40 mL). The combined organics were dried over Na₂SO₄, filtered and concentrated to dryness. The crude material was purified by flash silica chromatography (10 to 100% EtOAc in hexanes) to afford tert-butyl (S)-3-((8-fluoro-4-(4-(trifluoromethyl)phenyl)phthalazin-1-yl)amino)pyrrolidine-1-carboxylate (103 mg, 35% yield) as a white solid. m/z: (ES⁺) [M+H]⁺=477.

### (S)-8-fluoro-N-(pyrrolidin-3-yl)-4-(4-(trifluoromethyl)phenyl)phthalazin-1-amine

TFA (0.77 mL, 10 mmol) was added to a solution of tert-butyl (S)-3-((8-fluoro-4-(4-(trifluoromethyl)phenyl)phthalazin-1-yl)amino)pyrrolidine-1-carboxylate (103 mg, 0.220 mmol) in DCM (5 mL) and the reaction mixture stirred at rt for 2 hrs. The volatiles were removed under reduced pressure to afford (S)-8-fluoro-N-(pyrrolidin-3-yl)-4-(4-(trifluoromethyl)phenyl)phthalazin-1-amine, TFA (106 mg, 100% yield) as a yellow gum which was used directly in the next step without purification. m/z: (ES⁺) [M+H]⁺=377.

### (S)-1-(3-((8-fluoro-4-(4-(trifluoromethyl)phenyl)phthalazin-1-yl)amino)pyrrolidin-1-yl)prop-2-en-1-one

Acryloyl chloride (26 μL, 0.32 mmol) was added to a solution of (S)-8-fluoro-N-(pyrrolidin-3-yl)-4-(4-(trifluoromethyl)phenyl)phthalazin-1-amine, TFA (106 mg, 0.220 mmol) in THF (6 mL) and saturated aq. NaHCO₃ (1.5 mL) at 0° C. and the reaction stirred for 30 min. The reaction mixture was diluted with water and extracted with DCM (2×30 mL). The combined organics were dried over Na₂SO₄, filtered and concentrated to dryness. The crude material was purified on a reverse phase C18 column (0 to 100% MeCN in H₂O w/0.1% HCO₂H) to afford (S)-1-(3-((8-fluoro-4-(4-(trifluoromethyl)phenyl)phthalazin-1-yl)amino)pyrrolidin-1-yl)prop-2-en-1-one (Example 13, 71 mg, 76% yield) as a white amorphous solid. ¹H NMR (500 MHz, DMSO-d6) δ 2.00-2.49 (2H, m), 3.45-4.18 (4H, m), 4.64-5.02 (1H, m), 5.54-5.76 (1H, m), 6.17 (1H, ddt), 6.49-6.71 (1H, m), 6.90 (1H, ddd), 7.62 (1H, br d), 7.78 (1H, dt), 7.83-8.07 (5H, m); m/z: (ES⁺) [M+H]=431.

### Example 14: 1-((3R,4S)-3-((8-fluoro-4-(4-(trifluoromethyl)phenyl)phthalazin-1-yl)amino)-4-hydroxypyrrolidin-1-yl)prop-2-en-1-one

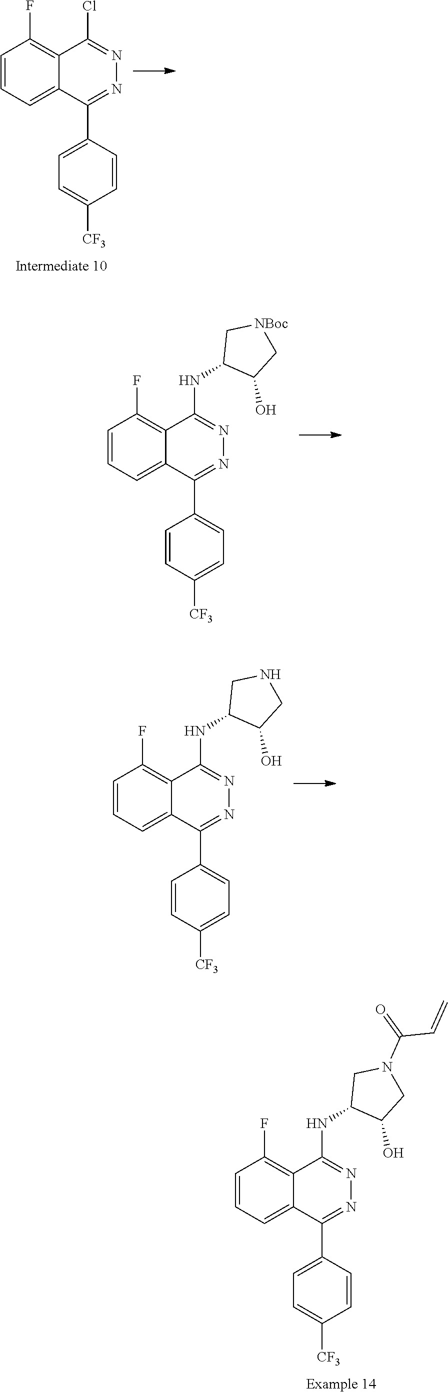 [SMILES: Fc1cccc2c(-c3ccc(C(F)(F)F)cc3)nnc(Cl)c12 ; CC(C)(C)OC(=O)N1C[C@H](O)[C@H](Nc2nnc(-c3ccc(C(F)(F)F)cc3)c3cccc(F)c23)C1 ; O[C@H]1CNC[C@H]1Nc1nnc(-c2ccc(C(F)(F)F)cc2)c2cccc(F)c12 ; C=CC(=O)N1C[C@H](O)[C@H](Nc2nnc(-c3ccc(C(F)(F)F)cc3)c3cccc(F)c23)C1]

### tert-butyl (3R,4S)-3-((8-fluoro-4-(4-(trifluoromethyl)phenyl)phthalazin-1-yl)amino)-4-hydroxypyrrolidine-1-carboxylate

DIPEA (0.428 mL, 2.45 mmol) was added to a solution of tert-butyl (3R,4S)-3-amino-4-hydroxypyrrolidine-1-carboxylate (161 mg, 0.800 mmol) and 4-chloro-5-fluoro-1-(4-(trifluoromethyl)phenyl)phthalazine (Intermediate 10, 200 mg, 0.61 mmol) in DMSO (2.5 mL). The reaction mixture was heated to 90° C. and stirred for 15 hrs. The reaction mixture was cooled to rt, diluted with water (30 mL), and extracted with DCM (3×40 mL). The combined organics were dried over Na₂SO₄, filtered and concentrated to dryness. The crude material was purified by flash silica chromatography (10 to 100% EtOAc in hexanes) to afford tert-butyl (3R,4S)-3-((8-fluoro-4-(4-(trifluoromethyl)phenyl)phthalazin-1-yl)amino)-4-hydroxypyrrolidine-1-carboxylate (101 mg, 34% yield) as a white solid. m/z: (ES⁺) [M+H]⁺=493.

### (3S,4R)-4-((8-fluoro-4-(4-(trifluoromethyl)phenyl)phthalazin-1-yl)amino)pyrrolidin-3-ol

TFA (0.76 mL, 9.8 mmol) was added to a solution of tert-butyl (3R,4S)-3-((8-fluoro-4-(4-(trifluoromethyl)phenyl)phthalazin-1-yl)amino)-4-hydroxypyrrolidine-1-carboxylate (101 mg, 0.200 mmol) in DCM (5 mL) and the reaction mixture stirred at rt for 2 hrs. The volatiles were removed under reduced pressure to afford (3S,4R)-4-((8-fluoro-4-(4-(trifluoromethyl)phenyl)phthalazin-1-yl)amino)pyrrolidin-3-ol (100 mg, 100% yield) as a yellow gum which was used directly in the next step without purification. m/z: (ES⁺) [M+H]⁺=393.

### 1-((3R,4S)-3-((8-fluoro-4-(4-(trifluoromethyl)phenyl)phthalazin-1-yl)amino)-4-hydroxypyrrolidin-1-yl)prop-2-en-1-one

Acryloyl chloride (25 μL, 0.31 mmol) was added to a solution of (3S,4R)-4-((8-fluoro-4-(4-(trifluoromethyl)phenyl)phthalazin-1-yl)amino)pyrrolidin-3-ol (100 mg, 0.21 mmol) in THF (6 mL) and saturated aq. NaHCO₃ (1.5 mL) at 0° C. and the reaction stirred for 30 min. The reaction mixture was diluted with water (10 mL) and extracted with DCM (2×30 mL). The organic layer was dried over Na₂SO₄, filtered and concentrated to dryness. The crude material was purified on a reverse phase C18 column (0 to 100% MeCN in H₂O w/0.1% HCO₂H) to afford 1-((3R,4S)-3-((8-fluoro-4-(4-(trifluoromethyl)phenyl)phthalazin-1-yl)amino)-4-hydroxypyrrolidin-1-yl)prop-2-en-1-one (Example 14, 58 mg, 64% yield) as a white amorphous solid. ¹H NMR (500 MHz, DMSO-d6) δ 3.38 (0.5H, br s), 3.45-3.68 (2H, m), 3.89 (0.5H, br dd), 4.03-4.29 (1H, m), 4.37-4.54 (1H, m), 4.65-4.94 (1H, m), 5.67 (1H, ddd), 5.84-5.99 (1H, m), 6.14 (1H, ddd), 6.58 (1H, ddd), 6.92-7.12 (1H, m), 7.62 (1H, dd), 7.70-7.98 (6H, m); m/z: (ES⁺) [M+H]⁺=447.

### Example 15: 1-((3S,4R)-3-((8-fluoro-4-(4-(trifluoromethyl)phenyl)phthalazin-1-yl)amino)-4-(hydroxymethyl)pyrrolidin-1-yl)prop-2-en-1-one

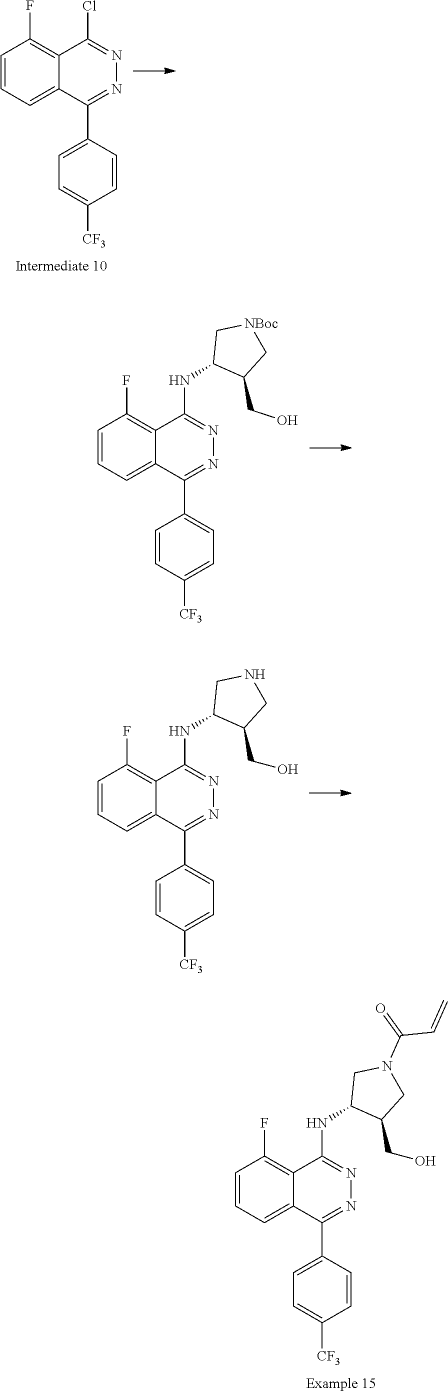 [SMILES: Fc1cccc2c(-c3ccc(C(F)(F)F)cc3)nnc(Cl)c12 ; CC(C)(C)OC(=O)N1C[C@@H](CO)[C@H](Nc2nnc(-c3ccc(C(F)(F)F)cc3)c3cccc(F)c23)C1 ; OC[C@@H]1CNC[C@H]1Nc1nnc(-c2ccc(C(F)(F)F)cc2)c2cccc(F)c12 ; C=CC(=O)N1C[C@@H](CO)[C@H](Nc2nnc(-c3ccc(C(F)(F)F)cc3)c3cccc(F)c23)C1]

### tert-butyl (3S,4R)-3-((8-fluoro-4-(4-(trifluoromethyl)phenyl)phthalazin-1-yl)amino)-4-(hydroxymethyl)pyrrolidine-1-carboxylate

DIPEA (0.428 mL, 2.45 mmol) was added to a solution of tert-butyl (3S,4R)-3-amino-4-(hydroxymethyl)pyrrolidine-1-carboxylate (159 mg, 0.730 mmol) and 4-chloro-5-fluoro-1-(4-(trifluoromethyl)phenyl)phthalazine (Intermediate 10, 200 mg, 0.61 mmol) in DMSO (2.5 mL). The reaction mixture was heated to 90° C. and stirred for 15 hrs. The reaction mixture was cooled to rt, diluted with water (30 mL), and extracted with DCM (3×40 mL). The combined organics were dried over Na₂SO₄, filtered and concentrated to dryness. The crude material was purified by flash silica chromatography (10 to 100% EtOAc in hexanes) to afford tert-butyl (3S,4R)-3-((8-fluoro-4-(4-(trifluoromethyl)phenyl)phthalazin-1-yl)amino)-4-(hydroxymethyl)pyrrolidine-1-carboxylate (92 mg, 30% yield) as a white solid. m/z: (ES⁺) [M+H]⁺=507.

### ((3R,4S)-4-((8-fluoro-4-(4-(trifluoromethyl)phenyl)phthalazin-1-yl)amino)pyrrolidin-3-yl)methanol

TFA (0.76 mL, 9.8 mmol) was added to a solution of tert-butyl (3S,4R)-3-((8-fluoro-4-(4-(trifluoromethyl)phenyl)phthalazin-1-yl)amino)-4-(hydroxymethyl)pyrrolidine-1-carboxylate (92 mg, 0.18 mmol) in DCM (5 mL) and the reaction mixture was stirred at rt for 2 hrs. The volatiles were removed under reduced pressure to afford ((3R,4S)-4-((8-fluoro-4-(4-(trifluoromethyl)phenyl)phthalazin-1-yl)amino)pyrrolidin-3-yl)methanol, TFA (90 mg, 100% yield) as a yellow gum which was used directly in the next step without purification. m/z: (ES⁺) [M+H]⁺=407.

### 1-((3S,4R)-3-((8-fluoro-4-(4-(trifluoromethyl)phenyl)phthalazin-1-yl)amino)-4-(hydroxymethyl)pyrrolidin-1-yl)prop-2-en-1-one

Acryloyl chloride (22 μL, 0.27 mmol) was added to a solution of ((3R,4S)-4-((8-fluoro-4-(4-(trifluoromethyl)phenyl)phthalazin-1-yl)amino)pyrrolidin-3-yl)methanol, TFA (90 mg, 0.18 mmol) in THF (6 mL) and saturated aq. NaHCO₃ (1.5 mL) at 0° C. and the reaction stirred for 30 min. The reaction mixture was diluted with water (10 mL) and extracted with DCM (2×30 mL). The combined organics were dried over Na₂SO₄, filtered and concentrated to dryness. The crude material was purified on a reverse phase C18 column (0 to 100% MeCN in H₂O w/0.1% HCO₂H) to afford 1-((3S,4R)-3-((8-fluoro-4-(4-(trifluoromethyl)phenyl)phthalazin-1-yl)amino)-4-(hydroxymethyl)pyrrolidin-1-yl)prop-2-en-1-one (Example 15, 58 mg, 68% yield) as a white amorphous solid. ¹H NMR (500 MHz, DMSO-d6) δ 2.58-2.80 (1H, m), 3.37-3.45 (1H, m), 3.46-3.69 (3H, m), 3.72-3.96 (1H, m), 4.10-4.39 (1H, m), 4.55-4.80 (1H, m), 4.86-5.06 (1H, m), 5.61-5.76 (1H, m), 6.16 (1H, ddd), 6.47-6.74 (1H, m), 7.05 (1H, ddd), 7.62 (1H, d), 7.72-7.84 (1H, m), 7.82-7.99 (5H, m); m/z: (ES⁺) [M+H]⁺=461.

### Example 16: (S)-1-(3-((5-(5-(trifluoromethyl)pyridin-2-yl)pyrido[2,3-d]pyridazin-8-yl)amino)pyrrolidin-1-yl)prop-2-en-1-one

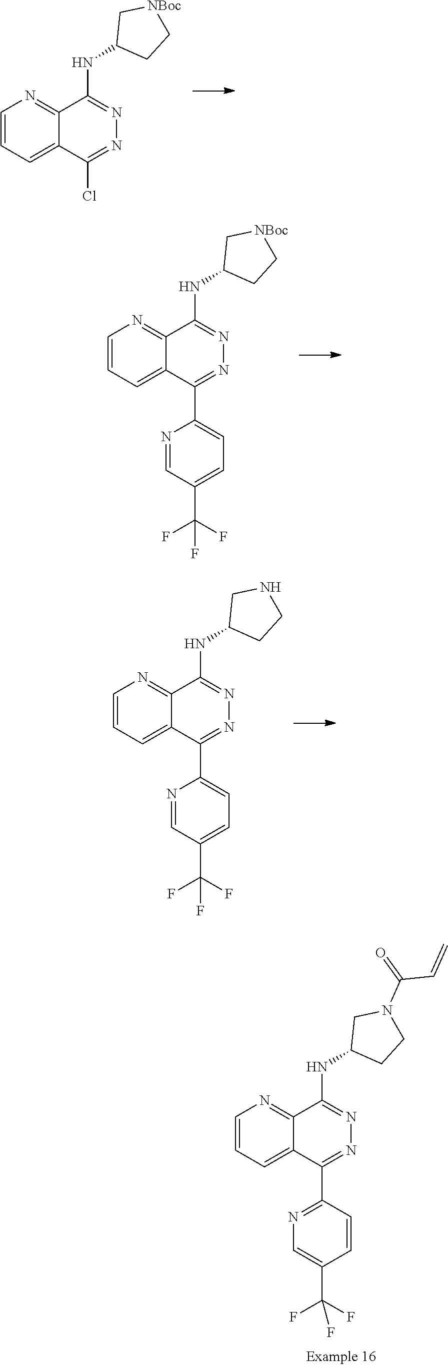

### tert-butyl (S)-3-((5-(5-(trifluoromethyl)pyridin-2-yl)pyrido[2,3-d]pyridazin-8-yl)amino)pyrrolidine-1-carboxylate

(5-(Trifluoromethyl)pyridin-2-yl)boronic acid (109 mg, 0.570 mmol), tert-butyl (S)-3-((5-chloropyrido[2,3-d]pyridazin-8-yl)amino)pyrrolidine-1-carboxylate (100 mg, 0.29 mmol), PdCl₂(dppf) (21 mg, 0.030 mmol) and Cs₂CO₃ (279 mg, 0.860 mmol) were diluted in 1,4-dioxane (10 mL) and water (1 mL) under an atmosphere of N₂. The reaction mixture was heated to 55° C. and stirred for 16 hrs. An additional portion of (5-(trifluoromethyl)pyridin-2-yl)boronic acid (218 mg, 1.14 mmol), PdCl₂(dppf) (42 mg, 0.060 mmol) and Cs₂CO₃ (279 mg, 0.860 mmol) was added and the reaction mixture stirred at 55° C. for another 24 hrs. The reaction mixture was cooled to rt, diluted with water (20 mL), and extracted with DCM (3×30 mL). The combined organics were dried over Na₂SO₄, filtered and concentrated to dryness. The crude material was purified by flash silica chromatography (0 to 100% EtOAc in hexanes) to afford tert-butyl (S)-3-((5-(5-(trifluoromethyl)pyridin-2-yl)pyrido[2,3-d]pyridazin-8-yl)amino)pyrrolidine-1-carboxylate (27 mg, 21% yield) as a white solid. m/z: (ES⁺) [M+H]⁺=461.

### (S)-1-(3-((5-(5-(trifluoromethyl)pyridin-2-yl)pyrido[2,3-d]pyridazin-8-yl)amino)pyrrolidin-1-yl)prop-2-en-1-one

tert-Butyl (S)-3-((5-(5-(trifluoromethyl)pyridin-2-yl)pyrido[2,3-d]pyridazin-8-yl)amino)pyrrolidine-1-carboxylate (27 mg, 0.060 mmol) was dissolved in HCl (4 M in dioxane, 2.0 mL, 8.0 mmol) and the reaction mixture stirred at rt for 1 h. The volatiles were removed under reduced pressure to afford (S)—N-(pyrrolidin-3-yl)-5-(5-(trifluoromethyl)pyridin-2-yl)pyrido[2,3-d]pyridazin-8-amine, HCl. The crude HCl salt was dissolved in THF (2 mL) and saturated aq. NaHCO₃ (0.5 mL). Acryloyl chloride (7 μL, 0.09 mmol) was added and the reaction mixture stirred at rt for 15 hrs. The reaction mixture was diluted with water (10 mL) and extracted with DCM (3×15 mL). The combined organics were dried over Na₂SO₄, filtered and concentrated to dryness. The crude material was purified on a reverse phase C18 column (0 to 100% MeCN in H₂O w/0.1% NH₄OH) to afford (S)-1-(3-((5-(5-(trifluoromethyl)pyridin-2-yl)pyrido[2,3-d]pyridazin-8-yl)amino)pyrrolidin-1-yl)prop-2-en-1-one (Example 16, 14 mg, 57% yield) as a white amorphous solid. ¹H NMR (500 MHz, DMSO-d6) δ 2.19-2.41 (2H, m), 3.45-4.12 (4H, m), 4.79-5.03 (1H, m), 5.66 (1H, ddd), 6.14 (1H, ddd), 6.47-6.75 (1H, m), 7.89-7.99 (1H, m), 8.05-8.21 (1H, m), 8.41 (2H, s), 9.07-9.18 (2H, m), 9.25 (1H, d); m/z: (ES⁺) [M+H]⁺=415.

### Example 17: (S)-1-(3-((8-(5-(trifluoromethyl)pyridin-2-yl)pyrido[3,4-b]pyrazin-5-yl)amino)pyrrolidin-1-yl)prop-2-en-1-one

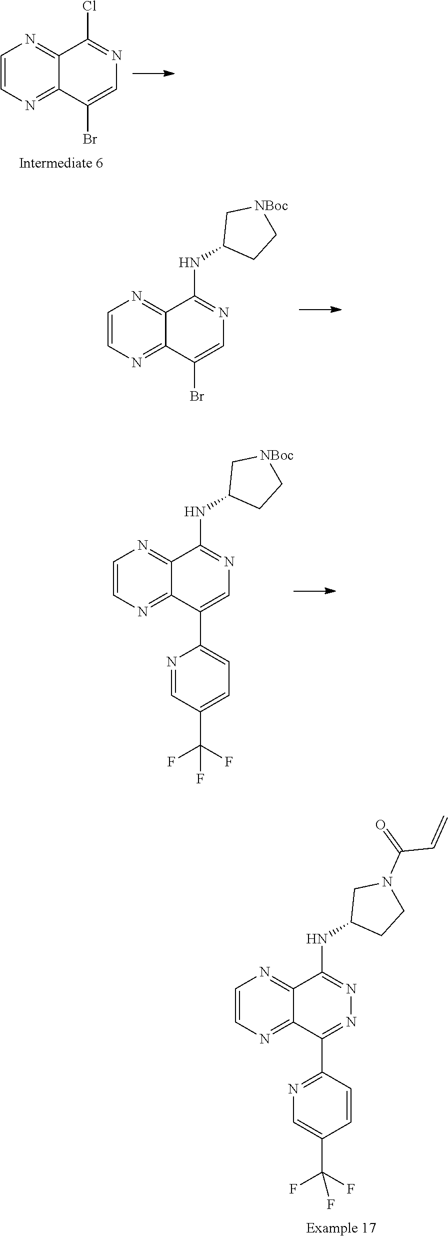 [SMILES: Clc1ncc(Br)c2nccnc12 ; CC(C)(C)OC(=O)N1CC[C@H](Nc2ncc(Br)c3nccnc23)C1 ; CC(C)(C)OC(=O)N1CC[C@H](Nc2ncc(-c3ccc(C(F)(F)F)cn3)c3nccnc23)C1 ; C=CC(=O)N1CC[C@H](Nc2nnc(-c3ccc(C(F)(F)F)cn3)c3nccnc23)C1]

### tert-butyl (S)-3-((8-bromopyrido[3,4-b]pyrazin-5-yl)amino)pyrrolidine-1-carboxylate

DIPEA (0.36 mL, 2.1 mmol) was added to a solution of tert-butyl (S)-3-aminopyrrolidine-1-carboxylate (210 mg, 1.1 mmol) and 8-bromo-5-chloropyrido[3,4-b]pyrazine (Intermediate 6, 250 mg, 1.02 mmol) in DMSO (6 mL). The reaction mixture was heated to 80° C. and stirred for 16 hrs. The reaction mixture was cooled to rt, diluted with water (25 mL), and extracted with DCM (3×50 mL). The combined organics were dried over Na₂SO₄, filtered and concentrated to dryness. The crude material was purified by flash silica chromatography (0 to 100% EtOAc in hexanes) to afford tert-butyl (S)-3-((8-bromopyrido[3,4-b]pyrazin-5-yl)amino)pyrrolidine-1-carboxylate (386 mg, 96% yield) as a bright-yellow solid. ¹H NMR (500 MHz, DMSO-d6) δ 1.39 (9H, br d), 2.03-2.27 (2H, m), 3.24-3.29 (1H, m), 3.31-3.38 (1H, m), 3.39-3.51 (1H, m), 3.58-3.68 (1H, m), 4.53-4.74 (1H, m), 8.13 (1H, d), 8.39 (1H, s), 8.88 (1H, d), 9.14 (1H, d); m/z: (ES⁺) [M+H]⁺=394.

### tert-butyl (S)-3-((8-(5-(trifluoromethyl)pyridin-2-yl)pyrido[3,4-b]pyrazin-5-yl)amino)pyrrolidine-1-carboxylate

2-(Tributylstannyl)-5-(trifluoromethyl)pyridine (451 mg, 1.03 mmol), tert-butyl (S)-3-((8-bromopyrido[3,4-b]pyrazin-5-yl)amino)pyrrolidine-1-carboxylate (340 mg, 0.86 mmol) and Pd(PPh₃)₄ (149 mg, 0.130 mmol) were diluted in toluene (10 mL) under an atmosphere of N₂. The reaction mixture was heated to 120° C. and stirred for 48 hrs. The reaction mixture was cooled to rt, diluted with saturated aq. NH₄Cl (30 mL), and extracted with EtOAc (3×50 mL). The combined organics were dried over Na₂SO₄, filtered and concentrated to dryness. The crude material was purified by flash silica chromatography (0 to 100% EtOAc in hexanes) to afford tert-butyl (S)-3-((8-(5-(trifluoromethyl)pyridin-2-yl)pyrido[3,4-b]pyrazin-5-yl)amino)pyrrolidine-1-carboxylate (378 mg, 95% yield) as a yellow solid. ¹H NMR (500 MHz, CDCl₃) δ 1.47-1.53 (9H, m), 2.06-2.20 (1H, m), 2.26-2.43 (1H, m), 3.31-3.69 (3H, m), 3.87 (1H, br dd), 4.90 (1H, br s), 7.06 (1H, br d), 7.91-8.07 (1H, m), 8.31 (1H, br d), 8.72 (1H, s), 8.90 (1H, br s), 9.00 (2H, br d); m/z: (ES⁺) [M+H]⁺=461.

### (S)-1-(3-((8-(5-(trifluoromethyl)pyridin-2-yl)pyrido[3,4-b]pyrazin-5-yl)amino)pyrrolidin-1-yl)prop-2-en-1-one

HCl (4 M in dioxane, 6.0 mL, 24 mmol) was added to a solution of tert-butyl (S)-3-((8-(5-(trifluoromethyl)pyridin-2-yl)pyrido[3,4-b]pyrazin-5-yl)amino)pyrrolidine-1-carboxylate (178 mg, 0.390 mmol) in MeOH (4 mL) at rt and the reaction mixture stirred for 1 h. The volatiles were removed under reduced pressure to afford (S)—N-(pyrrolidin-3-yl)-8-(5-(trifluoromethyl)pyridin-2-yl)pyrido[3,4-b]pyrazin-5-amine, HCl as a dark residue. The crude HCl salt was dissolved in THF (8 mL) and saturated aq. NaHCO₃ (2 mL). Acryloyl chloride (47 μL, 0.58 mmol) was added and the reaction mixture stirred at rt for 4 hrs. The reaction mixture was diluted with water and extracted with DCM (3×20 mL). The combined organics were dried over Na₂SO₄, filtered and concentrated to dryness. The crude material was purified on a reverse phase C18 column (0 to 100% MeCN in H₂O w/0.2% NH₄OH) to afford (S)-1-(3-((8-(5-(trifluoromethyl)75yrrolid-2-yl)pyrido[3,4-b]pyrazin-5-yl)amino)75yrrolidine-1-yl)prop-2-en-1-one (Example 17, 69 mg, 43% yield) as a yellow amorphous solid. ¹H NMR (500 MHz, DMSO-d6) δ 2.09-2.38 (2H, m), 3.45-4.09 (4H, m), 4.67-5.01 (1H, m), 5.55-5.76 (1H, m), 6.15 (1H, br dd), 6.48-6.76 (1H, m), 8.25 (1H, br d), 8.39-8.57 (2H, m), 8.85 (1H, s), 8.91 (1H, s), 9.03 (1H, br s), 9.13 (1H, s); m/z: (ES⁺) [M+H]⁺=414.

### Example 18: (S)-1-(3-((4-(5-(trifluoromethyl)pyridin-2-yl)phthalazin-1-yl)amino)pyrrolidin-1-yl)prop-2-en-1-one

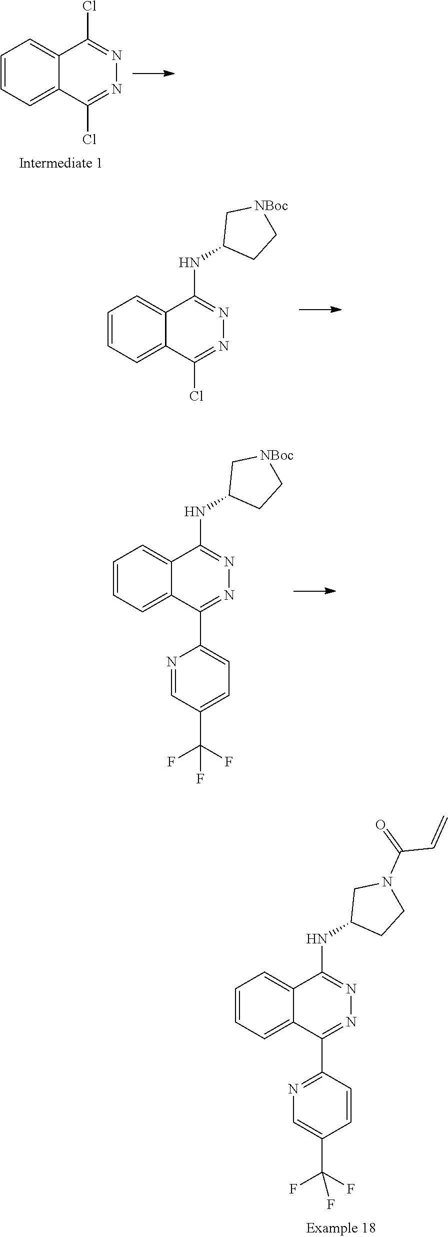 [SMILES: Clc1nnc(Cl)c2ccccc12 ; CC(C)(C)OC(=O)N1CC[C@H](Nc2nnc(Cl)c3ccccc23)C1 ; CC(C)(C)OC(=O)N1CC[C@H](Nc2nnc(-c3ccc(C(F)(F)F)cn3)c3ccccc23)C1 ; C=CC(=O)N1CC[C@H](Nc2nnc(-c3ccc(C(F)(F)F)cn3)c3ccccc23)C1]

### tert-butyl (S)-3-((4-chlorophthalazin-1-yl)amino)pyrrolidine-1-carboxylate

DIPEA (0.439 mL, 2.51 mmol) was added to a solution of tert-butyl (S)-3-aminopyrrolidine-1-carboxylate (257 mg, 1.38 mmol) and 1,4-dichlorophthalazine (Intermediate 1, 250 mg, 1.3 mmol) in DMSO (6 mL). The reaction mixture was heated to 85° C. and stirred for 20 hrs. The reaction mixture was cooled to rt and directly purified on a reverse phase C18 column (0 to 100% MeCN in H₂O w/0.1% HCO₂H) to afford tert-butyl (S)-3-((4-chlorophthalazin-1-yl)amino)pyrrolidine-1-carboxylate (353 mg, 81% yield) as a white solid. ¹H NMR (500 MHz, DMSO-d6) δ 1.41 (9H, br d), 1.98-2.12 (1H, m), 2.21-2.30 (1H, m), 3.33-3.43 (2H, m), 3.44-3.54 (1H, m), 3.64-3.82 (1H, m), 4.59-4.74 (1H, m), 7.61 (1H, br d), 7.98-8.06 (2H, m), 8.07-8.15 (1H, m), 8.44-8.53 (1H, m); m/z: (ES⁺) [M+H]⁺=349.

### tert-butyl (S)-3-((4-(5-(trifluoromethyl)pyridin-2-yl)phthalazin-1-yl)amino)pyrrolidine-1-carboxylate

2-(Tributylstannyl)-5-(trifluoromethyl)pyridine (383 mg, 0.880 mmol), tert-butyl (S)-3-((4-chlorophthalazin-1-yl)amino)pyrrolidine-1-carboxylate (255 mg, 0.730 mmol) and Pd(PPh₃)₄ (127 mg, 0.110 mmol) were diluted in toluene (2 mL) under an atmosphere of N₂. The reaction mixture was heated to 110° C. and stirred for 16 hrs. The reaction mixture was cooled to rt, diluted with saturated aq. NH₄Cl (50 mL), and extracted with ethyl acetate (3×60 mL). The combined organics were dried over Na₂SO₄, filtered and concentrated to dryness. The crude material was purified by flash silica chromatography (0 to 100% EtOAc in hexanes) to afford tert-butyl (S)-3-((4-(5-(trifluoromethyl)pyridin-2-yl)phthalazin-1-yl)amino)pyrrolidine-1-carboxylate (200 mg, 60% yield) as a light yellow solid. m/z: (ES⁺) [M+H]⁺=460.

### (S)-1-(3-((4-(5-(trifluoromethyl)pyridin-2-yl)phthalazin-1-yl)amino)pyrrolidin-1-yl)prop-2-en-1-one

HCl (4 M in dioxane, 6.0 mL, 24 mmol) was added to a solution of tert-butyl (S)-3-((4-(5-(trifluoromethyl)pyridin-2-yl)phthalazin-1-yl)amino)pyrrolidine-1-carboxylate (200 mg, 0.440 mmol) in MeOH (4 mL) and the reaction mixture stirred at rt for 1 h. The volatiles were removed under reduced pressure to afford (S)—N-(pyrrolidin-3-yl)-4-(5-(trifluoromethyl)pyridin-2-yl)phthalazin-1-amine, HCl as a light-yellow residue. The crude HCl salt was dissolved in THF (8 mL) and saturated aq. NaHCO₃ (2 mL). Acryloyl chloride (53 μL, 0.65 mmol) was added and the reaction mixture stirred at rt for 16 hrs. The reaction mixture was diluted with water (50 mL) and extracted with DCM (3×50 mL). The combined organics were dried over Na₂SO₄, filtered and concentrated to dryness. The crude material was purified on a reverse phase C18 column (0 to 100% MeCN in H₂O w/0.2% NH₄OH) to afford (S)-1-(3-((4-(5-(trifluoromethyl)pyridin-2-yl)phthalazin-1-yl)amino)pyrrolidin-1-yl)prop-2-en-1-one (Example 18, 114 mg, 63% yield) as a white amorphous solid. ¹H NMR (500 MHz, DMSO-d6) δ 2.00-2.43 (2H, m), 3.47-4.13 (4H, m), 4.68-5.05 (1H, m), 5.67 (1H, ddd), 6.15 (1H, ddd), 6.46-6.77 (1H, m), 7.78 (1H, br dd), 7.84-7.98 (2H, m), 8.28 (1H, dd), 8.39 (1H, br d), 8.48 (1H, br dd), 8.62 (1H, br d), 9.12 (1H, s); m/z: (ES⁺) [M+H]⁺=414.

### Example 19: (S)-1-(3-((4-(4-(trifluoromethyl)phenyl)phthalazin-1-yl)amino)pyrrolidin-1-yl)prop-2-yn-1-one

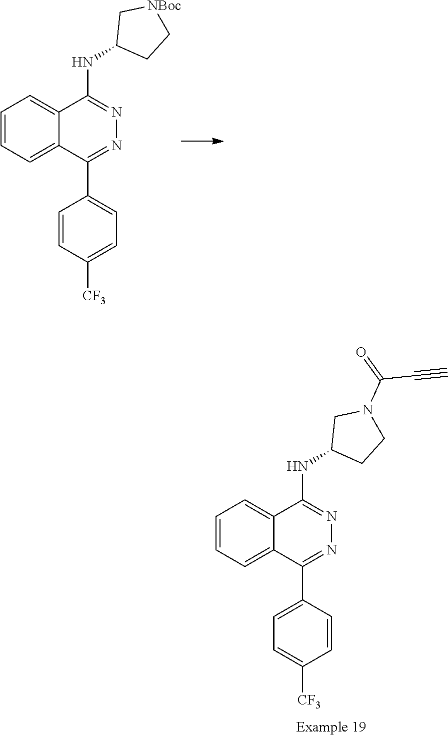 [SMILES: CC(C)(C)OC(=O)N1CC[C@H](Nc2nnc(-c3ccc(C(F)(F)F)cc3)c3ccccc23)C1 ; C#CC(=O)N1CC[C@H](Nc2nnc(-c3ccc(C(F)(F)F)cc3)c3ccccc23)C1]

DIPEA (177 μL, 1.01 mmol) was added to a solution of (S)—N-(pyrrolidin-3-yl)-4-(4-(trifluoromethyl)phenyl)phthalazin-1-amine, HCl (100 mg, 0.25 mmol), propiolic acid (21 mg, 0.30 mmol) and HATU (116 mg, 0.300 mmol) in DMF (4 mL) and the reaction mixture stirred at rt for 15 hrs. The reaction mixture was diluted with DCM (100 mL) and washed with saturated aq. NaHCO₃ (15 mL). The organic layer was dried over Na₂SO₄, filtered and concentrated to dryness. The crude material was purified on a reverse phase column (0 to 100% MeCN in H₂O w/0.1% HCO₂H) to afford (S)-1-(3-((4-(4-(trifluoromethyl)phenyl)phthalazin-1-yl)amino)pyrrolidin-1-yl)prop-2-yn-1-one (Example 19, 57 mg, 55% yield) as a light-yellow amorphous solid. ¹H NMR (500 MHz, DMSO-d6) δ 2.13-2.27 (1H, m), 2.28-2.44 (1H, m), 3.49-3.67 (1.5H, m), 3.72-3.92 (2H, m), 4.02-4.17 (0.5H, m), 4.45-4.49 (1H, d), 4.73-4.92 (1H, m), 7.75-8.06 (8H, m), 8.54 (1H, br d); m/z: (ES⁺) [M+H]⁺=411.

### Example 20: (S)-1-(3-((4-(4-(trifluoromethyl)phenyl)phthalazin-1-yl)amino)pyrrolidin-1-yl)but-2-yn-1-one

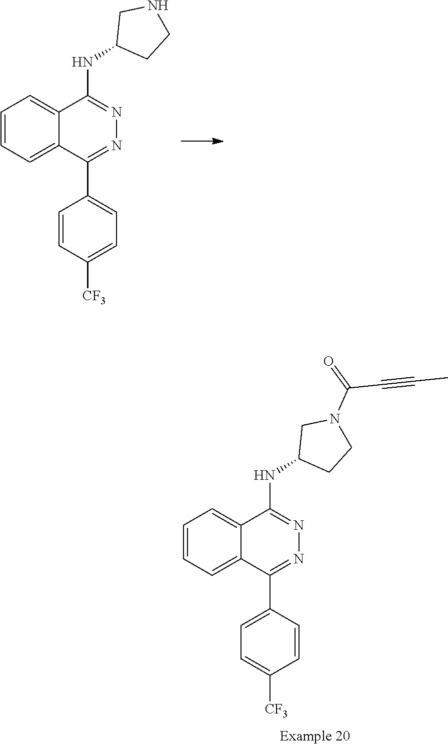 [SMILES: FC(F)(F)c1ccc(-c2nnc(N[C@H]3CCNC3)c3ccccc23)cc1 ; CC#CC(=O)N1CC[C@H](Nc2nnc(-c3ccc(C(F)(F)F)cc3)c3ccccc23)C1]

DIPEA (177 μL, 1.01 mmol) was added to a solution of (S)—N-(pyrrolidin-3-yl)-4-(4-(trifluoromethyl)phenyl)phthalazin-1-amine, HCl (100 mg, 0.25 mmol), but-2-ynoic acid (26 mg, 0.30 mmol) and HATU (116 mg, 0.300 mmol) in DMF (4 mL) and the reaction mixture stirred at rt for 15 hrs. The reaction mixture was diluted with DCM (100 mL) and washed with saturated aq. NaHCO₃ (15 mL). The organic layer was dried over Na₂SO₄, filtered and concentrated to dryness. The crude material was purified a on reverse phase column (0 to 100% MeCN in H₂O w/0.1% HCO₂H) to afford (S)-1-(3-((4-(4-(trifluoromethyl)phenyl)phthalazin-1-yl)amino)pyrrolidin-1-yl)but-2-yn-1-one (Example 20, 59 mg, 55% yield) as a white amorphous solid. ¹H NMR (500 MHz, DMSO-d6) δ 1.89-2.09 (3H, m), 2.18 (1H, br d), 2.33 (1H, br d), 3.44-3.66 (1.5H, m), 3.67-3.92 (2H, m), 4.09 (0.5H, br dd), 4.84 (1H, br d), 7.69 (1H, br s), 7.74-8.05 (7H, m), 8.51 (1H, br d); m/z: (ES⁺) [M+H]⁺=425.

### Example 21: (S)-4-(4-(trifluoromethyl)phenyl)-N-(1-(vinylsulfonyl)pyrrolidin-3-yl)phthalazin-1-amine

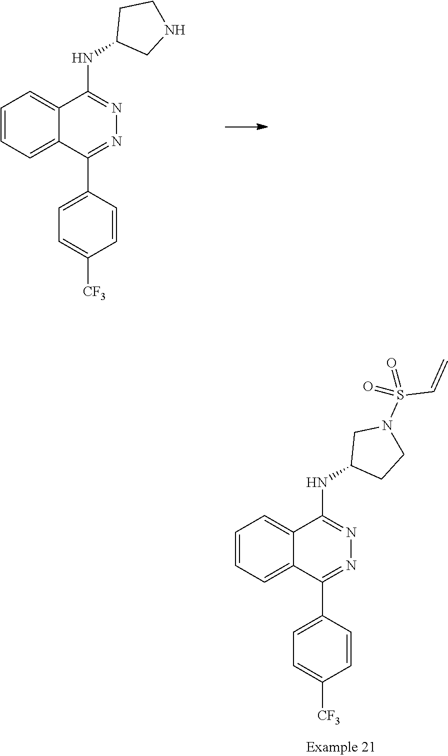 [SMILES: FC(F)(F)c1ccc(-c2nnc(N[C@@H]3CCNC3)c3ccccc23)cc1 ; C=CS(=O)(=O)N1CC[C@H](Nc2nnc(-c3ccc(C(F)(F)F)cc3)c3ccccc23)C1]

A solution of ethenesulfonyl chloride (32 mg, 0.25 mmol) in DCM (1 mL) was added dropwise to a solution of (S)—N-(pyrrolidin-3-yl)-4-(4-(trifluoromethyl)phenyl)phthalazin-1-amine, HCl (100 mg, 0.25 mmol) and DIPEA (177 μL, 1.01 mmol) in DCM (5 mL) at 0° C. The reaction mixture stirred at 0° C. for 1 h. The reaction mixture was diluted with DCM (100 mL) and washed with water (10 mL) and brine (10 mL). The organic layer was dried over Na₂SO₄, filtered and concentrated to dryness. The crude material was purified on a reverse phase C18 column (0 to 100% MeCN in H₂O w/0.1% HCO₂H) to yield impure material which was further purified on a reverse phase C18 column (0 to 100% MeCN in H₂O w/0.2% NH₄OH) to afford (S)-4-(4-(trifluoromethyl)phenyl)-N-(1-(vinylsulfonyl)pyrrolidin-3-yl)phthalazin-1-amine (Example 21, 21 mg, 18% yield) as a white amorphous solid. ¹H NMR (500 MHz, DMSO-d6) δ 2.17 (1H, dq), 2.34 (1H, dq), 3.23-3.31 (1H, m), 3.36-3.40 (1H, m), 3.47-3.57 (1H, m), 3.70 (1H, dd), 4.70-4.88 (1H, m), 6.01-6.20 (2H, m), 6.89 (1H, dd), 7.57 (1H, br d), 7.80 (1H, d), 7.83-8.03 (6H, m), 8.47 (1H, d); m/z: (ES⁺) [M+H]⁺=449.

### Example 22: 1-((3S,4R)-3-(hydroxymethyl)-4-((4-(4-(trifluoromethyl)phenyl)phthalazin-1-yl)amino)pyrrolidin-1-yl)prop-2-en-1-one

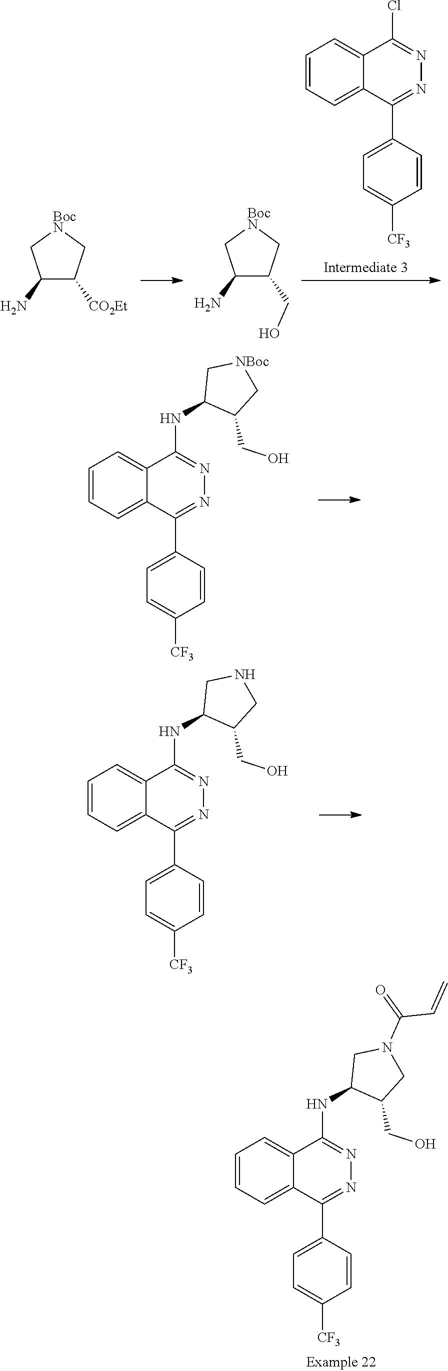

### tert-butyl (3R,4S)-3-amino-4-(hydroxymethyl)pyrrolidine-1-carboxylate

LiBH₄ (2M in THF, 1.40 mL, 2.85 mmol) was added to a solution of 1-(tert-butyl) 3-ethyl (3S,4R)-4-aminopyrrolidine-1,3-dicarboxylate, HCl (420 mg, 1.42 mmol) in THF (8 mL) at 0° C. The reaction mixture was warmed to rt and stirred for 2 hrs. The volatiles were removed under reduced pressure and the resulting residue was diluted in MeOH (3 mL) and the resulting mixture was heated to 90° C. and stirred for 2 hrs. The reaction mixture was cooled to rt, diluted with saturated aq. NaHCO₃ (6 mL), and extracted with a 2:1 CHCl₃/MeOH solution (3×12 mL). The combined organics were dried over Na₂SO₄, filtered and concentrated to dryness to afford tert-butyl (3R,4S)-3-amino-4-(hydroxymethyl)pyrrolidine-1-carboxylate (254 mg, 82% yield) as an amber oil. ¹H NMR (500 MHz, DMSO-d6) δ 1.39 (9H, s), 1.61 (2H, br s), 1.78-1.95 (1H, m), 2.83 (1H, br d), 2.95-3.11 (2H, m), 3.33-3.37 (1H, m), 3.38-3.47 (2H, m), 3.49 (1H, br dd), 4.52-4.68 (1H, m); m/z: (ES⁺) [M+H]⁺=217.

### tert-butyl (3S,4R)-3-(hydroxymethyl)-4-((4-(4-(trifluoromethyl)phenyl)phthalazin-1-yl)amino)pyrrolidine-1-carboxylate

DIPEA (0.42 mL, 2.4 mmol) was added to a solution of 1-chloro-4-(4-(trifluoromethyl)phenyl)phthalazine (Intermediate 3, 150 mg, 0.49 mmol) and tert-butyl (3R,4S)-3-amino-4-(hydroxymethyl)pyrrolidine-1-carboxylate (252 mg, 1.17 mmol) in DMSO (2.5 mL). The reaction mixture was heated to 85° C. and stirred for 90 hrs. The reaction mixture was cooled to rt, diluted with water (5 mL), and extracted with EtOAc (3×10 mL). The combined organics were dried over Na₂SO₄, filtered and concentrated to dryness. The crude material was purified by flash silica chromatography (20-100% EtOAc in hexanes) to afford tert-butyl (3S,4R)-3-(hydroxymethyl)-4-((4-(4-(trifluoromethyl)phenyl)phthalazin-1-yl)amino)pyrrolidine-1-carboxylate (63 mg, 27% yield) as an off-white foam. ¹H NMR (400 MHz, DMSO-d6) δ 1.41 (9H, br d), 2.55-2.63 (1H, m), 3.20-3.28 (2H, m), 3.43-3.54 (1H, m), 3.56-3.63 (2H, m), 3.78-3.94 (1H, m), 4.60-4.69 (1H, m), 4.85 (1H, t), 7.58 (1H, d), 7.77-7.81 (1H, m), 7.84-7.98 (6H, m), 8.50 (1H, d); m/z: (ES⁺) [M+H]⁺=488.

### ((3S,4R)-4-((4-(4-(trifluoromethyl)phenyl)phthalazin-1-yl)amino)pyrrolidin-3-yl)methanol

HCl (4M in 1,4-dioxane, 0.96 mL, 3.8 mmol) was added to a solution of tert-butyl (3S,4R)-3-(hydroxymethyl)-4-((4-(4-(trifluoromethyl)phenyl)phthalazin-1-yl)amino)pyrrolidine-1-carboxylate (63 mg, 0.13 mmol) in MeOH (1.5 mL) at 0° C. The reaction mixture was warmed to rt and stirred for 2 hrs. The reaction mixture was diluted with saturated aq. NaHCO₃ (6 mL) and extracted with a 2:1 CHCl₃/MeOH solution (3×12 mL). The combined organics were dried over Na₂SO₄, filtered and concentrated to dryness to afford ((3S,4R)-4-((4-(4-(trifluoromethyl)phenyl)phthalazin-1-yl)amino)pyrrolidin-3-yl)methanol (24 mg, 48% yield) as a tan gum. m/z: (ES⁺) [M+H]⁺=389.2.

### 1-((3S,4R)-3-(hydroxymethyl)-4-((4-(4-(trifluoromethyl)phenyl)phthalazin-1-yl)amino)pyrrolidin-1-yl)prop-2-en-1-one

A solution of acryloyl chloride (5 μL, 0.06 mmol) in DCM (0.1 mL) was added dropwise to a solution of ((3S,4R)-4-((4-(4-(trifluoromethyl)phenyl)phthalazin-1-yl)amino)pyrrolidin-3-yl)methanol (24 mg, 0.062 mmol) and DIPEA (20 μL, 0.12 mmol) in DCM (0.8 mL) at 0° C. The reaction mixture was stirred at 0° C. for 30 min. The reaction mixture was diluted with brine (5 mL) and the layers were separated. The aq. layer was extracted with DCM (3×10 mL) and the combined organics were dried over Na₂SO₄, filtered and concentrated to dryness. The crude material was purified by reverse phase HPLC (25 to 50% MeCN in water w/0.1% HCO₂H) to afford 1-((3S,4R)-3-(hydroxymethyl)-4-((4-(4-(trifluoromethyl)phenyl)phthalazin-1-yl)amino)pyrrolidin-1-yl)prop-2-en-1-one (Example 22, 15 mg, 55% yield) as a white amorphous solid. ¹H NMR (500 MHz, DMSO-d6) δ 2.57-2.75 (1H, m), 3.40-3.54 (2H, m), 3.54-3.67 (2H, m), 3.74-4.24 (2H, m), 4.62-4.81 (1H, m), 4.86-5.02 (1H, m), 5.63-5.73 (1H, m), 6.16 (1H, ddd), 6.55-6.70 (1H, m), 7.66 (1H, dd), 7.76-7.86 (1H, m), 7.86-7.98 (6H, m), 8.51 (1H, d); m/z: (ES⁺) [M+H]⁺=443.1.

### Example 23: 1-((3R,4S)-3-(hydroxymethyl)-4-((4-(4-(trifluoromethyl)phenyl)phthalazin-1-yl)amino)pyrrolidin-1-yl)prop-2-en-1-one

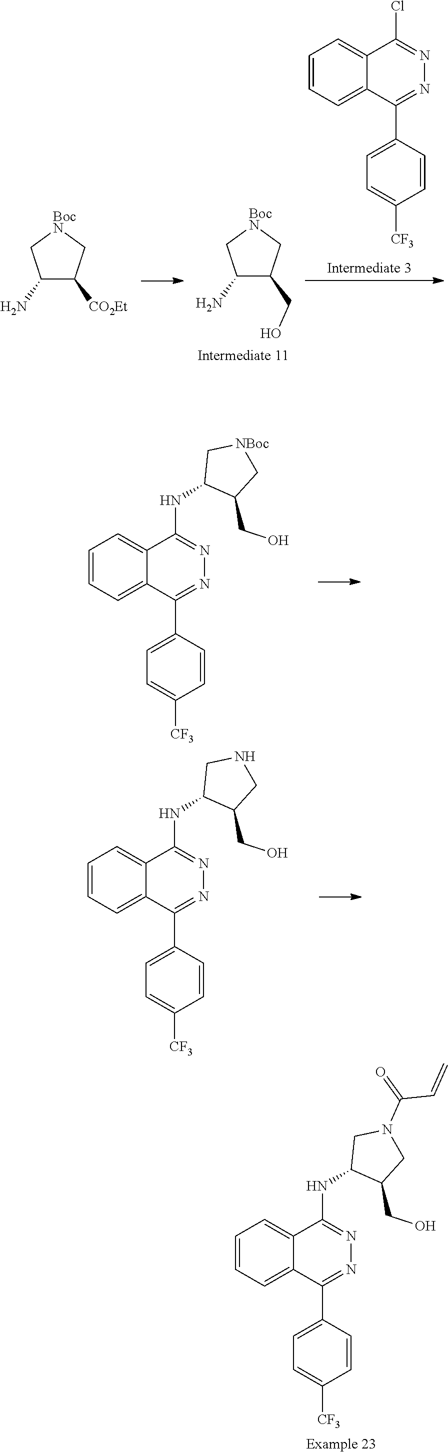

### Intermediate 11: tert-butyl (3S,4R)-3-amino-4-(hydroxymethyl)pyrrolidine-1-carboxylate

LiBH₄ (2M in THF, 3.90 mL, 7.74 mmol) was added to a solution of 1-(tert-butyl) 3-ethyl (3R,4S)-4-aminopyrrolidine-1,3-dicarboxylate (1.00 g, 3.87 mmol) in THF (20 mL) at 0° C. The reaction mixture was warmed to rt and stirred for 16 hrs. The volatiles were removed under reduced pressure and the resulting residue was diluted in MeOH (8 mL) and the resulting mixture was heated to 90° C. and stirred for 2 hrs. The reaction mixture was cooled to rt, diluted with saturated aq. NaHCO₃ (6 mL), and extracted with a 2:1 CHCl₃/MeOH solution (3×12 mL). The combined organics were dried over Na₂SO₄, filtered and concentrated to dryness to afford tert-butyl (3S,4R)-3-amino-4-(hydroxymethyl)pyrrolidine-1-carboxylate (Intermediate 11, 730 mg, 87% yield) as an amber oil that was lyophilized to a white sticky solid. ¹H NMR (500 MHz, DMSO-d6) δ 1.39 (9H, s), 1.54-1.67 (2H, m), 1.80-1.93 (1H, m), 2.78-2.87 (1H, m), 2.95-3.12 (2H, m), 3.29-3.33 (1H, m), 3.38-3.46 (2H, m), 3.46-3.52 (1H, m), 4.61-4.69 (1H, m); m/z: (ES⁺) [M+H]⁺=217.

### tert-butyl (3R,4S)-3-(hydroxymethyl)-4-((4-(4-(trifluoromethyl)phenyl)phthalazin-1-yl)amino)pyrrolidine-1-carboxylate

DIPEA (0.45 mL, 2.59 mmol) was added to a solution of 1-chloro-4-(4-(trifluoromethyl)phenyl)phthalazine (Intermediate 3, 200 mg, 0.65 mmol) and tert-butyl (3S,4R)-3-amino-4-(hydroxymethyl)pyrrolidine-1-carboxylate (210 mg, 0.97 mmol) in DMSO (3.5 mL). The reaction mixture was heated to 85° C. and stirred for 90 hrs. The reaction mixture was cooled to rt, diluted with water (5 mL), and extracted with EtOAc (3×10 mL). The combined organics were dried over Na₂SO₄, filtered and concentrated to dryness. The crude material was purified by flash silica chromatography (20-100% EtOAc in hexanes) to afford tert-butyl (3R,4S)-3-(hydroxymethyl)-4-((4-(4-(trifluoromethyl)phenyl)phthalazin-1-yl)amino)pyrrolidine-1-carboxylate (90 mg, 28% yield) as an off-white foam. ¹H NMR (500 MHz, DMSO-d6) δ 1.42 (9H, s), 2.55-2.64 (1H, m), 3.25-3.30 (2H, m), 3.49 (1H, br s), 3.60 (2H, dt), 3.84-3.94 (1H, m), 4.65 (1H, br d), 4.86 (1H, t), 7.59 (1H, br d), 7.80 (1H, d), 7.86-7.98 (6H, m), 8.51 (1H, d); m/z: (ES⁺) [M+H]⁺=489.

### ((3R,4S)-4-((4-(4-(trifluoromethyl)phenyl)phthalazin-1-yl)amino)pyrrolidin-3-yl)methanol

HCl (4 M in 1,4-dioxane, 1.5 mL, 6.1 mmol) was added to a solution of tert-butyl (3R,4S)-3-(hydroxymethyl)-4-((4-(4-(trifluoromethyl)phenyl)phthalazin-1-yl)amino)pyrrolidine-1-carboxylate (100 mg, 0.20 mmol) in MeOH (1.5 mL) at 0° C. The reaction mixture was warmed to rt and stirred for 2 hrs. The reaction mixture was diluted with saturated aq. NaHCO₃ (6 mL) and extracted with a 2:1 CHCl₃/MeOH solution (3×12 mL). The combined organics were dried over Na₂SO₄, filtered and concentrated to dryness to afford ((3R,4S)-4-((4-(4-(trifluoromethyl)phenyl)phthalazin-1-yl)amino)pyrrolidin-3-yl)methanol (25 mg, 31% yield) as a tan solid. m/z: (ES⁺) [M+H]⁺=389.

### 1-((3R,4S)-3-(hydroxymethyl)-4-((4-(4-(trifluoromethyl)phenyl)phthalazin-1-yl)amino)pyrrolidin-1-yl)prop-2-en-1-one

A solution of acryloyl chloride (5 μL, 0.06 mmol) in DCM (0.2 mL) was added dropwise to a solution of ((3R,4S)-4-((4-(4-(trifluoromethyl)phenyl)phthalazin-1-yl)amino)pyrrolidin-3-yl)methanol (25 mg, 0.062 mmol) and DIPEA (30 μL, 0.19 mmol) in DCM (1.3 mL) at 0° C. The reaction mixture was stirred at 0° C. for 30 min. The reaction mixture was diluted with brine (5 mL) and the layers were separated. The aq. layer was extracted with DCM (3×10 mL) and the combined organics were dried over Na₂SO₄, filtered and concentrated to dryness. The crude material was purified on a reverse phase C18 column (2.5 to 25% MeCN in H₂O w/0.1% HCO₂H) to afford 1-((3R,4S)-3-(hydroxymethyl)-4-((4-(4-(trifluoromethyl)phenyl)phthalazin-1-yl)amino)pyrrolidin-1-yl)prop-2-en-1-one (Example 23, 22 mg, 77% yield) as a white amorphous solid. ¹H NMR (500 MHz, DMSO-d6) δ 2.56-2.73 (1H, m), 3.38-3.55 (2H, m), 3.55-3.67 (2H, m), 3.74-4.26 (2H, m), 4.62-4.82 (1H, m), 4.83-5.03 (1H, m), 5.63-5.73 (1H, m), 6.16 (1H, ddd), 6.55-6.70 (1H, m), 7.64 (1H, dd), 7.77-7.86 (1H, m), 7.86-7.99 (6H, m), 8.51 (1H, d); m/z: (ES⁺) [M+H]⁺=443.

### Example 24: 1-((3R,4S)-3-(hydroxymethyl)-4-((8-(4-(trifluoromethyl)phenyl)-1,6-naphthyridin-5-yl)amino)pyrrolidin-1-yl)prop-2-en-1-one

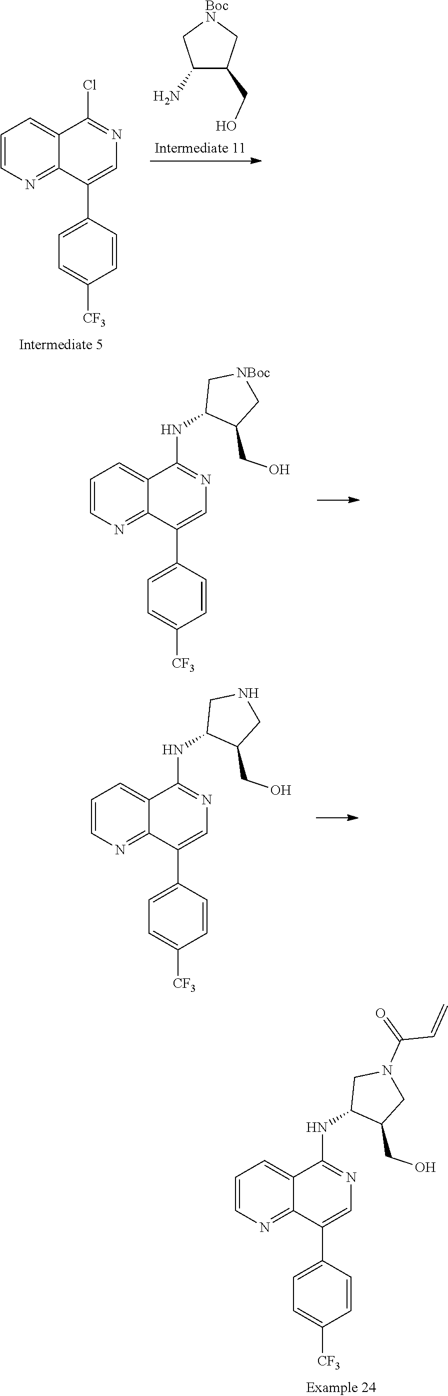 [SMILES: FC(F)(F)c1ccc(-c2cnc(Cl)c3cccnc23)cc1 ; CC(C)(C)OC(=O)N1C[C@@H](CO)[C@H](N)C1 ; CC(C)(C)OC(=O)N1C[C@@H](CO)[C@H](Nc2ncc(-c3ccc(C(F)(F)F)cc3)c3ncccc23)C1 ; OC[C@@H]1CNC[C@H]1Nc1ncc(-c2ccc(C(F)(F)F)cc2)c2ncccc12 ; C=CC(=O)N1C[C@@H](CO)[C@H](Nc2ncc(-c3ccc(C(F)(F)F)cc3)c3ncccc23)C1]

### tert-butyl (3R,4S)-3-(hydroxymethyl)-4-((8-(4-(trifluoromethyl)phenyl)-1,6-naphthyridin-5-yl)amino)pyrrolidine-1-carboxylate

DIPEA (0.26 mL, 1.46 mmol) was added to a solution of 5-chloro-8-(4-(trifluoromethyl)phenyl)-1,6-naphthyridine (Intermediate 5, 150 mg, 0.49 mmol) and tert-butyl (3S,4R)-3-amino-4-(hydroxymethyl)pyrrolidine-1-carboxylate (Intermediate 11, 158 mg, 0.73 mmol) in DMSO (1.6 mL). The reaction mixture was heated to 85° C. and stirred for 40 hrs. The reaction mixture was cooled to rt, diluted with water (5 mL), and extracted with EtOAc (3×10 mL). The combined organics were dried over Na₂SO₄, filtered and concentrated to dryness. The crude material was purified by flash silica chromatography (20-70% EtOAc in hexanes) to afford tert-butyl (3R,4S)-3-(hydroxymethyl)-4-((8-(4-(trifluoromethyl)phenyl)-1,6-naphthyridin-5-yl)amino)pyrrolidine-1-carboxylate (41 mg, 17% yield) as a yellow green solid. ¹H NMR (500 MHz, DMSO-d6) δ 1.37-1.46 (9H, m), 2.54-2.58 (1H, m), 3.17-3.28 (2H, m), 3.44-3.51 (1H, m), 3.51-3.66 (2H, m), 3.76-3.88 (1H, m), 4.49-4.70 (1H, m), 4.79 (1H, t), 7.56-7.65 (1H, m), 7.75-7.83 (3H, m), 7.83-7.90 (2H, m), 8.20-8.26 (1H, m), 8.80-8.92 (1H, m), 8.92-9.01 (1H, m); m/z: (ES⁺) [M+H]⁺=489.

### ((3R,4S)-4-((8-(4-(trifluoromethyl)phenyl)-1,6-naphthyridin-5-yl)amino)pyrrolidin-3-yl)methanol

HCl (4 M in 1,4-dioxane, 0.63 mL, 2.5 mmol) was added to a solution of tert-butyl (3R,4S)-3-(hydroxymethyl)-4-((8-(4-(trifluoromethyl)phenyl)-1,6-naphthyridin-5-yl)amino)pyrrolidine-1-carboxylate (41 mg, 0.08 mmol) in MeOH (1.2 mL) at 0° C. The reaction mixture was warmed to rt and stirred for 2 hrs. The reaction mixture was diluted with saturated aq. NaHCO₃ (6 mL) and extracted with a 2:1 CHCl₃/MeOH solution (3×12 mL). The combined organics were dried over Na₂SO₄, filtered and concentrated to dryness to afford ((3R,4S)-4-((8-(4-(trifluoromethyl)phenyl)-1,6-naphthyridin-5-yl)amino)pyrrolidin-3-yl)methanol (33 mg, 100% yield) as a green oil. m/z: (ES⁺) [M+H]⁺=389.

### 1-((3R,4S)-3-(hydroxymethyl)-4-((8-(4-(trifluoromethyl)phenyl)-1,6-naphthyridin-5-yl)amino)pyrrolidin-1-yl)prop-2-en-1-one

A solution of acryloyl chloride (7 μL, 0.08 mmol) in DCM (0.2 mL) was added dropwise to a mixture of ((3R,4S)-4-((8-(4-(trifluoromethyl)phenyl)-1,6-naphthyridin-5-yl)amino)pyrrolidin-3-yl)methanol (33 mg, 0.08 mmol) and DIPEA (50 μL, 0.25 mmol) in DCM (1.3 mL) at 0° C. The reaction mixture was stirred at this temperature for 0.5 h and then diluted with brine (5 mL). The phases were separated and the aqueous layer was extracted with DCM (3×10 mL). The combined organics were dried over Na₂SO₄, filtered and concentrated to dryness. The crude material was purified on a reverse phase C18 column (2.5 to 25% MeCN in H₂O w/0.1% HCO₂H) to afford 1-((3R,4S)-3-(hydroxymethyl)-4-((8-(4-(trifluoromethyl)phenyl)-1,6-naphthyridin-5-yl)amino)pyrrolidin-1-yl)prop-2-en-1-one (Example 24, 18 mg, 43% yield) as a light-yellow amorphous solid. ¹H NMR (500 MHz, DMSO-d6) δ 2.56-2.70 (1H, m), 3.34-3.45 (2H, m), 3.45-3.63 (3H, m), 3.74-4.19 (2H, m), 4.55-4.77 (1H, m), 4.77-5.02 (1H, m), 5.63-5.73 (1H, m), 6.16 (1H, ddd), 6.50-6.72 (1H, m), 7.59-7.67 (1H, m), 7.79 (2H, br d), 7.83-7.97 (3H, m), 8.89 (1H, br d), 8.99 (1H, br d); m/z: (ES⁺) [M+H]⁺=443.

### Example 25: 1-((3R,4S)-3-(hydroxymethyl)-4-((8-(4-(trifluoromethyl)phenyl)pyrido[2,3-d]pyridazin-5-yl)amino)pyrrolidin-1-yl)prop-2-en-1-one

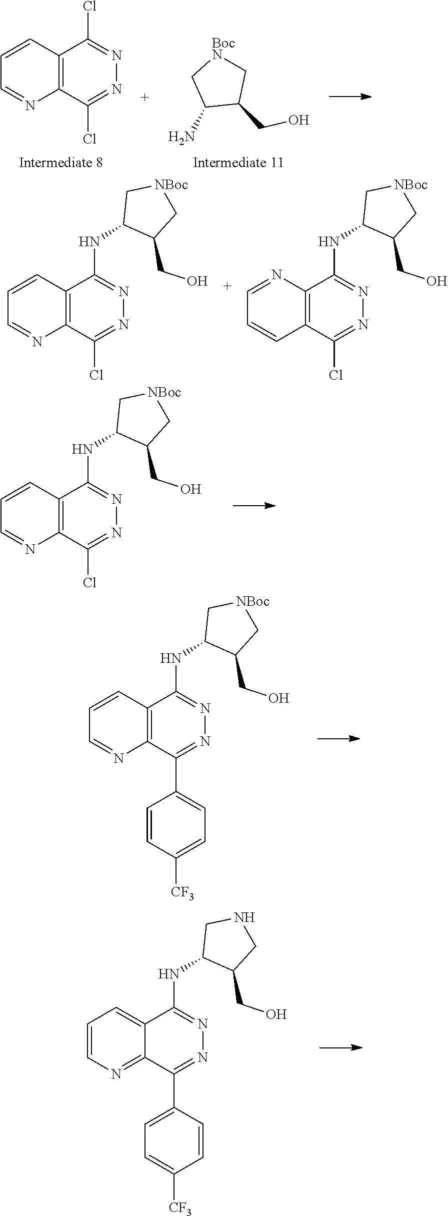

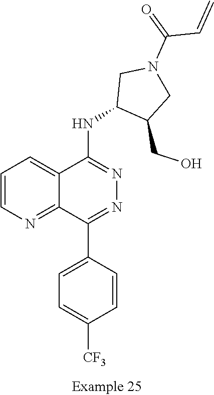 [SMILES: C=CC(=O)N1C[C@@H](CO)[C@H](Nc2nnc(-c3ccc(C(F)(F)F)cc3)c3ncccc23)C1]

### tert-butyl (3S,4R)-3-((8-chloropyrido[2,3-d]pyridazin-5-yl)amino)-4-(hydroxymethyl)pyrrolidine-1-carboxylate and tert-butyl (3S,4R)-3-((5-chloropyrido[2,3-d]pyridazin-8-yl)amino)-4-(hydroxymethyl)pyrrolidine-1-carboxylate

DIPEA (0.38 mL, 2.2 mmol) was added to a solution of 5,8-dichloropyrido[2,3-d]pyridazine (Intermediate 8, 220 mg, 1.1 mmol) and tert-butyl (3S,4R)-3-amino-4-(hydroxymethyl)pyrrolidine-1-carboxylate (Intermediate 11, 285 mg, 1.32 mmol) in DMSO (2.2 mL). The reaction mixture was heated to 85° C. and stirred for 20 hrs. The reaction mixture was cooled to rt, diluted with water (10 mL), and extracted with EtOAc (3×20 mL). The combined organics were dried over Na₂SO₄, filtered and concentrated to dryness. The crude material was purified on a reverse phase C18 column (5 to 30% MeCN in H₂O w/0.1% NH₄OH) to afford tert-butyl (3S,4R)-3-((8-chloropyrido[2,3-d]pyridazin-5-yl)amino)-4-(hydroxymethyl)pyrrolidine-1-carboxylate (56 mg, 13% yield) and tert-butyl (3S,4R)-3-((5-chloropyrido[2,3-d]pyridazin-8-yl)amino)-4-(hydroxymethyl)pyrrolidine-1-carboxylate (60 mg, 14% yield) as amber solids.

### tert-butyl (3S,4R)-3-((8-chloropyrido[2,3-d]pyridazin-5-yl)amino)-4-(hydroxymethyl)pyrrolidine-1-carboxylate

¹H NMR (500 MHz, DMSO-d6) δ 1.41 (9H, br d), 3.19-3.27 (2H, m), 3.41-3.49 (1H, m), 3.58 (3H, br d), 3.74-3.89 (1H, m), 4.47-4.58 (1H, m), 4.81 (1H, s), 7.81 (1H, d), 8.02 (1H, dd), 8.92 (1H, dd), 9.25 (1H, dd); m/z: (ES⁺) [M+H]⁺=380.

### tert-butyl (3S,4R)-3-((5-chloropyrido[2,3-d]pyridazin-8-yl)amino)-4-(hydroxymethyl)pyrrolidine-1-carboxylate

¹H NMR (500 MHz, DMSO-d6) δ 1.39-1.44 (9H, m), 2.62-2.69 (1H, m), 3.15-3.28 (2H, m), 3.42-3.50 (1H, m), 3.50-3.62 (2H, m), 3.76-3.87 (1H, m), 4.54 (1H, br t), 4.79 (1H, br s), 7.91 (1H, d), 8.06 (1H, dd), 8.49 (1H, dd), 9.22 (1H, dd); m/z: (ES⁺) [M+H]⁺=380.

### tert-butyl (3R,4S)-3-(hydroxymethyl)-4-((8-(4-(trifluoromethyl)phenyl)pyrido[2,3-d]pyridazin-5-yl)amino)pyrrolidine-1-carboxylate

tert-Butyl (3S,4R)-3-((8-chloropyrido[2,3-d]pyridazin-5-yl)amino)-4-(hydroxymethyl)pyrrolidine-1-carboxylate (56 mg, 0.15 mmol), (4-(trifluoromethyl)phenyl)boronic acid (56 mg, 0.29 mmol), PdCl₂(dppf) (27 mg, 0.04 mmol) and Cs₂CO₃ (192 mg, 0.59 mmol) were diluted in 1,4-dioxane (1 mL) and water (0.2 mL) under an atmosphere of N₂. The reaction mixture was heated to 90° C. and stirred for 2 hrs. The reaction mixture was cooled to rt and filtered through diatomaceous earth and the filtrate was concentrated. The resulting residue was diluted in water (10 mL) and extracted with EtOAc (3×20 mL). The combined organics were washed with brine (20 mL), dried over Na₂SO₄, filtered and concentrated to dryness. The crude material was purified by flash silica chromatography (10-100% EtOAc in hexanes) to afford tert-butyl (3R,4S)-3-(hydroxymethyl)-4-((8-(4-(trifluoromethyl)phenyl)pyrido[2,3-d]pyridazin-5-yl)amino)pyrrolidine-1-carboxylate (28 mg, 39% yield) as a yellow solid. ¹H NMR (500 MHz, DMSO-d6) δ 1.42 (9H, br d), 3.24-3.30 (3H, m), 3.44-3.54 (1H, m), 3.54-3.69 (2H, m), 3.80-3.95 (1H, m), 4.60-4.73 (1H, m), 4.85 (1H, t), 7.81-7.85 (1H, m), 7.85-7.90 (2H, m), 7.97 (1H, dd), 8.21 (2H, d), 8.88-8.96 (1H, m), 9.15-9.23 (1H, m); m/z: (ES⁺) [M+H]⁺=490.

### ((3R,4S)-4-((8-(4-(trifluoromethyl)phenyl)pyrido[2,3-d]pyridazin-5-yl)amino)pyrrolidin-3-yl)methanol

HCl (4 M in 1,4-dioxane, 0.35 mL, 1.4 mmol) was added to a solution of tert-butyl (3R,4S)-3-(hydroxymethyl)-4-((8-(4-(trifluoromethyl)phenyl)pyrido[2,3-d]pyridazin-5-yl)amino)pyrrolidine-1-carboxylate (28 mg, 0.06 mmol) in MeOH (1 mL) at 0° C. The reaction mixture was warmed to rt and stirred for 4 hrs. The mixture was diluted with saturated aq. NaHCO₃ (6 mL) and extracted with a 2:1 CHCl₃/MeOH solution (3×12 mL). The combined organics were dried over Na₂SO₄, filtered and concentrated to dryness to afford ((3R,4S)-4-((8-(4-(trifluoromethyl)phenyl)pyrido[2,3-d]pyridazin-5-yl)amino)pyrrolidin-3-yl)methanol (14 mg, 63% yield) as a green oil. m/z: (ES⁺) [M+H]⁺=390.

### 1-((3R,4S)-3-(hydroxymethyl)-4-((8-(4-(trifluoromethyl)phenyl)pyrido[2,3-d]pyridazin-5-yl)amino)pyrrolidin-1-yl)prop-2-en-1-one

A solution of acryloyl chloride (3 μL, 0.03 mmol) in DCM (0.1 mL) was added dropwise to a solution of ((3R,4S)-4-((8-(4-(trifluoromethyl)phenyl)pyrido[2,3-d]pyridazin-5-yl)amino)pyrrolidin-3-yl)methanol (14 mg, 0.04 mmol) and DIPEA (20 μL, 0.11 mmol) in DCM (0.9 mL) at 0° C. The reaction mixture was stirred at this temperature for 0.5 h and then diluted with brine (5 mL). The phases were separated and the aqueous layer was extracted with DCM (3×10 mL). The combined organics were dried over Na₂SO₄, filtered and concentrated to dryness. The crude material was purified on a reverse phase C18 column (2.5 to 25% MeCN in H₂O w/0.1% HCO₂H buffer) to afford 1-((3R,4S)-3-(hydroxymethyl)-4-((8-(4-(trifluoromethyl)phenyl)pyrido[2,3-d]pyridazin-5-yl)amino)pyrrolidin-1-yl)prop-2-en-1-one (Example 25, 12 mg, 75% yield) as a white amorphous solid. ¹H NMR (500 MHz, DMSO-d6) δ 2.60-2.78 (1H, m), 3.35-3.48 (2H, m), 3.48-3.56 (2H, m), 3.56-3.67 (2H, m), 3.74-4.26 (2H, m), 4.63-4.82 (1H, m), 5.63-5.76 (1H, m), 6.17 (1H, br t), 6.52-6.71 (1H, m), 7.89 (2H, br d), 8.00 (1H, br dd), 8.17-8.23 (2H, m), 9.03 (1H, br s), 9.22 (1H, br d); m/z: (ES⁺) [M+H]⁺=444.

### Example 26: 1-((3R,4S)-3-(hydroxymethyl)-4-((5-(4-(trifluoromethyl)phenyl)pyrido[2,3-d]pyridazin-8-yl)amino)pyrrolidin-1-yl)prop-2-en-1-one

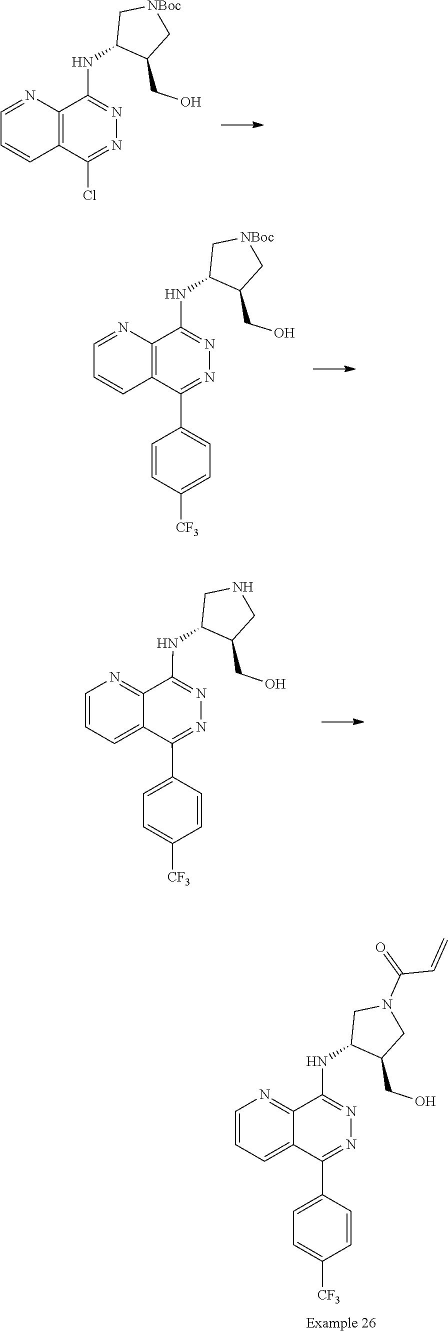 [SMILES: CC(C)(C)OC(=O)N1C[C@@H](CO)[C@H](Nc2nnc(Cl)c3cccnc23)C1 ; CC(C)(C)OC(=O)N1C[C@@H](CO)[C@H](Nc2nnc(-c3ccc(C(F)(F)F)cc3)c3cccnc23)C1 ; OC[C@@H]1CNC[C@H]1Nc1nnc(-c2ccc(C(F)(F)F)cc2)c2cccnc12 ; C=CC(=O)N1C[C@@H](CO)[C@H](Nc2nnc(-c3ccc(C(F)(F)F)cc3)c3cccnc23)C1]

### tert-butyl (3R,4S)-3-(hydroxymethyl)-4-((5-(4-(trifluoromethyl)phenyl)pyrido[2,3-d]pyridazin-8-yl)amino)pyrrolidine-1-carboxylate

tert-Butyl (3S,4R)-3-((5-chloropyrido[2,3-d]pyridazin-8-yl)amino)-4-(hydroxymethyl)pyrrolidine-1-carboxylate (60 mg, 0.16 mmol), (4-(trifluoromethyl)phenyl)boronic acid (60 mg, 0.32 mmol), PdCl₂(dppf) (29 mg, 0.04 mmol) and Cs₂CO₃ (206 mg, 0.63 mmol) were diluted in 1,4-dioxane (1 mL) and water (0.2 mL) under an atmosphere of N₂. The reaction mixture was heated to 90° C. and stirred for 2 hrs. The reaction mixture was cooled to rt and filtered through diatomaceous earth and the filtrate was concentrated. The resulting residue was diluted in water (10 mL) and extracted with EtOAc (3×20 mL). The combined organics were washed with brine (20 mL), dried over Na₂SO₄, filtered and concentrated to dryness. The crude material was purified by flash silica chromatography (20-100% EtOAc in hexanes) to afford tert-butyl (3R,4S)-3-(hydroxymethyl)-4-((5-(4-(trifluoromethyl)phenyl)pyrido[2,3-d]pyridazin-8-yl)amino)pyrrolidine-1-carboxylate (34 mg, 44% yield) as an orange oil. ¹H NMR (500 MHz, DMSO-d6) δ 1.39-1.46 (9H, m), 2.67-2.72 (1H, m), 3.15-3.27 (1H, m), 3.18-3.26 (1H, m), 3.44-3.54 (1H, m), 3.54-3.65 (2H, m), 3.79-3.95 (1H, m), 4.59-4.71 (1H, m), 4.83 (1H, t), 7.86 (1H, s), 7.89-7.97 (5H, m), 8.28 (1H, dd), 9.13-9.22 (1H, m). m/z: (ES⁺) [M+H]⁺=490.

### ((3R,4S)-4-((5-(4-(trifluoromethyl)phenyl)pyrido[2,3-d]pyridazin-8-yl)amino)pyrrolidin-3-yl)methanol

HCl (4 M in 1,4-dioxane, 0.43 mL, 1.7 mmol) was added to a solution of tert-butyl (3R,4S)-3-(hydroxymethyl)-4-((5-(4-(trifluoromethyl)phenyl)pyrido[2,3-d]pyridazin-8-yl)amino)pyrrolidine-1-carboxylate (34 mg, 0.07 mmol) in MeOH (1 mL) at 0° C. The reaction mixture was warmed to rt and stirred for 2 hrs. The reaction mixture was diluted with saturated aq. NaHCO₃ (6 mL) and extracted with a 2:1 CHCl₃/MeOH solution (3×12 mL). The combined organics were dried over Na₂SO₄, filtered and concentrated to dryness to afford ((3R,4S)-4-((5-(4-(trifluoromethyl)phenyl)pyrido[2,3-d]pyridazin-8-yl)amino)pyrrolidin-3-yl)methanol (26 mg, 96% yield) as an amber oil. m/z: (ES⁺) [M+H]⁺=390.

### 1-((3R,4S)-3-(hydroxymethyl)-4-((5-(4-(trifluoromethyl)phenyl)pyrido[2,3-d]pyridazin-8-yl)amino)pyrrolidin-1-yl)prop-2-en-1-one

A solution of acryloyl chloride (5 μL, 0.06 mmol) in DCM (0.2 mL) was added dropwise to a mixture of ((3R,4S)-4-((5-(4-(trifluoromethyl)phenyl)pyrido[2,3-d]pyridazin-8-yl)amino)pyrrolidin-3-yl)methanol (26 mg, 0.07 mmol) and DIPEA (35 μL, 0.20 mmol) in DCM (1 mL) at 0° C. The reaction mixture was stirred at this temperature for 0.5 h and then diluted with brine (5 mL). The phases were separated and the aqueous layer was extracted with DCM (3×10 mL). The combined organics were dried over Na₂SO₄, filtered and concentrated to dryness. The crude material was purified on a reverse phase C18 column (2.5 to 25% MeCN in H₂O w/0.1% HCO₂H) to afford 1-((3R,4S)-3-(hydroxymethyl)-4-((5-(4-(trifluoromethyl)phenyl)pyrido[2,3-d]pyridazin-8-yl)amino)pyrrolidin-1-yl)prop-2-en-1-one (Example 26, 14 mg, 47% yield) as a white amorphous solid. ¹H NMR (500 MHz, DMSO-d6) δ 2.65-2.83 (1H, m), 3.45-4.22 (6H, m), 4.64-4.83 (1H, m), 4.83-4.97 (1H, m), 5.61-5.75 (1H, m), 6.16 (1H, ddd), 6.53-6.69 (1H, m), 7.88-8.02 (6H, m), 8.29 (1H, br d), 9.15-9.24 (1H, m); m/z: (ES⁺) [M+H]⁺=444.

### Example 27: 1-((3R,4S)-3-(hydroxymethyl)-4-((8-(4-(trifluoromethyl)phenyl)pyrido[3,4-b]pyrazin-5-yl)amino)pyrrolidin-1-yl)prop-2-en-1-one

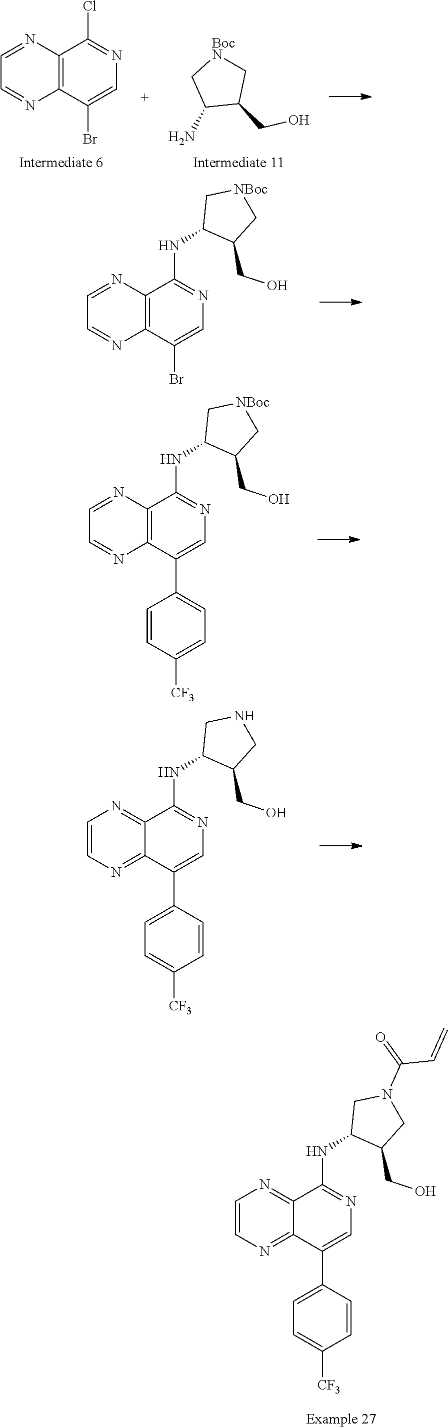

### tert-butyl (3S,4R)-3-((8-bromopyrido[3,4-b]pyrazin-5-yl)amino)-4-(hydroxymethyl)pyrrolidine-1-carboxylate

DIPEA (0.64 mL, 3.7 mmol) was added to a solution of 8-bromo-5-chloropyrido[3,4-b]pyrazine (Intermediate 6, 225 mg, 0.92 mmol) and tert-butyl (3S,4R)-3-amino-4-(hydroxymethyl)pyrrolidine-1-carboxylate (Intermediate 11, 398 mg, 1.84 mmol) in DMSO (3.2 mL). The reaction mixture was heated to 90° C. and stirred for 90 hrs. The reaction mixture was cooled to rt, diluted with water (5 mL), and extracted with EtOAc (3×15 mL). The combined organics were dried over Na₂SO₄, filtered and concentrated to dryness. The crude material was purified by flash silica chromatography (20-60% EtOAc in hexanes) to afford tert-butyl (3S,4R)-3-((8-bromopyrido[3,4-b]pyrazin-5-yl)amino)-4-(hydroxymethyl)pyrrolidine-1-carboxylate (268 mg, 69% yield) as a dark-orange foam. ¹H NMR (500 MHz, CDCl₃) δ 1.51 (9H, s), 2.26-2.35 (1H, m), 3.35-3.47 (2H, m), 3.70 (3H, br d), 3.93-4.05 (1H, m), 4.76 (1H, br s), 6.95 (1H, br d), 8.35-8.41 (1H, m), 8.72-8.77 (1H, m), 9.11 (1H, d); m/z: (ES⁺) [M+H]⁺=424.

### tert-butyl (3R,4S)-3-(hydroxymethyl)-4-((8-(4-(trifluoromethyl)phenyl)pyrido[3,4-b]pyrazin-5-yl)amino)pyrrolidine-1-carboxylate

tert-Butyl (3S,4R)-3-((8-bromopyrido[3,4-b]pyrazin-5-yl)amino)-4-(hydroxymethyl)pyrrolidine-1-carboxylate (150 mg, 0.35 mmol), (4-(trifluoromethyl)phenyl)boronic acid (134 mg, 0.71 mmol), PdCl₂(dppf) (65 mg, 0.09 mmol) and Cs₂CO₃ (461 mg, 1.41 mmol) were dissolved in 1,4-dioxane (2 mL) and water (0.4 mL) under an atmosphere of N₂. The reaction mixture was heated to 80° C. and stirred for 2 hrs. The reaction mixture was cooled to rt and filtered through diatomaceous earth and the filtrate was concentrated. The resulting residue was diluted with water (5 mL) and extracted with EtOAc (3×10 mL). The combined organics were washed with brine (5 mL), dried over Na₂SO₄, filtered and concentrated to dryness. The crude material was purified by flash silica chromatography (0-80% EtOAc in hexanes) to afford tert-butyl (3R,4S)-3-(hydroxymethyl)-4-((8-(4-(trifluoromethyl)phenyl)pyrido[3,4-b]pyrazin-5-yl)amino)pyrrolidine-1-carboxylate (142 mg, 82% yield) as a yellow-orange solid. ¹H NMR (500 MHz, DMSO-d6) δ 1.41 (9H, br d), 2.64 (1H, br d), 3.14-3.27 (2H, m), 3.47 (1H, br dd), 3.51-3.61 (2H, m), 3.80 (1H, dd), 4.55-4.68 (1H, m), 4.78 (1H, t), 7.81 (2H, d), 7.89 (2H, d), 8.20 (1H, d), 8.34 (1H, s), 8.91 (1H, d), 9.09 (1H, d); m/z: (ES⁺) [M+H]⁺=490.

### ((3R,4S)-4-((8-(4-(trifluoromethyl)phenyl)pyrido[3,4-b]pyrazin-5-yl)amino)pyrrolidin-3-yl)methanol

HCl (4 M in 1,4-dioxane, 1.0 mL, 4.0 mmol) was added to a solution of tert-butyl (3R,4S)-3-(hydroxymethyl)-4-((8-(4-(trifluoromethyl)phenyl)pyrido[3,4-b]pyrazin-5-yl)amino)pyrrolidine-1-carboxylate (77 mg, 0.16 mmol) in MeOH (2 mL) at 0° C. The reaction mixture was warmed to rt and stirred for 2 hrs. The reaction mixture was diluted with saturated aq. NaHCO₃ (6 mL) and extracted with a 2:1 CHCl₃/MeOH solution (3×12 mL). The combined organics were washed with saturated aq. NaHCO₃ (10 mL), dried over Na₂SO₄, filtered and concentrated to dryness to afford ((3R,4S)-4-((8-(4-(trifluoromethyl)phenyl)pyrido[3,4-b]pyrazin-5-yl)amino)pyrrolidin-3-yl)methanol (60 mg, 98% yield) as a brown oil. m/z: (ES⁺) [M+H]⁺=390.

### 1-((3R,4S)-3-(hydroxymethyl)-4-((8-(4-(trifluoromethyl)phenyl)pyrido[3,4-b]pyrazin-5-yl)amino)pyrrolidin-1-yl)prop-2-en-1-one

A solution of acryloyl chloride (11 μL, 0.14 mmol) in DCM (0.2 mL) was added dropwise to a mixture of ((3R,4S)-4-((8-(4-(trifluoromethyl)phenyl)pyrido[3,4-b]pyrazin-5-yl)amino)pyrrolidin-3-yl)methanol (60 mg, 0.15 mmol) and DIPEA (80 μL, 0.46 mmol) in DCM (1.2 mL) at 0° C. The mixture was stirred at this temperature for 0.5 h and then diluted with brine (5 mL). The phases were separated and the aqueous layer was extracted with DCM (3×10 mL). The combined organics were dried over Na₂SO₄, filtered and concentrated to dryness. The crude material was purified on a reverse phase C18 column (2.5 to 35% MeCN in H₂O w/0.1% HCO₂H) to afford 1-((3R,4S)-3-(hydroxymethyl)-4-((8-(4-(trifluoromethyl)phenyl)pyrido[3,4-b]pyrazin-5-yl)amino)pyrrolidin-1-yl)prop-2-en-1-one (Example 27, 25 mg, 37% yield) as a yellow amorphous solid. ¹H NMR (500 MHz, DMSO-d6) δ 2.61-2.81 (1H, m), 3.37-3.45 (1H, m), 3.47-3.64 (3H, m), 3.73-4.15 (2H, m), 4.59-4.78 (1H, m), 4.78-4.89 (1H, m), 5.63-5.73 (1H, m), 6.11-6.19 (1H, m), 6.51-6.69 (1H, m), 7.78-7.85 (2H, m), 7.89 (2H, br d), 8.25-8.33 (1H, m), 8.33-8.37 (1H, m), 8.89-8.94 (1H, m), 9.07-9.12 (1H, m); m/z: (ES⁺) [M+H]⁺=444.

### Example 28: 1-((3R,4S)-3-(methoxymethyl)-4-((4-(4-(trifluoromethyl)phenyl)phthalazin-1-yl)amino)pyrrolidin-1-yl)prop-2-en-1-one

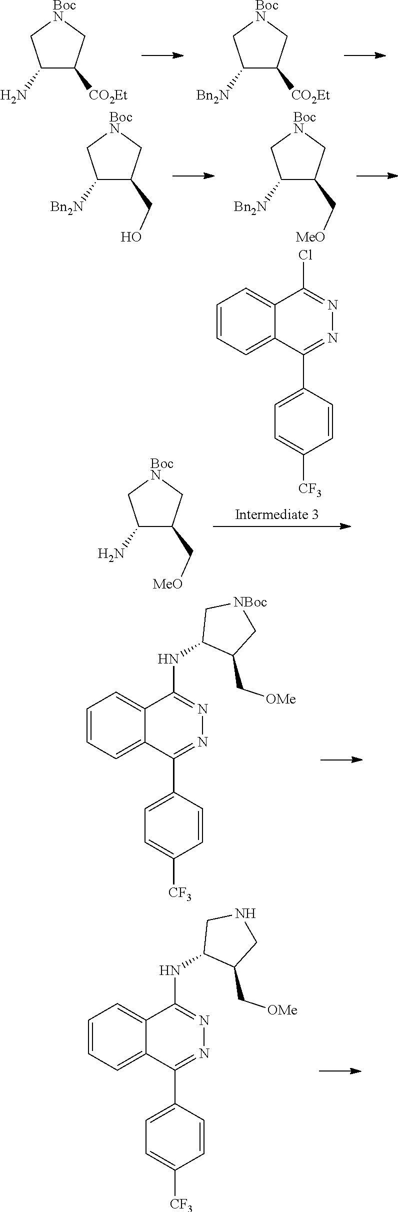

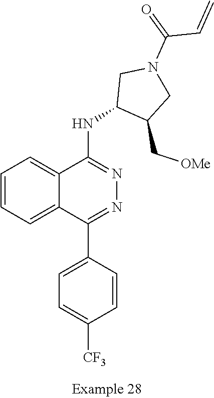 [SMILES: C=CC(=O)N1C[C@@H](COC)[C@H](Nc2nnc(-c3ccc(C(F)(F)F)cc3)c3ccccc23)C1]

### 1-(tert-butyl) 3-ethyl (3R,4S)-4-(dibenzylamino)pyrrolidine-1,3-dicarboxylate

Tetrabutylammonium iodide (172 mg, 0.46 mmol), benzyl bromide (0.41 mL, 3.41 mmol), and potassium carbonate (642 mg, 4.65 mmol) were added to a solution of 1-(tert-butyl) 3-ethyl (3R,4S)-4-aminopyrrolidine-1,3-dicarboxylate (400 mg, 1.55 mmol) in MeCN (12 mL). The reaction mixture was heated to 40° C. and stirred for 16 hrs. The reaction mixture was diluted with water (10 mL) and extracted with EtOAc (3×20 mL). The combined organics were dried over Na₂SO₄, filtered and concentrated to dryness. The crude material was purified by flash silica chromatography (0-20% EtOAc in hexanes) to afford 1-(tert-butyl) 3-ethyl (3R,4S)-4-(dibenzylamino)pyrrolidine-1,3-dicarboxylate (566 mg, 83% yield) as an amber oil. ¹H NMR (500 MHz, CDCl₃) δ 1.30 (3H, br d), 1.46 (9H, br s), 3.18-3.29 (1H, m), 3.31-3.57 (2H, m), 3.57-3.84 (7H, m), 4.10-4.28 (2H, m), 7.26 (2H, br d), 7.34 (8H, br d); m/z: (ES⁺) [M+H]⁺=439.

### tert-butyl (3S,4R)-3-(dibenzylamino)-4-(hydroxymethyl)pyrrolidine-1-carboxylate

A solution of LiBH₄ (2 M in THF, 1.90 ml, 3.87 mmol) was added slowly to a solution of 1-(tert-butyl) 3-ethyl (3R,4S)-4-(dibenzylamino)pyrrolidine-1,3-dicarboxylate (566 mg, 1.29 mmol) in THF (7 mL) at 0° C. under an atmosphere of N₂. The reaction mixture was warmed to rt and stirred for 16 hrs, and then heated to 50° C. with stirring for an additional 2 hrs. The reaction mixture was cooled to rt and the volatiles were removed under reduced pressure. The resulting residue was dissolved in MeOH (5 mL) and the mixture was heated to 90° C. and stirred for 2 hrs. The reaction mixture was cooled to rt and diluted with saturated aq. NaHCO₃ (6 mL). The phases were separated and the aq. layer was extracted with EtOAc (3×20 mL). The combined organics were dried over Na₂SO₄, filtered and concentrated to dryness. The crude material was purified by flash silica chromatography (10-30% EtOAc in hexanes) to afford tert-butyl (3S,4R)-3-(dibenzylamino)-4-(hydroxymethyl)pyrrolidine-1-carboxylate (332 mg, 65% yield) as a white foam. ¹H NMR (500 MHz, CDCl₃) δ 1.45-1.51 (9H, m), 2.65 (1H, br s), 2.84 (1H, br d), 3.24-3.35 (1H, m), 3.35-3.47 (4H, m), 3.47-3.64 (3H, m), 3.75 (1H, br s), 4.00 (2H, br d), 7.29-7.33 (2H, m), 7.36 (8H, br s); m/z: (ES⁺) [M+H]⁺=397.

### tert-butyl (3S,4R)-3-(dibenzylamino)-4-(methoxymethyl)pyrrolidine-1-carboxylate

NaH (60% dispersion in mineral oil, 48 mg, 1.2 mmol) was added to a solution of tert-butyl (3S,4R)-3-(dibenzylamino)-4-(hydroxymethyl)pyrrolidine-1-carboxylate (320 mg, 0.81 mmol) in THF (6 mL) at 0° C. under an atmosphere of N₂. The resulting mixture was stirred at 0° C. for 15 min. Iodomethane (0.060 mL, 0.97 mmol) was added and the reaction mixture was warmed to rt and stirred for 16 hrs. The reaction mixture was diluted with saturated aq. NH₄Cl (6 mL) and extracted with EtOAc (3×20 mL). The combined organics were dried over Na₂SO₄, filtered and concentrated to dryness. The crude material was purified by flash silica chromatography (0-20% EtOAc in hexanes) to afford tert-butyl (3S,4R)-3-(dibenzylamino)-4-(methoxymethyl)pyrrolidine-1-carboxylate (280 mg, 85% yield) as a clear oil. ¹H NMR (500 MHz, CDCl₃) δ 1.46 (9H, br s), 2.53-2.61 (1H, m), 2.96-3.13 (1H, m), 3.18-3.28 (1H, m), 3.30 (3H, s), 3.35-3.47 (1H, m), 3.47-3.68 (5H, m), 3.73-3.86 (2H, m), 7.23-7.28 (2H, m), 7.31-7.40 (8H, m); m/z: (ES⁺) [M+H]⁺=410.

### tert-butyl (3S,4R)-3-amino-4-(methoxymethyl)pyrrolidine-1-carboxylate

tert-Butyl (3S,4R)-3-(dibenzylamino)-4-(methoxymethyl)pyrrolidine-1-carboxylate (280 mg, 0.68 mmol) and Pd/C (10 wt %, 73 mg, 0.07 mmol) were diluted in THF (6 mL) and AcOH (0.080 mL, 1.4 mmol) under an atmosphere of N₂. The reaction mixture headspace was then purged with H₂ and the mixture was stirred at rt under an atmosphere of H₂ for 16 hrs. The reaction mixture was filtered through diatomaceous earth and the filtrate was concentrated to dryness to afford tert-butyl (3S,4R)-3-amino-4-(methoxymethyl)pyrrolidine-1-carboxylate (157 mg, 100% yield) as a gray solid. m/z: (ES⁺) [M+H]⁺=231.

### tert-butyl (3R,4S)-3-(methoxymethyl)-4-((4-(4-(trifluoromethyl)phenyl)phthalazin-1-yl)amino)pyrrolidine-1-carboxylate

DIPEA (0.18 mL, 1.04 mmol) was added to a solution of 1-chloro-4-(4-(trifluoromethyl)phenyl)phthalazine (Intermediate 3, 80 mg, 0.26 mmol) and tert-butyl (3S,4R)-3-amino-4-(methoxymethyl)pyrrolidine-1-carboxylate (107 mg, 0.47 mmol) in DMSO (1.2 mL). The reaction mixture was heated to 90° C. and stirred for 90 hrs. The reaction mixture was cooled to rt, diluted with water (5 mL), and extracted with EtOAc (3×10 mL). The combined organics were dried over Na₂SO₄, filtered and concentrated to dryness. The crude material was purified by flash silica chromatography (10-60% EtOAc in hexanes) to afford tert-butyl (3R,4S)-3-(methoxymethyl)-4-((4-(4-(trifluoromethyl)phenyl)phthalazin-1-yl)amino)pyrrolidine-1-carboxylate (39 mg, 30% yield) as an off-white solid. ¹H NMR (500 MHz, CDCl₃) δ 1.47 (9H, br s), 2.61-2.76 (1H, m), 3.18-3.33 (2H, m), 3.34-3.48 (3H, m), 3.50-3.70 (2H, m), 3.74 (1H, br d), 4.14 (1H, q), 4.38-4.60 (1H, m), 7.77-7.96 (8H, m); m/z: (ES⁺) [M+H]⁺=503.

### N-((3S,4R)-4-(methoxymethyl)pyrrolidin-3-yl)-4-(4-(trifluoromethyl)phenyl)phthalazin-1-amine

HCl (4 M in 1,4-dioxane, 0.49 mL, 1.9 mmol) was added to a solution of tert-butyl (3R,4S)-3-(methoxymethyl)-4-((4-(4-(trifluoromethyl)phenyl)phthalazin-1-yl)amino)pyrrolidine-1-carboxylate (39 mg, 0.08 mmol) in MeOH (1 mL) at 0° C. The reaction mixture was warmed to rt and stirred for 2 hrs. The volatiles were removed under reduced pressure to afford N-((3S,4R)-4-(methoxymethyl)pyrrolidin-3-yl)-4-(4-(trifluoromethyl)phenyl)phthalazin-1-amine, HCl (33 mg, 97% yield) as a tan gum. m/z: (ES⁺) [M+H]⁺=403.

### 1-((3R,4S)-3-(methoxymethyl)-4-((4-(4-(trifluoromethyl)phenyl)phthalazin-1-yl)amino)pyrrolidin-1-yl)prop-2-en-1-one

A solution of acryloyl chloride (6 μL, 0.07 mmol) in DCM (0.1 mL) was added dropwise to a solution of N-((3S,4R)-4-(methoxymethyl)pyrrolidin-3-yl)-4-(4-(trifluoromethyl)phenyl)phthalazin-1-amine, HCl (30 mg, 0.07 mmol) and Et₃N (0.030 mL, 0.21 mmol) in DCM (0.9 mL) at 0° C. The mixture was stirred at this temperature for 1 h and then diluted with brine (5 mL). The phases were separated and the aqueous layer was extracted with DCM (3×10 mL). The combined organics were dried over Na₂SO₄, filtered and concentrated to dryness. The crude material was purified on a reverse phase C18 column (5 to 30% MeCN in H₂O w/0.1% HCO₂H) to afford 1-((3R,4S)-3-(methoxymethyl)-4-((4-(4-(trifluoromethyl)phenyl)phthalazin-1-yl)amino)pyrrolidin-1-yl)prop-2-en-1-one (Example 28, 15 mg, 48% yield) as a white amorphous solid. ¹H NMR (500 MHz, DMSO-d6) δ 2.75-2.92 (1H, m), 3.32 (3H, s), 3.34-3.60 (4H, m), 3.76-4.25 (2H, m), 4.64-4.84 (1H, m), 5.61-5.75 (1H, m), 6.16 (1H, ddd), 6.53-6.72 (1H, m), 7.63-7.72 (1H, m), 7.79-7.83 (1H, m), 7.85-7.91 (3H, m), 7.91-7.95 (2H, m), 7.95-7.99 (1H, m), 8.45-8.55 (1H, m); m/z: (ES⁺) [M+H]⁺=457.

### Example 29: enantiomer 1 of cis-1-acryloyl-4-((4-(4-(trifluoromethyl)phenyl)phthalazin-1-yl)amino)pyrrolidine-3-carbonitrile

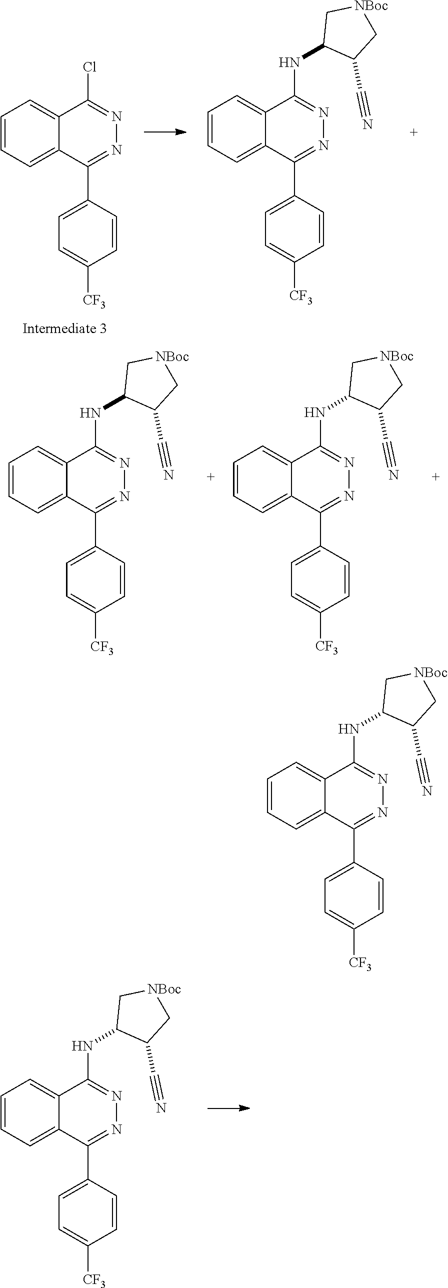 [SMILES: FC(F)(F)c1ccc(-c2nnc(Cl)c3ccccc23)cc1 ; CC(C)(C)OC(=O)N1C[C@H](Nc2nnc(-c3ccc(C(F)(F)F)cc3)c3ccccc23)[C@@H](C#N)C1 ; CC(C)(C)OC(=O)N1C[C@H](Nc2nnc(-c3ccc(C(F)(F)F)cc3)c3ccccc23)[C@@H](C#N)C1 ; CC(C)(C)OC(=O)N1C[C@H](C#N)[C@H](Nc2nnc(-c3ccc(C(F)(F)F)cc3)c3ccccc23)C1 ; CC(C)(C)OC(=O)N1C[C@H](C#N)[C@H](Nc2nnc(-c3ccc(C(F)(F)F)cc3)c3ccccc23)C1 ; CC(C)(C)OC(=O)N1C[C@H](C#N)[C@H](Nc2nnc(-c3ccc(C(F)(F)F)cc3)c3ccccc23)C1]

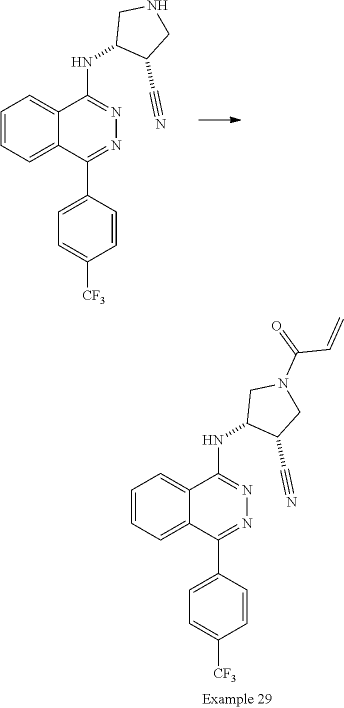 [SMILES: N#C[C@H]1CNC[C@H]1Nc1nnc(-c2ccc(C(F)(F)F)cc2)c2ccccc12 ; C=CC(=O)N1C[C@H](C#N)[C@H](Nc2nnc(-c3ccc(C(F)(F)F)cc3)c3ccccc23)C1]

### Enantiomer 1 of cis-tert-butyl 3-cyano-4-((4-(4-(trifluoromethyl)phenyl)phthalazin-1-yl)amino)pyrrolidine-1-carboxylate

1-Chloro-4-(4-(trifluoromethyl)phenyl)phthalazine (Intermediate 3, 800 mg, 2.59 mmol), tert-butyl 3-amino-4-cyanopyrrolidine-1-carboxylate (821 mg, 3.89 mmol), Cs₂CO₃ (2.111 g, 6.48 mmol) and [1,3-bis(2,6-di-3-pentylphenyl)imidazol-2-ylidene](3-chloropyridyl)dichloropalladium(II) (252 mg, 0.26 mmol) were diluted in 1,4-dioxane (12 mL) under an atmosphere of N₂. The reaction mixture was heated to 85° C. and stirred for 2 hrs. The reaction mixture was cooled to rt and filtered through diatomaceous earth. The filtrate was concentrated and the resulting residue was purified by flash silica chromatography (10-60% EtOAc in hexanes) to afford the racemic product. The racemic material was subjected to chiral SFC (Chiralpak IJ 4.6 mm×150 mm, 5 μm; Mobile phase=10% MeOH (w/0.2% NH₄OH):CO₂, UV detection @ 220 nm, Flow rate=4 mL/min, Column temperature=40° C., Outlet Pressure=100 bar) to afford enantiomer 1 of trans-1-acryloyl-4-((4-(4-(trifluoromethyl)phenyl)phthalazin-1-yl)amino)pyrrolidine-3-carbonitrile (Peak A, 110 mg, 9% yield), enantiomer 2 of trans-1-acryloyl-4-((4-(4-(trifluoromethyl)phenyl)phthalazin-1-yl)amino)pyrrolidine-3-carbonitrile (Peak B, 99 mg, 8% yield), enantiomer 1 of cis-1-acryloyl-4-((4-(4-(trifluoromethyl)phenyl)phthalazin-1-yl)amino)pyrrolidine-3-carbonitrile (Peak C, 92 mg, 7% yield), and enantiomer 2 of cis-1-acryloyl-4-((4-(4-(trifluoromethyl)phenyl)phthalazin-1-yl)amino)pyrrolidine-3-carbonitrile (Peak D, 106 mg, 8% yield) as light orange solids.

### enantiomer 1 of trans-tert-butyl 3-cyano-4-((4-(4-(trifluoromethyl)phenyl)phthalazin-1-yl)amino)pyrrolidine-1-carboxylate

¹H NMR (500 MHz, Methanol-d4) δ 1.53 (9H, s), 3.55-3.71 (1H, m), 3.72-3.81 (2H, m), 3.86 (1H, br d), 4.08 (1H, br s), 5.18-5.25 (1H, m), 7.84-7.88 (2H, m), 7.88-7.95 (4H, m), 7.96-8.02 (1H, m), 8.39 (1H, d); m/z: (ES⁺) [M+H]⁺=484.

### enantiomer 2 of trans-tert-butyl 3-cyano-4-((4-(4-(trifluoromethyl)phenyl)phthalazin-1-yl)amino)pyrrolidine-1-carboxylate

¹H NMR (500 MHz, Methanol-d4) δ 1.53 (9H, s), 3.55-3.71 (1H, m), 3.72-3.81 (2H, m), 3.86 (1H, br d), 4.08 (1H, br s), 5.18-5.25 (1H, m), 7.84-7.88 (2H, m), 7.88-7.95 (4H, m), 7.96-8.02 (1H, m), 8.39 (1H, d); m/z: (ES⁺) [M+H]⁺=484.

### enantiomer 1 of cis-tert-butyl 3-cyano-4-((4-(4-(trifluoromethyl)phenyl)phthalazin-1-yl)amino)pyrrolidine-1-carboxylate

¹H NMR (500 MHz, Methanol-d4) δ 1.54 (9H, s), 3.72-3.85 (2H, m), 3.91 (1H, dd), 3.99 (1H, br s), 4.21 (1H, br s), 5.11-5.20 (1H, m), 7.84-7.96 (6H, m), 7.98-8.03 (1H, m), 8.50 (1H, br d); m/z: (ES⁺) [M+H]⁺=484.

### enantiomer 2 of cis-tert-butyl 3-cyano-4-((4-(4-(trifluoromethyl)phenyl)phthalazin-1-yl)amino)pyrrolidine-1-carboxylate

¹H NMR (500 MHz, Methanol-d4) δ 1.54 (9H, s), 3.72-3.85 (2H, m), 3.91 (1H, dd), 3.99 (1H, br s), 4.21 (1H, br s), 5.11-5.20 (1H, m), 7.84-7.96 (6H, m), 7.98-8.03 (1H, m), 8.50 (1H, br d); m/z: (ES⁺) [M+H]⁺=484.

### Enantiomer 1 of cis-4-((4-(4-(trifluoromethyl)phenyl)phthalazin-1-yl)amino)pyrrolidine-3-carbonitrile

HCl (4 M in 1,4-dioxane, 0.71 mL, 2.9 mmol) was added to a solution of enantiomer 1 of cis-tert-butyl 3-cyano-4-((4-(4-(trifluoromethyl)phenyl)phthalazin-1-yl)amino)pyrrolidine-1-carboxylate (92 mg, 0.19 mmol) in 1,4-dioxane (3 mL) at 0° C. The reaction mixture was warmed to rt and stirred for 2 hrs. The volatiles were removed under reduced pressure to afford enantiomer 1 of cis-4-((4-(4-(trifluoromethyl)phenyl)phthalazin-1-yl)amino)pyrrolidine-3-carbonitrile, HCl (78 mg, 98% yield) as a white solid, which was used directly without purification. m/z: (ES⁺) [M+H]⁺=384.

### Enantiomer 1 of cis-1-acryloyl-4-((4-(4-(trifluoromethyl)phenyl)phthalazin-1-yl)amino)pyrrolidine-3-carbonitrile

A solution of acryloyl chloride (16 μL, 0.20 mmol) in DCM (0.5 mL) was added dropwise to a solution of enantiomer 1 of cis-4-((4-(4-(trifluoromethyl)phenyl)phthalazin-1-yl)amino)pyrrolidine-3-carbonitrile, HCl (78 mg, 0.19 mmol) and DIPEA (0.10 mL, 0.56 mmol) in DCM (2.5 mL) at 0° C. The mixture was stirred at this temperature for 0.5 h and then diluted with brine (5 mL). The phases were separated and the aqueous layer was extracted with DCM (3×10 mL). The combined organics were dried over Na₂SO₄, filtered and concentrated to dryness. The crude material was purified on a reverse phase C18 column (5 to 45% MeCN in H₂O) to afford enantiomer 1 of cis-1-acryloyl-4-((4-(4-(trifluoromethyl)phenyl)phthalazin-1-yl)amino)pyrrolidine-3-carbonitrile (Example 29, 40 mg, 49% yield) as a white solid. ¹H NMR (500 MHz, DMSO-d6) δ 3.78-3.92 (1H, m), 3.92-4.11 (2H, m), 4.13-4.31 (2H, m), 5.13-5.35 (1H, m), 5.73-5.83 (1H, m), 6.16-6.32 (1H, m), 6.57-6.74 (1H, m), 7.84-7.98 (6H, m), 7.98-8.10 (2H, m), 8.52-8.64 (1H, m); m/z: (ES⁺) [M+H]⁺=438.

### Example 30: enantiomer 1 of trans-1-acryloyl-4-((4-(4-(trifluoromethyl)phenyl)phthalazin-1-yl)amino)pyrrolidine-3-carbonitrile

### Example 31: enantiomer 1 of trans-1-acryloyl-4-((4-(4-(trifluoromethyl)phenyl)phthalazin-1-yl)amino)pyrrolidine-3-carboxamide

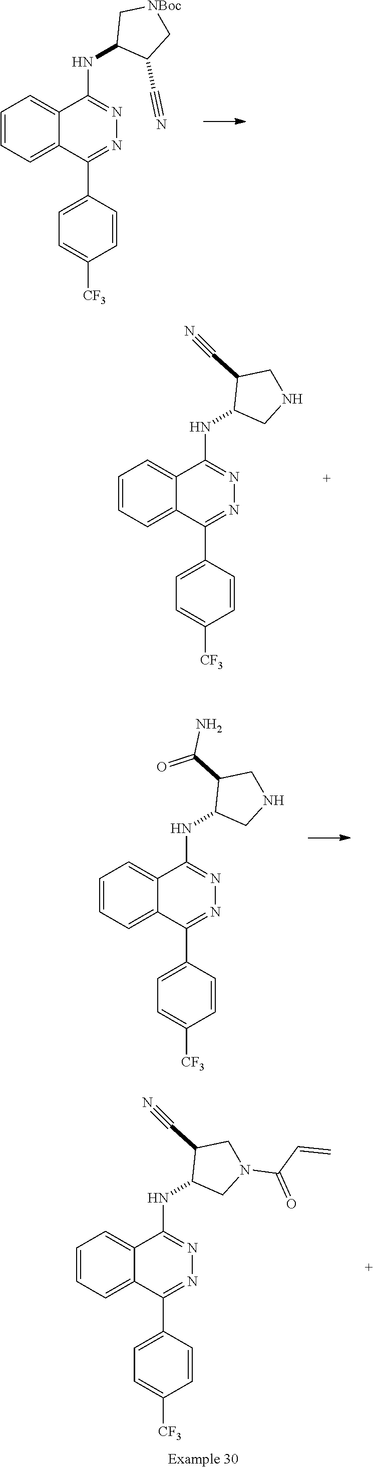

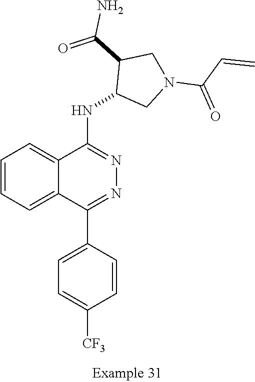 [SMILES: C=CC(=O)N1C[C@H](Nc2nnc(-c3ccc(C(F)(F)F)cc3)c3ccccc23)[C@@H](C(N)=O)C1]

### enantiomer 1 of trans-4-((4-(4-(trifluoromethyl)phenyl)phthalazin-1-yl)amino)pyrrolidine-3-carbonitrile and enantiomer 1 of trans-4-((4-(4-(trifluoromethyl)phenyl)phthalazin-1-yl)amino)pyrrolidine-3-carboxamide

HCl (4 M in 1,4-dioxane, 0.85 mL, 3.41 mmol) was added to a solution of enantiomer 1 of trans-tert-butyl 3-cyano-4-((4-(4-(trifluoromethyl)phenyl)phthalazin-1-yl)amino)pyrrolidine-1-carboxylate (110 mg, 0.23 mmol) in 1,4-dioxane (3 mL) at 0° C. The reaction mixture was warmed to rt and stirred for 2 hrs. The volatiles were removed under reduced pressure to afford a white amorphous solid (89 mg) which was a mixture of enantiomer 1 of trans-4-((4-(4-(trifluoromethyl)phenyl)phthalazin-1-yl)amino)pyrrolidine-3-carbonitrile, HCl (m/z: (ES⁺) [M+H]⁺=384) and enantiomer 1 of trans-4-((4-(4-(trifluoromethyl)phenyl)phthalazin-1-yl)amino)pyrrolidine-3-carboxamide, HCl (m/z: (ES⁺) [M+H]⁺=402). The mixture of products was carried forward and used without purification.

### enantiomer 1 of trans-1-acryloyl-4-((4-(4-(trifluoromethyl)phenyl)phthalazin-1-yl)amino)pyrrolidine-3-carbonitrile and enantiomer 1 of trans-1-acryloyl-4-((4-(4-(trifluoromethyl)phenyl)phthalazin-1-yl)amino)pyrrolidine-3-carboxamide

A solution of acryloyl chloride (18 μL, 0.22 mmol) in DCM (0.25 mL) was added dropwise to a mixture of enantiomer 1 of trans-4-((4-(4-(trifluoromethyl)phenyl)phthalazin-1-yl)amino)pyrrolidine-3-carbonitrile, HCl and enantiomer 1 of trans-4-((4-(4-(trifluoromethyl)phenyl)phthalazin-1-yl)amino)pyrrolidine-3-carboxamide, HCl (89 mg combined mass) and DIPEA (0.11 mL, 0.64 mmol) in DCM (2.5 mL) at 0° C. The reaction mixture was stirred at this temperature for 0.5 h and then diluted with brine (5 mL). The phases were separated and the aqueous layer was extracted with DCM (3×10 mL). The combined organics were dried over Na₂SO₄, filtered and concentrated to dryness. The crude material was purified on a reverse phase C18 column (5 to 50% MeCN in H₂O) to afford enantiomer 1 of trans-1-acryloyl-4-((4-(4-(trifluoromethyl)phenyl)phthalazin-1-yl)amino)pyrrolidine-3-carbonitrile (Example 30, 12 mg) and enantiomer 1 of trans-1-acryloyl-4-((4-(4-(trifluoromethyl)phenyl)phthalazin-1-yl)amino)pyrrolidine-3-carboxamide (Example 31, 30 mg) as white amorphous solids.

### enantiomer 1 of trans-1-acryloyl-4-((4-(4-(trifluoromethyl)phenyl)phthalazin-1-yl)amino)pyrrolidine-3-carbonitrile

¹H NMR (500 MHz, DMSO-d6) δ 3.63-4.31 (5H, m), 5.10-5.32 (1H, m), 5.68-5.85 (1H, m), 6.15-6.33 (1H, m), 6.66 (1H, td), 7.82-7.87 (1H, m), 7.87-7.91 (2H, m), 7.92-7.96 (3H, m), 7.96-8.03 (2H, m), 8.46 (1H, t); m/z: (ES⁺) [M+H]⁺=438.

### enantiomer 1 of trans-1-acryloyl-4-((4-(4-(trifluoromethyl)phenyl)phthalazin-1-yl)amino)pyrrolidine-3-carboxamide

¹H NMR (500 MHz, DMSO-d6) δ 3.40-3.61 (1H, m), 3.69-3.82 (1H, m), 3.85-4.06 (3H, m), 5.10-5.21 (1H, m), 5.65-5.73 (1H, m), 6.13-6.20 (1H, m), 6.54-6.71 (1H, m), 7.06 (1H, br s), 7.49 (1H, br d), 7.54-7.66 (1H, m), 7.82 (1H, br d), 7.86-8.01 (6H, m), 8.35 (1H, br d); m/z: (ES⁺) [M+H]⁺=456.

### Example 32: 1-((3S,4R)-3-hydroxy-4-((8-(4-(trifluoromethyl)phenyl)pyrido[2,3-d]pyridazin-5-yl)amino)pyrrolidin-1-yl)prop-2-en-1-one

### Example 33: 1-((3S,4R)-3-hydroxy-4-((5-(4-(trifluoromethyl)phenyl)pyrido[2,3-d]pyridazin-8-yl)amino)pyrrolidin-1-yl)prop-2-en-1-one

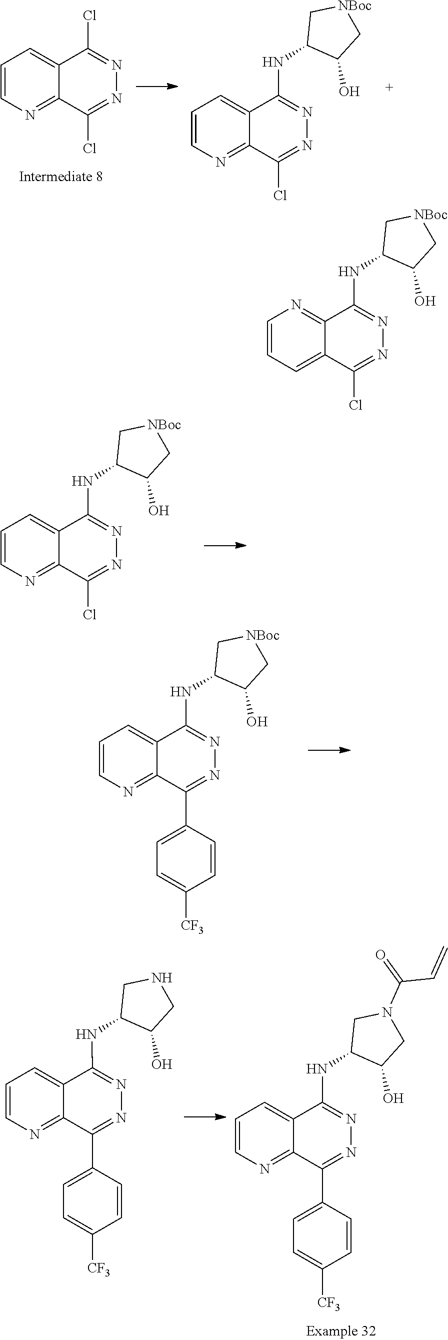

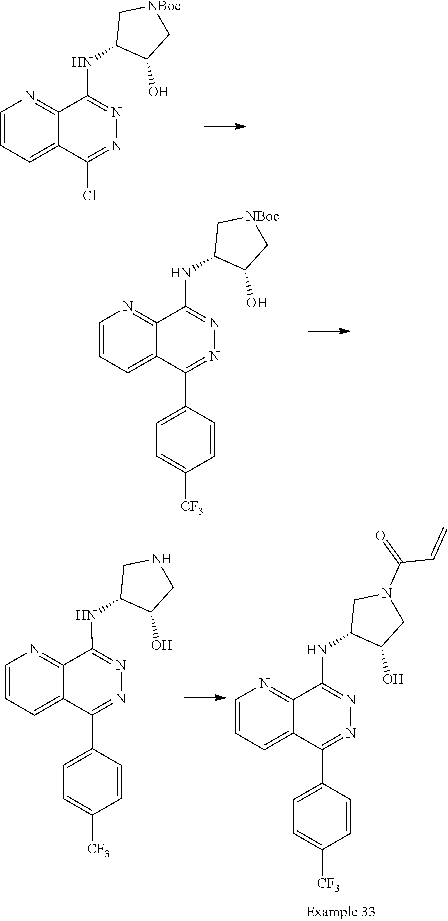 [SMILES: CC(C)(C)OC(=O)N1C[C@H](O)[C@H](Nc2nnc(Cl)c3cccnc23)C1 ; CC(C)(C)OC(=O)N1C[C@H](O)[C@H](Nc2nnc(-c3ccc(C(F)(F)F)cc3)c3cccnc23)C1 ; O[C@H]1CNC[C@H]1Nc1nnc(-c2ccc(C(F)(F)F)cc2)c2cccnc12 ; C=CC(=O)N1C[C@H](O)[C@H](Nc2nnc(-c3ccc(C(F)(F)F)cc3)c3cccnc23)C1]

### tert-butyl (3R,4S)-3-((8-chloropyrido[2,3-d]pyridazin-5-yl)amino)-4-hydroxypyrrolidine-1-carboxylate and tert-butyl (3R,4S)-3-((5-chloropyrido[2,3-d]pyridazin-8-yl)amino)-4-hydroxypyrrolidine-1-carboxylate

DIPEA (389 μL, 2.22 mmol) was added to a solution of 5,8-dichloropyrido[2,3-d]pyridazine (Intermediate 8, 149 mg, 0.745 mmol) and tert-butyl (3R,4S)-3-amino-4-hydroxypyrrolidine-1-carboxylate (150 mg, 0.74 mmol) in DMSO (3 mL). The reaction mixture was heated to 90° C. and stirred for 24 hrs. The reaction mixture was cooled to rt and loaded directly on a C18 column for reverse phase purification (10-100% MeCN in H₂O w/0.1% HCO₂H) to afford tert-butyl (3R,4S)-3-((8-chloropyrido[2,3-d]pyridazin-5-yl)amino)-4-hydroxypyrrolidine-1-carboxylate (78 mg, 29% yield) as an orange dry film and the regioisomer tert-butyl (3R,4S)-3-((5-chloropyrido[2,3-d]pyridazin-8-yl)amino)-4-hydroxypyrrolidine-1-carboxylate (79 mg g, 29% yield) as a brown dry film.

### tert-butyl (3R,4S)-3-((8-chloropyrido[2,3-d]pyridazin-5-yl)amino)-4-hydroxypyrrolidine-1-carboxylate

¹H NMR (500 MHz, DMSO-d6) δ 1.43 (9H, s), 3.36-3.39 (1H, m), 3.44-3.53 (2H, m), 3.62-3.71 (1H, m), 4.44-4.51 (1H, m), 4.65 (1H, br s), 5.21 (1H, br s), 7.61 (1H, br dd), 8.02 (1H, br dd), 8.94-9.01 (1H, m), 9.25 (1H, br d); m/z: (ES⁺) [M+H]⁺=366.

### tert-butyl (3R,4S)-3-((5-chloropyrido[2,3-d]pyridazin-8-yl)amino)-4-hydroxypyrrolidine-1-carboxylate

¹H NMR (500 MHz, DMSO-d6) δ 1.41 (9H, br d), 3.18-3.27 (1H, m), 3.34-3.40 (1H, m), 3.51-3.59 (1H, m), 3.79-3.90 (1H, m), 4.35-4.44 (1H, m), 4.52-4.62 (1H, m), 5.58-5.96 (1H, m), 7.37 (1H, br d), 8.08 (1H, dd), 8.51 (1H, d), 9.23 (1H, br d); m/z: (ES⁺) [M+H]⁺=366.

### tert-butyl (3S,4R)-3-hydroxy-4-((8-(4-(trifluoromethyl)phenyl)pyrido[2,3-d]pyridazin-5-yl)amino)pyrrolidine-1-carboxylate

tert-Butyl (3R,4S)-3-((8-chloropyrido[2,3-d]pyridazin-5-yl)amino)-4-hydroxypyrrolidine-1-carboxylate (77.8 mg, 0.213 mmol), 4,4,5,5-tetramethyl-2-(4-(trifluoromethyl)phenyl)-1,3,2-dioxaborolane (81 mg, 0.30 mmol), PdCl₂(dppf) (15.6 mg, 0.0213 mmol) and Cs₂CO₃ (139 mg, 0.427 mmol) were diluted in 1,4-dioxane (8 mL) and water (2 mL) under an atmosphere of N₂. The reaction mixture was heated to 80° C. and stirred for 2 hrs. The reaction mixture was cooled to rt, diluted with water (15 mL), and extracted with EtOAc (2×20 mL). The combined organics were dried over Na₂SO₄, filtered and concentrated to dryness. The crude material was purified by flash silica chromatography (30-100% EtOAc in hexanes) to afford tert-butyl (3S,4R)-3-hydroxy-4-((8-(4-(trifluoromethyl)phenyl)pyrido[2,3-d]pyridazin-5-yl)amino)pyrrolidine-1-carboxylate (57 mg, 56% yield) as a beige dry film. ¹H NMR (500 MHz, DCM-d2) δ 1.50 (9H, br s), 2.82-3.06 (1H, m), 3.34-3.45 (1H, m), 3.53-3.63 (1H, m), 3.72 (1H, br dd), 4.03-4.17 (1H, m), 4.65-4.76 (1H, m), 4.99-5.08 (1H, m), 5.80-6.11 (1H, m), 7.76-7.84 (3H, m), 8.24 (2H, d), 8.33 (1H, br d), 9.19 (1H, br d); m/z: (ES⁺) [M+H]⁺=476.

### (3S,4R)-4-((8-(4-(trifluoromethyl)phenyl)pyrido[2,3-d]pyridazin-5-yl)amino)pyrrolidin-3-ol hydrochloride

HCl (4 M in dioxane, 89 μL, 0.36 mmol) was added to a solution of tert-butyl (3S,4R)-3-hydroxy-4-((8-(4-(trifluoromethyl)phenyl)pyrido[2,3-d]pyridazin-5-yl)amino)pyrrolidine-1-carboxylate (56.5 mg, 0.119 mmol) in DCM (5 mL) and the reaction mixture stirred at rt for 24 hrs. The volatiles were removed under reduced pressure to afford (3S,4R)-4-((8-(4-(trifluoromethyl)phenyl)pyrido[2,3-d]pyridazin-5-yl)amino)pyrrolidin-3-ol hydrochloride (49 mg, 100% yield) as a light pink dry film which was carried forward without purification. m/z: (ES⁺) [M+H]⁺=376.

### 1-((3S,4R)-3-hydroxy-4-((8-(4-(trifluoromethyl)phenyl)pyrido[2,3-d]pyridazin-5-yl)amino)pyrrolidin-1-yl)prop-2-en-1-one

A solution of acryloyl chloride (0.014 mL, 0.18 mmol) in THF (0.5 mL) was added dropwise to a solution of (3S,4R)-4-((8-(4-(trifluoromethyl)phenyl)pyrido[2,3-d]pyridazin-5-yl)amino)pyrrolidin-3-ol hydrochloride (49 mg, 0.12 mmol) in THF (4 mL) and saturated aq. NaHCO₃ (1.5 mL) at 0° C. The reaction mixture was stirred at 0° C. for 20 min. The reaction mixture was diluted with water (10 mL) and extracted with EtOAc (3×15 mL). The combined organics were dried over Na₂SO₄, filtered and concentrated to dryness. The crude residue was purified by reverse phase purification (C18: 0-100% MeCN in H₂O w/0.1% HCO₂H) to afford 1-((3S,4R)-3-hydroxy-4-((8-(4-(trifluoromethyl)phenyl)pyrido[2,3-d]pyridazin-5-yl)amino)pyrrolidin-1-yl)prop-2-en-1-one (Example 32, 49 mg, 96% yield) as a white amorphous solid. ¹H NMR (500 MHz, DMSO-d6) δ 3.57-3.72 (2H, m), 3.74-4.13 (2H, m), 4.52-4.69 (1H, m), 4.79-4.99 (1H, m), 5.35 (1H, t), 5.71 (1H, dt), 6.19 (1H, dd), 6.63 (1H, dt), 7.68 (1H, dd), 7.83-7.92 (2H, m), 7.94-8.01 (1H, m), 8.17-8.28 (2H, m), 9.01 (1H, td), 9.20 (1H, dt); m/z: (ES⁺) [M+H]⁺=430.

### tert-butyl (3S,4R)-3-hydroxy-4-((5-(4-(trifluoromethyl)phenyl)pyrido[2,3-d]pyridazin-8-yl)amino)pyrrolidine-1-carboxylate

tert-Butyl (3R,4S)-3-((5-chloropyrido[2,3-d]pyridazin-8-yl)amino)-4-hydroxypyrrolidine-1-carboxylate (79.1 mg, 0.216 mmol), 4,4,5,5-tetramethyl-2-(4-(trifluoromethyl)phenyl)-1,3,2-dioxaborolane (82 mg, 0.30 mmol), PdCl₂(dppf) and Cs₂CO₃ (141 mg, 0.433 mmol) were diluted in 1,4-dioxane (8 mL) and water (2 mL) under an atmosphere of N₂. The reaction mixture was heated to 80° C. and stirred for 2 hrs. The reaction mixture was cooled to rt and diluted with EtOAc (20 mL). The organic layer was washed with water (15 mL), dried over Na₂SO₄, filtered, and concentrated to dryness. The crude material was purified by flash silica chromatography (30-100% EtOAc in hexanes) to afford tert-butyl (3S,4R)-3-hydroxy-4-((5-(4-(trifluoromethyl)phenyl)pyrido[2,3-d]pyridazin-8-yl)amino)pyrrolidine-1-carboxylate (62 mg, 60% yield) as a beige dry film. ¹H NMR (500 MHz, DCM-d2) δ 1.50 (9H, br s), 2.97 (1H, br s), 3.39-3.61 (2H, m), 3.66-3.77 (1H, m), 3.97-4.09 (1H, m), 4.70 (1H, br d), 4.99 (1H, br s), 7.04-7.22 (1H, m), 7.77 (1H, dd), 7.86 (4H, s), 8.23 (1H, br d), 9.10 (1H, br d); m/z: (ES⁺) [M+H]⁺=476.

### (3S,4R)-4-((5-(4-(trifluoromethyl)phenyl)pyrido[2,3-d]pyridazin-8-yl)amino)pyrrolidin-3-ol hydrochloride

HCl (4 M in dioxane, 95 μL, 0.38 mmol) was added to a solution of tert-butyl (3S,4R)-3-hydroxy-4-((5-(4-(trifluoromethyl)phenyl)pyrido[2,3-d]pyridazin-8-yl)amino)pyrrolidine-1-carboxylate (60.0 mg, 0.126 mmol) in DCM (5 mL) and the reaction mixture stirred at rt for 4 hrs. The reaction mixture was concentrated under reduced pressure to afford (3S,4R)-4-((5-(4-(trifluoromethyl)phenyl)pyrido[2,3-d]pyridazin-8-yl)amino)pyrrolidin-3-ol hydrochloride (54 mg, 100% yield) as a white dry film, which was carried forward without purification. m/z: (ES⁺) [M+H]⁺=376.

### 1-((3S,4R)-3-hydroxy-4-((5-(4-(trifluoromethyl)phenyl)pyrido[2,3-d]pyridazin-8-yl)amino)pyrrolidin-1-yl)prop-2-en-1-one

A solution of acryloyl chloride (16 μL, 0.20 mmol) in THF (0.5 mL) was added dropwise to a solution of (3S,4R)-4-((5-(4-(trifluoromethyl)phenyl)pyrido[2,3-d]pyridazin-8-yl)amino)pyrrolidin-3-ol hydrochloride (53.8 mg, 0.131 mmol) in THF (3 mL) and saturated aq. NaHCO₃ (1.0 mL) at 0° C. under an atmosphere of N₂. The reaction mixture was stirred at 0° C. for 30 min. The reaction mixture was diluted with water (10 mL) and extracted with EtOAc (3×15 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 35%) to afford 1-((3S,4R)-3-hydroxy-4-((5-(4-(trifluoromethyl)phenyl)pyrido[2,3-d]pyridazin-8-yl)amino)pyrrolidin-1-yl)prop-2-en-1-one (Example 33, 29 mg, 52% yield) as an off-white amorphous solid. ¹H NMR (500 MHz, DMSO-d6) δ 3.40-3.95 (3H, m), 4.03-4.26 (1H, m), 4.41-4.58 (1H, m), 4.69-4.90 (1H, m), 5.69 (1H, br t), 5.76-5.95 (1H, m), 6.10-6.24 (1H, m), 6.61 (1H, br dd), 7.46 (1H, br d), 7.88-8.01 (5H, m), 8.31 (1H, br d), 9.21 (1H, br s); m/z: (ES⁺) [M+H]⁺=430.

### Example 34: 1-((3S,4R)-3-hydroxy-4-((8-(4-(trifluoromethyl)phenyl)-1,6-naphthyridin-5-yl)amino)pyrrolidin-1-yl)prop-2-en-1-one

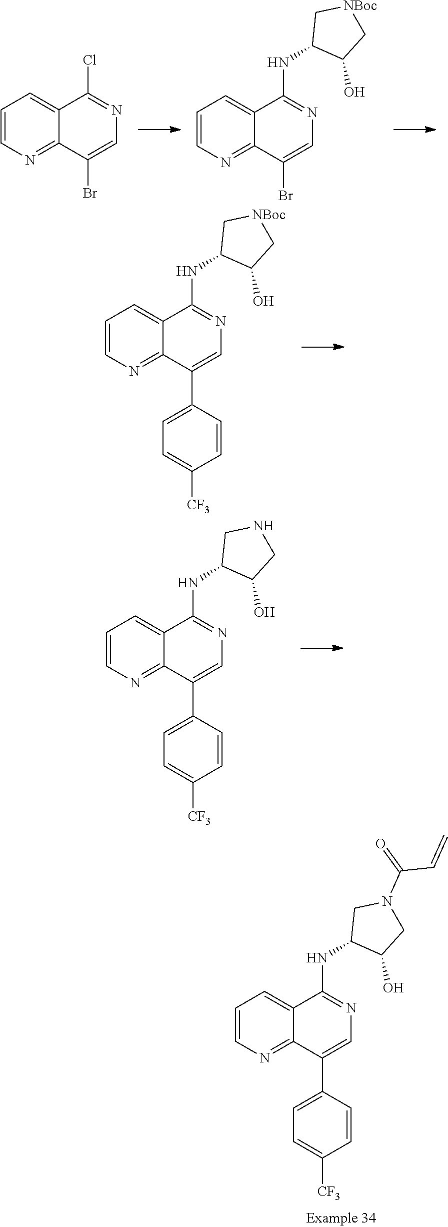

### tert-butyl (3R,4S)-3-((8-bromo-1,6-naphthyridin-5-yl)amino)-4-hydroxypyrrolidine-1-carboxylate

DIPEA (207 μL, 1.19 mmol) was added to a solution of 8-bromo-5-chloro-1,6-naphthyridine (96 mg, 0.40 mmol) and tert-butyl (3R,4S)-3-amino-4-hydroxypyrrolidine-1-carboxylate (80 mg, 0.4 mmol) in DMSO (3 mL). The reaction mixture was heated to 90° C. and stirred for 30 hrs. The reaction mixture was cooled to rt and loaded directly on a C18 column for reverse phase purification (10-100% MeCN in H₂O w/0.1% HCO₂H) to give tert-butyl (3R,4S)-3-((8-bromo-1,6-naphthyridin-5-yl)amino)-4-hydroxypyrrolidine-1-carboxylate (0.102 g, 63% yield) as an orange dry film. ¹H NMR (500 MHz, DMSO-d6) δ 1.43 (9H, s), 3.36 (1H, br dd), 3.40-3.50 (2H, m), 3.63 (1H, br t), 4.36-4.42 (1H, m), 4.60 (1H, br s), 5.22 (1H, br s), 7.54 (1H, br s), 7.65 (1H, br dd), 8.33 (1H, s), 8.88-8.90 (1H, m), 9.05 (1H, br d); m/z: (ES⁺) [M+H]⁺=409.

### tert-butyl (3S,4R)-3-hydroxy-4-((8-(4-(trifluoromethyl)phenyl)-1,6-naphthyridin-5-yl)amino)pyrrolidine-1-carboxylate

tert-Butyl (3R,4S)-3-((8-bromo-1,6-naphthyridin-5-yl)amino)-4-hydroxypyrrolidine-1-carboxylate (102 mg, 0.250 mmol), 4,4,5,5-tetramethyl-2-(4-(trifluoromethyl)phenyl)-1,3,2-dioxaborolane (95 mg, 0.35 mmol), PdCl₂(dppf) (18 mg, 0.025 mmol) and Cs₂CO₃ (163 mg, 0.500 mmol) were diluted in 1,4-dioxane (8 mL) and water (2 mL) under an atmosphere of N₂. The reaction mixture was heated to 80° C. and stirred for 2 hrs. The reaction mixture was cooled to rt, diluted with water (15 mL), and extracted with EtOAc (2×20 mL). The combined organics were dried over Na₂SO₄, filtered and concentrated to dryness. The crude material was purified by flash silica chromatography (30-100% EtOAc in hexanes) to afford tert-butyl (3S,4R)-3-hydroxy-4-((8-(4-(trifluoromethyl)phenyl)-1,6-naphthyridin-5-yl)amino)pyrrolidine-1-carboxylate (79 mg, 67% yield) as a yellow solid. ¹H NMR (500 MHz, DCM-d2) δ 1.50 (9H, br s), 2.57-2.96 (1H, m), 3.31-3.42 (1H, m), 3.49-3.60 (1H, m), 3.67-3.73 (1H, m), 4.02 (1H, br dd), 4.59 (1H, br s), 4.89 (1H, br s), 5.90-6.09 (1H, m), 7.51 (1H, dd), 7.73-7.78 (2H, m), 7.82-7.87 (2H, m), 8.26 (1H, s), 8.30 (1H, br d), 8.98-9.06 (1H, m); m/z: (ES⁺) [M+H]⁺=475.

### (3S,4R)-4-((8-(4-(trifluoromethyl)phenyl)-1,6-naphthyridin-5-yl)amino)pyrrolidin-3-ol hydrochloride

HCl (4 M in dioxane, 123 μL, 0.493 mmol) was added to a solution of tert-butyl (3S,4R)-3-hydroxy-4-((8-(4-(trifluoromethyl)phenyl)-1,6-naphthyridin-5-yl)amino)pyrrolidine-1-carboxylate (78 mg, 0.16 mmol) in DCM (5 mL) and the reaction mixture stirred at rt for 4 hrs. The reaction mixture was concentrated to dryness to afford (3S,4R)-4-((8-(4-(trifluoromethyl)phenyl)-1,6-naphthyridin-5-yl)amino)pyrrolidin-3-ol hydrochloride (68 mg, 100% yield) as a faint yellow dry film, which was carried forward without purification. m/z: (ES⁺) [M+H]⁺=375.

### 1-((3S,4R)-3-hydroxy-4-((8-(4-(trifluoromethyl)phenyl)-1,6-naphthyridin-5-yl)amino)pyrrolidin-1-yl)prop-2-en-1-one

A solution of acryloyl chloride (0.020 mL, 0.25 mmol) in THF (0.5 mL) was added dropwise to a solution of (3S,4R)-4-((8-(4-(trifluoromethyl)phenyl)-1,6-naphthyridin-5-yl)amino)pyrrolidin-3-ol hydrochloride (68 mg, 0.17 mmol) in THF (4 mL) and saturated aq. NaHCO₃ (1.5 mL) at 0° C. under an atmosphere of N₂. The reaction mixture was stirred at 0° C. for 20 min. The reaction mixture was diluted with water (10 mL) and extracted with EtOAc (3×15 mL). The combined organics were dried over Na₂SO₄, filtered and concentrated to dryness. The crude material was purified by reverse phase purification (C18: 0-100% MeCN in H₂O w/0.1% HCO₂H) to afford 1-((3S,4R)-3-hydroxy-4-((8-(4-(trifluoromethyl)phenyl)-1,6-naphthyridin-5-yl)amino)pyrrolidin-1-yl)prop-2-en-1-one (Example 34, 60 mg, 84% yield) as a yellow amorphous solid. ¹H NMR (500 MHz, DMSO-d6) δ 3.65-4.12 (4H, m), 4.48-4.65 (1H, m), 4.71-4.89 (1H, m), 5.72 (1H, dt), 6.12-6.25 (1H, m), 6.62 (1H, dd), 7.74 (1H, br s), 7.79-7.91 (4H, m), 8.14 (1H, br d), 9.00-9.25 (2H, m); m/z: (ES⁺) [M+H]⁺=429.

### Example 35: 1-((3S,4R)-3-hydroxy-4-((8-(5-(trifluoromethyl)pyridin-2-yl)-1,6-naphthyridin-5-yl)amino)pyrrolidin-1-yl)prop-2-en-1-one

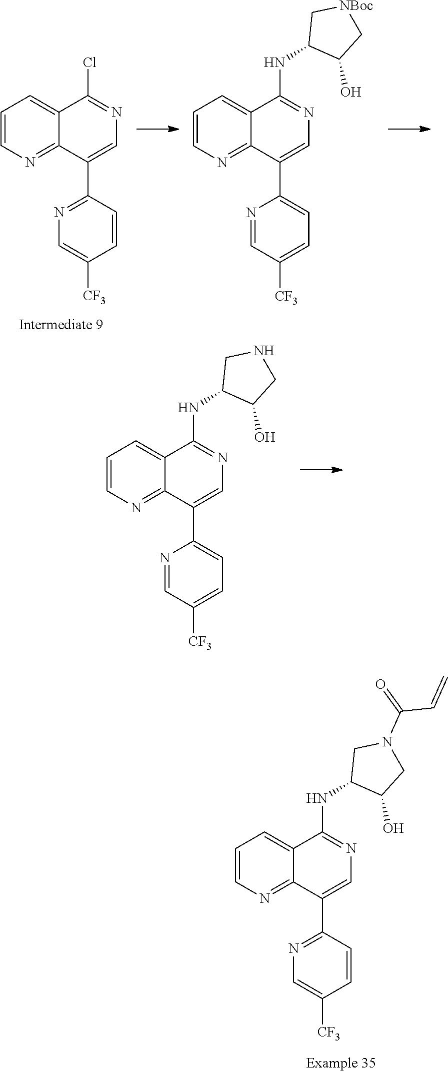 [SMILES: FC(F)(F)c1ccc(-c2cnc(Cl)c3cccnc23)nc1 ; CC(C)(C)OC(=O)N1C[C@H](O)[C@H](Nc2ncc(-c3ccc(C(F)(F)F)cn3)c3ncccc23)C1 ; O[C@H]1CNC[C@H]1Nc1ncc(-c2ccc(C(F)(F)F)cn2)c2ncccc12 ; C=CC(=O)N1C[C@H](O)[C@H](Nc2ncc(-c3ccc(C(F)(F)F)cn3)c3ncccc23)C1]

### tert-butyl (3S,4R)-3-hydroxy-4-((8-(5-(trifluoromethyl)pyridin-2-yl)-1,6-naphthyridin-5-yl)amino)pyrrolidine-1-carboxylate

DIPEA (110 μL, 0.63 mmol) was added to a solution of 5-chloro-8-(5-(trifluoromethyl)pyridin-2-yl)-1,6-naphthyridine (Intermediate 9, 65.3 mg, 0.211 mmol) and tert-butyl (3R,4S)-3-amino-4-hydroxypyrrolidine-1-carboxylate (55.4 mg, 0.274 mmol) in DMSO (0.5 mL). The resulting mixture was heated to 90° C. and stirred for 21 hrs. The reaction mixture was cooled to rt, diluted with water (10 mL), and extracted with EtOAc (2×15 mL). The combined organics were dried over Na₂SO₄, filtered and concentrated to dryness to afford tert-butyl (3S,4R)-3-hydroxy-4-((8-(5-(trifluoromethyl)pyridin-2-yl)-1,6-naphthyridin-5-yl)amino)pyrrolidine-1-carboxylate as a brown dry film, which was carried forward without purification and assuming quantitative yield. m/z: (ES⁺) [M+H]⁺=476.

### (3S,4R)-4-((8-(5-(trifluoromethyl)pyridin-2-yl)-1,6-naphthyridin-5-yl)amino)pyrrolidin-3-ol hydrochloride

HCl (4 M in dioxane, 160 μL, 0.63 mmol) was added to a solution of tert-butyl (3S,4R)-3-hydroxy-4-((8-(5-(trifluoromethyl)pyridin-2-yl)-1,6-naphthyridin-5-yl)amino)pyrrolidine-1-carboxylate (100 mg, 0.21 mmol) in DCM (5 mL) and the resulting mixture was stirred at rt for 18 hrs. The reaction mixture was concentrated to dryness to afford (3S,4R)-4-((8-(5-(trifluoromethyl)pyridin-2-yl)-1,6-naphthyridin-5-yl)amino)pyrrolidin-3-ol hydrochloride as a dark brown solid, which was carried forward without purification and assuming quantitative yield. m/z: (ES⁺) [M+H]⁺=376.

### 1-((3S,4R)-3-hydroxy-4-((8-(5-(trifluoromethyl)pyridin-2-yl)-1,6-naphthyridin-5-yl)amino)pyrrolidin-1-yl)prop-2-en-1-one

A solution of acryloyl chloride (0.026 mL, 0.32 mmol) in THF (0.5 mL) was added dropwise to a solution of (3S,4R)-4-((8-(5-(trifluoromethyl)pyridin-2-yl)-1,6-naphthyridin-5-yl)amino)pyrrolidin-3-ol hydrochloride (87 mg, 0.21 mmol) in THF (6 mL) and saturated aq. NaHCO₃ (2.0 mL) at 0° C. under an atmosphere of N₂. The reaction mixture was stirred at 0° C. for 10 min. The reaction mixture was diluted with water (10 mL) and extracted with EtOAc (2×15 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 30%) to afford 1-((3S,4R)-3-hydroxy-4-((8-(5-(trifluoromethyl)pyridin-2-yl)-1,6-naphthyridin-5-yl)amino)pyrrolidin-1-yl)prop-2-en-1-one (Example 35, 40 mg, 44% yield) as a yellow amorphous solid. ¹H NMR (500 MHz, DMSO-d6) δ 3.54-3.70 (2H, m), 3.71-4.08 (2H, m), 4.44-4.57 (1H, m), 4.75-4.95 (1H, m), 5.38 (1H, br s), 5.70 (1H, dt), 6.18 (1H, dd), 6.61 (1H, dt), 7.65 (1H, ddd), 7.81 (1H, t), 8.23 (1H, br d), 8.53 (1H, dd), 8.75 (1H, s), 8.94-9.00 (1H, m), 9.02 (1H, d), 9.04-9.07 (1H, m); m/z: (ES⁺) [M+H]⁺=430.

### Example 36: 1-((3S,4R)-3-fluoro-4-((8-(5-(trifluoromethyl)pyridin-2-yl)-1,6-naphthyridin-5-yl)amino)pyrrolidin-1-yl)prop-2-en-1-one

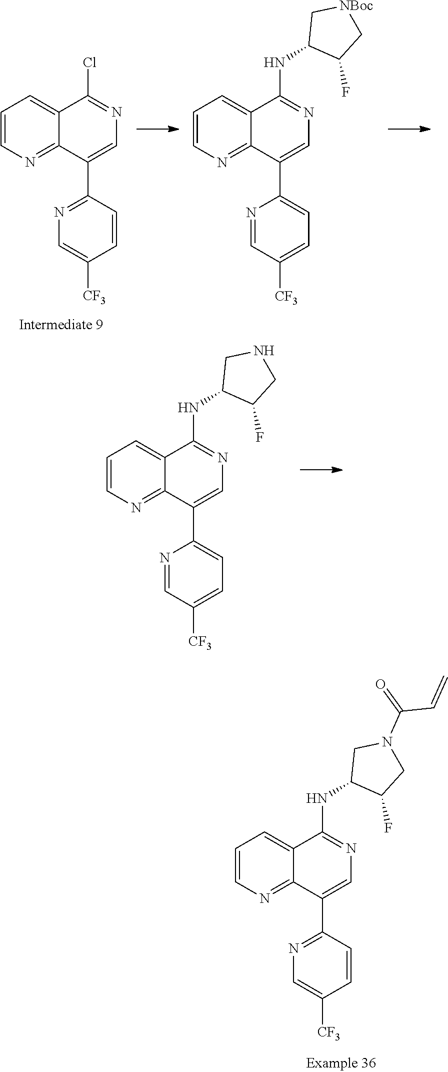 [SMILES: FC(F)(F)c1ccc(-c2cnc(Cl)c3cccnc23)nc1 ; CC(C)(C)OC(=O)N1C[C@H](F)[C@H](Nc2ncc(-c3ccc(C(F)(F)F)cn3)c3ncccc23)C1 ; F[C@H]1CNC[C@H]1Nc1ncc(-c2ccc(C(F)(F)F)cn2)c2ncccc12 ; C=CC(=O)N1C[C@H](F)[C@H](Nc2ncc(-c3ccc(C(F)(F)F)cn3)c3ncccc23)C1]

### tert-butyl (3S,4R)-3-fluoro-4-((8-(5-(trifluoromethyl)pyridin-2-yl)-1,6-naphthyridin-5-yl)amino)pyrrolidine-1-carboxylate

DIPEA (120 μL, 0.68 mmol) was added to a solution of 5-chloro-8-(5-(trifluoromethyl)pyridin-2-yl)-1,6-naphthyridine (Intermediate 9, 70.0 mg, 0.226 mmol) and tert-butyl (3R,4S)-3-amino-4-fluoropyrrolidine-1-carboxylate (69.3 mg, 0.339 mmol) in DMSO (0.5 mL). The resulting mixture was heated to 90° C. and stirred for 21 hrs. The reaction mixture was cooled to rt, diluted with water (15 mL), and extracted with EtOAc (2×20 mL). The combined organics were dried over Na₂SO₄, filtered and concentrated to dryness to afford tert-butyl (3S,4R)-3-fluoro-4-((8-(5-(trifluoromethyl)pyridin-2-yl)-1,6-naphthyridin-5-yl)amino)pyrrolidine-1-carboxylate as a brown dry film, which was carried forward without purification and assuming quantitative yield. m/z: (ES⁺) [M+H]⁺=478.

### N-((3R,4S)-4-fluoropyrrolidin-3-yl)-8-(5-(trifluoromethyl)pyridin-2-yl)-1,6-naphthyridin-5-amine hydrochloride

HCl (4 M in dioxane, 339 μL, 1.36 mmol) was added to a solution of tert-butyl (3S,4R)-3-fluoro-4-((8-(5-(trifluoromethyl)pyridin-2-yl)-1,6-naphthyridin-5-yl)amino)pyrrolidine-1-carboxylate (108 mg, 0.226 mmol) in DCM (5 mL) and the reaction mixture was stirred at rt for 36 hrs. The reaction mixture was concentrated to dryness to afford N-((3R,4S)-4-fluoropyrrolidin-3-yl)-8-(5-(trifluoromethyl)pyridin-2-yl)-1,6-naphthyridin-5-amine hydrochloride as a brown oil, which was carried forward without purification and assuming quantitative yield. m/z: (ES⁺) [M+H]⁺=378.

### 1-((3S,4R)-3-fluoro-4-((8-(5-(trifluoromethyl)pyridin-2-yl)-1,6-naphthyridin-5-yl)amino)pyrrolidin-1-yl)prop-2-en-1-one

A solution of acryloyl chloride (28 μL, 0.34 mmol) in THF (0.5 mL) was added dropwise to a solution of N-((3R,4S)-4-fluoropyrrolidin-3-yl)-8-(5-(trifluoromethyl)pyridin-2-yl)-1,6-naphthyridin-5-amine hydrochloride (94 mg, 0.23 mmol) in THF (10 mL) and saturated aq. NaHCO₃ (3.0 mL) at 0° C. under an atmosphere of N₂. The reaction mixture was stirred at 0° C. for 10 min. The reaction mixture was diluted with water (15 mL) and extracted with EtOAc (2×20 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 30%) to afford 1-((3S,4R)-3-fluoro-4-((8-(5-(trifluoromethyl)pyridin-2-yl)-1,6-naphthyridin-5-yl)amino)pyrrolidin-1-yl)prop-2-en-1-one (Example 36, 39 mg, 40% yield) as a yellow amorphous solid. ¹H NMR (500 MHz, DMSO-d6) δ 3.65-3.93 (2H, m), 3.96-4.26 (2H, m), 4.94-5.21 (1H, m), 5.38-5.60 (1H, m), 5.71-5.79 (1H, m), 6.22 (1H, ddd), 6.58-6.70 (1H, m), 7.68 (1H, ddd), 8.10 (1H, dd), 8.24 (1H, dd), 8.52 (1H, dd), 8.76 (1H, s), 8.99 (1H, ddd), 9.04 (1H, d), 9.08 (1H, dt); m/z: (ES⁺) [M+H]⁺=432.

### Example 37: 1-((3R,4S)-3-(hydroxymethyl)-4-((8-(5-(trifluoromethyl)pyridin-2-yl)-1,6-naphthyridin-5-yl)amino)pyrrolidin-1-yl)prop-2-en-1-one

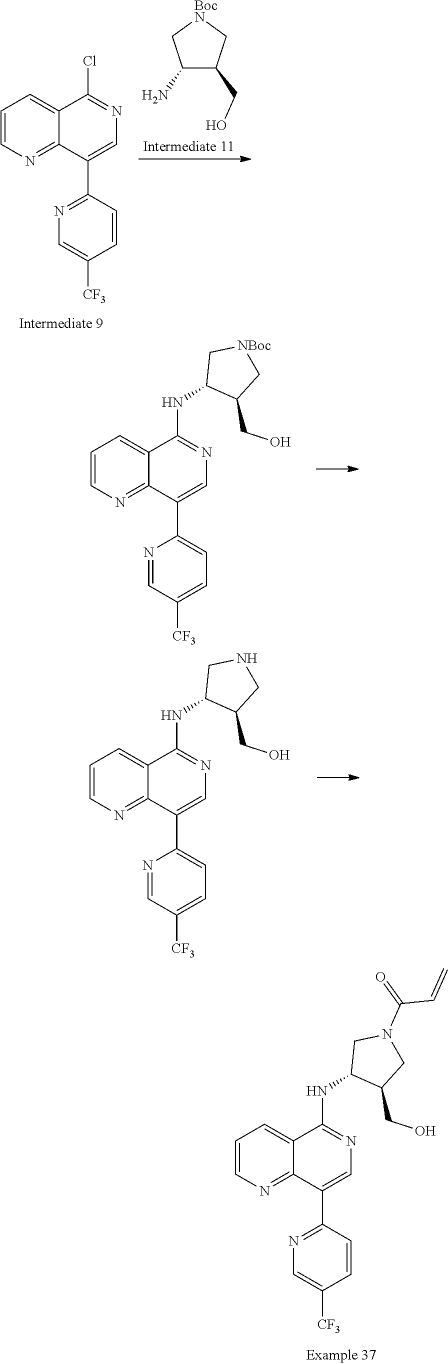

### tert-butyl (3R,4S)-3-(hydroxymethyl)-4-((8-(5-(trifluoromethyl)pyridin-2-yl)-1,6-naphthyridin-5-yl)amino)pyrrolidine-1-carboxylate

DIPEA (96 μL, 0.55 mmol) was added to a solution of 5-chloro-8-(5-(trifluoromethyl)pyridin-2-yl)-1,6-naphthyridine (Intermediate 9, 57 mg, 0.18 mmol) and tert-butyl (3S,4R)-3-amino-4-(hydroxymethyl)pyrrolidine-1-carboxylate (Intermediate 11, 80 mg, 0.37 mmol) in DMSO (0.5 mL). The resulting mixture was heated to 85° C. and stirred for 20 hrs. The reaction mixture was cooled to rt, diluted with water (10 mL), and extracted with EtOAc (2×15 mL). The combined organics were dried over Na₂SO₄, filtered and concentrated to dryness to afford tert-butyl (3R,4S)-3-(hydroxymethyl)-4-((8-(5-(trifluoromethyl)pyridin-2-yl)-1,6-naphthyridin-5-yl)amino)pyrrolidine-1-carboxylate as a brown oil which was carried forward without purification and assuming quantitative yield. m/z: (ES⁺) [M+H]⁺=490.

### ((3R,4S)-4-((8-(5-(trifluoromethyl)pyridin-2-yl)-1,6-naphthyridin-5-yl)amino)pyrrolidin-3-yl)methanol hydrochloride

HCl (4 M in dioxane, 0.14 mL, 0.55 mmol) was added to a solution of tert-butyl (3R,4S)-3-(hydroxymethyl)-4-((8-(5-(trifluoromethyl)pyridin-2-yl)-1,6-naphthyridin-5-yl)amino)pyrrolidine-1-carboxylate (90 mg, 0.18 mmol) in DCM (5 mL) and the reaction mixture was stirred at rt for 15 hrs.

The reaction mixture was concentrated to dryness to afford ((3R,4S)-4-((8-(5-(trifluoromethyl)pyridin-2-yl)-1,6-naphthyridin-5-yl)amino)pyrrolidin-3-yl)methanol hydrochloride as a brown dry film which was carried forward without purification and assuming quantitative yield. m/z: (ES⁺) [M+H]⁺=390.

### 1-((3R,4S)-3-(hydroxymethyl)-4-((8-(5-(trifluoromethyl)pyridin-2-yl)-1,6-naphthyridin-5-yl)amino)pyrrolidin-1-yl)prop-2-en-1-one

A solution of acryloyl chloride (0.021 mL, 0.26 mmol) in THF (1 mL) was added dropwise to a solution of ((3R,4S)-4-((8-(5-(trifluoromethyl)pyridin-2-yl)-1,6-naphthyridin-5-yl)amino)pyrrolidin-3-yl)methanol hydrochloride (78 mg, 0.18 mmol) in THF (5 mL) and saturated aq. NaHCO₃ (2 mL) at 0° C. under an atmosphere of N₂. The reaction mixture was stirred at 0° C. for 10 min. The reaction mixture was diluted with water (10 mL) and extracted with EtOAc (2×15 mL). The combined organics were dried over Na₂SO₄, filtered and concentrated in vacuo. The crude material was purified by reverse phase purification (C18: 0-30% MeCN in H₂O w/0.1% HCO₂H) to afford 1-((3R,4S)-3-(hydroxymethyl)-4-((8-(5-(trifluoromethyl)pyridin-2-yl)-1,6-naphthyridin-5-yl)amino)pyrrolidin-1-yl)prop-2-en-1-one (Example 37, 19.8 mg, 24% yield) as a yellow amorphous solid. ¹H NMR (500 MHz, DMSO-d6) δ 2.54-2.69 (1H, m), 3.36-3.44 (1H, m), 3.46-3.64 (3H, m), 3.76-4.19 (2H, m), 4.62-4.80 (1H, m), 4.85 (1H, br d), 5.62-5.74 (1H, m), 6.15 (1H, ddd), 6.54-6.69 (1H, m), 7.65 (1H, dt), 8.05 (1H, br dd), 8.23 (1H, dd), 8.53 (1H, d), 8.75 (1H, s), 8.91 (1H, br d), 9.02 (1H, s), 9.05 (1H, br d); m/z: (ES⁺) [M+H]⁺=444.

### Example 38: (S)-1-(3-((8-(5-(trifluoromethyl)pyridin-2-yl)-1,6-naphthyridin-5-yl)amino)pyrrolidin-1-yl)prop-2-en-1-one

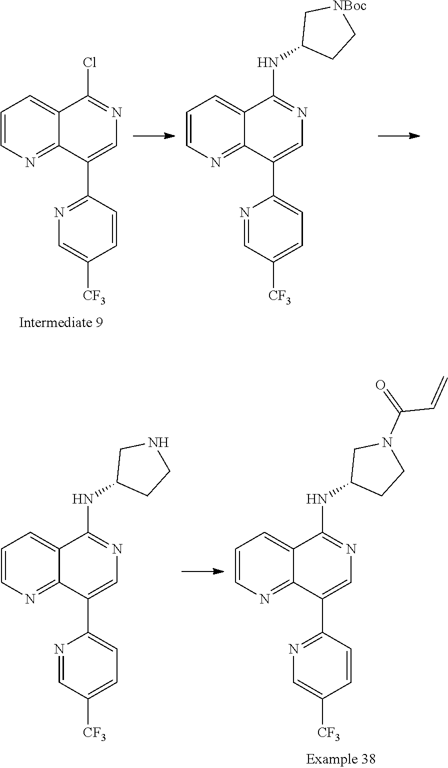 [SMILES: FC(F)(F)c1ccc(-c2cnc(Cl)c3cccnc23)nc1 ; CC(C)(C)OC(=O)N1CC[C@H](Nc2ncc(-c3ccc(C(F)(F)F)cn3)c3ncccc23)C1 ; FC(F)(F)c1ccc(-c2cnc(N[C@H]3CCNC3)c3cccnc23)nc1 ; C=CC(=O)N1CC[C@H](Nc2ncc(-c3ccc(C(F)(F)F)cn3)c3ncccc23)C1]

### tert-butyl (S)-3-((8-(5-(trifluoromethyl)pyridin-2-yl)-1,6-naphthyridin-5-yl)amino)pyrrolidine-1-carboxylate

DIPEA (0.085 mL, 0.48 mmol) was added to a solution of 5-chloro-8-(5-(trifluoromethyl)pyridin-2-yl)-1,6-naphthyridine (Intermediate 9, 50.0 mg, 0.161 mmol) and tert-butyl (S)-3-aminopyrrolidine-1-carboxylate (39.1 mg, 0.210 mmol) in DMSO (2 mL). The resulting mixture was heated to 85° C. and stirred for 20 hrs. The reaction mixture was cooled to rt, diluted with water (10 mL), and extracted with EtOAc (3×15 mL). The combined organics were dried over Na₂SO₄, filtered and concentrated to dryness to afford tert-butyl (S)-3-((8-(5-(trifluoromethyl)pyridin-2-yl)-1,6-naphthyridin-5-yl)amino)pyrrolidine-1-carboxylate as a brown oil which was carried forward without purification and assuming quantitative yield. m/z: (ES⁺) [M+H]⁺=460.

### (S)—N-(pyrrolidin-3-yl)-8-(5-(trifluoromethyl)pyridin-2-yl)-1,6-naphthyridin-5-amine hydrochloride

HCl (4 M in dioxane, 0.571 mL, 2.29 mmol) was added to a solution of tert-butyl (S)-3-((8-(5-(trifluoromethyl)pyridin-2-yl)-1,6-naphthyridin-5-yl)amino)pyrrolidine-1-carboxylate (70 mg, 0.15 mmol) in DCM (5 mL) and the reaction mixture was stirred at rt for 7 hrs. The reaction mixture was concentrated to dryness to afford (S)—N-(pyrrolidin-3-yl)-8-(5-(trifluoromethyl)pyridin-2-yl)-1,6-naphthyridin-5-amine hydrochloride which was carried forward without purification and assuming quantitative yield. m/z: (ES⁺) [M+H]⁺=360.

### (S)-1-(3-((8-(5-(trifluoromethyl)pyridin-2-yl)-1,6-naphthyridin-5-yl)amino)pyrrolidin-1-yl)prop-2-en-1-one

A solution of acryloyl chloride (0.017 mL, 0.21 mmol) in THF (0.5 mL) was added dropwise to a solution of (S)—N-(pyrrolidin-3-yl)-8-(5-(trifluoromethyl)pyridin-2-yl)-1,6-naphthyridin-5-amine hydrochloride (60.3 mg, 0.152 mmol) in THF (3 mL) and saturated aq. NaHCO₃ (1.0 mL) at 0° C. under an atmosphere of N₂. The reaction mixture was stirred at 0° C. for 5 min. The reaction mixture was diluted with water (10 mL) and extracted with EtOAc (3×15 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 40%) to afford (S)-1-(3-((8-(5-(trifluoromethyl)pyridin-2-yl)-1,6-naphthyridin-5-yl)amino)pyrrolidin-1-yl)prop-2-en-1-one (Example 38, 26.5 mg, 42% yield) as a yellow amorphous solid. ¹H NMR (500 MHz, DMSO-d6) δ 2.06-2.39 (2H, m), 3.50-3.59 (1H, m), 3.61-3.67 (1H, m), 3.68-3.81 (1H, m), 3.81-4.06 (1H, m), 4.77-4.93 (1H, m), 5.68 (1H, ddd), 6.16 (1H, ddd), 6.55-6.69 (1H, m), 7.64 (1H, dt), 8.06 (1H, dd), 8.23 (1H, br d), 8.52 (1H, d), 8.76 (1H, s), 8.91 (1H, ddd), 9.01-9.06 (2H, m); m/z: (ES⁺) [M+H]⁺=414.

### Example 39: (S)-1-(3-((8-(4-(trifluoromethyl)phenyl)-1,6-naphthyridin-5-yl)amino)pyrrolidin-1-yl)prop-2-en-1-one

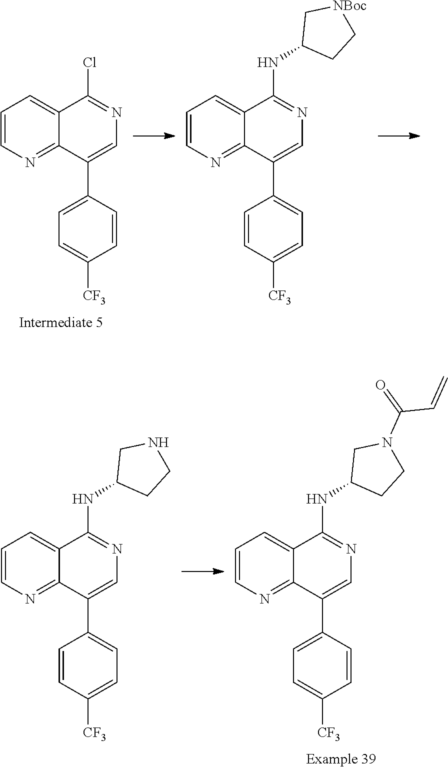 [SMILES: FC(F)(F)c1ccc(-c2cnc(Cl)c3cccnc23)cc1 ; CC(C)(C)OC(=O)N1CC[C@H](Nc2ncc(-c3ccc(C(F)(F)F)cc3)c3ncccc23)C1 ; FC(F)(F)c1ccc(-c2cnc(N[C@H]3CCNC3)c3cccnc23)cc1 ; C=CC(=O)N1CC[C@H](Nc2ncc(-c3ccc(C(F)(F)F)cc3)c3ncccc23)C1]

### tert-butyl (S)-3-((8-(4-(trifluoromethyl)phenyl)-1,6-naphthyridin-5-yl)amino)pyrrolidine-1-carboxylate

DIPEA (0.144 mL, 0.828 mmol) was added to a solution of 5-chloro-8-(4-(trifluoromethyl)phenyl)-1,6-naphthyridine (Intermediate 5, 85 mg, 0.28 mmol) and tert-butyl (S)-3-aminopyrrolidine-1-carboxylate (66.7 mg, 0.358 mmol) in DMSO (1 mL). The resulting mixture was heated to 85° C. and stirred for 14 hrs. Additional tert-butyl (S)-3-aminopyrrolidine-1-carboxylate (35 mg, 0.19 mmol) was added and the resulting mixture was further heated to 90° C. and stirred for an additional 6 hrs. The reaction mixture was cooled to rt, diluted with water (15 mL), and extracted with EtOAc (3×20 mL). The combined organics were dried over Na₂SO₄, filtered and concentrated to dryness to afford tert-butyl (S)-3-((8-(4-(trifluoromethyl)phenyl)-1,6-naphthyridin-5-yl)amino)pyrrolidine-1-carboxylate as a brown oil which was carried forward as a mixture without purification and assuming quantitative yield. m/z: (ES⁺) [M+H]⁺=459.

### (S)—N-(pyrrolidin-3-yl)-8-(4-(trifluoromethyl)phenyl)-1,6-naphthyridin-5-amine hydrochloride

HCl (4 M in dioxane, 0.687 mL, 2.75 mmol) was added to a solution of tert-butyl (S)-3-((8-(4-(trifluoromethyl)phenyl)-1,6-naphthyridin-5-yl)amino)pyrrolidine-1-carboxylate (126 mg, 0.275 mmol) in DCM (6 mL) and the reaction mixture was stirred at rt for 7 hrs. The reaction mixture was concentrated to dryness to afford (S)—N-(pyrrolidin-3-yl)-8-(4-(trifluoromethyl)phenyl)-1,6-naphthyridin-5-amine hydrochloride which was carried forward without purification and assuming quantitative yield. m/z: (ES⁺) [M+H]⁺=359.

### (S)-1-(3-((8-(4-(trifluoromethyl)phenyl)-1,6-naphthyridin-5-yl)amino)pyrrolidin-1-yl)prop-2-en-1-one

A solution of acryloyl chloride (0.031 mL, 0.39 mmol) in THF (0.5 mL) was added dropwise to a solution of (S)—N-(pyrrolidin-3-yl)-8-(4-(trifluoromethyl)phenyl)-1,6-naphthyridin-5-amine hydrochloride (109 mg, 0.276 mmol) in THF (3 mL) and saturated aq. NaHCO₃ (1.0 mL) at 0° C. under an atmosphere of N₂. The reaction mixture was stirred at 0° C. for 20 min. The reaction mixture was diluted with water (10 mL) and extracted with EtOAc (3×15 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 40%) to afford (S)-1-(3-((8-(4-(trifluoromethyl)phenyl)-1,6-naphthyridin-5-yl)amino)pyrrolidin-1-yl)prop-2-en-1-one (Example 39, 34.1 mg, 30% yield) as a beige amorphous solid. ¹H NMR (500 MHz, DMSO-d6) δ 2.06-2.38 (2H, m), 3.50-3.58 (1H, m), 3.60-3.67 (1H, m), 3.68-4.06 (2H, m), 4.70-4.89 (1H, m), 5.68 (1H, ddd), 6.16 (1H, ddd), 6.55-6.69 (1H, m), 7.61 (1H, dt), 7.78 (21H, d), 7.82-7.91 (31H, m), 8.26 (1H, s), 8.82-8.93 (1H, m), 8.94-9.02 (1H, m); m/z: (ES⁺) [M+H]⁺=413.

### Example 40: N-(1-(((4-(4-(trifluoromethyl)phenyl)phthalazin-1-yl)amino)methyl)cyclopropyl)acrylamide

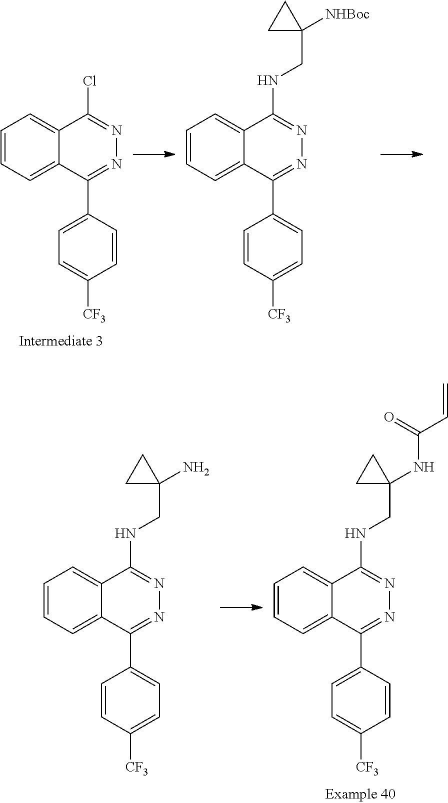 [SMILES: FC(F)(F)c1ccc(-c2nnc(Cl)c3ccccc23)cc1 ; CC(C)(C)OC(=O)NC1(CNc2nnc(-c3ccc(C(F)(F)F)cc3)c3ccccc23)CC1 ; NC1(CNc2nnc(-c3ccc(C(F)(F)F)cc3)c3ccccc23)CC1 ; C=CC(=O)NC1(CNc2nnc(-c3ccc(C(F)(F)F)cc3)c3ccccc23)CC1]

### tert-butyl (1-(((4-(4-(trifluoromethyl)phenyl)phthalazin-1-yl)amino)methyl)cyclopropyl)carbamate

DIPEA (0.204 mL, 1.17 mmol) was added to a solution of 1-chloro-4-(4-(trifluoromethyl)phenyl)phthalazine (Intermediate 3, 120 mg, 0.39 mmol) and tert-butyl (1-(aminomethyl)cyclopropyl)carbamate (87 mg, 0.47 mmol) in DMSO (1 mL). The resulting mixture was heated to 85° C. and stirred for 66 hrs. The reaction mixture was cooled to rt, diluted with water (10 mL), and extracted with EtOAc (2×15 mL). The combined organics were dried over Na₂SO₄, filtered and concentrated to dryness to give tert-butyl (1-(((4-(4-(trifluoromethyl)phenyl)phthalazin-1-yl)amino)methyl)cyclopropyl)carbamate as an orange solid which was carried forward without purification and assuming quantitative yield. m/z: (ES⁺) [M+H]⁺=459.

### N-((1-aminocyclopropyl)methyl)-4-(4-(trifluoromethyl)phenyl)phthalazin-1-amine hydrochloride

HCl (4 M in dioxane, 0.971 mL, 3.88 mmol) was added to a solution of tert-butyl (1-(((4-(4-(trifluoromethyl)phenyl)phthalazin-1-yl)amino)methyl)cyclopropyl)carbamate (178 mg, 0.388 mmol) in DCM (5 mL) and the reaction mixture was stirred at rt for 23 hrs. The reaction mixture was concentrated to dryness to give N-((1-aminocyclopropyl)methyl)-4-(4-(trifluoromethyl)phenyl)phthalazin-1-amine hydrochloride as an off-white solid which was carried forward without purification and assuming quantitative yield. m/z: (ES⁺) [M+H]⁺=359.

### N-(1-(((4-(4-(trifluoromethyl)phenyl)phthalazin-1-yl)amino)methyl)cyclopropyl)acrylamide

A solution of acryloyl chloride (0.022 mL, 0.27 mmol) in THF (1 mL) was added dropwise to a solution of N-((1-aminocyclopropyl)methyl)-4-(4-(trifluoromethyl)phenyl)phthalazin-1-amine hydrochloride (77 mg, 0.20 mmol) in THF (8 mL) and saturated aq. NaHCO₃ (2 mL) at 0° C. under an atmosphere of N₂. The reaction mixture was stirred at 0° C. for 15 min. The reaction mixture was diluted with water (10 mL) and extracted with EtOAc (2×15 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 40%) to afford N-(1-(((4-(4-(trifluoromethyl)phenyl)phthalazin-1-yl)amino)methyl)cyclopropyl)acrylamide (Example 40, 51.7 mg, 64% yield) as an off-white amorphous solid. ¹H NMR (500 MHz, DMSO-d6) δ 0.74-0.82 (2H, m), 0.97-1.05 (2H, m), 3.83 (2H, d), 5.59-5.66 (1H, m), 6.14-6.25 (2H, m), 7.81 (1H, d), 7.84-7.88 (2H, m), 7.88-7.94 (3H, m), 7.99 (1H, t), 8.21 (1H, br s), 8.33 (1H, d), 8.70 (1H, s); m/z: (ES⁺) [M+H]⁺=413.

### Example 41: N-(1-(((4-(4-(trifluoromethyl)phenyl)phthalazin-1-yl)amino)methyl)cyclobutyl)acrylamide

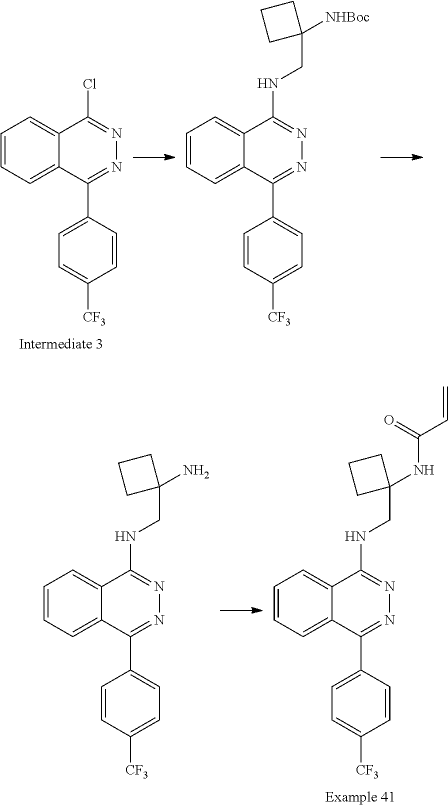 [SMILES: FC(F)(F)c1ccc(-c2nnc(Cl)c3ccccc23)cc1 ; CC(C)(C)OC(=O)NC1(CNc2nnc(-c3ccc(C(F)(F)F)cc3)c3ccccc23)CCC1 ; NC1(CNc2nnc(-c3ccc(C(F)(F)F)cc3)c3ccccc23)CCC1 ; C=CC(=O)NC1(CNc2nnc(-c3ccc(C(F)(F)F)cc3)c3ccccc23)CCC1]

### tert-butyl (1-(((4-(4-(trifluoromethyl)phenyl)phthalazin-1-yl)amino)methyl)cyclobutyl)carbamate

DIPEA (0.17 mL, 0.97 mmol) was added to a solution of 1-chloro-4-(4-(trifluoromethyl)phenyl)phthalazine (Intermediate 3, 100.0 mg, 0.3240 mmol) and tert-butyl (1-(aminomethyl)cyclobutyl)carbamate (78 mg, 0.39 mmol) in DMSO (1 mL). The resulting mixture was heated to 85° C. and stirred for 17 hrs. The reaction mixture was cooled to rt, diluted with water (10 mL), and extracted with EtOAc (2×15 mL). The combined organics were dried over Na₂SO₄, filtered and concentrated to dryness to afford tert-butyl (1-(((4-(4-(trifluoromethyl)phenyl)phthalazin-1-yl)amino)methyl)cyclobutyl)carbamate which was carried forward without purification and assuming quantitative yield. m/z: (ES⁺) [M+H]⁺=473.

### N-((1-aminocyclobutyl)methyl)-4-(4-(trifluoromethyl)phenyl)phthalazin-1-amine hydrochloride

HCl (4 M in dioxane, 0.405 mL, 1.62 mmol) was added to a solution of tert-butyl (1-(((4-(4-(trifluoromethyl)phenyl)phthalazin-1-yl)amino)methyl)cyclobutyl)carbamate (153 mg, 0.324 mmol) in DCM (8 mL) and the reaction mixture was stirred at rt for 48 hrs. The reaction mixture was concentrated to dryness to afford N-((1-aminocyclobutyl)methyl)-4-(4-(trifluoromethyl)phenyl)phthalazin-1-amine hydrochloride as a light brown dry film which was carried forward without purification and assuming quantitative yield. m/z: (ES⁺) [M+H]⁺=373.

### N-(1-(((4-(4-(trifluoromethyl)phenyl)phthalazin-1-yl)amino)methyl)cyclobutyl)acrylamide

A solution of acryloyl chloride (0.019 mL, 0.23 mmol) in THF (1 mL) was added dropwise to a solution of N-((1-aminocyclobutyl)methyl)-4-(4-(trifluoromethyl)phenyl)phthalazin-1-amine hydrochloride (68 mg, 0.17 mmol) in THF (6 mL) and saturated aq. NaHCO₃ (1.5 mL) at 0° C. under an atmosphere of N₂. The reaction mixture was stirred at 0° C. for 25 min. The reaction mixture was diluted with water (15 mL) and extracted with EtOAc (2×20 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 35%) to afford N-(1-(((4-(4-(trifluoromethyl)phenyl)phthalazin-1-yl)amino)methyl)cyclobutyl)acrylamide (Example 41, 43.7 mg, 62% yield) as an off-white amorphous solid. ¹H NMR (500 MHz, DMSO-d6) δ 1.84-1.94 (2H, m), 2.25-2.39 (4H, m), 4.01 (2H, br d), 5.59-5.65 (1H, m), 6.12-6.18 (1H, m), 6.22-6.29 (1H, m), 7.82 (1H, d), 7.85-7.94 (5H, m), 7.97-8.01 (1H, m), 8.14 (1H, s), 8.35 (1H, d), 8.68 (1H, s); m/z: (ES⁺) [M+H]⁺=427.

### Example 42: N-((1-((4-(4-(trifluoromethyl)phenyl)phthalazin-1-yl)amino)cyclobutyl)methyl)acrylamide

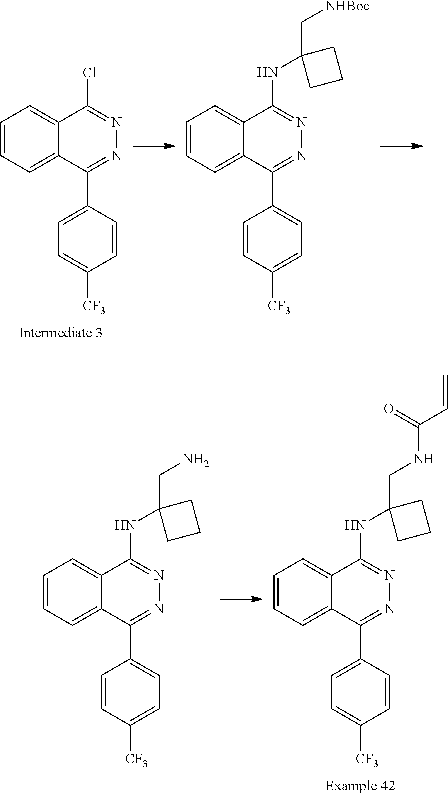 [SMILES: FC(F)(F)c1ccc(-c2nnc(Cl)c3ccccc23)cc1 ; CC(C)(C)OC(=O)NCC1(Nc2nnc(-c3ccc(C(F)(F)F)cc3)c3ccccc23)CCC1 ; NCC1(Nc2nnc(-c3ccc(C(F)(F)F)cc3)c3ccccc23)CCC1 ; C=CC(=O)NCC1(Nc2nnc(-c3ccc(C(F)(F)F)cc3)c3ccccc23)CCC1]

### tert-butyl ((1-((4-(4-(trifluoromethyl)phenyl)phthalazin-1-yl)amino)cyclobutyl)methyl)carbamate

1-Chloro-4-(4-(trifluoromethyl)phenyl)phthalazine (120 mg, 0.39 mmol), tert-butyl ((1-aminocyclobutyl)methyl)carbamate (Intermediate 3, 93 mg, 0.47 mmol), [1,3-bis(2,6-di-3-pentylphenyl)imidazol-2-ylidene](3-chloropyridyl)dichloropalladium(II) (30.8 mg, 0.0389 mmol), and Cs₂CO₃ (253 mg, 0.777 mmol) were diluted in 1,4-dioxane (5 mL) under an atmosphere of N₂. The reaction mixture was heated to 100° C. and stirred for 6 hrs. The reaction mixture was cooled to rt, diluted with water (15 mL), and extracted with EtOAc (2×20 mL). The combined organics were dried over Na₂SO₄, filtered and concentrated to dryness to afford tert-butyl ((1-((4-(4-(trifluoromethyl)phenyl)phthalazin-1-yl)amino)cyclobutyl)methyl)carbamate as a brown dry film which was carried forward without purification and assuming quantitative yield. m/z: (ES⁺) [M+H]⁺=473.

### N-(1-(aminomethyl)cyclobutyl)-4-(4-(trifluoromethyl)phenyl)phthalazin-1-amine hydrochloride

HCl (4 M in dioxane, 0.974 mL, 3.89 mmol) was added to a solution of tert-butyl ((1-((4-(4-(trifluoromethyl)phenyl)phthalazin-1-yl)amino)cyclobutyl)methyl)carbamate (184 mg, 0.389 mmol) in DCM (5 mL) and the reaction mixture stirred at rt for 22 hrs. The reaction mixture was concentrated to dryness to afford N-(1-(aminomethyl)cyclobutyl)-4-(4-(trifluoromethyl)phenyl)phthalazin-1-amine hydrochloride as an off-white solid which was carried forward without purification and assuming quantitative yield. m/z: (ES⁺) [M+H]⁺=373.

### N-((1-((4-(4-(trifluoromethyl)phenyl)phthalazin-1-yl)amino)cyclobutyl)methyl)acrylamide

A solution of acryloyl chloride (0.022 mL, 0.27 mmol) in THF (1 mL) was added dropwise to a solution of N-(1-(aminomethyl)cyclobutyl)-4-(4-(trifluoromethyl)phenyl)phthalazin-1-amine hydrochloride (79 mg, 0.19 mmol) in THF (8 mL) and saturated aq. NaHCO₃ (2 mL) at 0° C. under an atmosphere of N₂. The reaction mixture was stirred at 0° C. for 20 min. The reaction mixture was diluted with water (15 mL) and extracted with EtOAc (2×20 mL). The combined organics were dried over Na₂SO₄, filtered and concentrated to dryness. The crude material was purified by reverse phase purification (C18: 0-100% MeCN in H₂O w/0.1% HCO₂H, with isocratic elution at 40%) to afford N-((1-((4-(4-(trifluoromethyl)phenyl)phthalazin-1-yl)amino)cyclobutyl)methyl)acrylamide (Example 42, 15.6 mg, 19% yield) as a white amorphous solid. ¹H NMR (500 MHz, DMSO-d6) δ 1.82-1.93 (2H, m), 2.31 (2H, ddd), 2.40-2.48 (2H, m), 3.96 (2H, d), 5.54-5.61 (1H, m), 6.10 (1H, dd), 6.28 (1H, dd), 7.51 (1H, s), 7.78-7.82 (1H, m), 7.84-7.89 (3H, m), 7.90-7.94 (3H, m), 8.24 (1H, t), 8.50 (1H, d); m/z: (ES⁺) [M+H]⁺=427.

### Example 43: 1-((3S,4R)-3-fluoro-4-((8-(4-(trifluoromethyl)phenyl)pyrido[3,4-b]pyrazin-5-yl)amino)pyrrolidin-1-yl)prop-2-en-1-one

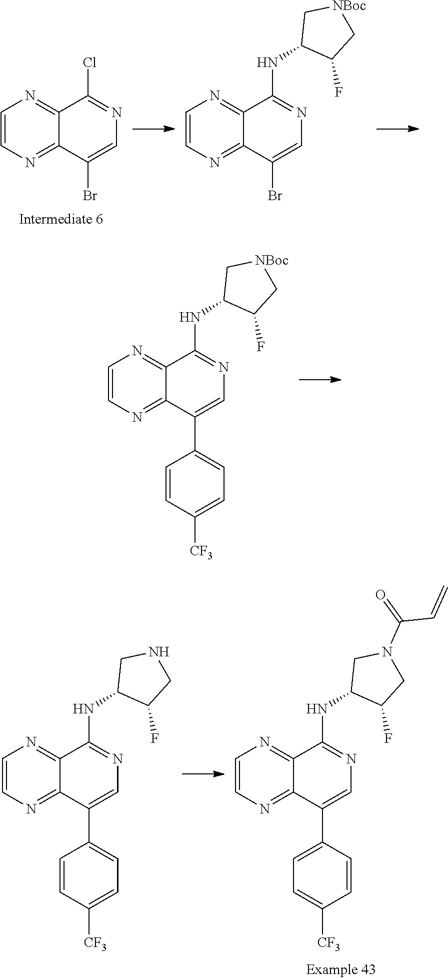 [SMILES: Clc1ncc(Br)c2nccnc12 ; CC(C)(C)OC(=O)N1C[C@H](F)[C@H](Nc2ncc(Br)c3nccnc23)C1 ; CC(C)(C)OC(=O)N1C[C@H](F)[C@H](Nc2ncc(-c3ccc(C(F)(F)F)cc3)c3nccnc23)C1 ; F[C@H]1CNC[C@H]1Nc1ncc(-c2ccc(C(F)(F)F)cc2)c2nccnc12 ; C=CC(=O)N1C[C@H](F)[C@H](Nc2ncc(-c3ccc(C(F)(F)F)cc3)c3nccnc23)C1]

### tert-butyl (3R,4S)-3-((8-bromopyrido[3,4-b]pyrazin-5-yl)amino)-4-fluoropyrrolidine-1-carboxylate

DIPEA (0.44 mL, 2.5 mmol) was added to a solution of 8-bromo-5-chloropyrido[3,4-b]pyrazine (Intermediate 6, 0.29 g, 1.2 mmol) and tert-butyl (3R,4S)-3-amino-4-fluoropyrrolidine-1-carboxylate (0.20 g, 1.0 mmol) in DMSO (3 mL). The reaction mixture was heated to 90° C. and stirred for 16 hrs. The reaction mixture was cooled to rt and diluted with DCM (50 mL) and water (20 mL). The phases were separated and the aqueous layer was extracted with DCM (2×50 mL). The combined organics were dried over Na₂SO₄, filtered and concentrated to dryness. The resulting residue was purified by flash silica chromatography (0 to 25% EtOAc in hexanes) to afford tert-butyl (3R,4S)-3-((8-bromopyrido[3,4-b]pyrazin-5-yl)amino)-4-fluoropyrrolidine-1-carboxylate (263 mg, 64% yield) as a yellow solid. ¹H NMR (500 MHz, DMSO-d6) δ 1.42 (9H, s), 3.47-3.75 (3H, m), 3.76-3.82 (1H, m), 4.73-4.93 (1H, m), 5.27-5.48 (1H, m), 7.86-8.02 (1H, m), 8.43 (1H, s), 8.94 (1H, d), 9.18 (1H, d); m/z: (ES⁺) [M+H]⁺=412.

### tert-butyl (3S,4R)-3-fluoro-4-((8-(4-(trifluoromethyl)phenyl)pyrido[3,4-b]pyrazin-5-yl)amino)pyrrolidine-1-carboxylate

tert-Butyl (3R,4S)-3-((8-bromopyrido[3,4-b]pyrazin-5-yl)amino)-4-fluoropyrrolidine-1-carboxylate (263 mg, 0.638 mmol), (4-(trifluoromethyl)phenyl)boronic acid (182 mg, 0.958 mmol), Cs₂CO₃ (624 mg, 1.92 mmol), and Pd(dppf)Cl₂—CH₂Cl₂ (78 mg, 0.096 mmol) were diluted in 1,4-dioxane (4 mL) and water (1 mL) under an atmosphere of N₂. The reaction mixture was heated to 95° C. and stirred for 2 hrs. The reaction mixture was cooled to rt and diluted with EtOAc (50 mL) and water (50 mL). The phases were separated and the aqueous layer was extracted with EtOAc (2×50 mL). The combined organics were washed with water (2×25 mL), dried over Na₂SO₄, filtered and concentrated to dryness. The resulting residue was purified by flash silica chromatography (0 to 25% EtOAc in hexanes) to afford tert-butyl (3S,4R)-3-fluoro-4-((8-(4-(trifluoromethyl)phenyl)pyrido[3,4-b]pyrazin-5-yl)amino)pyrrolidine-1-carboxylate (260 mg, 85% yield) as a yellow solid. ¹H NMR (500 MHz, DMSO-d6) δ 1.43 (9H, s), 3.49-3.79 (3H, m), 3.84 (1H, td), 4.84-5.05 (1H, m), 5.30-5.49 (1H, m), 7.82 (2H, d), 7.86-8.00 (3H, m), 8.36 (1H, s), 8.94 (1H, d), 9.12 (1H, d); m/z: (ES⁺) [M+H]⁺=478.

### N-((3R,4S)-4-fluoropyrrolidin-3-yl)-8-(4-(trifluoromethyl)phenyl)pyrido[3,4-b]pyrazin-5-amine hydrochloride

HCl (4M in dioxane, 2.0 mL, 8.0 mmol) was added to a solution of tert-butyl (3S,4R)-3-fluoro-4-((8-(4-(trifluoromethyl)phenyl)pyrido[3,4-b]pyrazin-5-yl)amino)pyrrolidine-1-carboxylate (0.14 g, 0.30 mmol) in MeOH (1 mL) and the reaction mixture was stirred at room temperature for 1 h. The solvent was removed under reduced pressure and the resulting oil was dried under high vacuum to afford N-((3R,4S)-4-fluoropyrrolidin-3-yl)-8-(4-(trifluoromethyl)phenyl)pyrido[3,4-b]pyrazin-5-amine hydrochloride (124 mg, 100% yield) as a yellow solid, which was used in the next step without further purification. ¹H NMR (500 MHz, DMSO-d6) δ 3.59-3.73 (4H, m), 4.96-5.15 (1H, m), 5.41-5.61 (1H, m), 7.82-7.92 (4H, m), 8.33-8.46 (2H, m), 8.99 (1H, d), 9.16 (1H, d), 9.75 (2H, br s); m/z: (ES⁺) [M+H]⁺=378.

### 1-((3S,4R)-3-fluoro-4-((8-(4-(trifluoromethyl)phenyl)pyrido[3,4-b]pyrazin-5-yl)amino)pyrrolidin-1-yl)prop-2-en-1-one

A solution of acryloyl chloride (29 μL, 0.36 mmol) in DCM (1 mL) was added dropwise to a solution of N-((3R,4S)-4-fluoropyrrolidin-3-yl)-8-(4-(trifluoromethyl)phenyl)pyrido[3,4-b]pyrazin-5-amine hydrochloride (0.12 g, 0.30 mmol) and triethylamine (0.17 mL, 1.2 mmol) in DCM (5 mL) at 0° C. The reaction mixture was allowed to stir at 0° C. for 1 h. After warming up to room temperature, the reaction mixture was diluted with DCM (25 mL) and water (25 mL). The phases were separated and the aqueous layer was extracted with DCM (2×25 mL). The combined organics were dried over Na₂SO₄, filtered and concentrated to dryness. The resulting residue was purified by flash silica chromatography (0 to 75% EtOAc in hexanes) to afford 1-((3S,4R)-3-fluoro-4-((8-(4-(trifluoromethyl)phenyl)pyrido[3,4-b]pyrazin-5-yl)amino)pyrrolidin-1-yl)prop-2-en-1-one (Example 43, 91 mg, 70% yield) as a yellow amorphous solid. ¹H NMR (500 MHz, DMSO-d6) δ 3.60-4.24 (4H, m), 4.89-5.18 (1H, m), 5.36-5.58 (1H, m), 5.71-5.76 (1H, m), 6.20 (1H, dt), 6.61 (1H, ddd), 7.79-7.85 (2H, m), 7.87-7.91 (2H, m), 7.92-8.06 (1H, m), 8.37 (1H, d), 8.94-8.97 (1H, m), 9.13 (1H, dd); m/z: (ES⁺) [M+H]⁺=432.

### Example 44: 1-((3S,4R)-3-fluoro-4-((8-(4-(trifluoromethyl)phenyl)-1,6-naphthyridin-5-yl)amino)pyrrolidin-1-yl)prop-2-en-1-one

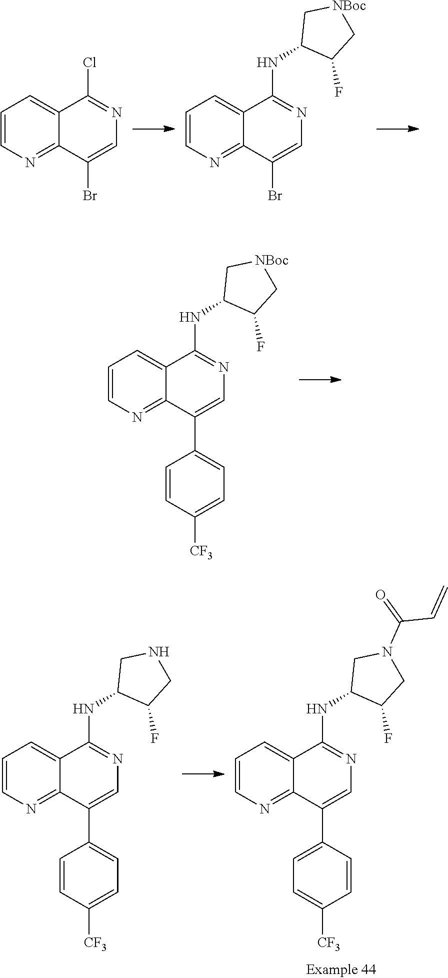 [SMILES: Clc1ncc(Br)c2ncccc12 ; CC(C)(C)OC(=O)N1C[C@H](F)[C@H](Nc2ncc(Br)c3ncccc23)C1 ; CC(C)(C)OC(=O)N1C[C@H](F)[C@H](Nc2ncc(-c3ccc(C(F)(F)F)cc3)c3ncccc23)C1 ; F[C@H]1CNC[C@H]1Nc1ncc(-c2ccc(C(F)(F)F)cc2)c2ncccc12 ; C=CC(=O)N1C[C@H](F)[C@H](Nc2ncc(-c3ccc(C(F)(F)F)cc3)c3ncccc23)C1]

### tert-butyl (3R,4S)-3-((8-bromo-1,6-naphthyridin-5-yl)amino)-4-fluoropyrrolidine-1-carboxylate

DIPEA (0.44 mL, 2.5 mmol) was added to a solution of 8-bromo-5-chloro-1,6-naphthyridine (0.29 g, 1.2 mmol) and tert-butyl (3R,4S)-3-amino-4-fluoropyrrolidine-1-carboxylate (0.20 g, 1.0 mmol) in DMSO (3 mL). The reaction mixture was heated to 90° C. and stirred for 22 hrs. The reaction mixture was cooled to rt and diluted with EtOAc (50 mL) and water (50 mL). The phases were separated and the aqueous layer was extracted with EtOAc (2×50 mL). The combined organics were washed with water (2×25 mL), dried over Na₂SO₄, filtered and concentrated to dryness. The resulting residue was purified by flash silica chromatography (0 to 50% EtOAc in hexanes) to afford tert-butyl (3R,4S)-3-((8-bromo-1,6-naphthyridin-5-yl)amino)-4-fluoropyrrolidine-1-carboxylate (183 mg, 45% yield) as a white solid. ¹H NMR (500 MHz, DMSO-d6) δ 1.43 (9H, d), 3.48-3.71 (3H, m), 3.76 (1H, t), 4.71-4.91 (1H, m), 5.26-5.46 (1H, m), 7.67 (1H, dd), 7.81-7.87 (1H, m), 8.35 (1H, s), 8.89 (1H, br d), 9.05-9.10 (1H, m); m/z: (ES⁺) [M+H]⁺=411.

### tert-butyl (3S,4R)-3-fluoro-4-((8-(4-(trifluoromethyl)phenyl)-1,6-naphthyridin-5-yl)amino)pyrrolidine-1-carboxylate

tert-Butyl (3R,4S)-3-((8-bromo-1,6-naphthyridin-5-yl)amino)-4-fluoropyrrolidine-1-carboxylate (0.18 g, 0.44 mmol), (4-(trifluoromethyl)phenyl)boronic acid (0.13 g, 0.67 mmol), Cs₂CO₃ (0.44 g, 1.3 mmol), and Pd(dppf)Cl₂—CH₂Cl₂ (55 mg, 0.067 mmol) were diluted in 1,4-dioxane (4 mL) and water (1 mL) under an atmosphere of N₂. The reaction mixture was heated to 95° C. and stirred for 2 hrs. The reaction mixture was cooled to rt and diluted with EtOAc (50 mL) and water (50 mL). The phases were separated and the aqueous layer was extracted with EtOAc (2×50 mL). The combined organics were washed with water (2×25 mL), dried over Na₂SO₄, filtered and concentrated to dryness. The resulting residue was purified by flash silica chromatography (0 to 25% EtOAc in hexanes) to afford tert-butyl (3S,4R)-3-fluoro-4-((8-(4-(trifluoromethyl)phenyl)-1,6-naphthyridin-5-yl)amino)pyrrolidine-1-carboxylate (168 mg, 79% yield) as a white solid. ¹H NMR (500 MHz, DMSO-d6) δ 1.44 (9H, d), 3.52-3.83 (4H, m), 4.82-5.02 (1H, m), 5.32-5.52 (1H, m), 7.64 (1H, dd), 7.79 (2H, d), 7.83-7.88 (3H, m), 8.25 (1H, s), 8.90-8.96 (1H, m), 8.99 (1H, d); m/z: (ES⁺) [M+H]⁺=477.

### N-((3R,4S)-4-fluoropyrrolidin-3-yl)-8-(4-(trifluoromethyl)phenyl)-1,6-naphthyridin-5-amine hydrochloride

HCl (4 M in dioxane, 2.0 mL, 8.0 mmol) was added to a solution of tert-butyl (3R,4S)-3-((8-bromo-1,6-naphthyridin-5-yl)amino)-4-fluoropyrrolidine-1-carboxylate (168 mg, 0.350 mmol) in MeOH (1 mL) and the reaction mixture was stirred at room temperature for 1 h. The solvent was removed under reduced pressure and the resulting oil was dried under high vacuum to afford N-((3R,4S)-4-fluoropyrrolidin-3-yl)-8-(4-(trifluoromethyl)phenyl)-1,6-naphthyridin-5-amine hydrochloride (144 mg, 99% yield) as a white solid, which was used in the next step without purification. ¹H NMR (500 MHz, DMSO-d6) δ 3.61-3.75 (4H, m), 4.98-5.18 (1H, m), 5.44-5.64 (1H, m), 7.67-7.79 (1H, m), 7.80-7.89 (4H, m), 8.21 (1H, s), 8.41-8.71 (1H, m), 9.04-9.12 (2H, m), 9.56-9.80 (1H, m), 9.90-10.04 (1H, m); m/z: (ES⁺) [M+H]⁺=377.

### 1-((3S,4R)-3-fluoro-4-((8-(4-(trifluoromethyl)phenyl)-1,6-naphthyridin-5-yl)amino)pyrrolidin-1-yl)prop-2-en-1-one

A solution of acryloyl chloride (34 μL, 0.38 mmol) in DCM (1 mL) was added dropwise to a solution of N-((3R,4S)-4-fluoropyrrolidin-3-yl)-8-(4-(trifluoromethyl)phenyl)-1,6-naphthyridin-5-amine hydrochloride (144 mg, 0.350 mmol) and Et₃N (0.20 mL, 1.4 mmol) in DCM (5 mL) at 0° C. The reaction mixture was stirred at 0° C. for 1 h. The reaction mixture was diluted with DCM (25 mL) and water (25 mL). The phases were separated and the aqueous layer was extracted with DCM (2×25 mL). The combined organics were dried over Na₂SO₄, filtered and concentrated to dryness. The resulting residue was purified by flash silica chromatography (0 to 75% EtOAc in hexanes) to afford 1-((3S,4R)-3-fluoro-4-((8-(4-(trifluoromethyl)phenyl)-1,6-naphthyridin-5-yl)amino)pyrrolidin-1-yl)prop-2-en-1-one (Example 44, 93 mg, 62% yield) as a white amorphous solid. ¹H NMR (500 MHz, DMSO-d6) δ 3.60-4.25 (4H, m), 4.87-5.18 (1H, m), 5.36-5.60 (1H, m), 5.74 (1H, td), 6.21 (1H, ddd), 6.55-6.70 (1H, m), 7.65 (1H, ddd), 7.76-7.82 (2H, m), 7.85-7.94 (3H, m), 8.26 (1H, d), 8.91-8.97 (1H, m), 9.00 (1H, dt); m/z: (ES⁺) [M+H]⁺=431.

### Example 45: (S)-1-(3-((8-(4-(trifluoromethyl)phenyl)pyrido[3,4-b]pyrazin-5-yl)amino)pyrrolidin-1-yl)prop-2-en-1-one

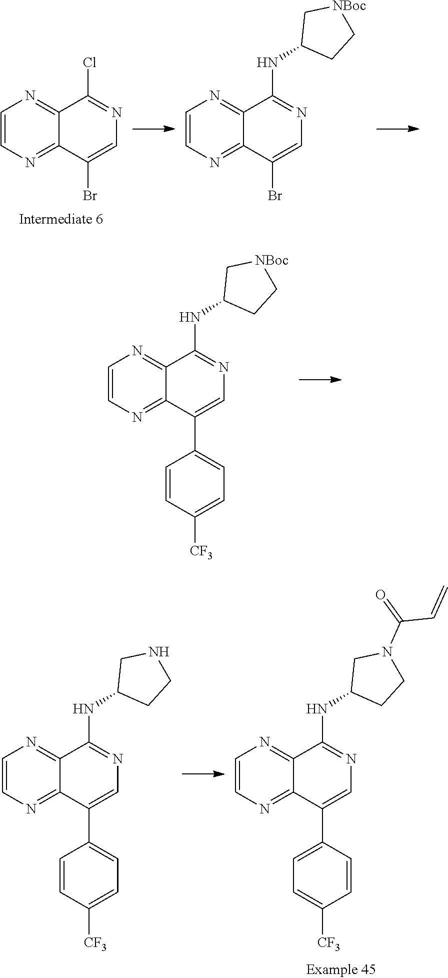 [SMILES: Clc1ncc(Br)c2nccnc12 ; CC(C)(C)OC(=O)N1CC[C@H](Nc2ncc(Br)c3nccnc23)C1 ; CC(C)(C)OC(=O)N1CC[C@H](Nc2ncc(-c3ccc(C(F)(F)F)cc3)c3nccnc23)C1 ; FC(F)(F)c1ccc(-c2cnc(N[C@H]3CCNC3)c3nccnc23)cc1 ; C=CC(=O)N1CC[C@H](Nc2ncc(-c3ccc(C(F)(F)F)cc3)c3nccnc23)C1]

### tert-butyl (S)-3-((8-bromopyrido[3,4-b]pyrazin-5-yl)amino)pyrrolidine-1-carboxylate

DIPEA (0.857 mL, 4.91 mmol) was added to a solution of tert-butyl (S)-3-aminopyrrolidine-1-carboxylate (1.524 g, 8.182 mmol) and 8-bromo-5-chloropyrido[3,4-b]pyrazine (Intermediate 6, 0.400 g, 1.64 mmol) in DMSO (24 mL). The resulting mixture was heated to 100° C. and stirred for 2 hrs. The reaction mixture was cooled to rt, diluted with DCM (20 mL), and washed with brine (3×50 mL). The organic layer was dried over Na₂SO₄, filtered and concentrated to dryness. The crude material was purified on a reverse phase C18 column (5 to 80% MeCN in H₂O w/0.05% TFA) to afford tert-butyl (S)-3-((8-bromopyrido[3,4-b]pyrazin-5-yl)amino)pyrrolidine-1-carboxylate (600 mg, 93% yield) as a yellow solid. ¹H NMR (300 MHz, DMSO-d6) δ 1.38 (9H, d), 2.02-2.20 (2H, m), 3.25-3.36 (2H, m), 3.39-3.50 (1H, m), 3.57-3.68 (1H, m), 4.55-4.67 (1H, m), 8.21 (1H, d), 8.38 (1H, s), 8.89 (1H, d), 9.14 (1H, d); m/z: (ES⁺) [M+H]⁺=394.

### tert-butyl (S)-3-((8-(4-(trifluoromethyl)phenyl)pyrido[3,4-b]pyrazin-5-yl)amino)pyrrolidine-1-carboxylate

tert-Butyl (S)-3-((8-bromopyrido[3,4-b]pyrazin-5-yl)amino)pyrrolidine-1-carboxylate (0.30 g, 0.76 mmol), (4-(trifluoromethyl)phenyl)boronic acid (145 mg, 0.763 mmol), Cs₂CO₃ (744 mg, 2.28 mmol) and bis(di-tert-butyl(4-dimethylaminophenyl)phosphine)dichloropalladium(II) (53.9 mg, 0.0761 mmol) were diluted in 1,4-dioxane (10 mL) and water (1 mL) under an atmosphere of N₂. The resulting mixture was heated to 40° C. and stirred for 2 hrs. The reaction mixture was cooled to rt and the volatiles were removed under reduced pressure. The resulting residue was purified by preparative HPLC (XBridge Prep C18 OBD column: 50 mm×100 mm, 5 μm; using decreasingly polar mixtures of water (containing 0.1% formic acid) and MeCN as eluents) to afford tert-butyl (S)-3-((8-(4-(trifluoromethyl)phenyl)pyrido[3,4-b]pyrazin-5-yl)amino)pyrrolidine-1-carboxylate (130 mg, 37% yield) as a yellow solid. ¹H NMR (400 MHz, DMSO-d6) δ 1.41 (9H, d), 2.10-2.25 (2H, m), 3.33-3.38 (2H, m), 3.46-3.54 (1H, m), 3.64-3.73 (1H, m), 4.69-4.80 (1H, m), 7.78-7.84 (2H, m), 7.87-7.91 (2H, m), 8.16 (1H, d), 8.35 (1H, s), 8.90 (1H, d), 9.08 (1H, d); m/z: (ES⁺) [M+H]⁺=460.

### (S)—N-(pyrrolidin-3-yl)-8-(4-(trifluoromethyl)phenyl)pyrido[3,4-b]pyrazin-5-amine hydrochloride

tert-Butyl (S)-3-((8-(4-(trifluoromethyl)phenyl)pyrido[3,4-b]pyrazin-5-yl)amino)pyrrolidine-1-carboxylate (130 mg, 0.28 mmol) was dissolved in HCl (4 M in dioxane, 0.5 mL, 2 mmol) and the reaction mixture was stirred at rt for 2 hrs. The reaction mixture was concentrated to dryness to afford (S)—N-(pyrrolidin-3-yl)-8-(4-(trifluoromethyl)phenyl)pyrido[3,4-b]pyrazin-5-amine hydrochloride (100 mg, 89% yield) as a yellow solid. ¹H NMR (400 MHz, DMSO-d6) δ 2.16-2.25 (1H, m), 2.33-2.43 (1H, m), 3.24-3.33 (1H, m), 3.53-3.62 (1H, m), 3.64-3.68 (1H, m), 3.69-3.73 (1H, m), 4.93-5.03 (1H, m), 7.80-7.92 (4H, m), 8.24 (1H, s), 9.01 (1H, s), 9.19 (1H, s), 9.31 (1H, s), 9.57 (1H, s); m/z: (ES⁺) [M+H]⁺=360.

### (S)-1-(3-((8-(4-(trifluoromethyl)phenyl)pyrido[3,4-b]pyrazin-5-yl)amino)pyrrolidin-1-yl)prop-2-en-1-one

Acryloyl chloride (27.6 μL, 0.341 mmol) was added dropwise to a solution of (S)—N-(pyrrolidin-3-yl)-8-(4-(trifluoromethyl)phenyl)pyrido[3,4-b]pyrazin-5-amine hydrochloride (0.090 g, 0.23 mmol) and Na₂CO₃ (48.2 mg, 0.455 mmol) in THF (8 mL) and water (2 mL) at 0° C. The reaction mixture was warmed to rt and stirred for 12 hrs. The reaction mixture was diluted with water (50 mL) and extracted with DCM (3×50 mL). The combined organics were dried over Na₂SO₄, filtered and concentrated to dryness. The crude material was purified by preparative HPLC (Sunfire prep C18 column: 30×150 mm, 5 μm; Mobile Phase A: Water (0.1% formic acid), Mobile Phase B: MeCN; Flow rate: 60 mL/min; Gradient: 40% B to 58% B; Wavelength: 254/220 nm) to afford (S)-1-(3-((8-(4-(trifluoromethyl)phenyl)pyrido[3,4-b]pyrazin-5-yl)amino)pyrrolidin-1-yl)prop-2-en-1-one (Example 45, 21 mg, 22% yield) as a yellow amorphous solid. ¹H NMR (400 MHz, DMSO-d6) δ 2.11-2.37 (2H, m), 3.47-3.67 (2H, m), 3.68-3.80 (1H, m), 3.82-4.01 (1H, m), 4.74-4.88 (1H, m), 5.57-5.66 (1H, m), 6.06-6.13 (1H, m), 6.47-6.61 (1H, m), 7.71-7.76 (2H, m), 7.79 (1H, s), 7.83-7.89 (2H, m), 8.32 (1H, s), 8.83 (1H, d), 9.02 (1H, d); m/z: (ES⁺) [M+H]⁺=414.

### Example 46: 1-((3R,4S)-3-((dimethylamino)methyl)-4-((4-(4-(trifluoromethyl)phenyl)phthalazin-1-yl)amino)pyrrolidin-1-yl)prop-2-en-1-one

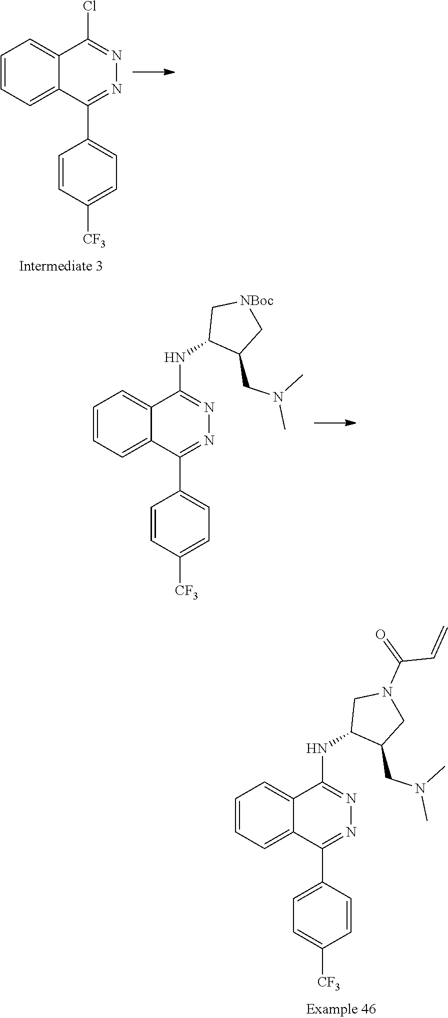 [SMILES: FC(F)(F)c1ccc(-c2nnc(Cl)c3ccccc23)cc1 ; CN(C)C[C@@H]1CN(C(=O)OC(C)(C)C)C[C@H]1Nc1nnc(-c2ccc(C(F)(F)F)cc2)c2ccccc12 ; C=CC(=O)N1C[C@@H](CN(C)C)[C@H](Nc2nnc(-c3ccc(C(F)(F)F)cc3)c3ccccc23)C1]

### tert-butyl (3R,4S)-3-((dimethylamino)methyl)-4-((4-(4-(trifluoromethyl)phenyl)phthalazin-1-yl)amino)pyrrolidine-1-carboxylate

tert-Butyl (3S,4R)-3-amino-4-((dimethylamino)methyl)pyrrolidine-1-carboxylate (177 mg, 0.727 mmol), 1-chloro-4-(4-(trifluoromethyl)phenyl)phthalazine (Intermediate 3, 150 mg, 0.49 mmol), Cs₂CO₃ (475 mg, 1.46 mmol) and [1,3-bis(2,6-di-3-pentylphenyl)imidazol-2-ylidene](3-chloropyridyl)dichloropalladium(II) (47.2 mg, 0.0486 mmol) were diluted in 1,4-dioxane (10 mL) under an atmosphere of N₂. The resulting mixture was heated to 90° C. and stirred for 3 hrs. The reaction mixture was cooled to rt and the volatiles were removed under reduced pressure. The crude material was purified by preparative HPLC (XBridge Prep C18 OBD column: 50 mm×100 mm, μm; using decreasingly polar mixtures of water (w/0.5% NH₄HCO₃) and MeCN as eluents) to afford tert-butyl (3R,4S)-3-((dimethylamino)methyl)-4-((4-(4-(trifluoromethyl)phenyl)phthalazin-1-yl)amino)pyrrolidine-1-carboxylate (150 mg, 60% yield) as a white amorphous solid. ¹H NMR (400 MHz, DMSO-d6) δ 1.41 (9H, d), 2.18 (6H, s), 2.30-2.40 (2H, m), 2.62-2.73 (1H, m), 3.11-3.29 (2H, m), 3.59-3.70 (1H, m), 3.79-3.92 (1H, m), 4.62 (1H, t), 7.59 (1H, t), 7.80 (1H, d), 7.84-7.98 (6H, m), 8.47 (1H, d); m/z: (ES⁺) [M+H]⁺=516.

### 1-((3R,4S)-3-((dimethylamino)methyl)-4-((4-(4-(trifluoromethyl)phenyl)phthalazin-1-yl)amino)pyrrolidin-1-yl)prop-2-en-1-one

tert-Butyl (3R,4S)-3-((dimethylamino)methyl)-4-((4-(4-(trifluoromethyl)phenyl)phthalazin-1-yl)amino)pyrrolidine-1-carboxylate (130 mg, 0.25 mmol) was dissolved in HCl (4 M in dioxane, 10 mL, mmol) and the resulting mixture was stirred at rt for 2 hrs. The reaction mixture was concentrated to dryness and the resulting residue was dissolved in DMF (10 mL). HATU (144 mg, 0.379 mmol), DIPEA (0.176 mL, 1.01 mmol) and acrylic acid (21.8 mg, 0.303 mmol) were added and the reaction stirred at rt for 2 hrs. The reaction mixture was diluted with DCM (100 mL) and washed with water (3×50 mL). The organic layer was dried over Na₂SO₄, filtered and concentrated to dryness. The crude material was purified by preparative HPLC (XBridge Shield RP18 OBD column: 30×150 mm, 5 μm; Mobile Phase A: Water (10 mM NH₄HCO₃+0.1% NH₃—H₂O); Mobile Phase B: MeCN; Flow rate: 60 mL/min; Gradient: 18% B to 66% B over 10 min; Wavelength: 254/220 nm) to afford 1-((3R,4S)-3-((dimethylamino)methyl)-4-((4-(4-(trifluoromethyl)phenyl)phthalazin-1-yl)amino)pyrrolidin-1-yl)prop-2-en-1-one (Example 46, 1.5 mg, 1% yield) as a white amorphous solid. ¹H NMR (400 MHz, DMSO-d6) δ 2.20 (6H, s), 2.29-2.39 (1H, m), 2.43 (1H, d), 2.71 (1H, s), 3.30-3.61 (2H, m), 3.79-4.16 (2H, m), 4.72 (1H, s), 5.61 (1H, d), 6.10 (1H, dd), 6.55 (1H, s), 7.33 (1H, s), 7.76 (1H, d), 7.81-7.86 (5H, m), 7.86-7.94 (1H, m), 8.40 (1H, d); m/z: (ES⁺) [M+H]⁺=470.

### Example 47: 1-((2R,4S)-2-(hydroxymethyl)-4-((4-(4-(trifluoromethyl)phenyl)phthalazin-1-yl)amino)pyrrolidin-1-yl)prop-2-en-1-one

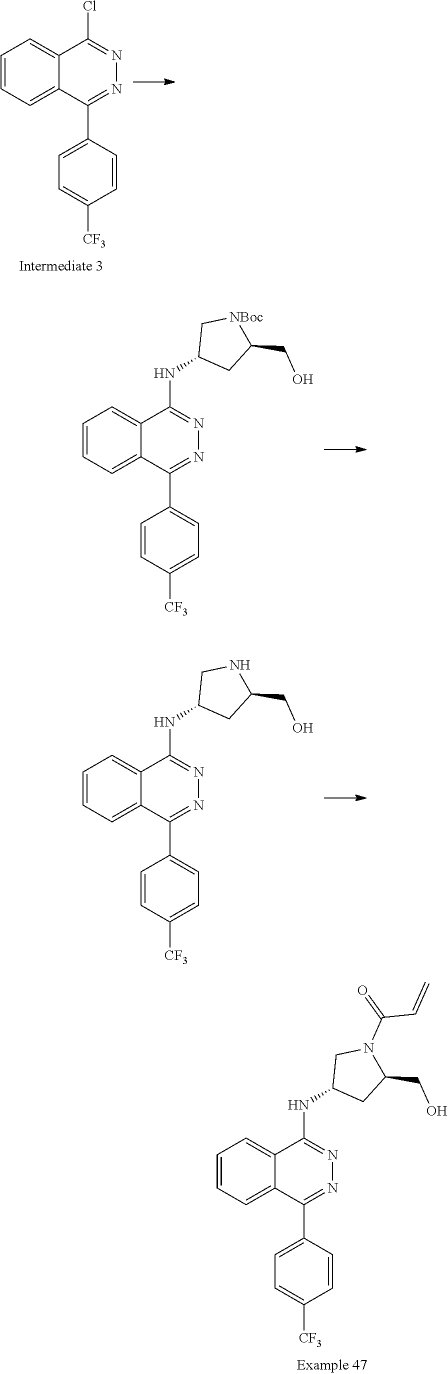 [SMILES: FC(F)(F)c1ccc(-c2nnc(Cl)c3ccccc23)cc1 ; CC(C)(C)OC(=O)N1C[C@@H](Nc2nnc(-c3ccc(C(F)(F)F)cc3)c3ccccc23)C[C@@H]1CO ; OC[C@H]1C[C@H](Nc2nnc(-c3ccc(C(F)(F)F)cc3)c3ccccc23)CN1 ; C=CC(=O)N1C[C@@H](Nc2nnc(-c3ccc(C(F)(F)F)cc3)c3ccccc23)C[C@@H]1CO]

### tert-butyl (2R,4S)-2-(hydroxymethyl)-4-((4-(4-(trifluoromethyl)phenyl)phthalazin-1-yl)amino)pyrrolidine-1-carboxylate

1-Chloro-4-(4-(trifluoromethyl)phenyl)phthalazine (Intermediate 3, 0.40 g, 1.3 mmol), tert-butyl (2R,4S)-4-amino-2-(hydroxymethyl)pyrrolidine-1-carboxylate (420 mg, 1.9 mmol), Cs₂CO₃ (1.267 g, 3.889 mmol) and [1,3-bis(2,6-di-3-pentylphenyl)imidazol-2-ylidene](3-chloropyridyl)dichloropalladium(II) (126 mg, 0.130 mmol) were diluted in 1,4-dioxane (15 mL) under an atmosphere of N₂. The resulting mixture was heated to 90° C. and stirred for 12 hrs. The reaction mixture was cooled to rt and the volatiles were removed under reduced pressure. The resulting residue was purified by preparative HPLC (XBridge Prep C18 OBD column: 50 mm×100 mm, 5 μm; using decreasingly polar mixtures of water (w/0.5% NH₄HCO₃) and MeCN as eluents) to afford tert-butyl (2R,4S)-2-(hydroxymethyl)-4-((4-(4-(trifluoromethyl)phenyl)phthalazin-1-yl)amino)pyrrolidine-1-carboxylate (450 mg, 71% yield) as a white solid. ¹H NMR (400 MHz, DMSO-d6) δ 1.36-1.43 (9H, m), 2.06-2.21 (1H, m), 2.31-2.45 (1H, m), 3.39-3.62 (2H, m), 3.39-3.62 (1H, m), 3.76 (1H, dt), 3.96 (1H, s), 4.87 (1H, t), 4.89-5.01 (1H, m), 7.47-7.56 (1H, m), 7.79 (1H, d), 7.84-7.95 (6H, m), 8.47 (1H, d); m/z: (ES⁺) [M+H]⁺=489.

### ((2R,4S)-4-((4-(4-(trifluoromethyl)phenyl)phthalazin-1-yl)amino)pyrrolidin-2-yl)methanol 2,2,2-trifluoroacetate

TFA (5 mL) was added to a solution of tert-butyl (2R,4S)-2-(hydroxymethyl)-4-((4-(4-(trifluoromethyl)phenyl)phthalazin-1-yl)amino)pyrrolidine-1-carboxylate (440 mg, 0.90 mmol) in DCM (5 mL) and the reaction mixture was stirred at rt for 3 hrs. The reaction mixture was concentrated and the resulting residue was purified by preparative HPLC (XBridge Shield RP18 OBD column: 50 mm×100 mm, 5 μm; using decreasingly polar mixtures of water (w/0.5% NH₄HCO₃) and MeCN as eluents) to afford ((2R,4S)-4-((4-(4-(trifluoromethyl)phenyl)phthalazin-1-yl)amino)pyrrolidin-2-yl)methanol 2,2,2-trifluoroacetate (200 mg, 44% yield) as a white solid. ¹H NMR (400 MHz, DMSO-d6) δ 1.77-2.01 (2H, m), 2.81-2.94 (1H, m), 3.38 (3H, s), 3.46-3.61 (1H, m), 3.70-4.07 (1H, m), 4.33-4.61 (1H, m), 4.65-4.77 (1H, m), 7.46 (1H, d), 7.74-7.82 (1H, m), 7.82-7.99 (6H, m), 8.50 (1H, d); m/z: (ES⁺) [M+H]⁺=389.

### 1-((2R,4S)-2-(hydroxymethyl)-4-((4-(4-(trifluoromethyl)phenyl)phthalazin-1-yl)amino)pyrrolidin-1-yl)prop-2-en-1-one

A solution of acryloyl chloride (20.8 μL, 0.257 mmol) in THF (1 mL) was added dropwise to a solution of ((2R,4S)-4-((4-(4-(trifluoromethyl)phenyl)phthalazin-1-yl)amino)pyrrolidin-2-yl)methanol 2,2,2-trifluoroacetate (100 mg, 0.2 mmol) and sodium bicarbonate (64.9 mg, 0.773 mmol) in THF (9 mL) and water (1 mL) at 0° C. The reaction mixture was stirred at 0° C. for 30 min. The volatiles were removed under reduced pressure and the resulting residue was diluted in water (50 mL) and extracted with DCM (3×50 mL). The combined organics were dried over Na₂SO₄, filtered and concentrated to dryness. The crude material was purified by preparative HPLC (YMC-Actus Triart C18 ExRS: 30 mm×150 mm, 5 μm; Mobile Phase A: Water (10 mM NH₄HCO₃+0.1% NH₃—H₂O), Mobile Phase B: MeCN; Flow rate: 60 mL/min; Gradient: 36% B to 56% B; Wavelength: 254/220 nm) to afford 1-((2R,4S)-2-(hydroxymethyl)-4-((4-(4-(trifluoromethyl)phenyl)phthalazin-1-yl)amino)pyrrolidin-1-yl)prop-2-en-1-one (Example 47, 65 mg, 74% yield) as a white amorphous solid. ¹H NMR (400 MHz, DMSO-d6) δ 2.14-2.42 (2H, m), 3.56 (2H, s), 3.61-3.72 (1H, m), 3.97 (1H, s), 4.30 (1H, s), 4.66 (1H, s), 4.96-5.11 (1H, m), 5.60 (1H, d), 6.04-6.16 (1H, m), 6.57 (1H, s), 7.27 (1H, d), 7.76 (1H, d), 7.81-7.90 (6H, m), 8.40 (1H, d); m/z: (ES⁺) [M+H]⁺=443.

### Example 48: 1-((2S,4S)-2-(hydroxymethyl)-4-((4-(4-(trifluoromethyl)phenyl)phthalazin-1-yl)amino)pyrrolidin-1-yl)prop-2-en-1-one

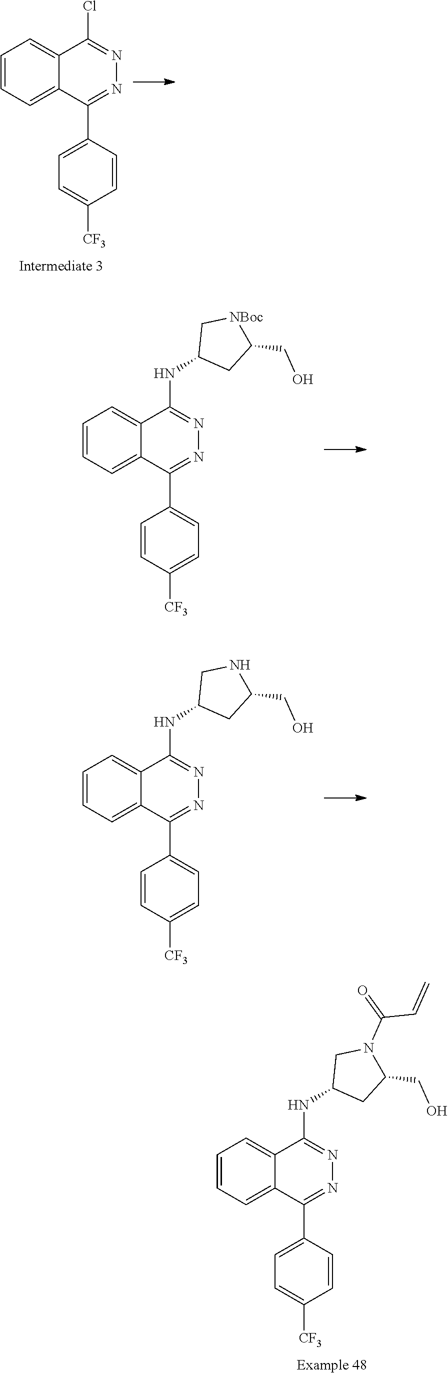 [SMILES: FC(F)(F)c1ccc(-c2nnc(Cl)c3ccccc23)cc1 ; CC(C)(C)OC(=O)N1C[C@@H](Nc2nnc(-c3ccc(C(F)(F)F)cc3)c3ccccc23)C[C@H]1CO ; OC[C@@H]1C[C@H](Nc2nnc(-c3ccc(C(F)(F)F)cc3)c3ccccc23)CN1 ; C=CC(=O)N1C[C@@H](Nc2nnc(-c3ccc(C(F)(F)F)cc3)c3ccccc23)C[C@H]1CO]

### tert-butyl (2S,4S)-2-(hydroxymethyl)-4-((4-(4-(trifluoromethyl)phenyl)phthalazin-1-yl)amino)pyrrolidine-1-carboxylate

1-Chloro-4-(4-(trifluoromethyl)phenyl)phthalazine (Intermediate 3, 0.500 g, 1.62 mmol), tert-butyl (2S,4S)-4-amino-2-(hydroxymethyl)pyrrolidine-1-carboxylate (420 mg, 1.9 mmol), Cs₂CO₃ (1.583 g, 4.859 mmol) and [1,3-bis(2,6-di-3-pentylphenyl)imidazol-2-ylidene](3-chloropyridyl)dichloropalladium(II) (157 mg, 0.162 mmol) were diluted in 1,4-dioxane (10 mL) under an atmosphere of N₂. The resulting mixture was heated to 90° C. and stirred for 16 hrs. The reaction mixture was cooled to rt and the volatiles were removed under reduced pressure. The resulting residue was purified by preparative HPLC (XBridge Prep Phenyl OBD column: 50 mm×100 mm, 5 μm; using decreasingly polar mixtures of water (w/0.5% NH₄HCO₃) and MeCN as eluents) to afford tert-butyl (2S,4S)-2-(hydroxymethyl)-4-((4-(4-(trifluoromethyl)phenyl)phthalazin-1-yl)amino)pyrrolidine-1-carboxylate (560 mg, 71% yield) as a brown solid. ¹H NMR (400 MHz, DMSO-d6) δ 1.42 (9H, s), 2.08-2.26 (1H, m), 2.44-2.49 (1H, m), 3.18-3.29 (1H, m), 3.55-3.65 (1H, m), 3.65-3.78 (1H, m), 3.78-4.02 (2H, m), 4.73 (1H, s), 5.30 (1H, s), 7.74-8.02 (8H, m), 8.29-8.39 (1H, m); m/z: (ES⁺) [M+H]⁺=489.

### ((2S,4S)-4-((4-(4-(trifluoromethyl)phenyl)phthalazin-1-yl)amino)pyrrolidin-2-yl)methanol hydrochloride

tert-Butyl (2S,4S)-2-(hydroxymethyl)-4-((4-(4-(trifluoromethyl)phenyl)phthalazin-1-yl)amino)pyrrolidine-1-carboxylate (560 mg, 1.2 mmol) was dissolved in HCl (37% aq.) and the reaction mixture was stirred at rt for 16 hrs. The reaction mixture was concentrated to dryness to afford ((2S,4S)-4-((4-(4-(trifluoromethyl)phenyl)phthalazin-1-yl)amino)pyrrolidin-2-yl)methanol hydrochloride (450 mg, 92% yield) as a brown solid. ¹H NMR (400 MHz, DMSO-d6) δ 2.08-2.18 (1H, m), 2.66-2.76 (1H, m), 3.14-3.17 (4H, m), 3.63-3.69 (1H, m), 5.00-5.10 (1H, m), 7.88-7.96 (3H, m), 8.00-8.08 (2H, m), 8.12-8.24 (2H, m), 9.24 (1H, d), 9.47 (1H, s), 10.13 (1H, s), 10.37 (1H, s); m/z: (ES⁺) [M+H]⁺=389.

### 1-((2S,4S)-2-(hydroxymethyl)-4-((4-(4-(trifluoromethyl)phenyl)phthalazin-1-yl)amino)pyrrolidin-1-yl)prop-2-en-1-one

Acryloyl chloride (19 μL, 0.24 mmol) was added dropwise to a solution of ((2S,4S)-4-((4-(4-(trifluoromethyl)phenyl)phthalazin-1-yl)amino)pyrrolidin-2-yl)methanol hydrochloride (100 mg, 0.24 mmol) and NaHCO₃ (59.3 mg, 0.706 mmol) in THF (10 mL) and water (2 mL) at 0° C. The reaction mixture was warmed to rt and stirred for 15 min. The volatiles were removed under reduced pressure and the resulting residue was purified by preparative HPLC (YMC-Actus Triart C18 ExRS: 30 mm×150 mm, 5 μm; Mobile Phase A: Water (10 mM NH₄HCO₃+0.1% NH₃—H₂O), Mobile Phase B: MeCN; Flow rate: 60 mL/min; Gradient: 34% B to 56% B; Wavelength: 254/220 nm) to afford 1-((2S,4S)-2-(hydroxymethyl)-4-((4-(4-(trifluoromethyl)phenyl)phthalazin-1-yl)amino)pyrrolidin-1-yl)prop-2-en-1-one (Example 48, 35 mg, 34% yield) as a white amorphous solid. ¹H NMR (400 MHz, DMSO-d6) δ 2.07-2.22 (1H, m), 2.52-2.61 (1H, m), 3.43-3.57 (1H, m), 3.59-3.70 (1H, m), 3.70-3.81 (1H, m), 4.11-4.33 (2H, m), 4.76-4.88 (1H, m), 4.91-5.04 (1H, m), 5.55-5.69 (1H, m), 6.06-6.20 (1H, m), 6.49-6.71 (1H, m), 7.57 (1H, s), 7.72-7.94 (7H, m), 8.25-8.35 (1H, m); m/z: (ES⁺) [M+H]⁺=443.

### Example 49: 1-((3S,4S)-3-(hydroxymethyl)-4-((4-(4-(trifluoromethyl)phenyl)phthalazin-1-yl)amino)pyrrolidin-1-yl)prop-2-en-1-one

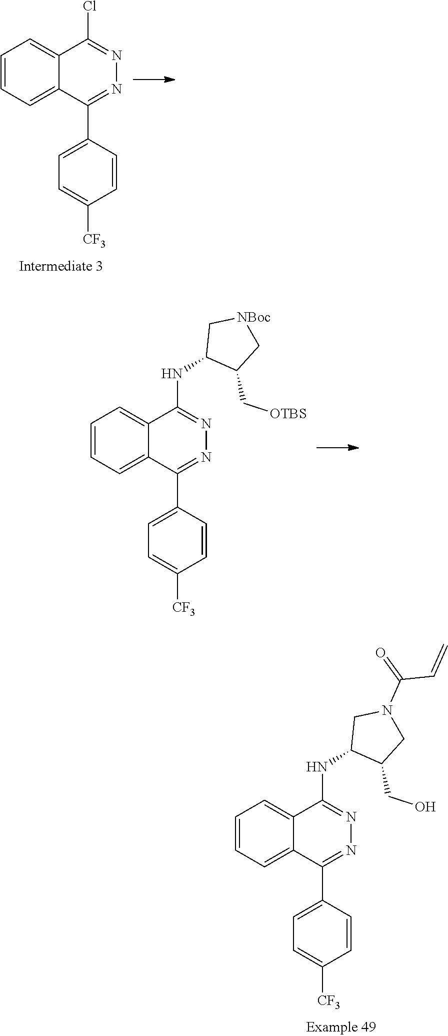 [SMILES: FC(F)(F)c1ccc(-c2nnc(Cl)c3ccccc23)cc1 ; CC(C)(C)OC(=O)N1C[C@H](CO[Si](C)(C)C(C)(C)C)[C@H](Nc2nnc(-c3ccc(C(F)(F)F)cc3)c3ccccc23)C1 ; C=CC(=O)N1C[C@H](CO)[C@H](Nc2nnc(-c3ccc(C(F)(F)F)cc3)c3ccccc23)C1]

### tert-butyl (3S,4S)-3-(((tert-butyldimethylsilyl)oxy)methyl)-4-((4-(4-(trifluoromethyl)phenyl)phthalazin-1-yl)amino)pyrrolidine-1-carboxylate

1-Chloro-4-(4-(trifluoromethyl)phenyl)phthalazine (Intermediate 3, 0.10 g, 0.32 mmol), tert-butyl (3S,4S)-3-amino-4-(((tert-butyldimethylsilyl)oxy)methyl)pyrrolidine-1-carboxylate (107 mg, 0.324 mmol), Cs₂CO₃ (317 mg, 0.973 mmol) and [1,3-bis(2,6-di-3-pentylphenyl)imidazol-2-ylidene](3-chloropyridyl)dichloropalladium(II) (31.5 mg, 0.0323 mmol) were diluted in 1,4-dioxane (10 mL) under an atmosphere of N₂. The resulting mixture was heated to 90° C. and stirred for 16 hrs. The reaction mixture was cooled to rt and the volatiles were removed under reduced pressure. The resulting residue was purified by preparative HPLC (XBridge Prep C18 OBD column: 50 mm×100 mm, 5 μm; using decreasingly polar mixtures of water (w/0.1% NH₄HCO₃) and MeCN as eluents) to afford tert-butyl (3S,4S)-3-(((tert-butyldimethylsilyl)oxy)methyl)-4-((4-(4-(trifluoromethyl)phenyl)phthalazin-1-yl)amino)pyrrolidine-1-carboxylate (80 mg, 41% yield) as a yellow solid. ¹H NMR (400 MHz, DMSO-d6) δ −0.12-−0.03 (6H, m), 0.77 (9H, d), 1.42 (9H, d), 2.79-2.91 (1H, m), 3.41-3.48 (1H, m), 3.51-3.65 (3H, m), 3.66-3.76 (2H, m), 4.97 (1H, q), 7.37 (1H, t), 7.78-7.83 (1H, m), 7.84-7.98 (6H, m), 8.51 (1H, d); m/z: (ES⁺) [M+H]⁺=603.

### 1-((3S,4S)-3-(hydroxymethyl)-4-((4-(4-(trifluoromethyl)phenyl)phthalazin-1-yl)amino)pyrrolidin-1-yl)prop-2-en-1-one

tert-Butyl (3S,4S)-3-(((tert-butyldimethylsilyl)oxy)methyl)-4-((4-(4-(trifluoromethyl)phenyl)phthalazin-1-yl)amino)pyrrolidine-1-carboxylate (0.080 g, 0.13 mmol) was dissolved in HCl (4 M in dioxane, 10 mL, 40 mmol) and the reaction mixture was stirred at rt for 2 hrs. The reaction mixture was concentrated to dryness and the resulting residue was dissolved in THF (6 mL) and water (2 mL). Sodium carbonate (14 mg, 0.13 mmol) was added and the reaction mixture was cooled to 0° C. A solution of acryloyl chloride (11 μL, 0.13 mmol) in THF (0.1 mL) was added dropwise and the reaction mixture was stirred at 0° C. for 1 h. The reaction mixture was concentrated and the residue was diluted in EtOAc (100 mL) and washed with water (2×50 mL). The organic layer was dried over Na₂SO₄, filtered and concentrated to dryness. The crude material was purified by preparative HPLC (Xselect CSH C18 OBD column: 30 mm×150 mm, 5 μm; Mobile Phase A: water (w/0.1% HCO₂H), Mobile Phase B: MeCN; Flow rate: 60 mL/min; Gradient: 19% B to 33% B; Wavelength: 254/220 nm) to afford 1-((3S,4S)-3-(hydroxymethyl)-4-((4-(4-(trifluoromethyl)phenyl)phthalazin-1-yl)amino)pyrrolidin-1-yl)prop-2-en-1-one (Example 49, 16 mg, 27% yield) as a white amorphous solid. ¹H NMR (400 MHz, DMSO-d6) δ 2.66-2.84 (1H, m), 3.50-4.08 (6H, m), 4.78 (1H, s), 5.01 (1H, s), 5.62 (1H, d), 6.07-6.16 (1H, m), 6.56 (1H, s), 7.30-7.43 (1H, m), 7.75-7.79 (1H, m), 7.81-7.92 (6H, m), 8.31-8.43 (1H, m); m/z: (ES⁺) [M+H]⁺=443.

### Example 50: (S)-1-(3-((4-(4-(trifluoromethyl)phenyl)phthalazin-1-yl)amino)piperidin-1-yl)prop-2-en-1-one

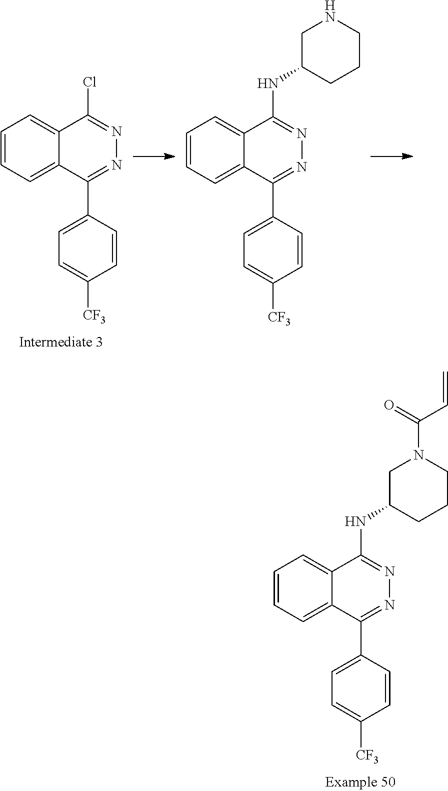 [SMILES: FC(F)(F)c1ccc(-c2nnc(Cl)c3ccccc23)cc1 ; FC(F)(F)c1ccc(-c2nnc(N[C@H]3CCCNC3)c3ccccc23)cc1 ; C=CC(=O)N1CCC[C@H](Nc2nnc(-c3ccc(C(F)(F)F)cc3)c3ccccc23)C1]

### (S)—N-(piperidin-3-yl)-4-(4-(trifluoromethyl)phenyl)phthalazin-1-amine

1-Chloro-4-(4-(trifluoromethyl)phenyl)phthalazine (Intermediate 3, 0.700 g, 2.27 mmol) and tert-butyl (S)-3-aminopiperidine-1-carboxylate (908 mg, 4.54 mmol) were dissolved in DMSO (1 mL) and the reaction mixture was heated to 140° C. and stirred for 16 hrs. The reaction mixture was cooled to rt, diluted in water (10 mL), and extracted with EtOAc (3×10 mL). The combined organics were dried over Na₂SO₄, filtered and concentrated to dryness. The crude material was purified on a reverse phase C18 column (20 to 60% MeCN in water) to afford (S)—N-(piperidin-3-yl)-4-(4-(trifluoromethyl)phenyl)phthalazin-1-amine (183 mg, 22% yield) as a brown solid. ¹H NMR (300 MHz, DMSO-d6) δ 1.76-2.07 (4H, m), 2.96 (2H, s), 3.46 (2H, s), 4.68 (1H, s), 7.91 (2H, d), 8.02 (2H, d), 8.16 (2H, p), 9.41 (2H, d), 9.99 (1H, s), 10.30 (1H, s); m/z: (ES⁺) [M+H]⁺=373.

### (S)-1-(3-((4-(4-(trifluoromethyl)phenyl)phthalazin-1-yl)amino)piperidin-1-yl)prop-2-en-1-one

Acryloyl chloride (33 μL, 0.40 mmol) was added to a solution of (S)—N-(piperidin-3-yl)-4-(4-(trifluoromethyl)phenyl)phthalazin-1-amine (0.15 g, 0.40 mmol) and Et₃N (168 μL, 1.21 mmol) in DCM (15 mL) and the reaction mixture was stirred at rt for 2 hrs. The volatiles were removed under reduced pressure and the resulting residue was purified by preparative HPLC (YMC-Actus Triart C18 ExRS: 30 mm×150 mm, 5 μm; Mobile Phase A: water (10 mM NH₄HCO₃+0.1% NH₃·H₂O); Mobile Phase B: MeCN; Flow rate: 60 mL/min; Gradient: 35% to 68%) to afford (S)-1-(3-((4-(4-(trifluoromethyl)phenyl)phthalazin-1-yl)amino)piperidin-1-yl)prop-2-en-1-one (Example 50, 31 mg, 18% yield) as a yellow amorphous solid. ¹H NMR (300 MHz, DMSO-d6) δ 1.55 (1H, s), 1.68-1.95 (2H, m), 2.16 (1H, s), 2.77-3.00 (1H, m), 3.04-3.27 (1H, m), 3.16-4.31 (3H, m), 5.65 (1H, dd), 6.10 (1H, t), 6.69-6.94 (1H, m), 7.52 (1H, brs), 7.76-8.03 (7H, m), 8.51 (1H, d); m/z: (ES⁺) [M+H]⁺=427.

### Biological Data

The data in Table 1 were generated using the Examples of the present specification and the assays described below.

### (i) TEAD4 FRET

Compounds were dosed with a final DMSO concentration of 1% (v/v). Compound IC₅₀ values were assessed following a 10-point, half-log₁₀ dilution schema starting at 100 μM compound concentration. Specifically, human TEAD protein from TEAD4(217-434) was cloned into an overexpression vector, expressed as an N-terminal HIS-TEV-Avi-tagged fusion protein in E coli, and subsequently purified, then protein was chemically depalmitoylated & biotinylated. The assay was performed in 384-well LV plates (384-well black, medium binding, PS, HIBASE, GREINER #784076) and run in the presence and absence of the compound of interest. Each well of 5 μL assay mixture contained 10 mM Tris-HCl (pH 7.5), 100 mM NaCl, 0.05 mM EDTA, 1 mM TECP, 1% DMSO, 0.03% Pluronic acid F127, 20 nM dePal Avi TEAD4 (217-434)-depalmitoylated & biotinylated protein, 0.8 nM Streptavidin Terbium cryptate (CisBio #610SATLB), 625 nM FAM labelled Probe A. Reactions were incubated at 25° C. for 120 min before reading on a PHERASTAR FSX Plate Reader (337 520 490 HTRF module required) (Supplier BMP).

The data file from the PHERAstar FSX contains both the acceptor (520 nm) and donor channels (490 nm) (“Channel A”=acceptor channel (520 nm), “Channel B”=donor channel (490 nm)). The ratio of the donor and acceptor (Channel A/Channel B) is calculated within Genedata Assay Analyzer. Subsequently, the dose-response of the ratio to testing compound concentration was fitted to a select fit model that will provide the best fit quality using automatic parameter (SMARTFIT) to derive IC₅₀ values for each testing compound.

Probe A is 3′,6′-dihydroxy-3-oxo-N-{8-[2-(5-{2-[4-(trifluoromethyl)anilino]phenyl}-2H-tetrazol-2-yl)acetamido]octyl}-3H-spiro[[2]benzofuran-1,9′-xanthene]-5-carboxamide;

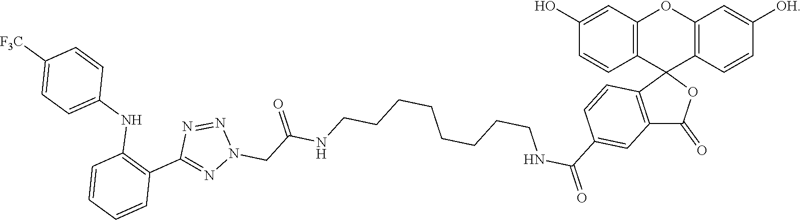 [SMILES: O=C(Cn1nnc(-c2ccccc2Nc2ccc(C(F)(F)F)cc2)n1)NCCCCCCCCNC(=O)c1ccc2c(c1)C(=O)OC21c2ccc(O)cc2Oc2cc(O)ccc21]

### (ii) TEAD Reporter Assay

MCF7-Tead cell line was obtained from BPS BIOSCIENCE (catalog number 60618) and was maintained in DMEM containing 10% fetal calf serum, 2 mM glutamine, and 400 μg/ml G418. Cells were grown in a humidified incubator at 37° C. with 5% CO₂. Cells were distributed to flat bottom white polystyrene TC treated 384 well plates at a density of 3,500 cells/well in 30 uL. Cells were incubated for 24 hours at 37° C. with 5% CO₂. Cells were acoustically dosed using an Echo 555, with compounds serially diluted in 100% DMSO. Plates were incubated for an additional 24 hours. Cells were examined for luciferase activity through the addition of 30 μL Bright-Glo luciferase (Promega catalog number E2620). Plates were incubated for 10 minutes at room temperature and luminescence was read using Tecan plate reader. The data obtained with each compound was exported to GENEDATA software to perform curve fitting analysis. IC₅₀ value was calculated based on the concentration of compound that is required to give a 50% effect compared to DMSO control.

(iii) Control Reporter (MCF7-Luciferase)

MCF7-Luciferase cell line was obtained from GENTARGET (catalog number SC050-L) and was maintained in DMEM containing 10% fetal calf serum, 2 mM glutamine. Cells were grown in a humidified incubator at 37° C. with 5% CO₂. Cells were distributed to flat bottom white polystyrene TC treated 384 well plates at a density of 3,500 cells/well in 30 μL. Cells were incubated for 24 hours at 37° C. with 5% CO₂. Cells were acoustically dosed using an Echo 555, with compounds serially diluted in 100% DMSO. Plates were incubated for an additional 24 hours. Cells were examined for luciferase activity through the addition of 30 μL Bright-Glo luciferase (Promega catalog number E2620). Plates were incubated for 10 minutes at room temperature and luminescence was read using Tecan plate reader. The data obtained with each compound was exported to GENEDATA software to perform curve fitting analysis. IC₅₀ value was calculated based on the concentration of compound that is required to give a 50% effect compared to DMSO control.

### (iv) Solubility

In order for an oral compound to reach the site of action, and in order for oral absorption from the gut to occur, that compound must be in solution, and therefore compounds which possess high intrinsic solubility may be more suitable for pharmaceutical use. The thermodynamic solubility of a research compound is measured under standard conditions. It is a shake-flask approach that uses 10 mM DMSO solutions which are supplied from the Compound Managements liquid store and is a high throughput method. The dried compounds are equilibrated in an aqueous phosphate buffer (pH 7.4) for 24 hours at 25° C., the portion with the dissolved compound is then separated from the remains. The solutions are analyzed and quantified using UPLC/MS/MS, QC-samples are incorporated in each assay-run to ensure the quality of the assay.

### (v) Protein Binding Measurements in Human Plasma (Hu PPB)

Human plasma protein binding is a key factor in controlling the amount of free (unbound) drug available for binding to target and hence plays an important role in the observed efficacy of drugs in vivo. Therefore, compounds which possess high free fraction (low levels of plasma protein binding) may exhibit enhanced efficacy relative to a compound with similar potency and exposure levels. The automated equilibrium dialysis assay in human plasma uses the RED (Rapid Equilibrium Dialysis) Device and sample handling. The assay generally runs over two to three days including delivery of results. After dialysis for 18 hours, plasma and buffer samples are prepared for analysis by liquid chromatography and mass spectrometry. Samples are generally tested in singlicates and quantified by LC/MSMS by using a 7-point calibration curve in plasma. The compounds are pooled together in plasma pools up to 10 compounds. Three reference compounds are used in each run, Propranolol, Metoprolol and Warfarin. Warfarin is used as a control in each pool and Propranolol and Metoprolol are placed randomly in each run. An in-house Excel macro is used for preparation of files for the robot and the mass spectrometer and is also used for the calculations of fraction unbound (fu %) in plasma.

TABLE 1

| | TEAD4 | TEAD | control | | |
|---|---|---|---|---|---|
| | FRET | reporter | reporter | Solubility | Hu PPB |
| Example # | IC₅₀ (μM) | IC₅₀ (μM) | IC₅₀ (μM) | (μM) | (% free) |
| 1 | 0.093 | 0.067 | 27.263 | 209.0 | NV |
| 2 | 0.056 | 0.022 | 12.877 | 188.0 | NV |
| 3 | 0.093 | 0.067 | 27.263 | 10.0 | NV |
| 4 | 0.410 | 0.192 | 26.025 | 230.0 | NV |
| 5 | 0.027 | 0.062 | >31.670 | 244.0 | 7.7 |
| 6 | 0.078 | 0.297 | 29.904 | 158.0 | 6.2 |
| 7 | 0.033 | 0.081 | 28.395 | 8.6 | 9.0 |
| 8 | 0.056 | 0.072 | >31.670 | 32.0 | 12.0 |
| 9 | 0.021 | 0.060 | >31.670 | <0.8 | 4.7 |
| 10 | 0.032 | 0.015 | 15.574 | 67.0 | NV |
| 11 | 0.033 | 0.156 | 10.204 | 9.0 | NV |
| 12 | 0.052 | 0.099 | 23.507 | 370.0 | NV |
| 13 | 0.041 | 0.030 | >31.670 | 40.0 | 6.1 |
| 14 | 0.049 | 0.026 | 19.200 | 39.0 | 7.7 |
| 15 | 0.025 | 0.015 | 30.840 | 135.0 | 11.0 |
| 16 | 0.270 | 0.354 | >31.670 | 114.0 | 25.0 |
| 17 | 0.061 | 0.020 | >31.670 | 151.0 | 15.0 |
| 18 | 0.200 | 0.255 | >31.670 | 47.0 | 10.0 |
| 19 | 0.026 | 0.515 | 0.977 | 38.0 | NV |
| 20 | 0.025 | 0.432 | 22.481 | 33.0 | 2.0 |
| 21 | 0.042 | 0.929 | 15.128 | 34.0 | NV |
| 22 | 0.950 | 0.957 | >31.670 | 65.0 | 5.4 |
| 23 | 0.042 | 0.014 | >31.670 | 48.0 | 4.7 |
| 24 | 0.023 | 0.016 | >31.670 | 32.0 | 1.9 |
| 25 | 0.057 | 0.025 | >31.670 | 32.0 | 12.0 |
| 26 | 0.076 | 0.042 | >31.670 | 175.0 | 26.0 |
| 27 | 0.029 | 0.023 | >31.670 | 63.0 | 6.0 |
| 28 | 0.061 | 0.071 | >31.670 | 168.0 | 3.9 |
| 29 | 0.130 | 0.094 | 22.238 | 7.0 | NV |
| 30 | 0.100 | 0.143 | 13.000 | 191.0 | NV |
| 31 | 0.073 | 0.058 | 31.082 | 534.0 | NV |
| 32 | 0.042 | 0.065 | >31.670 | 52.0 | 16.0 |
| 33 | 0.068 | 0.107 | >31.670 | 365.0 | 17.0 |
| 34 | 0.017 | 0.009 | >31.670 | 44.0 | NV |
| 35 | 0.079 | 0.034 | >31.670 | 801.0 | NV |
| 36 | 0.051 | 0.011 | >31.670 | 82.0 | NV |
| 37 | 0.180 | 0.033 | >31.670 | 155.0 | 17.0 |
| 38 | 0.064 | 0.034 | >31.670 | 191.0 | 12.5 |
| 39 | 0.013 | 0.005 | >31.670 | 20.0 | 1.4 |
| 40 | 0.250 | 0.351 | >31.670 | 3.0 | 1.6 |
| 41 | 0.072 | 0.152 | 21.495 | 72.0 | 0.9 |
| 42 | 0.070 | 0.672 | 19.622 | 19.0 | 0.8 |
| 43 | 0.012 | 0.011 | 13.334 | 16.0 | NV |
| 44 | 0.016 | 0.022 | 17.996 | 3.0 | NV |
| 45 | 0.012 | 0.011 | 19.020 | 13.6 | 3.9 |
| 46 | 0.830 | 0.283 | 19.446 | | |
| 47 | 0.042 | 0.017 | 20.694 | 13.0 | 8.3 |
| 48 | 0.026 | 0.026 | 16.097 | 2.4 | 5.9 |
| 49 | 0.059 | 0.057 | >31.670 | 164.7 | 9.8 |
| 50 | 0.030 | 0.187 | 28.967 | <1.6 | 5.4 |

NV means “No Value”.

### Comparative Examples

Comparative Example A was prepared in a manner analogous to Example 38.

| | Structure | Compound Name |
|---|---|---|
| Comparative Example A | 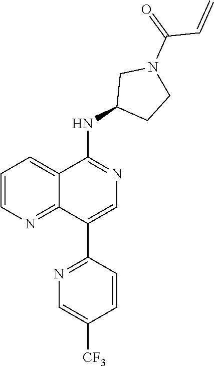 [SMILES: C=CC(=O)N1CC[C@@H](Nc2ncc(-c3ccc(C(F)(F)F)cn3)c3ncccc23)C1] | (R)-1-(3-((8-(5-(trifluoromethyl)pyridin-2-yl)- 1,6-naphthyridin-5-yl)amino)pyrrolidin-1- yl)prop-2-en-1-one |

TABLE 2

| Comparative | TEAD4 FRET | TEAD reporter | control reporter |
|---|---|---|---|
| Example | IC₅₀ (μM) | IC₅₀ (μM) | IC₅₀ (μM) |
| A | 1.900 | 0.592 | >31.670 |

WO2022037568A1 describes certain bicyclic compounds as being useful for the treatment of cancer. Comparative data for three compounds of WO2022037568A1 are provided below. The use of “&1” indicates a racemic mixture of enantiomers.

| | Structure | Compound Name |
|---|---|---|
| Comparative Example B | 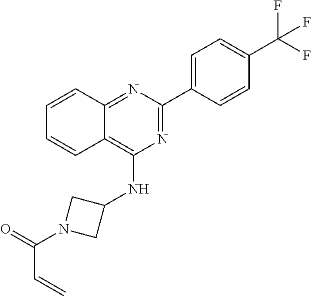 [SMILES: C=CC(=O)N1CC(Nc2nc(-c3ccc(C(F)(F)F)cc3)nc3ccccc23)C1] | 1-[3-({2-[4-(trifluoromethyl)phenyl]-4- quinazolinyl}amino)-1-azetidinyl]-2-propen- 1-one |
| Comparative Example C | 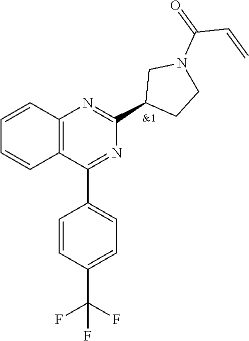 [SMILES: C=CC(=O)N1CC[C@@H](c2nc(-c3ccc(C(F)(F)F)cc3)c3ccccc3n2)C1] | 1-(3-{4-[4-(trifluoromethyl)phenyl]-2- quinazolinyl}-1-pyrrolidinyl)-2-propen-1-one |
| Comparative Example D | 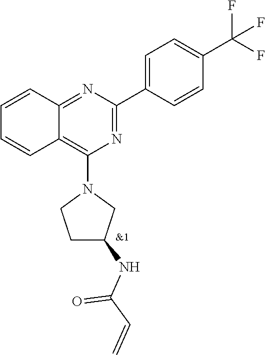 [SMILES: C=CC(=O)N[C@H]1CCN(c2nc(-c3ccc(C(F)(F)F)cc3)nc3ccccc23)C1] | N-(1-{2-[4-(trifluoromethyl)phenyl]-4- quinazolinyl}-3-pyrrolidinyl)acrylamide |

TABLE 3

| | TEAD4 | TEAD | control | | |
|---|---|---|---|---|---|
| Comparative | FRET | reporter | reporter | Solubility | Hu PPB |
| Example | IC₅₀ (μM) | IC₅₀ (μM) | IC₅₀ (μM) | (μM) | (% free) |
| B | 0.030 | 0.161 | >31.670 | 2.0 | NV |
| C | 0.055 | 0.260 | 22.720 | 66.0 | 1.1 |
| D | 2.900 | 1.781 | >31.670 | 0.6 | 0.2 |

### Synthesis of Probe A

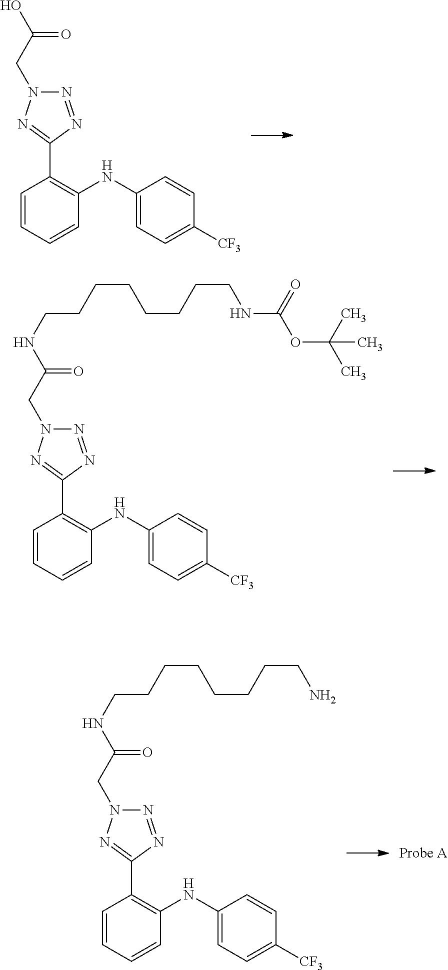

### tert-butyl (8-(2-(5-(2-((4-(trifluoromethyl)phenyl)amino)phenyl)-2H-tetrazol-2-yl)acetamido)octyl)carbamate

1-[Bis(dimethylamino)methylene]-1H-1,2,3-triazolo[4,5-b]pyridinium 3-oxid hexafluorophosphate (HATU, 110 mg, 0.29 mmol) and DIPEA (84 μL, 0.48 mmol) were added to a solution of 2-(5-(2-((4-(trifluoromethyl)phenyl)amino)phenyl)-2H-tetrazol-2-yl)acetic acid (70 mg, 0.19 mmol, disclosed in WO2018204532, the contents of which are incorporated by reference) and tert-butyl (8-aminooctyl)carbamate (71 mg, 0.29 mmol) in DMF (1.8 mL) at 0° C., and the reaction mixture was stirred for 3 hrs while slowly warming to rt. The crude reaction mixture was diluted with EtOAc (20 mL) and washed with saturated aq. NaHCO₃ (2×20 mL), saturated aq. NH₄Cl (2×20 mL) and brine (20 mL). The organic layer was dried over Na₂SO₄, filtered and concentrated to dryness. The crude material was purified by flash silica chromatography (0-50% EtOAc in Hex) to afford tert-butyl (8-(2-(5-(2-((4-(trifluoromethyl)phenyl)amino)phenyl)-2H-tetrazol-2-yl)acetamido)octyl)carbamate (53.7 mg, 47% yield) as a white solid. ¹H NMR (500 MHz, DMSO-d6) δ 1.22 (9H, br s), 1.29-1.38 (9H, m), 1.41 (3H, br d), 2.87 (2H, q), 3.09 (2H, q), 5.49 (2H, s), 6.72 (1H, br t), 7.13-7.19 (1H, m), 7.24 (2H, br d), 7.45-7.50 (1H, m), 7.52-7.59 (3H, m), 8.03 (1H, dd), 8.40 (1H, br t), 8.77 (1H, s); m/z: (ES⁺) [M+H]⁺=590.

### N-(8-aminooctyl)-2-(5-(2-((4-(trifluoromethyl)phenyl)amino)phenyl)-2H-tetrazol-2-yl)acetamide

TFA (0.50 mL, 6.5 mmol) was added to a solution of tert-butyl (8-(2-(5-(2-((4-(trifluoromethyl)phenyl)amino)phenyl)-2H-tetrazol-2-yl)acetamido)octyl)carbamate (50 mg, 0.08 mmol) in DCM (1 mL) at 0° C. and the reaction mixture was stirred for 45 min. The reaction mixture was concentrated to dryness to afford N-(8-aminooctyl)-2-(5-(2-((4-(trifluoromethyl)phenyl)amino)phenyl)-2H-tetrazol-2-yl)acetamide TFA as a pale blue oil.

### 3′,6′-dihydroxy-3-oxo-N-(8-(2-(5-(2-((4-(trifluoromethyl)phenyl)amino)phenyl)-2H-tetrazol-2-yl)acetamido)octyl)-3H-spiro[isobenzofuran-1,9′-xanthene]-5-carboxamide (Probe A)

HATU (61 mg, 0.16 mmol) and DIPEA (86 μL, 0.49 mmol) were added to a solution of 5-carboxyfluorescein (60 mg, 0.16 mmol) and N-(8-aminooctyl)-2-(5-(2-((4-(trifluoromethyl)phenyl)amino)phenyl)-2H-tetrazol-2-yl)acetamide (60 mg, 0.12 mmol) in DMF (1.1 mL) at 0° C. The reaction mixture was stirred at 0° C. for 2 hrs before warming to rt with stirring for an additional 1 h. The reaction mixture was diluted with EtOAc (50 mL) and washed with saturated aq. NH₄ (2×30 mL) and brine (30 mL). The organic layer was dried over Na₂SO₄, filtered and concentrated to dryness. The resulting residue was purified by preparative HPLC (Column=Xbridge C18, 4.6 mm×50 mm, 5 μm; Gradient=13 to 95% MeCN in H₂O w/0.2% NH₄OH over 4 min; Flow rate=0.6 mL/min; UV detection @ 254) to afford 3′,6′-dihydroxy-3-oxo-N-(8-(2-(5-(2-((4-(trifluoromethyl)phenyl)amino)phenyl)-2H-tetrazol-2-yl)acetamido)octyl)-3H-spiro[isobenzofuran-1,9′-xanthene]-5-carboxamide (82 mg, 79%) as a bright red solid. ¹H NMR (500 MHz, DMSO-d6) δ 1.29 (9H, br d), 1.39-1.48 (2H, m), 1.49-1.59 (2H, m), 2.53 (2H, br s), 3.10 (2H, q), 5.50 (2H, s), 6.45 (2H, br d), 6.54 (2H, br s), 6.58 (2H, s), 7.12-7.19 (1H, m), 7.24 (2H, d), 7.28 (1H, d), 7.43-7.50 (1H, m), 7.54 (3H, t), 8.03 (1H, dd), 8.11 (1H, br d), 8.43 (1H, s), 8.50 (1H, br t), 8.72 (1H, br t), 8.78 (1H, s); m/z: (ES⁺) [M+H]⁺=848.

The above description of illustrative embodiments is intended only to acquaint others skilled in the art with the Applicant's specification, its principles, and its practical application so that others skilled in the art may readily adapt and apply the specification in its numerous forms, as they may be best suited to the requirements of a particular use. This description and its specific examples, while indicating embodiments of this specification, are intended for purposes of illustration only. This specification, therefore, is not limited to the illustrative embodiments described in this specification, and may be variously modified. In addition, it is to be appreciated that various features of the specification that are, for clarity reasons, described in the context of separate embodiments, also may be combined to form a single embodiment. Conversely, various features of the specification that are, for brevity reasons, described in the context of a single embodiment, also may be combined to form sub-combinations thereof.

1. A compound of Formula (I): 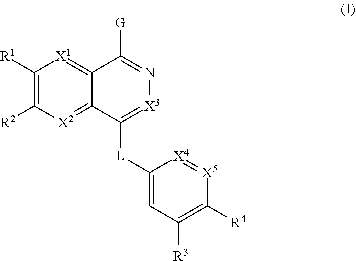
wherein:
X¹ is CH, C(C₁₋₄ alkyl), CF or N;
X² and X³ are independently selected from CH and N;
either X⁴ is CH and X⁵ is selected from CR⁵ and N, or X⁴ is N and X⁵ is CR⁵;
L is a covalent bond, O, NH, CH₂ or CH₂CH₂;
R¹ and R² are independently selected from H, C₁₋₄ alkoxy, —SO₂(C₁₋₄alkyl), —SO₂NH(C₁₋₄alkyl), —CN and Rⁱ, wherein Rⁱ is C₁₋₄ alkyl optionally substituted with —CN or C₁₋₄ alkoxy;
R³, R⁴ and R⁵ are independently selected from H, C₁₋₄ fluoroalkyl, C₁₋₄alkoxy, —S(C₁₋₄ alkyl), —O(C₁₋₄ fluoroalkyl), —S(C₁₋₄ fluoroalkyl), F, Cl, C₃₋₄ fluorocycloalkyl, R^{j} and R^{k}, wherein R^{j} is C₃₋₄ cycloalkyl optionally substituted with —CN, C₁₋₄ alkoxy or C₁₋₄ fluoroalkyl and R^{k} is C₁₋₄ alkyl optionally substituted with —CN or C₁₋₄ alkoxy;
G is selected from 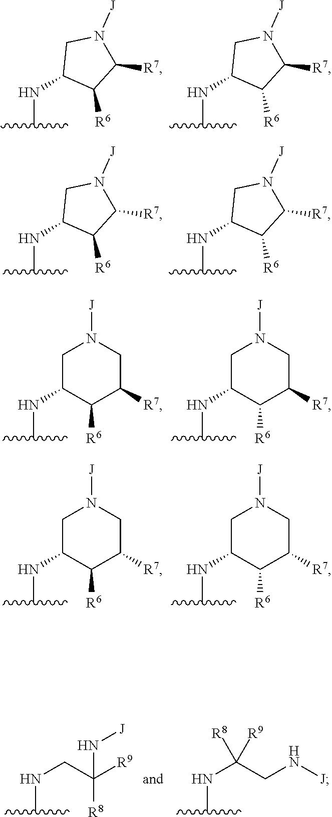
R⁶ is H, OH, F, —CN, C(═O)NH₂, N(C₁₋₄ alkyl)₂, C₁₋₄ alkoxy, C₁₋₄ fluoroalkyl, R¹⁰ or R¹¹;
R⁷ is H, C₁₋₄ alkoxy, R¹⁰ or R¹¹;
either R⁸ and R⁹ are independently selected from H, R¹⁰ and R¹¹;
or R⁸ and R⁹, together with the carbon atom to which they are attached, form a cyclopropane or cyclobutane ring;
each R¹⁰ is independently C₁₋₄alkyl optionally substituted with C₁₋₄alkoxy, N(C₁₋₄alkyl)₂ or OH;
each R¹¹ is independently C₃₋₄ cycloalkyl optionally substituted with C₁₋₄ alkoxy or OH;
J is selected from 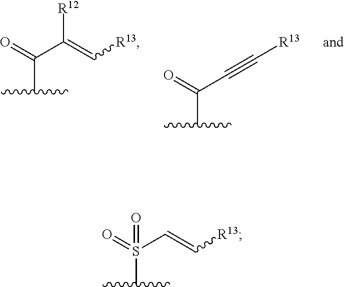
R¹² is H or F;
R¹³ is H, CH₂F or CH₃;
wherein a C₁₋₄ fluoroalkyl is a saturated linear or branched hydrocarbon radical having 1 to 4 carbon atoms, with at least one hydrogen atom substituted for a fluorine atom; and
wherein a C₃₋₄ fluorocycloalkyl is a saturated cyclic hydrocarbon radical having 3 or 4 carbon atoms, with at least one hydrogen atom substituted for a fluorine atom,
or a pharmaceutically acceptable salt thereof.

2. A compound of Formula (I) or a pharmaceutically acceptable salt thereof, as claimed in claim 1, wherein X¹ is CH.

3. A compound of Formula (I) or a pharmaceutically acceptable salt thereof, as claimed in claim 1, wherein X¹ is N.

4. A compound of Formula (I) or a pharmaceutically acceptable salt thereof, as claimed in any one of claims 1 to 3, wherein X² is CH and X³ is N.

5. A compound of Formula (I) or a pharmaceutically acceptable salt thereof, as claimed in any one of claims 1 to 3, wherein X² is N and X³ is CH.

6. A compound of Formula (I) or a pharmaceutically acceptable salt thereof, as claimed in any one of claims 1 to 5, wherein X⁴ is N and X⁵ is CH.

7. A compound of Formula (I) or a pharmaceutically acceptable salt thereof, as claimed in any one of claims 1 to 6, wherein L is a covalent bond.

8. A compound of Formula (I) or a pharmaceutically acceptable salt thereof, as claimed in any one of claims 1 to 7, wherein R⁴ is C₁₋₄ fluoroalkyl, —O(C₁₋₄ fluoroalkyl) or —S(C₁₋₄ fluoroalkyl).

9. A compound of Formula (I) or a pharmaceutically acceptable salt thereof, as claimed in any one of claims 1 to 7, wherein R⁴ is CF₂H, CF₃, OCF₃ or SCF₃.

10. A compound of Formula (I) or a pharmaceutically acceptable salt thereof, as claimed in any one of claims 1 to 9, wherein R³ and R⁵ are both H.

11. A compound of Formula (I) or a pharmaceutically acceptable salt thereof, as claimed in any one of claims 1 to 10, wherein R¹ and R² are both H.

12. A compound of Formula (I) or a pharmaceutically acceptable salt thereof, as claimed in any one of claims 1 to 11, wherein G is selected from

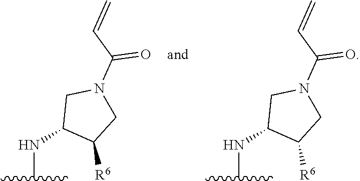

13. A compound of Formula (I) or a pharmaceutically acceptable salt thereof, as claimed in any one of claims 1 to 12, wherein R⁶ is H, OH, F, CH₂OH or CH₂OCH₃.

14. A pharmaceutical composition comprising a compound of Formula (I) or a pharmaceutically acceptable salt thereof, as claimed in any one of claims 1 to 13, and a pharmaceutically acceptable excipient.

15. A compound of Formula (I) or a pharmaceutically acceptable salt thereof, as claimed in any one of claims 1 to 13, for use in therapy.

16. A compound of Formula (I) or a pharmaceutically acceptable salt thereof, as claimed in any one of claims 1 to 13, for use in the treatment of cancer.

17. A method of treating cancer in a patient comprising administering to the patient a compound of Formula (I) or a pharmaceutically acceptable salt thereof, as claimed in any one of claims 1 to 13.
